SEQUENCE LISTING (771 sequences)

SEQ ID NO: 1 - DNA, 401 bp, Homo sapiens
atgcatttgaatggaggaagactatttggggcccctgggaaacagggtggcacagagcagtttcctggcctcgaagaggatgcagttttccaagatgtggggtttgcttgtgcatctccatgcgcagggcagggggctcagcacagtaataaccagagtcacacaccataggcagatccctggctctgcttggagggacaragattcagagctgggcggatcatcaatagaccatctacaccaagcccctgactcctgaactacattctttccatcatacccgtcactccatgtctattttctcagtattgaatgagaaaggaaatgaatgtttactgcatctaattatgtgccaggtgcctcattaagtttataatggaaatcacacagtatgataatag

SEQ ID NO: 2 - DNA, 401 bp, Homo sapiens
agtcacacaccataggcagatccctggctctgcttggagggacaaagattcagagctgggcggatcatcaatagaccatctacaccaagcccctgactcctgaactacattctttccatcatacccgtcactccatgtctattttctcagtattgaatgagaaaggaaatgaatgtttactgcatctaattatgtgccagrtgcctcattaagtttataatggaaatcacacagtatgataatagccaatatttaagaagaatctagtaggtgcaggcatgagtctaattgttggatacacattaacttatatttaattctcacaatagccccaagtggtaggctgtggtgagtatctgagacagaggcacagagaaatgttcttagccagcaagcaaaag

SEQ ID NO: 3 - DNA, 401 bp, Homo sapiens
acctccaactgttatcggccaagtcctgatgacattgtgaattctttgaggcaaagactatactcctttgactctatatcctcagagtcttgataccatcctggcacacagcagaagctcaaaaatgcttgatgagtgaaggatacacatttataaatccactgtttaaatagagtttagggttttcaaagggcccaggcwtatgaacctcctttgtaaatcaccccaaatcaaatacaatgttcacaataacatacaaggatgggctccatcaaactaattcaaatgcatagctgcagcctgttctttgcttttattttagtttttcgtattgggtagatttttttatgggggcgatgttttggagtactgtggtacaatgttctgcaccataaagaaat

SEQ ID NO: 4 - DNA, 401 bp, Homo sapiens
gtgaaggatacacatttataaatccactgtttaaatagagtttagggttttcaaagggcccaggcatatgaacctcctttgtaaatcaccccaaatcaaatacaatgttcacaataacatacaaggatgggctccatcaaactaattcaaatgcatagctgcagcctgttctttgcttttattttagtttttcgtattggrtagatttttttatgggggcgatgttttggagtactgtggtacaatgttctgcaccataaagaaattttagtgtttagaagtcagaaattgagtctgactgtgaactacagcaaagtgcatgcatgtaggctgtgcattgaccacagaatgcaaatgctgagccattataatatatctagatctacacaggtaggcagata

SEQ ID NO: 5 - DNA, 401 bp, Homo sapiens
acaataccacacatctacaactatctgatccttgacaaacctgacaaaaacaagaaatggggaaaggattccctatttaacaaatggtgctgggaaaactggctagccatatgtacaaagctgaaactggatcccttccttacacttatataaaaattaattcaagatggattaaagacttaaatgttacacctaaaaccrtaaaaaccctagaagaagacctaggcaataccattcaggacataggcatgggcaaggacttcacgtctaaaacaccaaaagcaacggcaacaaaagccaaaattgagaaatgggatctaattaaactaaagagcttctgcacagcaaaagaaactaccatcagagtgaacaggcaacctacacaatgggagaaaattttt

SEQ ID NO: 6 - DNA, 401 bp, Homo sapiens
catgctgctataaagacacatgcaaatgtatgtttattgtggcactattcacaatagcaaagacttggaaccaacccaaatgtccatcaatgatagactggattaagaaaatgtggcacatatagaccatggaatactatacagccacaaaaaaggatgaattcatgtcctttgtaggtacatggatgaagctggaaaccrtcattctgagcaaactatcgcaaggacagaaaaccaaacactgcatgttctgactcattggtgggaactgaacaatgagaacacttggacacaggttgggaaacaccacacaccggggcctgtcatgggggtgaggggagcagggagggatagcctgaggagaaatacctaacgtaaatgatgagttaatgggtgcagca

SEQ ID NO: 7 - DNA, 401 bp, Homo sapiens
atgacatatgctcatatctcctctgaaaaacactcagggagccacggagaagaggggcaaagcatttttccagcttgaaccctttggagtggtgtccgagttggaggaaaaggccagccatcctgctaaatgcaccagtgaaagaaaatattctggcaatgtgagatggaaaaaatagaacctaccagaatgactcttagsaagagtactataaatgcttagagggaaaatgaagtcacttagggacaaggaggtgagaccctccttcccccaaaggttaagcttaactaaccccggatatttgataaccaaggaagatgagtccatttgactcaaggtcttttgattcttgaatgcagtaggcaaatttggggccaggaattaccaaggacaatagaaaa

SEQ ID NO: 8 - DNA, 401 bp, Homo sapiens
atagagtcaatgtctgactagcatccatctgtcttctcccagtaacatcactctgggtttcctttgggaaacactcctccccagtcttcattcaagtggtttggatggggctaaatctacgccctatttgtagaggtagatgtatggtgcagacctaccttgcatttcatctccttggacagagtgtttgcttccaaggtrgccacaggacccaagctggatcaatgagatacaaccttaggatttttgatggaactcttgggaaagagaggctcttcttcaatggttcctaagccttgaaatgtatgtctagggataccattctgtcacctcatgcccagagcctgccagagagtaatctcaacacaggaaggagagagctgacatatggtagcaggtag

SEQ ID NO: 9 - DNA, 401 bp, Homo sapiens
tctctgccctgccctgtctccgggcaccagcgggtcatgtcactgcaaacaaataacatggggaagcctgccaggagaaatgacaagggtatttgccagttgagaaggcttctaccagtcagtctggactgggacatcaaagagttccctcttcttgacttggatttctaacacattagccagagcctttttttttttttytttttagacggagtgtcactctgtcacccaagctgtagtgcagtagcaagatcttggctcactgtaacctccacctcccaggttcagtgattcccccacctcagcctcccaagtagctgggattacaggtgcatgccaccatgcctggctaatttttatttttatttttaatttttttttgtattttagtagagaagttt

SEQ ID NO: 10 - DNA, 401 bp, Homo sapiens
gtcatttacattcactacacagttatttttctggttttctagcaaaatcttttgaaaatataagtcaaaactagtattatccctatttggggaaacaagaaactgaggtgtggacctccttagatcatgaatgcagaggaagaaaggcttacagccattattaatccctgaatctgtcatcattcttctttccagaaatcrgcacggtggcttgcaggccaatactggcaaatcctctttgtagcaagtccctacaactaactacaatggaatcaagtttttattattgctttaaaaaactcaatgttgtattggtggagataataccaaccccacagtcacggcaactgaaggctcttattgaaaggatgctccaagctttcttattgaaagcttggagg

SEQ ID NO: 11 - DNA, 401 bp, Homo sapiens
ttattattgctttaaaaaactcaatgttgtattggtggagataataccaaccccacagtcacggcaactgaaggctcttattgaaaggatgctccaagctttcttattgaaagcttggaggaaggttcaatattatcacttattactgatctttggtattctttgtttaaaaggtatcattccagtcttatatatatatayatatacacagatacatgtatacatttgctattattacacatattttatgttgtttttttcatattcatattcacgtaacatttttaaagccctgaaggcaaaacaagctgttacctttctattgtgatcaaaattcatagctacaaattagattgcacgttcaccataatcatcttcaccatcctcataatcaatacaaa

SEQ ID NO: 12 - DNA, 401 bp, Homo sapiens
actccatgtctcacatccaggtcaggctgatgcaagaggtgggttcctatggtcttgggcgctttatgtctgtgactttgcaggatttagccctgctcctggctgctttcacaggctggtgttgagtgtctgtggcttttccaggcacacagtgcaagctgtcagtagatctaccattctgaggtccagaggatggtggcygtcttctcacagctccactaggcagtaccacagtagggactctgtgtgggggctccaacctcacatttcccttccgtaatgtcctagcagaggttctccatgagggccccacccctgcagcaaacttctgcctgggcatccatgcatttctatacatcctctgaaatctagctggaggtttccaaaccccaattcttgac

SEQ ID NO: 13 - DNA, 401 bp, Homo sapiens
ctgtacattggccctttcagccatggctagagctgctgggattcagggcaccaagtccctaggctgcacacagcagggagaccttggacccagcccactaaaccactttctcctaggcctccaggcctataatgggaggggctgcagtgaagacctctgacaggccctggagacattttccctattgtctttgggaataayattcagctcctcgttacttatgtaaatttacgcagccagcttgagtttctctccagaaaatgggtttttcttttctgtcacatagtcaggctgaaaattttccaaacttttatgctctgcttctcttatgaaactgaatgcctttaacagcatccaagtcacatcttgaatgctttgttgcttagcaatttcttccacaa

SEQ ID NO: 14 - DNA, 401 bp, Homo sapiens
taaaacataacaagagtcacctttgctccagttcccaacgagttcctcatctccatctgagaccacctcaggctgggccttattgtccatatcgctatcaggcttttggtcaaaaccattcgacaagtctctagaaagtttcacactttcccacattttcctgttttctcctgagacctccaaactgttccaacttccgcytgttacccagttccaaagccacttccacatttttgggtatcttttcagcaacacctcactctactggtaccaatttactgtattagttcattttcacactgctgataaagacaaacctgagactgggcaatttacaaaagaaagaggtttattggacttacaattccatgtggctggggaggcctcacaatcatggcaga

SEQ ID NO: 15 - DNA, 401 bp, Homo sapiens
ggttttaccttttaagaatagattagctaaagagaacacactgcatgtcagttttcccagagcctcaccataagcacagcttttcctcaacattatgaaaaaatgaaacttttccttagcaacattgcatagttattttttacattcctgtcctcttaaagggtgccccatttcctttccctctcatctccccttttattyccctttctgccaccccagccaagtggtcttcatccattcccttgcaaaattagttatctctttttttgccacccccaccccaaaattaattttctataaaagccgatactcatcatcattacagtgtagctgagatataaacattaatgtgcaccctttcgtacagcacttctctagcattttgccagggaacaaaggtt

SEQ ID NO: 16 - DNA, 401 bp, Homo sapiens
acagatgttttaaatagatcaactcatttaatgcactagtacaaacatgtgaaatatccccattctactggtgtgaaaactgaggccaggagaggtcaaattggaaattctacatccctgatcagagccactaaccaagatgcaaaaagcaacgaagaacctccacagctagtaactgacaacacgtaggtctttgttagyggccaacttatttgaccggagcatccttatggttattgaaggaaggttgtacattattgtggttccctaagcaggttgtacaacatttctctccctctctgaaaaggctgatgtcacttctctaggtgatgttttgtggtatgtgatagggctggatcacatatcttttagacatctattatttcattacgtaagtctta

SEQ ID NO: 17 - DNA, 401 bp, Homo sapiens
caggaaataccccacttctctactgagcagaagctctgggaggagaagctttgtgcaaatcccccctcacctcctctctctttctcacacacacaagcacacatacatatatgccacccaccaaatctaagggaatattaatattagaaacaaatattactttaaagatctgggaagaaatggagccaaataagatggacraacctaaaagaccacatggcaattctcaggacttactagcaaatatccatcctgctgaatgtgacaggactcagtggggtaggtgcgatggtatgggtagtgtgtatgagttcagctgtgtctctgtaaaattcatatgttcacattgtaacctctagtacttcagaatatgaccttacttggaaatgggctctttccag

SEQ ID NO: 18 - DNA, 401 bp, Homo sapiens
aaacaccggagcaatacttagtaaggaggttccaggcctccataaccatgttacgtagtattgatggagaaggtttcaagaactctcctcctaatccagtcccccaacatgctcttttcttagccaggaacttaagtaaacaatggcatagtcctggaaagtcctatgggttttatttcttcactgtggatctgccatatrttcatctgtattcagatgcttgaacataattttctgactaactatgagctcccaactaaaatttccaacataaggagactaagagattatctgaccccatgctcttatgtgatcggtgaaggaacatggacttatcagaagcaactagtaggaaagccactcagctgattgatacgttctgtctagtgtcttaactccaa

SEQ ID NO: 19 - DNA, 401 bp, Homo sapiens
cccaacatgctcttttcttagccaggaacttaagtaaacaatggcatagtcctggaaagtcctatgggttttatttcttcactgtggatctgccatatgttcatctgtattcagatgcttgaacataattttctgactaactatgagctcccaactaaaatttccaacataaggagactaagagattatctgaccccatgytcttatgtgatcggtgaaggaacatggacttatcagaagcaactagtaggaaagccactcagctgattgatacgttctgtctagtgtcttaactccaaatccaaggctttgtcctacacaccacattggtttcccgccacataaacaactttcggtgtgggaaatctgatttcattctcccttgctgtatctaaagttcc

SEQ ID NO: 20 - DNA, 401 bp, Homo sapiens
cttagccaggaacttaagtaaacaatggcatagtcctggaaagtcctatgggttttatttcttcactgtggatctgccatatgttcatctgtattcagatgcttgaacataattttctgactaactatgagctcccaactaaaatttccaacataaggagactaagagattatctgaccccatgctcttatgtgatcggtraaggaacatggacttatcagaagcaactagtaggaaagccactcagctgattgatacgttctgtctagtgtcttaactccaaatccaaggctttgtcctacacaccacattggtttcccgccacataaacaactttcggtgtgggaaatctgatttcattctcccttgctgtatctaaagttccctgcacttaaccgcct

SEQ ID NO: 21 - DNA, 401 bp, Homo sapiens
taaacaatggcatagtcctggaaagtcctatgggttttatttcttcactgtggatctgccatatgttcatctgtattcagatgcttgaacataattttctgactaactatgagctcccaactaaaatttccaacataaggagactaagagattatctgaccccatgctcttatgtgatcggtgaaggaacatggacttatsagaagcaactagtaggaaagccactcagctgattgatacgttctgtctagtgtcttaactccaaatccaaggctttgtcctacacaccacattggtttcccgccacataaacaactttcggtgtgggaaatctgatttcattctcccttgctgtatctaaagttccctgcacttaaccgcctcttcggtgagtaaggaat

SEQ ID NO: 22 - DNA, 401 bp, Homo sapiens
cttctttgcaaggagcaggtaaggtggcagctaggccacagtcggggcctgctcagggctgctccatcctgcatgctgaaagctgcccctgctgctggctgctggcccctaagggagcactgccgcaggagaggaagtggaagcgcaccagaggagaggaagtgcacggccagagaggaagcgcaaggcgggagaggaaaygcaaggcgggagaggaagtgcaaggcaggagaggaagtgcaaggcaggagaggaagcgcaaggcaggagaggaagccacagcaggagaagaagcacacagcagggctgtgagttgtatggccttttacagcccttccacccagaggattccttttgggtaccactctcccactccttaaaaaaactccactggcggccat

SEQ ID NO: 23 - DNA, 401 bp, Homo sapiens
caaggagcaggtaaggtggcagctaggccacagtcggggcctgctcagggctgctccatcctgcatgctgaaagctgcccctgctgctggctgctggcccctaagggagcactgccgcaggagaggaagtggaagcgcaccagaggagaggaagtgcacggccagagaggaagcgcaaggcgggagaggaaacgcaaggcrggagaggaagtgcaaggcaggagaggaagtgcaaggcaggagaggaagcgcaaggcaggagaggaagccacagcaggagaagaagcacacagcagggctgtgagttgtatggccttttacagcccttccacccagaggattccttttgggtaccactctcccactccttaaaaaaactccactggcggccatgcatagat

SEQ ID NO: 24 - DNA, 401 bp, Homo sapiens
ccttttacagcccttccacccagaggattccttttgggtaccactctcccactccttaaaaaaactccactggcggccatgcatagatgagattctaaatactttcgggctacctttctggaggtcagaaaacatggtcatatagcccaagccttgtaagtctgcatgggctactgaatttccatggatattttgtttttktaggcctaaacaagaaggctaaagttagaaagatagttgggctcggggggctcagcattctacaggcttgtatgactaaagaaaccacttccacccactccatccccttcccagtcaggagaaattcctggtcgatgagttagagagactggattggaataaaacaaaattcttcactggacatagctgtttggactgtg

SEQ ID NO: 25 - DNA, 401 bp, Homo sapiens
caagagtgtaccttgttttctgctaggatgctgattgataggaatatgtgcaattgacccccatgacatcactgagaaattggaggcacacagcccatggtgggcataggacccaaccgatgaatttgctccccagagactgtgctctcatccctgcactccctcatctctacaaggtcacccatcttagggatacacctytaggtctattcatatgacacctcagtggctcttagggctttgccaatacaaatagtgtttctgtaaatatctttatatgtctgtcttggtaaatatatggaattatatctatcgagcatattcttagcaatgcctagtcaatgtgtatatacaatttttaatttgaggggatattgtcaaattgctatagtagttagtca

SEQ ID NO: 26 - DNA, 401 bp, Homo sapiens
agtagttagtcaaattaaatgtacttgtatactatgacctggtaattctactctaagtagatagatttagatatagtgataaggatatagacatatcaaagaaatgctcacacatatccataaagggatatgtacaagagtgttcattatagttttgttattttattttattttatttgtactgtctttgtttggagcagyggtgagaagttggtgttggtgatagatcctcattgggagggttcttagataaaccgtggtggatgttcaccatgggattttgtgcagcagtttgaagcaaatgtatgcataatgatattgtaaatatagtgctgagtgaaaaaaataagatgtatgccataatgctacttttgtacattaaaaaaaaaaatccacaagaa

SEQ ID NO: 27 - DNA, 401 bp, Homo sapiens
aatgttcttttcttggaagaagttgttcctttgccaaacataaatgaaactttggctccaagatcagctcaaaatttactagctgataaattggaagtagggagtgcgttgtgaaggtgggaggagaagggagagggggagggaggttttcatttggggggcaagttcaaacttggaatcagaagctattaaagtgtctgrtgtgtgctgagtcatatggaggtgggggcaggggatgacgctggctccttgccagacacacgtactgcccaggttgatgaacgagcctccaaaggcactacaacaggattataatttcactagcattttaaacataatattagaagccacgtgcatgacaccgagcaagcaatcagcatgggcctcttgtgtttatgaga

SEQ ID NO: 28 - DNA, 401 bp, Homo sapiens
aaacagagaacaatggtgtcaattaacattttaaaagtctttctatgggccattcacagtgcaagtacttttcatgcattatctcagttaatcccttatagcaatttcataaaggcaatactattacagatgacaaaattgagactttgagaaaaagatcctgcaacctaataagcatcagaacctgggtttgaattgagrgagtctgactccactatgcttggcttcgccattggttagccaccctattgtagcatttttctttgctattgtgttttagctttttgggtccacttatgtttctcaccagactgtggtctctcaagttcgagtcatgttttagtcttcttagttccaggccatcattcagatacaagtaggcacttcatagatgtttcctg

SEQ ID NO: 29 - DNA, 401 bp, Homo sapiens
agaatggcgtgaacccgggaggcggagcttgcagtgagccaagattgtgccactgcactccagcctgggtgacagagagagactctgtctcaaaaaaaaaaaaaaaaaaaaaaaaaaagaccaggaacttcacaactgcatgactggccacatagatctggaaaatcattatttatcttttcagctgttgcatgcataaaytgccatattgccaaactagtgtttgtccgcttgtctgcaaagtaagaatcctttactcctgttactcatttttcctccctctcttcttttcttccttttgtttgttgcagaaggttgagtaaattgcactcctcagtccatggcccattatttatcttgtttgcttatttatttcatctctattccttcatcctagtttt

SEQ ID NO: 30 - DNA, 401 bp, Homo sapiens
ttcagctgttgcatgcataaattgccatattgccaaactagtgtttgtccgcttgtctgcaaagtaagaatcctttactcctgttactcatttttcctccctctcttcttttcttccttttgtttgttgcagaaggttgagtaaattgcactcctcagtccatggcccattatttatcttgtttgcttatttatttcatcyctattccttcatcctagtttttcttcctccctcccttaggcaaacactcttatttgtgtttaatttagatcctttggtttgtatgtattcttagaaaacattcatttttttaatccatgtatttttaatttaaaaaaatggtactttgctatatatctctttttgttccttacttttttcacccagcactgtttatcaat

SEQ ID NO: 31 - DNA, 401 bp, Homo sapiens
agcagcagtacacaagttcccacacccacaccaaccctttggcatgctgcaattctccaaccttgcagccattgcttctccatcgttctcctctttccacaatcttgctgcttcctcgggctgtttctgctctcactgcccacctaccccaccctgctgctacctgtcctcacttttttctctggcctcccctacaccccmtgtggttccttcatattcatggcttctgctttttgaataaacatgtttgaatattttctagtattcttttttcctccttgtctttctctttgaccttcaaacactctaattgggctccttagttgctacccagtctagcaacatctgtgaggtgaaattccgtttttttggggtttttttgtttttttttttttttgaga

SEQ ID NO: 32 - DNA, 401 bp, Homo sapiens
ttttttctctggcctcccctacaccccatgtggttccttcatattcatggcttctgctttttgaataaacatgtttgaatattttctagtattcttttttcctccttgtctttctctttgaccttcaaacactctaattgggctccttagttgctacccagtctagcaacatctgtgaggtgaaattccgtttttttgggktttttttgtttttttttttttttgagatggagtctcgctctgttgcccaggctggagtgcagtggcacgatcttggctcactgcaagctccgcctcccaggttcacgccattctcctgcctcagcctcccgagtagctgggactacaggtgcccaccaccatgcctggctaaatttttgtatttttagtagagacggagt

SEQ ID NO: 33 - DNA, 401 bp, Homo sapiens
ttggggtttttttgtttttttttttttttgagatggagtctcgctctgttgcccaggctggagtgcagtggcacgatcttggctcactgcaagctccgcctcccaggttcacgccattctcctgcctcagcctcccgagtagctgggactacaggtgcccaccaccatgcctggctaaatttttgtatttttagtagagayggagtttcaccgtgttggccaggatggtctcaatctcctgacctcatgatccacccatctcagcctcccaaagtgctgggattacaggcatgagaggtgaagttctttttttgggccacttcttatgactactgtggatcagctcagatgtctacccctggcccaacagatgtctccagggtgttgaaggtcaccggtct

SEQ ID NO: 34 - DNA, 401 bp, Homo sapiens
ctcccgagtagctgggactacaggtgcccaccaccatgcctggctaaatttttgtatttttagtagagacggagtttcaccgtgttggccaggatggtctcaatctcctgacctcatgatccacccatctcagcctcccaaagtgctgggattacaggcatgagaggtgaagttctttttttgggccacttcttatgactrctgtggatcagctcagatgtctacccctggcccaacagatgtctccagggtgttgaaggtcaccggtctaaactctattgacccctggggaaggagtatctgttacaggctctttccatagaagggggctatggctatggcaggcattcagagttctatgaactgctcagtatacacattcatttggtattttgttaaat

SEQ ID NO: 35 - DNA, 401 bp, Homo sapiens
agcctcccaaagtgctgggattacaggcatgagaggtgaagttctttttttgggccacttcttatgactactgtggatcagctcagatgtctacccctggcccaacagatgtctccagggtgttgaaggtcaccggtctaaactctattgacccctggggaaggagtatctgttacaggctctttccatagaagggggctrtggctatggcaggcattcagagttctatgaactgctcagtatacacattcatttggtattttgttaaattcaccatttaccaattttgatgtgaagtggccacagttacacaagaaaaacaccaaccttgaaaattattttatacataaatgttaccagaaagaggtcccaatccataccccaagagaggtttcttggat

SEQ ID NO: 36 - DNA, 401 bp, Homo sapiens
agtatctgttacaggctctttccatagaagggggctatggctatggcaggcattcagagttctatgaactgctcagtatacacattcatttggtattttgttaaattcaccatttaccaattttgatgtgaagtggccacagttacacaagaaaaacaccaaccttgaaaattattttatacataaatgttaccagaaagrggtcccaatccataccccaagagaggtttcttggatctcatgcaagaaagaattcagggcgagtccataaagtgaaaatgagtttattgggaaagtaaaggaataaagaatggctactccataaacaaagcagccccgagggtggctggttgcccatttttattgttatttcttgatgatatgctaaacaaggggtggat

SEQ ID NO: 37 - DNA, 401 bp, Homo sapiens
tacacattcatttggtattttgttaaattcaccatttaccaattttgatgtgaagtggccacagttacacaagaaaaacaccaaccttgaaaattattttatacataaatgttaccagaaagaggtcccaatccataccccaagagaggtttcttggatctcatgcaagaaagaattcagggcgagtccataaagtgaaartgagtttattgggaaagtaaaggaataaagaatggctactccataaacaaagcagccccgagggtggctggttgcccatttttattgttatttcttgatgatatgctaaacaaggggtggattattcatgcctcccctttttagaccatataggataacttcctgacgttgccatggcatttgtaaactgtcatggcgct

SEQ ID NO: 38 - DNA, 401 bp, Homo sapiens
cccattcttctgacattaaatttcactctggtaagatcttcactgacctctactctaggataggttctcctactgaatgtccccatctcctatcctcacacacaatacactttattataattaaatgtcatcctgtcctgcaagatatgagttacctgagtatataattgcacctgttcctttcactactgcatttctacygttcagcaaagtactttgcatctagtatgtgcccaaaaaatctgtggctgtctttgttgaatgacagagagggatatctctgggagattgagtttaaatccctgtcctgttatgacatctgagacaagtcatagttcctgtattatctacctcacagggtttttatatatgaaaatgttttttcaaattgtgaaggtcta

SEQ ID NO: 39 - DNA, 401 bp, Homo sapiens
tcctgcaagatatgagttacctgagtatataattgcacctgttcctttcactactgcatttctactgttcagcaaagtactttgcatctagtatgtgcccaaaaaatctgtggctgtctttgttgaatgacagagagggatatctctgggagattgagtttaaatccctgtcctgttatgacatctgagacaagtcatagytcctgtattatctacctcacagggtttttatatatgaaaatgttttttcaaattgtgaaggtctacaaaaataagacaaaagcatttgatgggaaaagacactcagataattttgtgcctcttcagagaaatttaaatgatttaaatgccaattgcttgagtaagtgaataaaataataccctcatcctaacataatttc

SEQ ID NO: 40 - DNA, 401 bp, Homo sapiens
atttctactgttcagcaaagtactttgcatctagtatgtgcccaaaaaatctgtggctgtctttgttgaatgacagagagggatatctctgggagattgagtttaaatccctgtcctgttatgacatctgagacaagtcatagttcctgtattatctacctcacagggtttttatatatgaaaatgttttttcaaattgtraaggtctacaaaaataagacaaaagcatttgatgggaaaagacactcagataattttgtgcctcttcagagaaatttaaatgatttaaatgccaattgcttgagtaagtgaataaaataataccctcatcctaacataatttcataaatcaaagattatcatcaaaggggtgttagagacggagagactctggagcttat

SEQ ID NO: 41 - DNA, 401 bp, Homo sapiens
aaatgttttttcaaattgtgaaggtctacaaaaataagacaaaagcatttgatgggaaaagacactcagataattttgtgcctcttcagagaaatttaaatgatttaaatgccaattgcttgagtaagtgaataaaataataccctcatcctaacataatttcataaatcaaagattatcatcaaaggggtgttagagacrgagagactctggagcttatcctttgttctacctggaaatgaattgacttggaatctatgtgacttgcccaagtcaaaagctgtaaacaaacagggctcagaaaaagggtcttctctaggcctacccttcacatactcagaagcatgtgcacttgtaggaggaagcctgtccagccgcacttggtctctgagtgcttacag

SEQ ID NO: 42 - DNA, 401 bp, Homo sapiens
caaaagcatttgatgggaaaagacactcagataattttgtgcctcttcagagaaatttaaatgatttaaatgccaattgcttgagtaagtgaataaaataataccctcatcctaacataatttcataaatcaaagattatcatcaaaggggtgttagagacggagagactctggagcttatcctttgttctacctggaaaygaattgacttggaatctatgtgacttgcccaagtcaaaagctgtaaacaaacagggctcagaaaaagggtcttctctaggcctacccttcacatactcagaagcatgtgcacttgtaggaggaagcctgtccagccgcacttggtctctgagtgcttacagcagccaagcagcctcctctttggctgctaccaagctgac

SEQ ID NO: 43 - DNA, 401 bp, Homo sapiens
aaatgaattgacttggaatctatgtgacttgcccaagtcaaaagctgtaaacaaacagggctcagaaaaagggtcttctctaggcctacccttcacatactcagaagcatgtgcacttgtaggaggaagcctgtccagccgcacttggtctctgagtgcttacagcagccaagcagcctcctctttggctgctaccaagcygacctttgattcgagcttcccacaagccattatttcttaaaatcatggaatcatcaaattctctagtgcttttttttttcatttttttcttttctctgtgcttcacactgacccatttcttctcattgacccatgaagaagtagcaaaaagagagatgtgcacactgagtcagagcctttatgtgctatgatccaatatg

SEQ ID NO: 44 - DNA, 401 bp, Homo sapiens
gtaaacaaacagggctcagaaaaagggtcttctctaggcctacccttcacatactcagaagcatgtgcacttgtaggaggaagcctgtccagccgcacttggtctctgagtgcttacagcagccaagcagcctcctctttggctgctaccaagctgacctttgattcgagcttcccacaagccattatttcttaaaatcayggaatcatcaaattctctagtgcttttttttttcatttttttcttttctctgtgcttcacactgacccatttcttctcattgacccatgaagaagtagcaaaaagagagatgtgcacactgagtcagagcctttatgtgctatgatccaatatgtaccacactcccattagttggctggaaagctatctggggttttgag

SEQ ID NO: 45 - DNA, 401 bp, Homo sapiens
tagcaaaaagagagatgtgcacactgagtcagagcctttatgtgctatgatccaatatgtaccacactcccattagttggctggaaagctatctggggttttgagaatctagacctctgatttccaagcaagttgaagttattcccaacacacatggcttcccagtaagaagaattctctatatttctgtgtgtgtgtgtrtgtgtgtgtgtgtttctggttgctacaggaattagaagcagtggcaaaaaaaataatcctaataaggagtcctttgtgcccagctgttataaaacaggatttttcacaagcatttactctctccccacaatgaagccttaaagtgcaaaagttcaccctgagcattgcccactgcttttctttaaaccttccaagagaca

SEQ ID NO: 46 - DNA, 401 bp, Homo sapiens
gttgaagttattcccaacacacatggcttcccagtaagaagaattctctatatttctgtgtgtgtgtgtgtgtgtgtgtgtgtttctggttgctacaggaattagaagcagtggcaaaaaaaataatcctaataaggagtcctttgtgcccagctgttataaaacaggatttttcacaagcatttactctctccccacaaygaagccttaaagtgcaaaagttcaccctgagcattgcccactgcttttctttaaaccttccaagagacacacaaggctcttatttttcagggcccatctggccataaattgcaatagacattaatttatgaaaacaaatgacaccagcctactccacgacattttcaaatgggagtattaggcagggcaataaagcagga

SEQ ID NO: 47 - DNA, 401 bp, Homo sapiens
agccacacagggaccctcaatatccctgttccaacacactctgctcttgcaaagttacggttatttgatcataagacatgaaattcacttttttggccagagtgtggcaggaaattaaacatatccaagcctttctccatgtgaccagaatcaccattctacctgcagagtttacatcaatggtgataaccacagatagarcaaaggagctggattatcaatactggcagatgatttagctggtggttaatccattctccccagagtctagaggagttatttcattcttgggactcttgtctaaaatttggtgtgtggaacttctagtagagtcaaggggatggtttacaggcatgcagaggaaaggtaattttaacctccttattatatacctgcaattg

SEQ ID NO: 48 - DNA, 401 bp, Homo sapiens
gtttacaggcatgcagaggaaaggtaattttaacctccttattatatacctgcaattgccagcattaagcagacaagcagtgccaactcaaataccataggaaagatgcaaaaagaaagggctgagtaatatgtctgtgactctaggcaagacagctggcctttctagttttctgtttttctggatgcttgaacctaatgyatcttagtttttcagacatgcatactgagggcagtaagacgcacatatacatacaagaaatctttgtgctctgagagataataggtataactttaaaactcattttcattttatttatttgtttattttttgagatagaatctcgctctgtcactcaggctggagtgcagtggttcaatcttggctcactgcaaccttct

SEQ ID NO: 49 - DNA, 401 bp, Homo sapiens
gtgccaactcaaataccataggaaagatgcaaaaagaaagggctgagtaatatgtctgtgactctaggcaagacagctggcctttctagttttctgtttttctggatgcttgaacctaatgtatcttagtttttcagacatgcatactgagggcagtaagacgcacatatacatacaagaaatctttgtgctctgagagayaataggtataactttaaaactcattttcattttatttatttgtttattttttgagatagaatctcgctctgtcactcaggctggagtgcagtggttcaatcttggctcactgcaaccttctcctgagttcaagcaatcctcgtgcttcagcctcccaagtagctgggattacaggcatgtgccactcattttcattttaa

SEQ ID NO: 50 - DNA, 401 bp, Homo sapiens
agagtaggacttaaaccttgagctcaatggaacttccttctcctcctactcaaatcttggtctcaggactcacctatttggacagctgagttgagagtctgacataaattcacctgcctagccaccagcaaggtacctacaaggaagcacagggctgccccatatcctctccaggatatcctttccgggccctccatactrggggaccttgcctagagaagtttatctcatagaatttcctatgaggaggagattaaagactatgatcctgcaattcccccgagagaacacagagccaagaaactaagaaacgatgtcatgtgtgaactgcatatggcatttaacactgtcccccatccactattaaatgtggttccatgaaagtcaggtcctcaacagtc

SEQ ID NO: 51 - DNA, 401 bp, Homo sapiens
agaagtttatctcatagaatttcctatgaggaggagattaaagactatgatcctgcaattcccccgagagaacacagagccaagaaactaagaaacgatgtcatgtgtgaactgcatatggcatttaacactgtcccccatccactattaaatgtggttccatgaaagtcaggtcctcaacagtcaggttagctagtcctyccactttggggaggggcaaataagcaagtggagagagaagatggaagtattgtattctttctttaccagtcaaagtgcaaacatgagaaggagagtataactgttcctcctctcagttggaatttccttactgggggaattcaggaaacaggaacataaccagtaaaagaaactttggcaagaccttcctcagaatgttt

SEQ ID NO: 52 - DNA, 401 bp, Homo sapiens
atgtcatgtgtgaactgcatatggcatttaacactgtcccccatccactattaaatgtggttccatgaaagtcaggtcctcaacagtcaggttagctagtcctcccactttggggaggggcaaataagcaagtggagagagaagatggaagtattgtattctttctttaccagtcaaagtgcaaacatgagaaggagagtrtaactgttcctcctctcagttggaatttccttactgggggaattcaggaaacaggaacataaccagtaaaagaaactttggcaagaccttcctcagaatgtttcaagactgggagagaacaacatcccaagtggggcagaattttaatttttctctcttgaaatgtttccaaccttggaactgcattcatggtaagatat

SEQ ID NO: 53 - DNA, 401 bp, Homo sapiens
cctaaaatgctcttgtatcagctctttacctggctgatttcccttcacccttctggtcatagattaaatatcatctcctccaacaggccttctcttatggccgcatctaaagcaaagacaattccctatgagattctatcccattactctgtttgtttacataatagcaattaacacattatcaaattttaataattatcwgattatcaaagaggaactcaaaagggaaattagaaaatattttgaactaaatgaaaataaaaatttgtggtatggcactgaagtagtactttgggaaaaatatattgccaaatgcttagattagaaaaagatacacatctcaaatcaattacttcagcttctgcctaaagaaaatgaaaaaaaaagatgcttaccagggg

SEQ ID NO: 54 - DNA, 401 bp, Homo sapiens
cattgtttaattaattttttatattgttttactcttatagttcatagatttttgttttgatttttatcatttcttttttgtttactttggatttaatttactcttctttttgcactttcttgatgattatggtatctcccctggtaagcatctttttttttcattttctttaggcagaagctgaagtaattgatttgagaygtgtatctttttctaatctaagcatttggcaatatatttttcccaaagtactacttcagtgccataccacaaatttttattttcatttagttcaaaatattttctaatttcccttttgagttcctctttgataatcagataattattaaaatttgataatgtgttaattgctattatgtaaacaaacagagtaatgggat

SEQ ID NO: 55 - DNA, 401 bp, Homo sapiens
ctttcttgctctctgatttttcctgatcagtctgattgaggttgattattgatgtttttaaagaaacaactattatttcattgtttaattaattttttatattgttttactcttatagttcatagatttttgttttgatttttatcatttcttttttgtttactttggatttaatttactcttctttttgcactttcttgrtgattatggtatctcccctggtaagcatctttttttttcattttctttaggcagaagctgaagtaattgatttgagatgtgtatctttttctaatctaagcatttggcaatatatttttcccaaagtactacttcagtgccataccacaaatttttattttcatttagttcaaaatattttctaatttcccttttgagtt

SEQ ID NO: 56 - DNA, 401 bp, Homo sapiens
gaaattagaaaatattttgaactaaatgaaaataaaaatttgtggtatggcactgaagtagtactttgggaaaaatatattgccaaatgcttagattagaaaaagatacacatctcaaatcaattacttcagcttctgcctaaagaaaatgaaaaaaaaagatgcttaccaggggagataccataatcatcaagaaagtgmaaaaagaagagtaaattaaatccaaagtaaacaaaaaagaaatgataaaaatcaaaacaaaaatctatgaactataagagtaaaacaatataaaaaattaattaaacaatgaaataatagttgtttctttaaaaacatcaataatcaacctcaatcagactgatcaggaaaaatcagagagcaagaaaggacacaaatta

SEQ ID NO: 57 - DNA, 401 bp, Homo sapiens
ccaaagtaaacaaaaaagaaatgataaaaatcaaaacaaaaatctatgaactataagagtaaaacaatataaaaaattaattaaacaatgaaataatagttgtttctttaaaaacatcaataatcaacctcaatcagactgatcaggaaaaatcagagagcaagaaaggacacaaattaccagtatcaggaatgagaggtrgcatcactacagattctaccatatgaaaaggacaataggaaaatattatatataactttatgccaataaattgacaacctaagtgacaggcaaattttttgaaagatattgtgttaatctgttctttgtgttgctatataaagaaatctcagaaactaggtaatttataaagaaaagagatttgattggctaatggtgct

SEQ ID NO: 58 - DNA, 401 bp, Homo sapiens
ttaaaaacatcaataatcaacctcaatcagactgatcaggaaaaatcagagagcaagaaaggacacaaattaccagtatcaggaatgagaggtagcatcactacagattctaccatatgaaaaggacaataggaaaatattatatataactttatgccaataaattgacaacctaagtgacaggcaaattttttgaaagayattgtgttaatctgttctttgtgttgctatataaagaaatctcagaaactaggtaatttataaagaaaagagatttgattggctaatggtgcttcaggctgtacaagcatggcaccagcgtttgctcagcttctggaaagcctcatggagcttttactcagggaagaaggcaaagcagcagcaagcacatcatatggtga

SEQ ID NO: 59 - DNA, 401 bp, Homo sapiens
tattatatataactttatgccaataaattgacaacctaagtgacaggcaaattttttgaaagatattgtgttaatctgttctttgtgttgctatataaagaaatctcagaaactaggtaatttataaagaaaagagatttgattggctaatggtgcttcaggctgtacaagcatggcaccagcgtttgctcagcttctggraagcctcatggagcttttactcagggaagaaggcaaagcagcagcaagcacatcatatggtgaaagtagagcaagagagagagtgagtgtgggttgtgggagaggttccagacacttttaaaaaaccagatctcaggagaacttactcactgtcgtgaagacacaaccaagccacgagggattcacccccattgcccaaa

SEQ ID NO: 60 - DNA, 401 bp, Homo sapiens
gggaagaaggcaaagcagcagcaagcacatcatatggtgaaagtagagcaagagagagagtgagtgtgggttgtgggagaggttccagacacttttaaaaaaccagatctcaggagaacttactcactgtcgtgaagacacaaccaagccacgagggattcacccccattgcccaaacacctcccactaagacccacctcyaacgttggggattatatttcaacatgagattttgcaggaacatatgctcaaactctatcaggcacaaactaccaaagctcactcaaggagaaataggtaaccttagcacaaataactttatatctaagaaaaaaattaaatttgtagttaaaaatcttcccacaaagaaaacttcaggcttagatgtcttcactggtgaa

SEQ ID NO: 61 - DNA, 401 bp, Homo sapiens
cagcagcaagcacatcatatggtgaaagtagagcaagagagagagtgagtgtgggttgtgggagaggttccagacacttttaaaaaaccagatctcaggagaacttactcactgtcgtgaagacacaaccaagccacgagggattcacccccattgcccaaacacctcccactaagacccacctccaacgttggggattayatttcaacatgagattttgcaggaacatatgctcaaactctatcaggcacaaactaccaaagctcactcaaggagaaataggtaaccttagcacaaataactttatatctaagaaaaaaattaaatttgtagttaaaaatcttcccacaaagaaaacttcaggcttagatgtcttcactggtgaattacattttagaaag

SEQ ID NO: 62 - DNA, 401 bp, Homo sapiens
ccagatctcaggagaacttactcactgtcgtgaagacacaaccaagccacgagggattcacccccattgcccaaacacctcccactaagacccacctccaacgttggggattatatttcaacatgagattttgcaggaacatatgctcaaactctatcaggcacaaactaccaaagctcactcaaggagaaataggtaacmttagcacaaataactttatatctaagaaaaaaattaaatttgtagttaaaaatcttcccacaaagaaaacttcaggcttagatgtcttcactggtgaattacattttagaaagaactaataccaattctacataaacaactctaaaagttgaaaaggacaaaatattttccaactctttttatgaaatcaggcttactct

SEQ ID NO: 63 - DNA, 401 bp, Homo sapiens
ttcaacatgagattttgcaggaacatatgctcaaactctatcaggcacaaactaccaaagctcactcaaggagaaataggtaaccttagcacaaataactttatatctaagaaaaaaattaaatttgtagttaaaaatcttcccacaaagaaaacttcaggcttagatgtcttcactggtgaattacattttagaaagaaytaataccaattctacataaacaactctaaaagttgaaaaggacaaaatattttccaactctttttatgaaatcaggcttactctgatacaaaagtcatacaaatacattataggaaaagaaaattacatactaactgtcatcaaacatagatgtaaaatgtctatacaaagttttagcaaatcaagttcaacaatatgta

SEQ ID NO: 64 - DNA, 401 bp, Homo sapiens
tgaaaaggacaaaatattttccaactctttttatgaaatcaggcttactctgatacaaaagtcatacaaatacattataggaaaagaaaattacatactaactgtcatcaaacatagatgtaaaatgtctatacaaagttttagcaaatcaagttcaacaatatgtacaaggaattttacaaatgaccaaatagggtttawcccagaactgcaagtttggcttaatatttgacgacaaatcaatgcaattcaccactattagcatactttaaaaaataaaactcatatgatatttctgatagataccaaaaaagtatttgacaaaattcagtgttcattccttaccaagaaattaaaattaaaataaaaaaaccacaaaccttcagccaaccaggaataga

SEQ ID NO: 65 - DNA, 401 bp, Homo sapiens
attttccaactctttttatgaaatcaggcttactctgatacaaaagtcatacaaatacattataggaaaagaaaattacatactaactgtcatcaaacatagatgtaaaatgtctatacaaagttttagcaaatcaagttcaacaatatgtacaaggaattttacaaatgaccaaatagggtttatcccagaactgcaagkttggcttaatatttgacgacaaatcaatgcaattcaccactattagcatactttaaaaaataaaactcatatgatatttctgatagataccaaaaaagtatttgacaaaattcagtgttcattccttaccaagaaattaaaattaaaataaaaaaaccacaaaccttcagccaaccaggaatagaagagaactgcctcaa

SEQ ID NO: 66 - DNA, 401 bp, Homo sapiens
aaattcagtgttcattccttaccaagaaattaaaattaaaataaaaaaaccacaaaccttcagccaaccaggaatagaagagaactgcctcaatcttacttaatggttaaaaagccaaatgtctttctcctaagacaaggtacaagaaaatcatgtctgctctaacagtttctatttactgtactatgggtttgagtcagrgcagtcggatgagagcaagaactaaaaaccatccagattggaaaagaagaagtgaaactttctttatttgcaggctacatgactgaatagtagaaaatcagatagaatctacaaaacatcaattggatttctatatggtaacaatgaaaaaatagaaattgatgttaaacataatattatttatagtaacatcaaaaata

SEQ ID NO: 67 - DNA, 401 bp, Homo sapiens
caagaaattaaaattaaaataaaaaaaccacaaaccttcagccaaccaggaatagaagagaactgcctcaatcttacttaatggttaaaaagccaaatgtctttctcctaagacaaggtacaagaaaatcatgtctgctctaacagtttctatttactgtactatgggtttgagtcagagcagtcggatgagagcaagaamtaaaaaccatccagattggaaaagaagaagtgaaactttctttatttgcaggctacatgactgaatagtagaaaatcagatagaatctacaaaacatcaattggatttctatatggtaacaatgaaaaaatagaaattgatgttaaacataatattatttatagtaacatcaaaaatatgaaatatttaaagacaattct

SEQ ID NO: 68 - DNA, 401 bp, Homo sapiens
tatttactgtactatgggtttgagtcagagcagtcggatgagagcaagaactaaaaaccatccagattggaaaagaagaagtgaaactttctttatttgcaggctacatgactgaatagtagaaaatcagatagaatctacaaaacatcaattggatttctatatggtaacaatgaaaaaatagaaattgatgttaaacakaatattatttatagtaacatcaaaaatatgaaatatttaaagacaattctgacaacatatatgaaagggctatgccctgaaaattacagaacattgttaaagaaatctttaaaaacccaaataaatggagagattacatcatgttcacgggtcagaacattcatgttgttaagatgtcagttctctcaagattgagctat

SEQ ID NO: 69 - DNA, 401 bp, Homo sapiens
aaagacagaattttgtaaattctgtaaagagaattaaaagataaaaggcagcataggagaacatatttgtaaataatatatctgacaaagaagctgtatttagaattcataaagaactctcaaaattcaataataagaaagcaagtaatcaaatacaaatggtcaaaagatttgaacagacagtcactaaagaaaaagtakggttggcaaataaatagacggaaagatgctcaacatcagcagttgttaggggaatgcaaatcaaactagaattagataccactacggagttattggaatggctaacattgaacacactgacccatttcatgtgttgtcaaagatatggagtgactgggacacacacattactgttgggcatagccactttggaaaacagt

SEQ ID NO: 70 - DNA, 401 bp, Homo sapiens
ccatgctgctatccaccctttctgtctggttttcccgagttgcagggattacacacccatttggattgtgagacttctcagaaccagcttcttcacagaaattctttctaactcttccagcccatggagctgcatgtcaagttacacattcttaaaaggttcatcactgactaggatctatctcccagttcctccacagtrctctccaaccctatcactcccctccaactcaacctactggcatcattgcctgctccaaactgtgagtgggaaagtgagagtagaaatttctgcatcaggatccataagctgaagtgagtgggagaaggacttgaatacagtctgttaacacctcagggagagttaattaaaacctgaggaaccagagataccagatagtc

SEQ ID NO: 71 - DNA, 401 bp, Homo sapiens
tcccctccaactcaacctactggcatcattgcctgctccaaactgtgagtgggaaagtgagagtagaaatttctgcatcaggatccataagctgaagtgagtgggagaaggacttgaatacagtctgttaacacctcagggagagttaattaaaacctgaggaaccagagataccagatagtcctagcatatctcaacaakggcaacttagcagtttttattagtgccacaatcatactaaagaaggaagtgagtggagaggtgcaaagaaaacaaaaagagcacagaagatgatggggaggacagaagcctagcttttgctgacttcttggactgcttagttgaaatccaggagttctgagaaagtttagtcatgcctgtccagctttgcccgtcgctgg

SEQ ID NO: 72 - DNA, 401 bp, Homo sapiens
cagggagagttaattaaaacctgaggaaccagagataccagatagtcctagcatatctcaacaatggcaacttagcagtttttattagtgccacaatcatactaaagaaggaagtgagtggagaggtgcaaagaaaacaaaaagagcacagaagatgatggggaggacagaagcctagcttttgctgacttcttggactgyttagttgaaatccaggagttctgagaaagtttagtcatgcctgtccagctttgcccgtcgctggactttttctgaattgccagtgccaaatatttgaaaatttttgagagattattttttgtggaaatatagccaaaagtttcagggaaatagacaagtttctaagaagctggactccaagaatgggttccgtgtgagta

SEQ ID NO: 73 - DNA, 401 bp, Homo sapiens
gaaaactgatacaaacttacaaaaataactcaccacctgcatttcatatcctcattttcttcttgtccatctatgcttcaacttgtacctctcttcccttggcaaccagaacctctgcattagcacgcagtgagcacacaaataagatgatcatttttcccgagttctctcatctccagcccatgatgttttactgttgcrtaagatttcactccagaatgtcctttgggcctgacattcttagacgtcaaacatcccatccctacttgatttcaaaactgttagtattccataagtaaaattaagtaggacccctaaggtaacgttttctttatctgctaattagtggctttctaactttgctagaccaagccctctaaaacacaattacacagggagta

SEQ ID NO: 74 - DNA, 401 bp, Homo sapiens
atctatgcttcaacttgtacctctcttcccttggcaaccagaacctctgcattagcacgcagtgagcacacaaataagatgatcatttttcccgagttctctcatctccagcccatgatgttttactgttgcataagatttcactccagaatgtcctttgggcctgacattcttagacgtcaaacatcccatccctacttsatttcaaaactgttagtattccataagtaaaattaagtaggacccctaaggtaacgttttctttatctgctaattagtggctttctaactttgctagaccaagccctctaaaacacaattacacagggagtacaagaaggggacatagaaaaagaaagcaaacatctaagtacattcagggatctctgtaacattcagac

SEQ ID NO: 75 - DNA, 401 bp, Homo sapiens
gttttactgttgcataagatttcactccagaatgtcctttgggcctgacattcttagacgtcaaacatcccatccctacttgatttcaaaactgttagtattccataagtaaaattaagtaggacccctaaggtaacgttttctttatctgctaattagtggctttctaactttgctagaccaagccctctaaaacacaawtacacagggagtacaagaaggggacatagaaaaagaaagcaaacatctaagtacattcagggatctctgtaacattcagacacagaatgttagaacttagaaggatcttaggcaatatggttcaatccttgccagcttcacaagttgtagcattagctaggtgagcttggacataccatttacactctctaagtttcatt

SEQ ID NO: 76 - DNA, 401 bp, Homo sapiens
ctaaggtaacgttttctttatctgctaattagtggctttctaactttgctagaccaagccctctaaaacacaattacacagggagtacaagaaggggacatagaaaaagaaagcaaacatctaagtacattcagggatctctgtaacattcagacacagaatgttagaacttagaaggatcttaggcaatatggttcaatycttgccagcttcacaagttgtagcattagctaggtgagcttggacataccatttacactctctaagtttcattcagtgacagagccaggtttgggacccagatattctgatacttttcttccatgaagcctttccaattacataacctacaagtgtgctcactctttagtgcaaatgtagaaaaacaagctttgtgtcca

SEQ ID NO: 77 - DNA, 401 bp, Homo sapiens
tttgctagaccaagccctctaaaacacaattacacagggagtacaagaaggggacatagaaaaagaaagcaaacatctaagtacattcagggatctctgtaacattcagacacagaatgttagaacttagaaggatcttaggcaatatggttcaatccttgccagcttcacaagttgtagcattagctaggtgagcttggrcataccatttacactctctaagtttcattcagtgacagagccaggtttgggacccagatattctgatacttttcttccatgaagcctttccaattacataacctacaagtgtgctcactctttagtgcaaatgtagaaaaacaagctttgtgtccatcgggcatgagcagtgtaccccctattgaaagtatttttgccag

SEQ ID NO: 78 - DNA, 401 bp, Homo sapiens
agtgacagagccaggtttgggacccagatattctgatacttttcttccatgaagcctttccaattacataacctacaagtgtgctcactctttagtgcaaatgtagaaaaacaagctttgtgtccatcgggcatgagcagtgtaccccctattgaaagtatttttgccagacttggttgctcatgcctgtaatcctagtgrctcagaggctgaggtaggaggatctcttgaggccaagagttcaggaagagcctgggcaacatagagagacccaaaatctaaaatcaataaatcaataaactatagtcaggtgtggtggggtgcacctggagttccaactacttgggaggctaaggcaggaggactgcttgagcccaagagtttgaggctgcagtgagcta

SEQ ID NO: 79 - DNA, 401 bp, Homo sapiens
tttgggacccagatattctgatacttttcttccatgaagcctttccaattacataacctacaagtgtgctcactctttagtgcaaatgtagaaaaacaagctttgtgtccatcgggcatgagcagtgtaccccctattgaaagtatttttgccagacttggttgctcatgcctgtaatcctagtgactcagaggctgaggyaggaggatctcttgaggccaagagttcaggaagagcctgggcaacatagagagacccaaaatctaaaatcaataaatcaataaactatagtcaggtgtggtggggtgcacctggagttccaactacttgggaggctaaggcaggaggactgcttgagcccaagagtttgaggctgcagtgagctatgattatcactgtac

SEQ ID NO: 80 - DNA, 401 bp, Homo sapiens
atagtcaggtgtggtggggtgcacctggagttccaactacttgggaggctaaggcaggaggactgcttgagcccaagagtttgaggctgcagtgagctatgattatcactgtactccagcccaggtgatgaaacaaaccctcatctctaaaactaaaataaaactaaagatagatagataaataaataaataaataaatawataaataagtgttctcagcacccacagctgcagctgtgaatggtcagatcagctccagagggccactgcgggagaggccccaaaacaataatacggagtccaccatcagctctgaaaccaccaagaagcagcagaccttttgggagagatcagcactgaccataagttttccttttctagagaactattttctctcgcaa

SEQ ID NO: 81 - DNA, 401 bp, Homo sapiens
tggtggggtgcacctggagttccaactacttgggaggctaaggcaggaggactgcttgagcccaagagtttgaggctgcagtgagctatgattatcactgtactccagcccaggtgatgaaacaaaccctcatctctaaaactaaaataaaactaaagatagatagataaataaataaataaataaatatataaataagtsttctcagcacccacagctgcagctgtgaatggtcagatcagctccagagggccactgcgggagaggccccaaaacaataatacggagtccaccatcagctctgaaaccaccaagaagcagcagaccttttgggagagatcagcactgaccataagttttccttttctagagaactattttctctcgcaagccctcatccc

SEQ ID NO: 82 - DNA, 401 bp, Homo sapiens
ataatacggagtccaccatcagctctgaaaccaccaagaagcagcagaccttttgggagagatcagcactgaccataagttttccttttctagagaactattttctctcgcaagccctcatcccttatcaggtccccagacccaacccccagcctcagctctagaaaagtgaatctttaaagacccaacacttgacctggmtggctaaggggtagagtgctgttcattcgtcatggaaaatttcctaaagaaaagaaatgcatttatcttaattctgcccttacgtttatacgattcttttcctgaggatttctactcttaatagaaaggagagaaagaggaagaaaagaaaatctttaaagaacatactttatagccctatgtactgggtcacggctgat

SEQ ID NO: 83 - DNA, 401 bp, Homo sapiens
tgctggaatatatttttaaatctccatgaataacttaagatctaggtcaagccaggggcagaatgaaataaaaataaagaaaataaaatgagagagatactgatttgattcattttcccatctgtgaataccccgagatgtaggctaaggagaaatgcttgttaatttgtgcaagggaattttttctctggtttactgtgytatccttccttttggcctttgtcgctcagttccaaacatcacagtacactgatgccttaaacctgctttgcaaatgctcatgtctagtattttgctttcttctttaagctcaaacctttgagttctcaggccttccatcagcttcatgaaatgcaccccagcattgctctcttgatctgtgtgtcctctcctgaacccta

SEQ ID NO: 84 - DNA, 401 bp, Homo sapiens
tataattttcaaaagtttaaaaacatcactttcataaaagtataaattcaacttattaattaatgagagaattagtaacctgttaatattgttacaaaaaagaatttgaagaacaaagtatgtagggtgacagaaatgttggtgtgaatttactaatagatgcaaaactggctaaaatccaataggaaaactaagtataawgtttggagcaataaaattgacaagacttttagctagaataatcaaaaaaaaaaaaaataaagaaggctcaaattataaagtcagaatggaagaagagacatcattaccaatcttacataaataaaaaaaaatttataagggaatgttatgaacaactacataccaatgaattacataatatacctgaattagataaattt

SEQ ID NO: 85 - DNA, 401 bp, Homo sapiens
agaattagtaacctgttaatattgttacaaaaaagaatttgaagaacaaagtatgtagggtgacagaaatgttggtgtgaatttactaatagatgcaaaactggctaaaatccaataggaaaactaagtataaagtttggagcaataaaattgacaagacttttagctagaataatcaaaaaaaaaaaaaataaagaaggytcaaattataaagtcagaatggaagaagagacatcattaccaatcttacataaataaaaaaaaatttataagggaatgttatgaacaactacataccaatgaattacataatatacctgaattagataaatttttagaagacacaaattacagaaaattcctcaagaaatatagaaataatgaataaatctttaacaagt

SEQ ID NO: 86 - DNA, 401 bp, Homo sapiens
tctagccagggcaattatttaagaaaaagaagtgaaaatgaaagtcaaaggttaaaaaggaaaaagtaaaactattccatttgcagatggcaagattttgtatatagaaactatttacaatttcattaaaaaactattaaaactggtaaatgagttgagcaagtttgcagaatacaagatcaatatgctaaaataacctgygtttctatgcatctgcagtaaaattgcaaaaatgaaattaagaaatcaattttgtttataataatattaaagagaacaaaatacttaggagtacatttaataaaatatacaccaacatcattgaaggaaattaaagatttaatgaatagaaagatagtccatgtccatgaatcagatgacttcatattttttaagatgag

SEQ ID NO: 87 - DNA, 401 bp, Homo sapiens
aagaagtgaaaatgaaagtcaaaggttaaaaaggaaaaagtaaaactattccatttgcagatggcaagattttgtatatagaaactatttacaatttcattaaaaaactattaaaactggtaaatgagttgagcaagtttgcagaatacaagatcaatatgctaaaataacctgcgtttctatgcatctgcagtaaaattrcaaaaatgaaattaagaaatcaattttgtttataataatattaaagagaacaaaatacttaggagtacatttaataaaatatacaccaacatcattgaaggaaattaaagatttaatgaatagaaagatagtccatgtccatgaatcagatgacttcatattttttaagatgagtatgcttcccaatttaacccacaagt

SEQ ID NO: 88 - DNA, 401 bp, Homo sapiens
aaaatgaaagtcaaaggttaaaaaggaaaaagtaaaactattccatttgcagatggcaagattttgtatatagaaactatttacaatttcattaaaaaactattaaaactggtaaatgagttgagcaagtttgcagaatacaagatcaatatgctaaaataacctgcgtttctatgcatctgcagtaaaattgcaaaaatkaaattaagaaatcaattttgtttataataatattaaagagaacaaaatacttaggagtacatttaataaaatatacaccaacatcattgaaggaaattaaagatttaatgaatagaaagatagtccatgtccatgaatcagatgacttcatattttttaagatgagtatgcttcccaatttaacccacaagtacaaaact

SEQ ID NO: 89 - DNA, 401 bp, Homo sapiens
ttttaagatgagtatgcttcccaatttaacccacaagtacaaaactatcccaatcaagttagcagctgcttctttgtagaaatcactaattggtccttaaattcatttagaaatgcaaaggaccccaaaaatctaaaacaatcttgaaaaacaacaataaaattagattactcaaacttcccaatttcaaaatttactacraatctatagtcaccaagaaagtgtggtactgacacaaggctagatgtatagatcaatgaaacaaaattgagagtgcataaataaaccatatttttatgatcagttgatgttcaacaaaagtgcagtggcaatttcatgaagaaagaatactctttttaacaaattgtggagggacatctggacatccacatacaaaagaa

SEQ ID NO: 90 - DNA, 401 bp, Homo sapiens
gatcaatgaaacaaaattgagagtgcataaataaaccatatttttatgatcagttgatgttcaacaaaagtgcagtggcaatttcatgaagaaagaatactctttttaacaaattgtggagggacatctggacatccacatacaaaagaataaatttggatctctaccacataatatatacagacataaactcaaaatgawtcataaacctaaatgtaagagctaaaacactaaaactcttagaagaaaacaaaaaataaatctttgtggctttatgtcaagcaatggtgtctttgatataacatcaaaagcacaggtaacaaaagaaataatataaattgcatttaaaattaaaaactcttattttgcaaataataccatcaagaagtgaaaaagacaaa

SEQ ID NO: 91 - DNA, 401 bp, Homo sapiens
aaatagttacctatacaaaaaaacaaaaatatagctatctatacaattgaatattatttggcaataaagaatgaagtattgatacatgttatagcataaatgaactttgaaaccattatgctaagtgacagaagtcagttttgaaagactgtatactgtatgtttacatttacataaaattttcagaataagcaaatctakagaaacagagagtagattggtggttgtctagggcttgggagatggggaaagcaaaacatgaaaaagaaagaaggatctgctaatagggatggggtttctttgtagggatgaggaggagcaataaaatggtctaaaattagattgctgtgctgtttgtacaagcttgtgcacatacggaacaaaactgaactgtaagcttt

SEQ ID NO: 92 - DNA, 401 bp, Homo sapiens
aaccattatgctaagtgacagaagtcagttttgaaagactgtatactgtatgtttacatttacataaaattttcagaataagcaaatctagagaaacagagagtagattggtggttgtctagggcttgggagatggggaaagcaaaacatgaaaaagaaagaaggatctgctaatagggatggggtttctttgtagggatraggaggagcaataaaatggtctaaaattagattgctgtgctgtttgtacaagcttgtgcacatacggaacaaaactgaactgtaagctttaaatggatgaattgtatggtataagaatcatatttcaatagagctgcttaaaatgtattgtttgggactatcttttctacagggtagaattcagtcacatctctattaga

SEQ ID NO: 93 - DNA, 401 bp, Homo sapiens
agcaaaacatgaaaaagaaagaaggatctgctaatagggatggggtttctttgtagggatgaggaggagcaataaaatggtctaaaattagattgctgtgctgtttgtacaagcttgtgcacatacggaacaaaactgaactgtaagctttaaatggatgaattgtatggtataagaatcatatttcaatagagctgcttmaaatgtattgtttgggactatcttttctacagggtagaattcagtcacatctctattagacaaaattcattgtatttctttaaggaggatcatttgcattactgtcagttttccacaagtaaaataacttagaaaattgaaattcagttaaagagaaacatctcaatataatcagatatccccagaggataccctagaca

SEQ ID NO: 94 - DNA, 401 bp, Homo sapiens
tatatttacctggctcattcccctttttcattccggtctctgctcaactgttagcctcttattaagagcttccctaatcacatgtattagttatgtttaacacattgtcccacaaagctgtggctttaaacaacaaatattatctcagagtttctctaagtcaggaatctaggattatcttacctgggtggttctggttcyggaaccttccttccaccgcagtcagaaggttggtaggggctgtaatcatctgaaggtctgactggggctggagaatctatttcctagatggctcctttacatggctgttggcaggaggcctccattccttgcgacatgaacctctttataaactgcttgagtatcctcacaacatgacagctaacttccccagagaagac

SEQ ID NO: 95 - DNA, 401 bp, Homo sapiens
tacctggctcattcccctttttcattccggtctctgctcaactgttagcctcttattaagagcttccctaatcacatgtattagttatgtttaacacattgtcccacaaagctgtggctttaaacaacaaatattatctcagagtttctctaagtcaggaatctaggattatcttacctgggtggttctggttccggaacyttccttccaccgcagtcagaaggttggtaggggctgtaatcatctgaaggtctgactggggctggagaatctatttcctagatggctcctttacatggctgttggcaggaggcctccattccttgcgacatgaacctctttataaactgcttgagtatcctcacaacatgacagctaacttccccagagaagacaatcta

SEQ ID NO: 96 - DNA, 401 bp, Homo sapiens
aggcctccattccttgcgacatgaacctctttataaactgcttgagtatcctcacaacatgacagctaacttccccagagaagacaatctaaaaacaggaggaggaagacacagtggctttattatccagcctgaaagccatacatcatcacttctactttattctatttgctagaagaagtcactaagccaagcccacaytcaaggggaggtagcttaggctgcaccttttgaagagagggtgaaagagtttgtggacctattttaaagcactataacacacccctatgcaaacacatctccttgtctggtactttctactgccttatttgatttttgcttccttcataacattggtattattatattatatatgtatgtgtgaattattgctttcccca

SEQ ID NO: 97 - DNA, 401 bp, Homo sapiens
tctaaaaacaggaggaggaagacacagtggctttattatccagcctgaaagccatacatcatcacttctactttattctatttgctagaagaagtcactaagccaagcccacattcaaggggaggtagcttaggctgcaccttttgaagagagggtgaaagagtttgtggacctattttaaagcactataacacacccctrtgcaaacacatctccttgtctggtactttctactgccttatttgatttttgcttccttcataacattggtattattatattatatatgtatgtgtgaattattgctttccccaccacaatataagatcaataaaaatattgcattatttcacactgaatactcagcacatataatagtacaagacatatattaattcaat

SEQ ID NO: 98 - DNA, 401 bp, Homo sapiens
tactttctactgccttatttgatttttgcttccttcataacattggtattattatattatatatgtatgtgtgaattattgctttccccaccacaatataagatcaataaaaatattgcattatttcacactgaatactcagcacatataatagtacaagacatatattaattcaatgactgttttgtttcctaattaaaygaacatatggatgaagttataaattacaggagcagaaccaggcttggaaactaggtacaatgactcctaacctatgctatttccccgtattttaccttagcggaattctaacggttgaatgcacctcaacagtgattctgcatgaggaaagggatcctcccagtcctccatgacatcactcaattgagacctcttccaca

SEQ ID NO: 99 - DNA, 401 bp, Homo sapiens
gcttcagaaatcctgctaaatcctgaataaacgggagtgaacaagcaaggtagcattcaaggtctcttactcctaaacccagttgcaatacttgtattaaaccctgggctccaggctgcttcagtgctatttaactttctctttacaaagagaagagaaaatgtgggggtatcagacatatcccacccttccaaggccccrtgcagactcttaaaagatgagggggtttttgtttgtgcatatggatcaaagaaagactttcttgaattttggtcagcagggtttgttttagatctgtgaagtgtggaagaggtctcaattgagtgatgtcatggaggactgggaggatccctttcctcatgcagaatcactgttgaggtgcattcaaccgttagaattcc

SEQ ID NO: 100 - DNA, 401 bp, Homo sapiens
aaatgtctcccatcacttttctttgtactaaaaagatcattgagaaatgcctttttttttcttttatacgcatcatcttcctagaatctgaaggctgctagagaagggagagaaacatgaaagtgagtcccatgctgattgttgttgaaatacctggaaagaaactggcaaagactatccaagggatgctaagacatgtcygaacttacaaaacagtagaacctgaataggaatttgaagttgtctatactctcctcagccacaccctgccattttacaggtatgaaaaataaaattttcagcaaattagtgatagatacaaagtatctggatgcccaatcctgtgtactttccattaccatctgtttcctaattgtgtgtggatttccttgctctccaga

SEQ ID NO: 101 - DNA, 401 bp, Homo sapiens
gcagggtgtggctgaggagagtatagacaacttcaaattcctattcaggttctactgttttgtaagttcggacatgtcttagcatcccttggatagtctttgccagtttctttccaggtatttcaacaacaatcagcatgggactcactttcatgtttctctcccttctctagcagccttcagattctaggaagatgatgygtataaaagaaaaaaaaaggcatttctcaatgatctttttagtacaaagaaaagtgatgggagacatttaagacaaactgcccacttcccgcacaatatgaggcaaaacaccaacttcaggggacgggagaatggaaatcttagctcttccctatcacctcccagccagtgggaagtcagaaagaatggtgactcctcac

SEQ ID NO: 102 - DNA, 401 bp, Homo sapiens
acaacaatcagcatgggactcactttcatgtttctctcccttctctagcagccttcagattctaggaagatgatgcgtataaaagaaaaaaaaaggcatttctcaatgatctttttagtacaaagaaaagtgatgggagacatttaagacaaactgcccacttcccgcacaatatgaggcaaaacaccaacttcaggggaygggagaatggaaatcttagctcttccctatcacctcccagccagtgggaagtcagaaagaatggtgactcctcacgcttacagagggcagtgttatttcaaagactatggtgagttccccagctctcagcaagggtaccatctcaccactccccttggtgccctcgatggagcttctcaatcaacaccaaaggagacgcg

SEQ ID NO: 103 - DNA, 401 bp, Homo sapiens
tgttcatatgtgccctctgattaattcacagggggaaattgtcttataactttccacctgaatgtctgcctctgcatagccatggagcagtgccctggaaacagtaacagtgatgataacaatgagaacaacacccactctgtttaagccctagccccaggcaacctccttacctgatgtgacaccagaaggaaaggacayggcattttctccctcacgaatgtctgggaaatcctatccttgcttgggtcttgggctgccatatttcaatcaaaagcaaaatggagaaagaagaatttaagaagttattgggaagcaaggaataagtgaaaggagagaaaactcagtttttttgctttacggcctgtgatatatgcaagctaaccaattaaagaatgtca

SEQ ID NO: 104 - DNA, 401 bp, Homo sapiens
tgattaattcacagggggaaattgtcttataactttccacctgaatgtctgcctctgcatagccatggagcagtgccctggaaacagtaacagtgatgataacaatgagaacaacacccactctgtttaagccctagccccaggcaacctccttacctgatgtgacaccagaaggaaaggacatggcattttctccctcasgaatgtctgggaaatcctatccttgcttgggtcttgggctgccatatttcaatcaaaagcaaaatggagaaagaagaatttaagaagttattgggaagcaaggaataagtgaaaggagagaaaactcagtttttttgctttacggcctgtgatatatgcaagctaaccaattaaagaatgtcagagctagaaacattctc

SEQ ID NO: 105 - DNA, 401 bp, Homo sapiens
tgatggttaggagttatggttgcagacagaaatgaacgggcctgaagaatggaagaaacaggtgagaaagccaagatgctgatttctactgagttttagaaaccatgatcaatagtattagtgactcttaaggagggcgataaaagggacagagtccaggatgttcccagatttctagcatgggagactggttacatctcrgtgacatgtattaagatgggagataccagagagggaacaggtcaagagtttgagactatgtgtcttatttgagaaggaaattcagttgcaacacccaataatcaattagggttggaacccaggagacaggttccgatttgagagacaacatgtactgtgggtgacaatcaaagccatgggaatggatgaggtctttgaga

SEQ ID NO: 106 - DNA, 401 bp, Homo sapiens
ctgcagaatctagggcccatggagcccacatgtgcttagccaggctctcagaccttttgctctttcctggtcaggatgaggtaggaaagtctccaacatatttgctctgatttgctctcctgcacgtcccaatatgaagtttgtaggctcctgaagcaaagtcagtgttctcagatacaacccagagtgaatgcctctagratcagcctctgctaagcgatttcccaaatgctccatatagcccatttgcagtgtcccgcattgaaggtctggtgtgcagatgtgtctctttagataagggtgctgctcttttgatatctgttaaatcacaatgacttggactgtctcatgtttagaagactcagaactttgtagaaaagatgagaggaaaagcgaagaga

SEQ ID NO: 107 - DNA, 401 bp, Homo sapiens
atgtgcttagccaggctctcagaccttttgctctttcctggtcaggatgaggtaggaaagtctccaacatatttgctctgatttgctctcctgcacgtcccaatatgaagtttgtaggctcctgaagcaaagtcagtgttctcagatacaacccagagtgaatgcctctagaatcagcctctgctaagcgatttcccaaaygctccatatagcccatttgcagtgtcccgcattgaaggtctggtgtgcagatgtgtctctttagataagggtgctgctcttttgatatctgttaaatcacaatgacttggactgtctcatgtttagaagactcagaactttgtagaaaagatgagaggaaaagcgaagagaggaaaaagagtaaggaagagcatggaaaa

SEQ ID NO: 108 - DNA, 401 bp, Homo sapiens
actcagaactttgtagaaaagatgagaggaaaagcgaagagaggaaaaagagtaaggaagagcatggaaaaagaaaagcagttaaaacaaacatctaaatagaagtctctgtgctttcaaagctttgatttctcaagcaaaacatttcttaagtgaaaaagccaccacttattcattctttatttttacctttctagtgtktggtgtaaagtaggagcataaatcatgtcccattaattcattcaagagtcctaatgctaattctatcaaaaaacccagcacaaaatgcccaagcattcattataacaggatttgagacccaatgtgctgctccttcatgcttctcctgagagcgattttccttggttacccccatcctccctttcattggcttccccagg

SEQ ID NO: 109 - DNA, 401 bp, Homo sapiens
gccagggctgtcccaacatattactcccataattttccactgtgggttcccagatgggttggatggtgctcatgattcttcagcttatttcatgccttatgcagccagctcctagggaggtatggaaaatgagagcccagtctggaacctcagaccttcagactgccaagggctggtaggatttccccagcatccaatctwcatccttataagggtctgctctgccaaggacccattctcttgatttcagctcaatgatcttgtctgccttggcagggcctgctcaccacaagcttggatgtcatggaattttggggaagaaagagcattaaatacttctccatgttacttcttgccctccctgaaacagttgactggactctaacacttggtttggggaa

SEQ ID NO: 110 - DNA, 401 bp, Homo sapiens
aaatgagagcccagtctggaacctcagaccttcagactgccaagggctggtaggatttccccagcatccaatcttcatccttataagggtctgctctgccaaggacccattctcttgatttcagctcaatgatcttgtctgccttggcagggcctgctcaccacaagcttggatgtcatggaattttggggaagaaagagsattaaatacttctccatgttacttcttgccctccctgaaacagttgactggactctaacacttggtttggggaagaattttaccccgaagccatctggcaaggctgaagacatgtctttttatattctgctttccttgggagtaataggccaagagagattcatgtttgtctcacaaagaggaggtgaccgtggtttctg

SEQ ID NO: 111 - DNA, 401 bp, Homo sapiens
gagagattcatgtttgtctcacaaagaggaggtgaccgtggtttctggcaacaccttgctttaaggcagtggagtcaagagtaaagaggctggaaaagcagaatacttactctacctccacccctcactcccctggggacttgtctatggtcaaatccctaaagactctcttttcacattgataaatcaggggcaagaacyacatagctcaccccaatttagtaaggccaaaacaagagaggagtagtgagcatttgagaagcactgacatggaaattataaacaatgaagccctctataaacaggctcttggctgagttttagaggtggatttcttcatctggtttggggcatgtcagttcatccatataactcctgcacgaatctgatccaaagtgtgc

SEQ ID NO: 112 - DNA, 401 bp, Homo sapiens
tttggggcatgtcagttcatccatataactcctgcacgaatctgatccaaagtgtgcaactcaattccttctgcatctccttccctcaccttgctcatttctataggacacagtatatttcagcacataactgcacatcaagtaaaatttacttgggaggggtggcagctaaccagaatttgagttcttgctcagcgacayggaatgtatgaagcaagggcctggctaaacagctttatataatacgaccgagtaaagtagggtagtaccaacaaagaatttgactaagcctttggagcttcttgtcctgaccaagttcacctgtagtgaggagagaacagccattcatggatcactcaggtggtgacaaggcgacgaggtaatgcctttactttcacagc

SEQ ID NO: 113 - DNA, 401 bp, Homo sapiens
cacattacttttgtgtttgtaaatgtatgtctgtctatataatctccccctctattctggcaggaaactcattttgtaatggaacaaaatcagtcagctagagtgtccactaaatcgctgttgcgatatacaaaggggctgggttgatatttgatgtcgaagaaagaaagagaaagtggccctgaagataagcctgggccragaggaaaacagatcaggcaccctgagacctgggaggcacaagcatgacgaatttctaccttctgtcttggtacaatgtcgactaaatgtgtctttccactttacatctcaaaacttaggtaatggggtaggaaagcttagtgtaactgagacagaattgaggacaatgaagtctgctggcctggcttcccgctgtaatg

SEQ ID NO: 114 - DNA, 401 bp, Homo sapiens
agggcaagagagttccagccctttaacaccctggtattgcattagggttgctcaacccaagggtcatgatgcataactctctggccatggagaatcgtttgtgtggctagaatttcctgctgtgcccccagcccaatgccaggaaatctctcctctgttttttttaattgatcttttgttcgtatgtgttgtttcttcttytctttgtaggctatgagaaattgaagtaaaagaggagggctagtgatctagtaggaactggatttcaattttgaatctgagtacttggtgacatgagatagggtgttgataggttcacccatcacctaacagcagagcaaggagaagataagatgggatgccaagcacaagaatagatttctgaagggtcccagggccca

SEQ ID NO: 115 - DNA, 401 bp, Homo sapiens
ccaggaaatctctcctctgttttttttaattgatcttttgttcgtatgtgttgtttcttcttttctttgtaggctatgagaaattgaagtaaaagaggagggctagtgatctagtaggaactggatttcaattttgaatctgagtacttggtgacatgagatagggtgttgataggttcacccatcacctaacagcagagsaaggagaagataagatgggatgccaagcacaagaatagatttctgaagggtcccagggcccatgaagaggggccagacccacacagatccctacagtaacttaggagctggcacaaggcagtgaatttaagggcagttggtccttcactctaggtatgtccctttatggtttggcagccaggtatatgccagcttttcag

SEQ ID NO: 116 - DNA, 401 bp, Homo sapiens
gaatagatttctgaagggtcccagggcccatgaagaggggccagacccacacagatccctacagtaacttaggagctggcacaaggcagtgaatttaagggcagttggtccttcactctaggtatgtccctttatggtttggcagccaggtatatgccagcttttcaggggcaggcattgaaagctgctgtgggaagcacrgctggcagaccctccagattctcgaaatttcaccactgtccctggtatgttggctaggcctgcctcagtcctttccctcttactgactctcaagaaagggatgccaacccgtctgctttgtctgggagacttccgctgtttctgagaaatactggctattgagaataccttccggtctctggtcctccccagttcaatca

SEQ ID NO: 117 - DNA, 401 bp, Homo sapiens
tggtttggcagccaggtatatgccagcttttcaggggcaggcattgaaagctgctgtgggaagcacagctggcagaccctccagattctcgaaatttcaccactgtccctggtatgttggctaggcctgcctcagtcctttccctcttactgactctcaagaaagggatgccaacccgtctgctttgtctgggagacttcygctgtttctgagaaatactggctattgagaataccttccggtctctggtcctccccagttcaatcagccacagacagggcacttgtttcctccttgcaccttaagggacaatccacccacctgcctcctcactcctcgctcgcaatgcttactcagcctaggctggagcccacctgaaatggggagggccaggacaactc

SEQ ID NO: 118 - DNA, 401 bp, Homo sapiens
attgagaataccttccggtctctggtcctccccagttcaatcagccacagacagggcacttgtttcctccttgcaccttaagggacaatccacccacctgcctcctcactcctcgctcgcaatgcttactcagcctaggctggagcccacctgaaatggggagggccaggacaactctcttcagaagtaaggggtgtgggygactttgtaatgtctgaggctttgggtttctgaaaatgtcactgtgtttttgctgagtcatggccagttttcctgatctccatttttcctcttactgcttttgccccttttgtctggtcctcattccttagaactcagatatcacaaaatgtctcctgggaatagatgtactgaggcactgctaccctctttctctctct

SEQ ID NO: 119 - DNA, 401 bp, Homo sapiens
tttggggtagggggaggagttgcaaaactttaagatatgggaaccacgacattttttaactcattagaccctggcttttagggccatggcttgtattttacacttgtttagaagctatgttcttcaaaaataaaattagaactcaccaggtgttttaattattctttacactcctgcttcaacacttttgcatcgtgcttsctatctacgtaacacgaagaatccccctccactgggacaaccatctagaaagggggaagagaaggagaggttaaggaagagactgagaggatcagatatctagagaaaatccatttgaagcaaggtagactggctttctgcctaattccccctttctcttagcaataacagtttcataattagctttagccagatttcta

SEQ ID NO: 120 - DNA, 401 bp, Homo sapiens
ctaattcagaaaaagcagaagaataatatgatacacatatcccctgctcatatttaaaatgttcactttttgtcacttaggcttcagatagtatatctcacagttaaaattgaaaaccattttgttcctcctctccatcctagtcttgcttctttctctccagcgatgaccactgtcagtaatttgatatatactattccrgtccatgtttttattagctcattacagattttatatatcagataatagatggtattgctttgtagttgtaaaattttggctgaataatacactgcatgaattgttttgcaatttgtttccagtaaacaatatgtttttgaactgtatattttcttgcatatagatacaatcccttccttttttttttttttttttttcct

SEQ ID NO: 121 - DNA, 401 bp, Homo sapiens
tttcaattttaactgtgagatatactatctgaagcctaagtgacaaaaagtgaacattttaaatatgagcaggggatatgtgtatcatattattcttctgctttttctgaattagaaaaaaataggagggtctttcccgggggcatcttcattgtgccatcctgctcatgtgatgggaagaaattcatccctggcgttatkgctcctaggaacatcctgtgggagatgaaagtatgtgtgctgttttgaaattaaaagcaaagcaggaaaagtcaagagtcctctttttcagagagaaaaacttgcagaacctgtggagttgttacccccatactgttgacaatcctcactcttcccttcccttctcagccagaaagcagcctttattatttattattttt

SEQ ID NO: 122 - DNA, 401 bp, Homo sapiens
gcctaagtgacaaaaagtgaacattttaaatatgagcaggggatatgtgtatcatattattcttctgctttttctgaattagaaaaaaataggagggtctttcccgggggcatcttcattgtgccatcctgctcatgtgatgggaagaaattcatccctggcgttattgctcctaggaacatcctgtgggagatgaaagtrtgtgtgctgttttgaaattaaaagcaaagcaggaaaagtcaagagtcctctttttcagagagaaaaacttgcagaacctgtggagttgttacccccatactgttgacaatcctcactcttcccttcccttctcagccagaaagcagcctttattatttattatttttattttttgtacagggctcccagccagcctcaag

SEQ ID NO: 123 - DNA, 401 bp, Homo sapiens
ggcgttattgctcctaggaacatcctgtgggagatgaaagtatgtgtgctgttttgaaattaaaagcaaagcaggaaaagtcaagagtcctctttttcagagagaaaaacttgcagaacctgtggagttgttacccccatactgttgacaatcctcactcttcccttcccttctcagccagaaagcagcctttattatttwttatttttattttttgtacagggctcccagccagcctcaagggtctatgtatgctagtgtgattgctgtgtttttctaaccacttcaaaggtcatgttcacttatctgtgaaaacaagttctcattccttgcaccctatttgccttcatgtctcaggaaatagggacgggcaaatcacttgccaggtgggaagaaaataa

SEQ ID NO: 124 - DNA, 401 bp, Homo sapiens
ataaaggaatgttgtacacacaccctactctgaagaccagagcggaaagggaacctatctgtgtatcccaagggggctgtgagcaggagagaaagacagaaatacagcagagagatgtgacttacatgtcctaatctccccactcccgaggtgaaccccaagatcagaaggaggcatagtgaacatttccaactagatatsgaaggaacctgagaatatttggaggaggagaccagcgacactgtttcccaaacacagcttaggggagactgggagcatcttggaagagccccacacagagccatgttcagcatggcctgagtgagggcggaaactttagcggtggagcaggaaagtgaccagctaggcaagcctccagcagcactgcttgcactccatgg

SEQ ID NO: 125 - DNA, 401 bp, Homo sapiens
gaggaggagaccagcgacactgtttcccaaacacagcttaggggagactgggagcatcttggaagagccccacacagagccatgttcagcatggcctgagtgagggcggaaactttagcggtggagcaggaaagtgaccagctaggcaagcctccagcagcactgcttgcactccatggcctgcccgggctgaacagtggrcctaagaattcataagtgctccagagagggacaaggagggactaagagggccctgagttaagaaaaagacatcacattgcccccaaatgccactggggaccagtggtgcagagtgaatcaatcggtgactgtgatacacagtttagtttgtcctctgtcataagaataaatatcagatatcataatcagggaaagcacaa

SEQ ID NO: 126 - DNA, 401 bp, Homo sapiens
actaagaaactggcagtacctggactacagattcagcaggaacaagtccacaaactgagcaggcagttaatgagcttagcacccatcatccctggctcagagtatggccctggaattctttgttgcaataaaatataaaatagaacctgaaaagaagcttggctatgagcaagtaaggcttccttcatttgcctggtcccrcaggctctctgttgcagtggcggcaaacccacccagatgggttgtgctctgcaacttcaagcagccctatcaccagtgagggtggtggcaatgctccaggccccttggggcaggggaggtaagacagcatgccatctgatgctgccccccttgctgacctgggcagcgggcacattcctcagttccctctgacccatggg

SEQ ID NO: 127 - DNA, 401 bp, Homo sapiens
caataaaatataaaatagaacctgaaaagaagcttggctatgagcaagtaaggcttccttcatttgcctggtcccacaggctctctgttgcagtggcggcaaacccacccagatgggttgtgctctgcaacttcaagcagccctatcaccagtgagggtggtggcaatgctccaggccccttggggcaggggaggtaagamagcatgccatctgatgctgccccccttgctgacctgggcagcgggcacattcctcagttccctctgacccatgggctgtcctgaactgaactctgctcagctccatcccctctgctcgcctgctccctgcagagtgagcccgccccttgctggctaggctttccctgccttatttggtttaaattacttttttgaacttt

SEQ ID NO: 128 - DNA, 401 bp, Homo sapiens
gctctctgttgcagtggcggcaaacccacccagatgggttgtgctctgcaacttcaagcagccctatcaccagtgagggtggtggcaatgctccaggccccttggggcaggggaggtaagacagcatgccatctgatgctgccccccttgctgacctgggcagcgggcacattcctcagttccctctgacccatgggctgycctgaactgaactctgctcagctccatcccctctgctcgcctgctccctgcagagtgagcccgccccttgctggctaggctttccctgccttatttggtttaaattacttttttgaactttggtctcattatttttggaccatcatttggtctggaagattttctttccacgggttccttctgttcttccttttcaccct

SEQ ID NO: 129 - DNA, 401 bp, Homo sapiens
acccagcctctctcattttaaaagtggggagtaggagtatttatctcactgcgttgttgtgcagcttaaatctgttgatatatgcaaagacttaggagactgattggtatatagtaatctctatataagtggttgctattattatttgctattctgagtgagaaaacaaaaatccagagagataaggtaacttgaaaaagytcccagctagtaaatggtagagctaggcctcaaatccaggattaattaactctaaattcaagtacttaattgttataatattctgcctcctcaggagacataaaaaatatagagacattagaaaacaaaaaacatcagagttactggagagaagtagggtgaatcagaaatacaggaacagaaactttgtggcagaaatc

SEQ ID NO: 130 - DNA, 401 bp, Homo sapiens
gagttaattaatcctggatttgaggcctagctctaccatttactagctgggagctttttcaagttaccttatctctctggatttttgttttctcactcagaatagcaaataataatagcaaccacttatatagagattactatataccaatcagtctcctaagtctttgcatatatcaacagatttaagctgcacaacaaygcagtgagataaatactcctactccccacttttaaaatgagagaggctgggtgcagtagctcatgtctataatcctagcacttcaagagatcggggtgggagaatcccttgagcccaggagttcaagaccagcctgggcaatgtagcgagacccagtctctacaaaaaacaaaaaacttagccaggagtggtggtgcaca

SEQ ID NO: 131 - DNA, 401 bp, Homo sapiens
tgtagcgagacccagtctctacaaaaaacaaaaaacttagccaggagtggtggtgcacacctgtggttccagctacatgggaggctgagacaggaggactgcttgaacaccggaggttgaggctgtagtgagccatgttagtgccactgcactccagcctgggttaaagagcatgacctagtctcaaaataataaataaayaaaccaaggcacagagaagttaagtaagctgccataatcacacagcttgtgaggagcaatgctgtgatctaacactatagagtctatgaccccaatcacttcatcatactgcctctttcccccgccccccaccccaccacccaggagtttaagagtggagatttaataggcagaagaaagagaaaggaaaacagctctct

SEQ ID NO: 132 - DNA, 401 bp, Homo sapiens
tttcattaagagacagtatgagggcctttcctctcagaagtcctctctctcacactagatagagagctgttttcctttctctttcttctgcctattaaatctccactcttaaactcctgggtggtggggtggggggcgggggaaagaggcagtatgatgaagtgattggggtcatagactctatagtgttagatcacagcwttgctcctcacaagctgtgtgattatggcagcttacttaacttctctgtgccttggtttatttatttattattttgagactaggtcatgctctttaacccaggctggagtgcagtggcactaacatggctcactacagcctcaacctccggtgttcaagcagtcctcctgtctcagcctcccatgtagctggaaccacag

SEQ ID NO: 133 - DNA, 401 bp, Homo sapiens
catatagtgagagaattaaacaccccctttctggtcctccttgggccaaacttctctcagtttcacaacttgttctagaacctagcccgtggtttacttacaaagtattccttttactttcttagctattaatagaacaaagcaaaacctctgtgagtacaaaattatgtgatccagagcctcagcctgtgtctacctgaytgtgacctagagagtcctgtccaaataattctccagatctaaagaaatcccccattgccataaactcatactggtcctaattctgtttccgcagccatatagttctacttctcattttattctccttcaaaaattgtgaagtatttctaatacaggatataaaaaattctagagaacaacttaaagcacacacatgtgcc

SEQ ID NO: 134 - DNA, 401 bp, Homo sapiens
aaaaattgtgaagtatttctaatacaggatataaaaaattctagagaacaacttaaagcacacacatgtgccacaaccaaaattttgaaaaatattaatgttatcatatatgtttcaggccttttaaaaataaattaaacactaaaatacaactgaagccccatcccctcattttcttcccctttttggtaaccccacacmtaaagatatgtatcccacttccatgagtttttatatttttagtacatttttatacaaaaacatttattattgttttgtgtacctggtaactttaaaaataaatgatgccatactgtttataagattctgctccttgctttttagtaaatcaatgtttattttcacttcttttgtgttttctacttttatgcttctttttt

SEQ ID NO: 135 - DNA, 401 bp, Homo sapiens
ctgtttataagattctgctccttgctttttagtaaatcaatgtttattttcacttcttttgtgttttctacttttatgcttcttttttaaatctttttttttctccttccctgtcttgtgttggattaatagggctttagctattctctccacatcaacgccttttcaccaatcagcttctgacttacacgttacattgtmtttctttccttctttctttcttttttttttttttgagacggagtcttactctgtcgcccaggctggagtgcagtggcgctgtcgcccaggctggagtgcagtggcgcaatcttggctcactgcaaactccacctcctgggttcacaccattctcctgcctcagcctcccaagtagctgggactacaggcgcccaacacca

SEQ ID NO: 136 - DNA, 401 bp, Homo sapiens
agattctgctccttgctttttagtaaatcaatgtttattttcacttcttttgtgttttctacttttatgcttcttttttaaatctttttttttctccttccctgtcttgtgttggattaatagggctttagctattctctccacatcaacgccttttcaccaatcagcttctgacttacacgttacattgtatttctttcyttctttctttcttttttttttttttgagacggagtcttactctgtcgcccaggctggagtgcagtggcgctgtcgcccaggctggagtgcagtggcgcaatcttggctcactgcaaactccacctcctgggttcacaccattctcctgcctcagcctcccaagtagctgggactacaggcgcccaacaccatgcctggct

SEQ ID NO: 137 - DNA, 401 bp, Homo sapiens
ctttttagtaaatcaatgtttattttcacttcttttgtgttttctacttttatgcttcttttttaaatctttttttttctccttccctgtcttgtgttggattaatagggctttagctattctctccacatcaacgccttttcaccaatcagcttctgacttacacgttacattgtatttctttccttctttctttctttyttttttttttgagacggagtcttactctgtcgcccaggctggagtgcagtggcgctgtcgcccaggctggagtgcagtggcgcaatcttggctcactgcaaactccacctcctgggttcacaccattctcctgcctcagcctcccaagtagctgggactacaggcgcccaacaccatgcctggctaatttttttgtattt

SEQ ID NO: 138 - DNA, 401 bp, Homo sapiens
acgccttttcaccaatcagcttctgacttacacgttacattgtatttctttccttctttctttcttttttttttttttgagacggagtcttactctgtcgcccaggctggagtgcagtggcgctgtcgcccaggctggagtgcagtggcgcaatcttggctcactgcaaactccacctcctgggttcacaccattctcctrcctcagcctcccaagtagctgggactacaggcgcccaacaccatgcctggctaatttttttgtatttttttttttttttttagtagagacagggtttcactgtgttagccaggatggtctcaatctcctgatctcatgatccacctgccttggcctcccaaagtgctgggattacaggtgtgagccaccatgcccggcct

SEQ ID NO: 139 - DNA, 401 bp, Homo sapiens
attgtatttctttccttctttctttcttttttttttttttgagacggagtcttactctgtcgcccaggctggagtgcagtggcgctgtcgcccaggctggagtgcagtggcgcaatcttggctcactgcaaactccacctcctgggttcacaccattctcctgcctcagcctcccaagtagctgggactacaggcgcccamcaccatgcctggctaatttttttgtatttttttttttttttttagtagagacagggtttcactgtgttagccaggatggtctcaatctcctgatctcatgatccacctgccttggcctcccaaagtgctgggattacaggtgtgagccaccatgcccggcctgtatttcttatattttaatggttaccgataaattgtta

SEQ ID NO: 140 - DNA, 401 bp, Homo sapiens
atcttggctcactgcaaactccacctcctgggttcacaccattctcctgcctcagcctcccaagtagctgggactacaggcgcccaacaccatgcctggctaatttttttgtatttttttttttttttttagtagagacagggtttcactgtgttagccaggatggtctcaatctcctgatctcatgatccacctgccttrgcctcccaaagtgctgggattacaggtgtgagccaccatgcccggcctgtatttcttatattttaatggttaccgataaattgttaaatatacatatatatacagtcttgcatcacttaacaacaggaatatgtttgagaaatgtctcatcaggcaattctgtcattgtgcaaacatcgtagagtgtacttatacaaccc

SEQ ID NO: 141 - DNA, 401 bp, Homo sapiens
tacaggcgcccaacaccatgcctggctaatttttttgtatttttttttttttttttagtagagacagggtttcactgtgttagccaggatggtctcaatctcctgatctcatgatccacctgccttggcctcccaaagtgctgggattacaggtgtgagccaccatgcccggcctgtatttcttatattttaatggttacygataaattgttaaatatacatatatatacagtcttgcatcacttaacaacaggaatatgtttgagaaatgtctcatcaggcaattctgtcattgtgcaaacatcgtagagtgtacttatacaaccctagatggtatagctgattacacatctaggctatatggtatagcccattgctgctaggctacaaacctgtacagc

SEQ ID NO: 142 - DNA, 401 bp, Homo sapiens
atctcatgatccacctgccttggcctcccaaagtgctgggattacaggtgtgagccaccatgcccggcctgtatttcttatattttaatggttaccgataaattgttaaatatacatatatatacagtcttgcatcacttaacaacaggaatatgtttgagaaatgtctcatcaggcaattctgtcattgtgcaaacatcrtagagtgtacttatacaaccctagatggtatagctgattacacatctaggctatatggtatagcccattgctgctaggctacaaacctgtacagcatgttttggtaatgaatactgtagaaaattgtaagtatttgtgtatctaaacatatttaaatatagaaaaggtatggtaaaaatatggtattataatcttctggg

SEQ ID NO: 143 - DNA, 401 bp, Homo sapiens
taaacaaaacagaaatcttagtatgttttaactagtttattgagcactccctaccaccaacattctttgatgctattgtctagacttttggccccattttttaatacaaaatttatttttatgtagttaatagttaatgcacttttactgaactactttacaaatttttctctcttgctcccaattgttctctctgatttytcttttctctttcttgaactaaatgcaagggtctgggagcagtaaattttcttagtctttttgtaacttaatgtatccttattttgccctcacaattgattaatttagctgggatgaaattctaggttgacttttttcttactgctatgaagatatcaactcatctgcaatgggggtctaatctagctgaagatcattct

SEQ ID NO: 144 - DNA, 401 bp, Homo sapiens
agtttattgagcactccctaccaccaacattctttgatgctattgtctagacttttggccccattttttaatacaaaatttatttttatgtagttaatagttaatgcacttttactgaactactttacaaatttttctctcttgctcccaattgttctctctgatttctcttttctctttcttgaactaaatgcaagggtytgggagcagtaaattttcttagtctttttgtaacttaatgtatccttattttgccctcacaattgattaatttagctgggatgaaattctaggttgacttttttcttactgctatgaagatatcaactcatctgcaatgggggtctaatctagctgaagatcattcttttgtgaatataatcattaatcctttgtgagta

SEQ ID NO: 145 - DNA, 401 bp, Homo sapiens
ggttttattttcatttgttctgtttgcattttatatgaactttcaccaaaagattcacgacttcaattctggaaacttacatcattctctatttcttttccctggaactcctaatcagatgcatactggagtctctcaatccatttctattccccttaacttcccatacaaacattaatatgtttgtatgcatatatatgyataatttttctttttgtctatattttgtttatatttctgagttctattttcccattgtaatagattatccagagaaacagaaccaatatggtgtgtatatatatagagagagatttgttataaggaattgtctgacacaattatggagactttcaagtttaaatctgcagtgtgggccagcaggcttgagatgcaggaca

SEQ ID NO: 146 - DNA, 401 bp, Homo sapiens
gaactcctaatcagatgcatactggagtctctcaatccatttctattccccttaacttcccatacaaacattaatatgtttgtatgcatatatatgtataatttttctttttgtctatattttgtttatatttctgagttctattttcccattgtaatagattatccagagaaacagaaccaatatggtgtgtatatatakagagagagatttgttataaggaattgtctgacacaattatggagactttcaagtttaaatctgcagtgtgggccagcaggcttgagatgcaggacagctgatggtgcagatgaagtcctattaagtgatctgctggagaatctcctcttacttgagagaagattggcttttgttctattcagtcctactcatctgatttg

SEQ ID NO: 147 - DNA, 401 bp, Homo sapiens
atttctgagttctattttcccattgtaatagattatccagagaaacagaaccaatatggtgtgtatatatatagagagagatttgttataaggaattgtctgacacaattatggagactttcaagtttaaatctgcagtgtgggccagcaggcttgagatgcaggacagctgatggtgcagatgaagtcctattaagtgawctgctggagaatctcctcttacttgagagaagattggcttttgttctattcagtcctactcatctgatttgaagagacctgctcatgttatggagggcaatctgctttactctgccaattgaaatgttaaattcacccaaaaatatccacacagaaacacccagaataatgtctgaccaaatatctgggcaacccatagc

SEQ ID NO: 148 - DNA, 401 bp, Homo sapiens
ttttgttctattcagtcctactcatctgatttgaagagacctgctcatgttatggagggcaatctgctttactctgccaattgaaatgttaaattcacccaaaaatatccacacagaaacacccagaataatgtctgaccaaatatctgggcaacccatagcccagtcaaattgccacataaaattaattatcactgccayttaaatactctctttttattgtttgctctaaactctacttcagaatttattccatttattattttttaaaatagcaattttttagtacacaatttactattgttactatattctcttatataggtttaatttttgcttttttgtttcatcatttcttattttatcttttaattttttattatactttatctctttgacaa

SEQ ID NO: 149 - DNA, 401 bp, Homo sapiens
atctgctttactctgccaattgaaatgttaaattcacccaaaaatatccacacagaaacacccagaataatgtctgaccaaatatctgggcaacccatagcccagtcaaattgccacataaaattaattatcactgccacttaaatactctctttttattgtttgctctaaactctacttcagaatttattccatttattrttttttaaaatagcaattttttagtacacaatttactattgttactatattctcttatataggtttaatttttgcttttttgtttcatcatttcttattttatcttttaattttttattatactttatctctttgacaatcctaaagacttcattttaaatcattttcaaattgtcctatcattttcatttcatctgacg

SEQ ID NO: 150 - DNA, 401 bp, Homo sapiens
tttattgtttgctctaaactctacttcagaatttattccatttattattttttaaaatagcaattttttagtacacaatttactattgttactatattctcttatataggtttaatttttgcttttttgtttcatcatttcttattttatcttttaattttttattatactttatctctttgacaatcctaaagacttcaytttaaatcattttcaaattgtcctatcattttcatttcatctgacgtgaatttattttcccacattttattttgttaagtgcttgtcttatggttaatattcctcacgtgtctcggattttggttttgtggttcatgttctatgagtccacttcaggttatatatgggcagtttgggatttctatcttggtgtgatatta

SEQ ID NO: 151 - DNA, 401 bp, Homo sapiens
gtttcatcatttcttattttatcttttaattttttattatactttatctctttgacaatcctaaagacttcattttaaatcattttcaaattgtcctatcattttcatttcatctgacgtgaatttattttcccacattttattttgttaagtgcttgtcttatggttaatattcctcacgtgtctcggattttggttttstggttcatgttctatgagtccacttcaggttatatatgggcagtttgggatttctatcttggtgtgatattaggaatatcagatttaatctctgtatcagtccattttcacactgctgataaagacatacctgagactgggtaatttatagagaaaaagaggtttaatggactcacagttccccgtggctgggcaggcct

SEQ ID NO: 152 - DNA, 401 bp, Homo sapiens
agaaatgagaaccaagcaaaaagggattcccattataaaaccaccagatctcctcaaacttattcactagcactaacaatatgggggaaccacacccaagattcaattatctccctccaggtcccttgtgcaacatgtgggaattatgggagctacaattcaagatgagatttgggtggggaaacagagaaaccatatcartctctaagctagccggtgccttgacccaggccttccctcaggggctgtgctccaccttcttctgccttcctagacatgcagttttatgtaagccatagattcatgttgcagcatttttccccagcctcttcatgacttgacagcaaaagcagaaatatggggatttctatacccaattttaaactaacagaattgcatat

SEQ ID NO: 153 - DNA, 401 bp, Homo sapiens
gtaaaggcctcaaaacgtgaagtgataacatggttttgactaaagttctcccatttctattctccctctattgttacagcttctctaatggttctctttgtattcctggcattctctggaggcttctgataaaaacagggtctttaattgccccccttttccagtcaggtccccaataagaaacagatgttgtactcaaakggggtaattgaggagagtttactaaagggactgcttgcaaagatgtagtccaagctaagataaattggaaaggattggtgaagctcccaagccaacaacaacaggcctagtagagaactatcactgtgctataggagagagctgcctggcaggagtgccagcctttgatagaggaacatggtccactactgaaccttaac

SEQ ID NO: 154 - DNA, 401 bp, Homo sapiens
caaaacgtgaagtgataacatggttttgactaaagttctcccatttctattctccctctattgttacagcttctctaatggttctctttgtattcctggcattctctggaggcttctgataaaaacagggtctttaattgccccccttttccagtcaggtccccaataagaaacagatgttgtactcaaagggggtaattraggagagtttactaaagggactgcttgcaaagatgtagtccaagctaagataaattggaaaggattggtgaagctcccaagccaacaacaacaggcctagtagagaactatcactgtgctataggagagagctgcctggcaggagtgccagcctttgatagaggaacatggtccactactgaaccttaacttggtaaaga

SEQ ID NO: 155 - DNA, 401 bp, Homo sapiens
aggagagtttactaaagggactgcttgcaaagatgtagtccaagctaagataaattggaaaggattggtgaagctcccaagccaacaacaacaggcctagtagagaactatcactgtgctataggagagagctgcctggcaggagtgccagcctttgatagaggaacatggtccactactgaaccttaacttggtaaagasagagaccaaggagtaaatactcccaactccttctcctgccctttgttcctctgcttcccatgggccaaactcaacaggaaatcagactgcaagggaccccttaacatagtcaagaaaggtcagcctctcagggcacagagtagggcaaggagtggatctggatgtggtaaagaaagaacacacagaatacagaatagccc

SEQ ID NO: 156 - DNA, 401 bp, Homo sapiens
tttactaaagggactgcttgcaaagatgtagtccaagctaagataaattggaaaggattggtgaagctcccaagccaacaacaacaggcctagtagagaactatcactgtgctataggagagagctgcctggcaggagtgccagcctttgatagaggaacatggtccactactgaaccttaacttggtaaagacagagacmaaggagtaaatactcccaactccttctcctgccctttgttcctctgcttcccatgggccaaactcaacaggaaatcagactgcaagggaccccttaacatagtcaagaaaggtcagcctctcagggcacagagtagggcaaggagtggatctggatgtggtaaagaaagaacacacagaatacagaatagcccaagacta

SEQ ID NO: 157 - DNA, 401 bp, Homo sapiens
gtccaagctaagataaattggaaaggattggtgaagctcccaagccaacaacaacaggcctagtagagaactatcactgtgctataggagagagctgcctggcaggagtgccagcctttgatagaggaacatggtccactactgaaccttaacttggtaaagacagagaccaaggagtaaatactcccaactccttctccygccctttgttcctctgcttcccatgggccaaactcaacaggaaatcagactgcaagggaccccttaacatagtcaagaaaggtcagcctctcagggcacagagtagggcaaggagtggatctggatgtggtaaagaaagaacacacagaatacagaatagcccaagactacatttctcaggtcacttccatcttcacatt

SEQ ID NO: 158 - DNA, 401 bp, Homo sapiens
aagataaattggaaaggattggtgaagctcccaagccaacaacaacaggcctagtagagaactatcactgtgctataggagagagctgcctggcaggagtgccagcctttgatagaggaacatggtccactactgaaccttaacttggtaaagacagagaccaaggagtaaatactcccaactccttctcctgccctttgwtcctctgcttcccatgggccaaactcaacaggaaatcagactgcaagggaccccttaacatagtcaagaaaggtcagcctctcagggcacagagtagggcaaggagtggatctggatgtggtaaagaaagaacacacagaatacagaatagcccaagactacatttctcaggtcacttccatcttcacattacagaaata

SEQ ID NO: 159 - DNA, 401 bp, Homo sapiens
acatctgtggtttcctctgttaggacatgctccccaccctgaaccctgcaccacacctctggctatcactagcctcattcccttggggccttcaggtcttaattcagcccaggtagttgctcctcttacatgctgcctttgcagtttcaaaggaggggaactgagagaaagtggtcacacagagatcaagaggggaggagmctttaggcaaggagtgcctcaatgcagttgataagcatccaaaagtttggattgggtttataattcagcaagaaatcagtaacttcctggagagaaatttcagggggctataaagtcagaaactgaacaacaggaggttgagtagcaaataagaataaagtgcagacagtgcctttagacaactctttcaagaaatgacc

SEQ ID NO: 160 - DNA, 401 bp, Homo sapiens
tggatgcttatcaactgcattgaggcactccttgcctaaagtctcctcccctcttgatctctgtgtgaccactttctctcagttcccctcctttgaaactgcaaaggcagcatgtaagaggagcaactacctgggctgaattaagacctgaaggccccaagggaatgaggctagtgatagccagaggtgtggtgcagggtkcagggtggggagcatgtcctaacagaggaaaccacagatgtggtatccattgcttcttatttgccatcagtgactgcatggttgaccaccaaactctatagggtcactctgaagaatagaaaaagctgaagtggtctagaagaaattagattttctataggaaagacataaagaaaacttggcagaggtatctgttttta

SEQ ID NO: 161 - DNA, 401 bp, Homo sapiens
aaagtctcctcccctcttgatctctgtgtgaccactttctctcagttcccctcctttgaaactgcaaaggcagcatgtaagaggagcaactacctgggctgaattaagacctgaaggccccaagggaatgaggctagtgatagccagaggtgtggtgcagggttcagggtggggagcatgtcctaacagaggaaaccacasatgtggtatccattgcttcttatttgccatcagtgactgcatggttgaccaccaaactctatagggtcactctgaagaatagaaaaagctgaagtggtctagaagaaattagattttctataggaaagacataaagaaaacttggcagaggtatctgtttttaaaaatacataaatctcggcctcgaccaacacttattt

SEQ ID NO: 162 - DNA, 401 bp, Homo sapiens
gtgaccctttcacccaagaccatttcttcatcccactagaacctcagttctcagcactgagcaatatcagctgacagcctttcatccagcaggacaacagaactgtgggaccccagcagagacaaggctgtcagtgaggcctgtcctggtgggctgtcatcccacgtggagaggtgagaaaagtagggtggaagaggaaaygatgctgaagactgaggaggcaggtggaattcaaagttggtctttttttttcttttccaaattaaagagatgttttctctgccacagcttctgggccctgatacagcaataagtcaaacaccagcattcatccccttgcaaatgggaaccacctccccaccttggagcacagacagattgtagttgagacgcccagtgga

SEQ ID NO: 163 - DNA, 401 bp, Homo sapiens
gaaccacctccccaccttggagcacagacagattgtagttgagacgcccagtggaaaaagctgagtatggctgggcagcctcacacaggcctttgcctctagatccccttgagctcccttcccaagagtccttgacaaatatgcagggctaagaggctgtccaaggaatgcagccgtctcagccattcattaccaaggctmaaaaccacaggggcaggaagagcagacagagctgggtaggatccgtctccggtgaggtttggaaatgggagatacccctgactgggcaggctgaattgatgtggtctggcctaggggcaaggggatggacagaatgaccatcaccactctcagccagcctggtgggtctatgaaaagagatgtttgattcattctcgggc

SEQ ID NO: 164 - DNA, 401 bp, Homo sapiens
ggcaagaatgataagaaggaacaaacctggaaccatgattactcattcatggtcttccagtttacactctactgtgatgaccatgaaagagaaaaagaattagtaaaaaagaaaaaaatacatagacagactgccctcccagacacagaaaaattattttcttacccataagaaaattccatcaaacatgaagattaaatrgggacctaaaatctgatacctaccagtatctggacacacctggatcctgtggaggtggctctgggcagacagacaagttggaggggcagatggaattagtcaagtagggaaagtcagagtgaataagtcaggaaagccctcccacaggaagaattttgagtaaggatttgtgaatgagaaatccagaataaaaagaggca

SEQ ID NO: 165 - DNA, 401 bp, Homo sapiens
tcatcttcagtcaacttcagtggacttttaaaattcaacaaatgatttttcaatgtctatctcaagccaggcacaggtttgttactggaaagcaacacaagatgttaacagaatggatgtttctgtcctagaagaaatgtctgcattctacaaagaaaaatggcagccaggcaccatggctcatgcctgtaatcccaacastttaggaggcaaagtcaggaggatcattttaaggccaggagttcaagaccatcctgggcaacatagcaaaacctgacttctacaaaaaaaatgtaaaacatagccaggcatggtggcttgcacctgtcatcccagctactcaggaggctgaagcaggacgattgcttaagcccaggagttggaggttaccatgagctatg

SEQ ID NO: 166 - DNA, 401 bp, Homo sapiens
cctttactgcagcctgttttatttatttatttatgacccgtattttgtgccgacctcctatatatcctgtgacttagaatgcctaaccctctggcaatgcagcccagtgggtcttagccttattttacccagttcctatttaagatggagttgctgtggttcaaatgcctctaacactggggcttataaaaagaaaattartaagatattcaatgttcggagtgagggctgtggataaaatgtcacacccacagacagaagagaggaaagaaaagtaagctattgaatgtttaagtgggacttaacagaggccaagatagagagagagagagacaaaaagaaatgggcagcgggagagcagcatgtctcctatgtgtgtgtgtctgtgtgtctgtgtgtgt

SEQ ID NO: 167 - DNA, 401 bp, Homo sapiens
ttatttatttatttatgacccgtattttgtgccgacctcctatatatcctgtgacttagaatgcctaaccctctggcaatgcagcccagtgggtcttagccttattttacccagttcctatttaagatggagttgctgtggttcaaatgcctctaacactggggcttataaaaagaaaattagtaagatattcaatgttcrgagtgagggctgtggataaaatgtcacacccacagacagaagagaggaaagaaaagtaagctattgaatgtttaagtgggacttaacagaggccaagatagagagagagagagacaaaaagaaatgggcagcgggagagcagcatgtctcctatgtgtgtgtgtctgtgtgtctgtgtgtgtgtgtgtgtctgtgtgtct

SEQ ID NO: 168 - DNA, 401 bp, Homo sapiens
cttagccttattttacccagttcctatttaagatggagttgctgtggttcaaatgcctctaacactggggcttataaaaagaaaattagtaagatattcaatgttcggagtgagggctgtggataaaatgtcacacccacagacagaagagaggaaagaaaagtaagctattgaatgtttaagtgggacttaacagaggcyaagatagagagagagagagacaaaaagaaatgggcagcgggagagcagcatgtctcctatgtgtgtgtgtctgtgtgtctgtgtgtgtgtgtgtgtctgtgtgtctgtgtgcgtgtgtgtgtgcatgcatgcaactctcatagttaattggcttgtgcccatggagtcggtaagagtgcggccactaagctcacgcctga

SEQ ID NO: 169 - DNA, 401 bp, Homo sapiens
gcatagcaacatcaaaaatcaaatggaccagggagatgagatatattggttcagaaagtatctggctgcaagtagcagaaaaagtaacgaagaatggcttaagctaaaagggtgttattttctcttagaataacaactgaataggtataacctcgagtctatgctaggtaggaaggaagtgaagtttggttaggggtgatkggtattcagctaatgcacttggcggataagaaaaacatgatgacttcagaggcatctggcaagcaagggagagagggccaaagaagaccagtgtggtctgtgcagggattcagtggttaccatatcagaggaaaagttttgggagacaaaatcacctgcaggtggccttgagacagggttgctaaagtgcaatgaggaaa

SEQ ID NO: 170 - DNA, 401 bp, Homo sapiens
cctcctcttttgaggtacagacttgcaatgccaccagcctcctttccttcctagctttgccatttgcaggccggttactgtccactcagggcaggctggtattggtatctgtgctgatgtgttctcacctctgctacctcttctattaatgttctagatgtatctgtcgggaaaggcagaagagggtggagtggggatgaytgagtttgtggttggcttggaacacccataatcccagcactttgggaggccgaggcaggaggattgcttgaggccaggaattggagaccagcctggtcagcatagcgggacctcatctctcccaaaaataaactaaaataaaataaaaataagagtttgtgatctctaagggatgctaagaaaggaacaaagtccagatg

SEQ ID NO: 171 - DNA, 401 bp, Homo sapiens
aaagaggaggcattggggtggcaacattgccaatgggctagatagaagtaagagtgagagccagaagaatctaccccactgtctgccaggtataccacagaagccaagcaaactgcttctttcctggggagggtctctgtgctccaagaaccatgctcatcctagcaacaggaagctctttagtgccagcctgttgcaatrgacttcaatggctatttctcctgttaatgccttttctgttttgctctctcaagatcttaactgcaactgcccctattactccaactgagattacaatttttgagaaacctgcaaacaggacagctttagagtgtaccccaaatacaaggacacagaatcaaattccatgaacccctataaatcatgaatccactggaatg

SEQ ID NO: 172 - DNA, 401 bp, Homo sapiens
cccaaatacaaggacacagaatcaaattccatgaacccctataaatcatgaatccactggaatggaagccccatgggggctagactttgctgtttgctcactgatgattcttgagtgataagaaccaggcctggcacatagtaggtaccttctatgtgtgttgaataagagcagctgtgtcagagagctgctccagcactrcacccagtaacatccctccctgccagtaatcaatcatcttgccaaagaccaggtcctttgactcttccttgcctctggtgaagtttctgtgcacctgaggttaggaagtgagctctcttcctcttcacagcctggcgtcctaggttccttgcctctcatagaactcgttcctatttctcacgaggtcttaacactcagac

SEQ ID NO: 173 - DNA, 401 bp, Homo sapiens
accttctatgtgtgttgaataagagcagctgtgtcagagagctgctccagcactgcacccagtaacatccctccctgccagtaatcaatcatcttgccaaagaccaggtcctttgactcttccttgcctctggtgaagtttctgtgcacctgaggttaggaagtgagctctcttcctcttcacagcctggcgtcctaggtwccttgcctctcatagaactcgttcctatttctcacgaggtcttaacactcagacacccagcttgccagcattcaaacatgatgctttcctctgcggcctggaagacaaccatacttgccttctcggggtcaaaacagcctcagagcctcccagaagccagctcttgggtttctgtcccgccccctcccctctgccacttg

SEQ ID NO: 174 - DNA, 401 bp, Homo sapiens
cagaaaggagaacagaggctagtgtggcaaaccttgcagcccggaaggaagcttagattttattcaaggtgcgctgggaagaacgtcactaaagaactctgaagaaggtcatgggttccctgagagaaatccctgggggccttactagagtgacgcagactcacccctgcctcatcccccaacttttgtcctcacctcctmcccccatgcccagtcccagcaaccggggggtgggtgggctaagatcatgtttaattatttcacagattcttccctcctctgataggggccatgaaaagacatacagttcaaatcagtccatttggcaacctctgagcaactggaaattttggtcaatagtcaacactcagcaaaaatgagatttttgaattattggaagt

SEQ ID NO: 175 - DNA, 401 bp, Homo sapiens
ctgacccactcaccttccagtattcgtcaagcatcccatccttttgaaatttacaagtttataatatttacatgccattttgtaaccataaggtctttttaactttttgaacatttatatgaatattgaaaaccaataaacagtatttgcaaagttctatttactccaaacctgatagtgtatttggatctagagagaaasaatagaggttatatgttgccacctaaaagaaaatgcttcaaagatcaagatcaagtagatgctccttttagattccattaattattcagaattatgccacagattatgacaaaaaggggaggaacataaatatacagtcttgtattatcactaagatggataactcgggaagaaggaatgtattacatagaaaaattacc

SEQ ID NO: 176 - DNA, 401 bp, Homo sapiens
ttttcttcctcaaatcccataaggaataaggcagatgccaaaggagacacatcctggtggtgattcgatgattattaataaagggagggaggggctattagaatagggagggaaagagcacaatccgaagtcttccccagttgactctgaagcactttctccatgcctataatcactgctctaagagcagcaatactataytctgctcacatgacctctgggcagtttactctctcaagtctatgttgggcatgttaggtccacagtggttgagattagttgtgtcctagctaagacctctttgcaagttgtttcaccatcaaaattatgtttcaactattttcattcctccctatctcaaatctcctaaccacccaaagaaactcacacaaatccaaacc

SEQ ID NO: 177 - DNA, 401 bp, Homo sapiens
catcctggtggtgattcgatgattattaataaagggagggaggggctattagaatagggagggaaagagcacaatccgaagtcttccccagttgactctgaagcactttctccatgcctataatcactgctctaagagcagcaatactatactctgctcacatgacctctgggcagtttactctctcaagtctatgttggrcatgttaggtccacagtggttgagattagttgtgtcctagctaagacctctttgcaagttgtttcaccatcaaaattatgtttcaactattttcattcctccctatctcaaatctcctaaccacccaaagaaactcacacaaatccaaaccactttgcacgactcaacatgcaaatatttccatgaactgagacagagaa

SEQ ID NO: 178 - DNA, 401 bp, Homo sapiens
agagaaagaggtaagtctatatgtgttagccgcctttctttccctactgtgcccaaagcacttgttttcacttgctcagtgagactcaaaagactaggtggccattctttttctctctgcagattagaaatcttacttttagcaaggttttgattctgttaaaatttcatttcaagaaatcatttagaagaaccctgtggyttgttgtaaaatttgcttgcgcagaaaactgaaatttttggttaactagtatcaggaatagagaggttcagctctttaaacactatcagtgacactcaatgatcagcccaaactagtggtcttgtaatgcctacttgtttgtaaacctgcaaacctgtgctgctggtttcggggagacaaaaagaaagaataaaccgtgg

SEQ ID NO: 179 - DNA, 401 bp, Homo sapiens
tggagattatttgttatcagcatagttttaggtatttcagtcttataatgattacacttttaattgtattttaaaattatatatacatttaattatacttaaattatattttaatttttatttaacttgttccctattgctgaattttcaggttgtttccatgtttcaattgcagtaaattacaaaaacaatccttgctckagtccctgtgtataacttgctgggggccaggccacggtttattctttctttttgtctccccgaaaccagcagcacaggtttgcaggtttacaaacaagtaggcattacaagaccactagtttgggctgatcattgagtgtcactgatagtgtttaaagagctgaacctctctattcctgatactagttaaccaaaaattt

SEQ ID NO: 180 - DNA, 401 bp, Homo sapiens
gaatggaatatatatatatatatatatatatatatgtatatatgtgtatatatatatatgtatatacatatatatatatacatatatatataaacatctccatctcctgttcatagcttccaaccagcatccctccaaggaggcaaccaatgtgaccagtttcttgtgtatccttctagagatatttgattcatttctctstgtgtgtgtgtgtgtgtgtgtgtgtgtgtgtgtgtgtctgatatggtttggctgtgtcccacctaaatctcatcttgaattgtagttcccataatccccatgtgtcatggaaaggaccaggtgagaggtaattgaatcacgggggcggttaccctcctgctgttctcatgatagtgagtgagttctcacataattggatg

SEQ ID NO: 181 - DNA, 401 bp, Homo sapiens
atctcctgttcatagcttccaaccagcatccctccaaggaggcaaccaatgtgaccagtttcttgtgtatccttctagagatatttgattcatttctctgtgtgtgtgtgtgtgtgtgtgtgtgtgtgtgtgtgtgtctgatatggtttggctgtgtcccacctaaatctcatcttgaattgtagttcccataatccccawgtgtcatggaaaggaccaggtgagaggtaattgaatcacgggggcggttaccctcctgctgttctcatgatagtgagtgagttctcacataattggatggtttcataaggggcttctccacctcctttgctctcattcttcttcttcctgctgccatgtgaagaaggacatgtttgcctccccttccaccatgattgtaa

SEQ ID NO: 182 - DNA, 401 bp, Homo sapiens
tttataagagttccaaccctttttatgactttttctccagcctctttcttattctccttttgcttttggtttcacagcatactttggcctgctccccttctctttgatttgagccaattcattgtttctcaggagaaactgactaagaagcaattgctctttggacacaaattattttcatggggtggtctcacttaatakcaagttctctccagcttctaaaggaaagagagaaataccactcaggtccccactttgagtgagaccaggaagagagtcagagttctgagaaacttcagaaatccatgctgtctcagaatggaggaccatgatccaccttccaggccctcataggtcattcccagtccagagatgtcaggagcatctgccttccatggtga

SEQ ID NO: 183 - DNA, 401 bp, Homo sapiens
accccactgggaacccagacaccatgttccaagtagcacaagaagatgacacatgcagggctccggccacagctccatcttgtgttccagcatcaacagctagacatgcaagtgaacaagtcttcaggtgatcccagcctttcagctgccccagctgatgctgagtgaagaagaaaaggcctgtccccacagagatctgtscacatttcagatgcacaagcaaaagaaatgttgttcttattttaaagctgctaagttttggagtggtgtgtcacacaccaataactggaacacctgacttttttgccttatcttttgtaacaggtgtaagtaccttcaattcttctgaaattcttgactcttatctcagaccttcattcctgcacacttcagttcttaag

SEQ ID NO: 184 - DNA, 401 bp, Homo sapiens
tgggccaggctttgtgactgccgtgacacataaaatgtggcatatgttatgctgtgtgactttcagggctaggtcgtaaaaactatttggcttgtgcctgcctttctctcaggacaccccactgggaacccagacaccatgttccaagtagcacaagaagatgacacatgcagggctccggccacagctccatcttgtgtyccagcatcaacagctagacatgcaagtgaacaagtcttcaggtgatcccagcctttcagctgccccagctgatgctgagtgaagaagaaaaggcctgtccccacagagatctgtgcacatttcagatgcacaagcaaaagaaatgttgttcttattttaaagctgctaagttttggagtggtgtgtcacacaccaataac

SEQ ID NO: 185 - DNA, 401 bp, Homo sapiens
gtggggtgtcctgagagaaaggcaggcacaagccaaatagtttttacgacctagccctgaaagtcacacagcataacatatgccacattttatgtgtcacggcagtcacaaagcctggcccatcttcaaggggagggaaattaggctccaacttttgatggaggaatgccaaataatttgcaggcgcattataaaaccacyacagtaggctacttttcaaagctaatttaggggcttcttcccaactcattactgggccagccctgagtcaagatgtaaatgtaggggcttccttgggccaacaaaaactccacaatggtggaatatcatttctcattatcctggcttctggaattcctgaatcatccagctcaccccagattctgtagcttaagacttag

SEQ ID NO: 186 - DNA, 401 bp, Homo sapiens
gccagcacacccctccctctgtctccagctcccccgacccatgatccctgctccccctttaattatttccagaagtgtttccattcttctttttttccatttttggaacccattttgttataagaagaatgtaattgttgctaacattttgatttccctgttcagtgagatttacatgacactgagtttctgtgatataaytcctcatttgcttccaagttttctgttgatgggcgtttaatctatgcagaatttcattatcatctgatttgctgtggcctcaacataaggaaagacttttcaggtgtcaactttttttttcgtctttcccttcctctttgccctctctctctctttcacttgctgtctctctcctctctgttgtccctctaaacacacat

SEQ ID NO: 187 - DNA, 401 bp, Homo sapiens
ataagatgactttgctgtaatcccacagtgtttctctctttaatccaaagaatgaaatacagaaaaacaaaaagcaaggcaaaaaaaaaaaatggttccttccaccatataaactggaacttctttcatccattgaatgacatgttttctcacttcttcccttcaccatggggacagggtggagaccatattccctcaagraacccaaggtcatgacattcatggtgaacaaactgcagcctccagaatgcatttccaattaacacacacaaagcgtttcctactgtctcttaaattatggttctatatttatcactgccagcacacccctccctctgtctccagctcccccgacccatgatccctgctccccctttaattatttccagaagtgtttccat

SEQ ID NO: 188 - DNA, 401 bp, Homo sapiens
atgagagtgttctcaacctgtctcatttctgagtctctttccttggcaaataagatgactttgctgtaatcccacagtgtttctctctttaatccaaagaatgaaatacagaaaaacaaaaagcaaggcaaaaaaaaaaaatggttccttccaccatataaactggaacttctttcatccattgaatgacatgttttctcrcttcttcccttcaccatggggacagggtggagaccatattccctcaaggaacccaaggtcatgacattcatggtgaacaaactgcagcctccagaatgcatttccaattaacacacacaaagcgtttcctactgtctcttaaattatggttctatatttatcactgccagcacacccctccctctgtctccagctccccc

SEQ ID NO: 189 - DNA, 401 bp, Homo sapiens
tgataaaaacacaggctcccaaaccaccccagacacacctaatcaagatttcttgggctgggagggtgggggtgaggagggcaggaaatctacatttataacaagctcccaggtatgtttatgtggccatcccagctctggtccccagacatttggaagccgctaaaaaccaaactcctcaaactccttagtttgtaactrtgtaaactaagacttggatatggaagtactgccaaaggccacacacagctggactagaactcaaatatgtcacttctcagtccagaattttttttactatacctgaatctccttcaaagcatgacagaggcctaaattctgtctaccacctcagtcattcctgagcctacatctagaaattccatggtcatgataattga

SEQ ID NO: 190 - DNA, 401 bp, Homo sapiens
tgtatcatgaacatttgggatcatttcatatttcataaatgggcttaactcacactcatgaggtcaactggaaggcctactggatttaattatgctagaaagggagtcaaccagaaacatcatgcatgaagcaggagagaggcggctgggagaaagtgagcaagcagaagggagatttgatgtgatatgaatcatcccacyaggatgaggggctgaaccatacagcaccccatggggggaaaaaaaacaaacaaaaaaaacaaccagcagtccctgagcccaacagaatccaaaggagattctcatcccgatctatttccaggaaggctttaggaagctggttgagtttatgtcacattggtgagtgcaaaataagccatgtaaaatgctacacactgttg

SEQ ID NO: 191 - DNA, 401 bp, Homo sapiens
cagcaccccatggggggaaaaaaaacaaacaaaaaaaacaaccagcagtccctgagcccaacagaatccaaaggagattctcatcccgatctatttccaggaaggctttaggaagctggttgagtttatgtcacattggtgagtgcaaaataagccatgtaaaatgctacacactgttgtgtccagatccttaaaaaaaawatatgataaagctatcgaaggtgttaatacacggaaactctttctttccatattagttagttctaagttctgagtctagggtatgaagtctacgggtttgtataacttgactcggggctgactttgatgggacaaagaacaagaatacatgtgaaatcatggaggagagacactgacagaaagtcacagagatgaaagaa

SEQ ID NO: 192 - DNA, 401 bp, Homo sapiens
gttagttctaagttctgagtctagggtatgaagtctacgggtttgtataacttgactcggggctgactttgatgggacaaagaacaagaatacatgtgaaatcatggaggagagacactgacagaaagtcacagagatgaaagaagaatgaggaactggtgagcacagacttcctatagtgacatggattcatggaacaayagaaggaacacactgactatatcaacccaggccagtaagataggtcagggcgaggcaactaggaattgctgtggcagaggctgctcaatttcttccaaatcatttctctttttcttccctaataatacactttttagctgagcatattgctactcataaaaaagataggattttccagccttctttgcaataagtgtggc

SEQ ID NO: 193 - DNA, 401 bp, Homo sapiens
gagaaagaaagagtgaatgagagtgagagtctgtgttcagctgtaaatttcccaacagcctgctctaatccttccagttttgaatcccacccttaaaaccttaatgacagagaaagggataaaatattatctggaccatgttctgttgacggtttgtcctgggctcattttagccatgataattcagtgatttttgcgcgygcgtgtgtgtgtgtgtgtgtgtgtgtgtgtgtgttttaagggagagacatcagcattctgtggtaccatttcataatgtagagtgtgtaaaagggaaggaatttggaagaatcaggctccttccagttggacatcaacttgaccgttgttttcagctcacttttatctggcagagtttctgtttgcttttaaatgtcaga

SEQ ID NO: 194 - DNA, 401 bp, Homo sapiens
gaaagaaagagtgaatgagagtgagagtctgtgttcagctgtaaatttcccaacagcctgctctaatccttccagttttgaatcccacccttaaaaccttaatgacagagaaagggataaaatattatctggaccatgttctgttgacggtttgtcctgggctcattttagccatgataattcagtgatttttgcgcgcgygtgtgtgtgtgtgtgtgtgtgtgtgtgtgtgttttaagggagagacatcagcattctgtggtaccatttcataatgtagagtgtgtaaaagggaaggaatttggaagaatcaggctccttccagttggacatcaacttgaccgttgttttcagctcacttttatctggcagagtttctgtttgcttttaaatgtcagaaa

SEQ ID NO: 195 - DNA, 401 bp, Homo sapiens
tagtttaagatgctattctgatgtacttaagctcaatactttcaagaacccatcaccttccaaaggcagaggctaagtctgggtgttcacagaggacaaaagcctgtgggctctcaggccacctgttcacctgagtgcccacagagacattcttcaagcatctggttatcagtggctatcaatccaagcagattgctgctraattgtcctttgctcctgtcacctgcctgtttaggcccaggggcttttcttactgcatttgcagatacaggttccattatgttgttctgtgccctgcaaaggcatggaaatctcagtaagggaccatatgctgagactcacagcacagatatttcctgcctgggattctgtataagaagcagggcctctctttacatgac

SEQ ID NO: 196 - DNA, 401 bp, Homo sapiens
atcctctggaagaaatgacagcagaaatgttctgactttttgaggaggagggatgccaggaaagtgagtagggaaaggactccagaagtaggacgactgagctggagacattgagatttaaggtgcccagatattttataaaggcttgctctccttatgggtataaaatacattttacctggctgcagccctcacctcatkgtttggcctgttgttctgcatgttatggtaagagttctgggttgtgctgtaatgaataggatgggagaaagcctcggctggcaggagtggttagcttgactgatctgaggcttggagaattatgacttaaggatgaggtggaaattggcacctcattgctttgtgaggctactctgacttgtgcctttccccttctgggt

SEQ ID NO: 197 - DNA, 401 bp, Homo sapiens
tctgaagacccccacatttaacttcttcctatctacaattatcctctggaagaaatgacagcagaaatgttctgactttttgaggaggagggatgccaggaaagtgagtagggaaaggactccagaagtaggacgactgagctggagacattgagatttaaggtgcccagatattttataaaggcttgctctccttatggrtataaaatacattttacctggctgcagccctcacctcatggtttggcctgttgttctgcatgttatggtaagagttctgggttgtgctgtaatgaataggatgggagaaagcctcggctggcaggagtggttagcttgactgatctgaggcttggagaattatgacttaaggatgaggtggaaattggcacctcattgct

SEQ ID NO: 198 - DNA, 401 bp, Homo sapiens
gctattgataggggctgggctccaacacaggtctgaagacccccacatttaacttcttcctatctacaattatcctctggaagaaatgacagcagaaatgttctgactttttgaggaggagggatgccaggaaagtgagtagggaaaggactccagaagtaggacgactgagctggagacattgagatttaaggtgcccaratattttataaaggcttgctctccttatgggtataaaatacattttacctggctgcagccctcacctcatggtttggcctgttgttctgcatgttatggtaagagttctgggttgtgctgtaatgaataggatgggagaaagcctcggctggcaggagtggttagcttgactgatctgaggcttggagaattatgactta

SEQ ID NO: 199 - DNA, 401 bp, Homo sapiens
gctttctcccatcctattcattacagcacaacccagaactcttaccataacatgcagaacaacaggccaaaccatgaggtgagggctgcagccaggtaaaatgtattttatacccataaggagagcaagcctttataaaatatctgggcaccttaaatctcaatgtctccagctcagtcgtcctacttctggagtcctttycctactcactttcctggcatccctcctcctcaaaaagtcagaacatttctgctgtcatttcttccagaggataattgtagataggaagaagttaaatgtgggggtcttcagacctgtgttggagcccagcccctatcaatagctgaataattctgagcttagtaatcagcatatgggaggcctccactaagtgtctgtca

SEQ ID NO: 200 - DNA, 401 bp, Homo sapiens
gtcctttccctactcactttcctggcatccctcctcctcaaaaagtcagaacatttctgctgtcatttcttccagaggataattgtagataggaagaagttaaatgtgggggtcttcagacctgtgttggagcccagcccctatcaatagctgaataattctgagcttagtaatcagcatatgggaggcctccactaagtrtctgtcaagctgaatccatccacaatctcaggaagatgctaccagtaatttgcaccttcagcacgtatttgttctaaagcatggctaccaactttttctgtgtgcgtgttatttttaaaaaccaagaaattttataaaccactgtcttatgattaatatctattgattaatagaatacatcagaacccaaagctatacga

SEQ ID NO: 201 - DNA, 401 bp, Homo sapiens
ggggtcttcagacctgtgttggagcccagcccctatcaatagctgaataattctgagcttagtaatcagcatatgggaggcctccactaagtgtctgtcaagctgaatccatccacaatctcaggaagatgctaccagtaatttgcaccttcagcacgtatttgttctaaagcatggctaccaactttttctgtgtgcgtrttatttttaaaaaccaagaaattttataaaccactgtcttatgattaatatctattgattaatagaatacatcagaacccaaagctatacgaattcagcaccccctgaaatatgactcatattttttaatcagaatatctactgtgtttaaaaagtcatagtacatatatgtgctcagtatatcatctattaaatctatt

SEQ ID NO: 202 - DNA, 401 bp, Homo sapiens
gtgtctgtcaagctgaatccatccacaatctcaggaagatgctaccagtaatttgcaccttcagcacgtatttgttctaaagcatggctaccaactttttctgtgtgcgtgttatttttaaaaaccaagaaattttataaaccactgtcttatgattaatatctattgattaatagaatacatcagaacccaaagctataygaattcagcaccccctgaaatatgactcatattttttaatcagaatatctactgtgtttaaaaagtcatagtacatatatgtgctcagtatatcatctattaaatctattataatactctgtttaaaatgcaaatttctgagacccaaccctagaatctctgatccagtggatttggaagtagagtcacagaatcaacat

SEQ ID NO: 203 - DNA, 401 bp, Homo sapiens
ctaaaaactaaagccctatttacttaaattatgaaagctgccatagtaaatagacatttaattcaagtaagcccccttgggacagtgtttttttttttttttttttaaccactattatagaaaacacaccaggccaagtgtggtggctcacacctataatcctagcactttgggaggccaaggcaggaggatcaccttatrtcaggagttcaagaccagcctggccaacatcgtgaaaccctgtctctactaaaaatacaaaaattagctgggcatgctggtgggctcctgtaatccctgctactcaggaggctgaggtaggagaatcactttaacccaggaggtgaagggtgcagtgagccgagattgaaccactgcactccaccctgggcgacagagtg

SEQ ID NO: 204 - DNA, 401 bp, Homo sapiens
aggagcccaccagcatgcccagctaatttttgtatttttagtagagacagggtttcacgatgttggccaggctggtcttgaactcctgatataaggtgatcctcctgccttggcctcccaaagtgctaggattataggtgtgagccaccacacttggcctggtgtgttttctataatagtggttaaaaaaaaaaaaaaaamaacactgtcccaagggggcttacttgaattaaatgtctatttactatggcagctttcataatttaagtaaatagggctttagtttttaggttgttaatactttgtattagctttgctcatcatctgtccttaacgttttgtgaggtacatgaaggtaactattatgaagattcgtgggaaatactggttcaatggctagg

SEQ ID NO: 205 - DNA, 401 bp, Homo sapiens
aagccacaggccatgcccagaccagcaacagtgatgccatggtcatccccatgtttattggagaaggggagctaaacttctagcactacatgagttgttcaattatggatctctggattgtgtgatttggaagacacacaactaaggaataactctggccaaaactgagtttgtaaaactggtgtagagccaatgagaagyaacacaatcttcacttctctttgcaaggctagaaatgacacttgcaggctttaagcctatactgtgaaagacaaagaaaattggaaagagtgttctttaatggacactcacaaggtactaaattccatttttatcacctctgcaatctgatgacctagccattgaaccagtatttcccacgaatcttcataatagttacc

SEQ ID NO: 206 - DNA, 401 bp, Homo sapiens
cagaaacccagcctaagtttgactcatcagagctcttaataatcaggttggaaattccacagcattattgaaaggggcaagttcctttctttttagacaaaatggattctgaaaccatccatattctctaattggaatcccaaatggacaagccacaggccatgcccagaccagcaacagtgatgccatggtcatccccaygtttattggagaaggggagctaaacttctagcactacatgagttgttcaattatggatctctggattgtgtgatttggaagacacacaactaaggaataactctggccaaaactgagtttgtaaaactggtgtagagccaatgagaagcaacacaatcttcacttctctttgcaaggctagaaatgacacttgcaggctt

SEQ ID NO: 207 - DNA, 401 bp, Homo sapiens
attagagaatatggatggtttcagaatccattttgtctaaaaagaaaggaacttgcccctttcaataatgctgtggaatttccaacctgattattaagagctctgatgagtcaaacttaggctgggtttctggcttcgcataactcccacattctttgagttttggattcctctagaatccatcacatctaagacccacartgacttcagttcctatattatattagagatctgaggagcatagcaagaatctgcttccctagccctagtttccatttaattcagagaacagaactcttaatttatatttcccttgacaaacaatggcttgacttacagagaaaaggcgtctcaggaacttggacaaacttgagaggagaactaaattagaggttaaataa

SEQ ID NO: 208 - DNA, 401 bp, Homo sapiens
cgtctcaggaacttggacaaacttgagaggagaactaaattagaggttaaataaaattcagccttcccacttcagccaggttctgccatctctagttactataaacaattgggattctgaaagtgttgaaaaaaatacattgacaatcccccccaaaggagaaaggaattataagaccattttgagcctctctgcttctcrgcagtaatatggttacatgggacaaatttagcccagagaaagacacctagctactgttccactaaacccttggaaaggggagtaaatggtcccaacctttgtcatttgtctgcaaagtcctcttctaagacatgcatggaatccataaaataataggcaatgtctcctggctctctctctccctcactccccaccacctc

SEQ ID NO: 209 - DNA, 401 bp, Homo sapiens
acaaacttgagaggagaactaaattagaggttaaataaaattcagccttcccacttcagccaggttctgccatctctagttactataaacaattgggattctgaaagtgttgaaaaaaatacattgacaatcccccccaaaggagaaaggaattataagaccattttgagcctctctgcttctcagcagtaatatggttayatgggacaaatttagcccagagaaagacacctagctactgttccactaaacccttggaaaggggagtaaatggtcccaacctttgtcatttgtctgcaaagtcctcttctaagacatgcatggaatccataaaataataggcaatgtctcctggctctctctctccctcactccccaccacctcacgccccacaaaagga

SEQ ID NO: 210 - DNA, 401 bp, Homo sapiens
gatcagggatagtgagtccatatgcaaaggctgctagcctggaagacatggctaaatgaccatagcttcccatgcagtctcaaattgcctctcaatgcagcctccaggagctgccaccgttgatctattgttaggcgaggttgagcctcctgggcatccattaggttatactgaaataggcaattgcagacctggcagatrggagttagaaaagagcaggggagctcctctgacattttgagggtgatggcatgagcagctaatctggtgcctaatgagcagggccacatcgaagatcgaaatgcttgaaccatggttgacctcaaattctaaggggtcatggcagtacactgttctctctgctactcaaagtagaagccacctctacagcccatcaagtt

SEQ ID NO: 211 - DNA, 401 bp, Homo sapiens
ctcaaattctaaggggtcatggcagtacactgttctctctgctactcaaagtagaagccacctctacagcccatcaagttccctgtccagtctctccttgagaacttccaggagcagggagctcactactttttgagtctgcttcattgccaggcagctctattaggaagtttttccttgagttgaggaaaaaacctaccygtctgtaacatgcacccacagagtctaattcagtcttctgagaccacacagtgggaaagaaataactagggaattgattaaaatcctgttgtcagatagaagagctgacagggatacccagtttactaactagcctgcagctattcagctcctgtggaagtccaacaaggtaagtctcaacacctagggcaggctttgca

SEQ ID NO: 212 - DNA, 401 bp, Homo sapiens
agtccaacaaggtaagtctcaacacctagggcaggctttgcacatggaaaagtggccacaaatgtgggaattataggaccagagaacctgagcatgatcagaagttctcaacagccaggaatataaagagtggaaaaaagaagttatcgctagggtcattcctattatttatgtataatttgttctcagtatcttctcaamacctgtaacagggcttggcatatatgagatgctcagtaaatacatgtggaaagaaggaatctctaatcagaaccttgttaatggctctaagttttcatcatctgatcatttatgtaaacaacatttaataaccactgcgtgtgtattaggcactggagagtttatagataaatgtgacatgagctcaggactgaaaatct

SEQ ID NO: 213 - DNA, 401 bp, Homo sapiens
tttgcacatggaaaagtggccacaaatgtgggaattataggaccagagaacctgagcatgatcagaagttctcaacagccaggaatataaagagtggaaaaaagaagttatcgctagggtcattcctattatttatgtataatttgttctcagtatcttctcaaaacctgtaacagggcttggcatatatgagatgctcartaaatacatgtggaaagaaggaatctctaatcagaaccttgttaatggctctaagttttcatcatctgatcatttatgtaaacaacatttaataaccactgcgtgtgtattaggcactggagagtttatagataaatgtgacatgagctcaggactgaaaatctgatggggaaggaaaaatatttacccaagtagccata

SEQ ID NO: 214 - DNA, 401 bp, Homo sapiens
gaagttatcgctagggtcattcctattatttatgtataatttgttctcagtatcttctcaaaacctgtaacagggcttggcatatatgagatgctcagtaaatacatgtggaaagaaggaatctctaatcagaaccttgttaatggctctaagttttcatcatctgatcatttatgtaaacaacatttaataaccactgcrtgtgtattaggcactggagagtttatagataaatgtgacatgagctcaggactgaaaatctgatggggaaggaaaaatatttacccaagtagccataagtgcactaagaaagggtcaagataagacatggtgagtcccctaaacacagggacgtattgtaaagagagttaacaaaaagccttgatgtaaaaactcttcaa

SEQ ID NO: 215 - DNA, 401 bp, Homo sapiens
ccttgatgtaaaaactcttcaactagtgtcactctgttaaacttgaagctcaatattccctcttcaattgacaagaacacagactaatgattcaaactaaaattctaagatgtgaatttcccgatggagctggagggagaaagtttcccatggagggtccttgtttctggctctctctttgcttcctggcaagacctcaaytggcactcctccgaacgacccaccctactgtaaacttctccccggcagctcaccttctccctgggcttgcaactgcgagtcgcatacctggctgcggccaagataatttcaggaaatggtactttggaaactcgtctttaaaatgaaaacagataacacagctggcagtggagccaaggaaaactccactttttctgaaa

SEQ ID NO: 216 - DNA, 401 bp, Homo sapiens
agggcggtgggaaaaaggttcctcaggttttgagggtgtgttctgcagccctgctgaggaacgtttgtgtatcctatgtgtgctggtgcatttattagaggatgtggtagtgatggcgttgggcaggtaggtttccggtgccaacaagctgcttgcttttcagaaaaagtggagttttccttggctccactgccagctgtkttatctgttttcattttaaagacgagtttccaaagtaccatttcctgaaattatcttggccgcagccaggtatgcgactcgcagttgcaagcccagggagaaggtgagctgccggggagaagtttacagtagggtgggtcgttcggaggagtgccaattgaggtcttgccaggaagcaaagagagagccagaaacaagga

SEQ ID NO: 217 - DNA, 401 bp, Homo sapiens
tataatggcagaaccccagtctagaatgggtttcctgacaagggcattcattcattcaataaatattgcttggtgctagacatctattgtgagcaccacagacatggtccttatcttcaaggactttaccatccactggaagaaagaaggattaaatagcttatcatagtatgtaactattttttcatgtgatgagtactrtgaagaaaaaacagagggcactttaatagaccttacacaaagacctaatttagcccaaggggtacagaaaagcatctctggggaagtctgaccctgaactgagaaggaaatgaagaacatgaatctgtcaggtaaagatgaggggaaagagggctccaggtaaagggacttcagacacagtggctctgcttcagggtttg

SEQ ID NO: 218 - DNA, 401 bp, Homo sapiens
ctgacaagggcattcattcattcaataaatattgcttggtgctagacatctattgtgagcaccacagacatggtccttatcttcaaggactttaccatccactggaagaaagaaggattaaatagcttatcatagtatgtaactattttttcatgtgatgagtactgtgaagaaaaaacagagggcactttaatagacctwacacaaagacctaatttagcccaaggggtacagaaaagcatctctggggaagtctgaccctgaactgagaaggaaatgaagaacatgaatctgtcaggtaaagatgaggggaaagagggctccaggtaaagggacttcagacacagtggctctgcttcagggtttggtcccaaacaaatgcctcttatgtagtatatctt

SEQ ID NO: 219 - DNA, 401 bp, Homo sapiens
aataactgaagccagatttatcttcaattcattatgacctctccagcccttaactttagacaaagcaaacactaccaaaaacaccccgaatggacttgctacaaaaatttgggtaaattttatttttctcatctctaaattagatgcataaagccaacctttgcagagaagtgactgtaaggaagaacaaataacttactygtcaaatttctggaggtcttcctgtagcaccatctggagagcccagtgctataattccatggatatgattgtgaggtcttgtgtgaggaatgtcttctttcaggcagctagaattccagcccactgatatttccatggacttggcatgttgacatccctcggactcagtttctctcctttgcaaaataagataaataata

SEQ ID NO: 220 - DNA, 401 bp, Homo sapiens
cttttctctcaggctcctggattcaaggggctgaatagatgggaattcctgctgtctctggagaagagcttacttcctttcccagacttcccaaccccacccaggatcacttgtcattgagattaacctttgtggagaggcacccaggagcgcaactctctgggagtgtgacagctttttcaggatatcaggaagtggagsgtgtgtgtgacccaggagggacacacatccatgttacagaagaaggctgaggctgcaaaacatgccccagaaaggaagcggatgttacaccttcgctacctcgctgctctgaaacaggaagacattcatttgtccttctgggcattcaacattccactcacaaactcccctcctggagtctctctgtggttcagtgaatc

SEQ ID NO: 221 - DNA, 401 bp, Homo sapiens
cacttgtcattgagattaacctttgtggagaggcacccaggagcgcaactctctgggagtgtgacagctttttcaggatatcaggaagtggagcgtgtgtgtgacccaggagggacacacatccatgttacagaagaaggctgaggctgcaaaacatgccccagaaaggaagcggatgttacaccttcgctacctcgctgytctgaaacaggaagacattcatttgtccttctgggcattcaacattccactcacaaactcccctcctggagtctctctgtggttcagtgaatcgtatgttccagaaaccagcccttcccccacctcaacccagaggacttactagttctgatgtgaaaggtattgattcagaagctcttgatggctgagggctccctgct

SEQ ID NO: 222 - DNA, 401 bp, Homo sapiens
ttgtgttcccttccattttcttcattgtaagcccctcctccgtcaaataaaccctaacttgggtttaagaccttcttggaagatgattctataccctccctttctagacctccacaaccctcactctcattcaaatgagagaatcactaaagggctccttgtccttgccccccagcagaccctcataaatccacagtgagkttttttttttaattaaagcgtccaaataattaatgatcaactccatttgtgccatagaaaaccaaaatgtcacacggttgaaaagacccaaccaaataattcagtgtaccctataaagctttaaaaaagcggagcccaagaggcctgcatgactggtttctgtgctgtctgaaaagcttggttgggttttgggcacttga

SEQ ID NO: 223 - DNA, 401 bp, Homo sapiens
ttggaagatgattctataccctccctttctagacctccacaaccctcactctcattcaaatgagagaatcactaaagggctccttgtccttgccccccagcagaccctcataaatccacagtgaggttttttttttaattaaagcgtccaaataattaatgatcaactccatttgtgccatagaaaaccaaaatgtcacasggttgaaaagacccaaccaaataattcagtgtaccctataaagctttaaaaaagcggagcccaagaggcctgcatgactggtttctgtgctgtctgaaaagcttggttgggttttgggcacttgatcggattccaggaataatgtgccctgagcacaaagggctcttttgatgtgtctgtgccatagctcctcccctttt

SEQ ID NO: 224 - DNA, 401 bp, Homo sapiens
caaatgagagaatcactaaagggctccttgtccttgccccccagcagaccctcataaatccacagtgaggttttttttttaattaaagcgtccaaataattaatgatcaactccatttgtgccatagaaaaccaaaatgtcacacggttgaaaagacccaaccaaataattcagtgtaccctataaagctttaaaaaagcrgagcccaagaggcctgcatgactggtttctgtgctgtctgaaaagcttggttgggttttgggcacttgatcggattccaggaataatgtgccctgagcacaaagggctcttttgatgtgtctgtgccatagctcctccccttttcctcctcactttacctgggatacacattctccttgtctcctaagttggaaaaaata

SEQ ID NO: 225 - DNA, 401 bp, Homo sapiens
ttttttttaattaaagcgtccaaataattaatgatcaactccatttgtgccatagaaaaccaaaatgtcacacggttgaaaagacccaaccaaataattcagtgtaccctataaagctttaaaaaagcggagcccaagaggcctgcatgactggtttctgtgctgtctgaaaagcttggttgggttttgggcacttgatcrgattccaggaataatgtgccctgagcacaaagggctcttttgatgtgtctgtgccatagctcctccccttttcctcctcactttacctgggatacacattctccttgtctcctaagttggaaaaaataacccatggaaaagaggaaaaaacagcagagggaaaatctaggctggaagtggggaaaaatgctgcagttctt

SEQ ID NO: 226 - DNA, 401 bp, Homo sapiens
ccccttttcctcctcactttacctgggatacacattctccttgtctcctaagttggaaaaaataacccatggaaaagaggaaaaaacagcagagggaaaatctaggctggaagtggggaaaaatgctgcagttcttagtctgagtttggttactaaagactagttattccagaaggaatagaagaagtgacatttatacawtgtctgtgaccagacaatgggaaatcgtaatgataagatgtctcttgtttgaatgtggtcccttgagctaaaggtaatgagtgagtcttcaagcaaggacttgccactttcaaacagtcagaggtatggcaggttactaaccctagaaatcaacatgaatttagatgcctaatccagccagagaatcttatagatagtga

SEQ ID NO: 227 - DNA, 401 bp, Homo sapiens
ggtcccttgagctaaaggtaatgagtgagtcttcaagcaaggacttgccactttcaaacagtcagaggtatggcaggttactaaccctagaaatcaacatgaatttagatgcctaatccagccagagaatcttatagatagtgagtccatcccgttggttttccatgggtaccagaatattcaggaaagatggaaagaaasaagacagaacttccattgctaggatgtccttaaaaagtggcagagcaaactagaggccaggagtgtataagcaatggccctgagagataaggattgtgggtctagccccagcatacagatgtccagattggcagcaaactgcagtaagctggtgctaatacagggctatttttgtctgtctgggctaacttgaagaggat

SEQ ID NO: 228 - DNA, 401 bp, Homo sapiens
gcttagatataaggtagaatctattttaaagctatgggatagttctgtgaatcacagaaaaaaaaaatgaactgttagacctcagaaaatgagagggcctaggatagcattcaggcacaaatcaactatctccatcaggacgatctctggacttacccttgtgtaacttctcttaagattcaaattcctcagagagcaggwcttgttggccctatttatctccggcccttggccacattgccagttaattgagaggcccaccaaaactgcatgcagtgccagaagagtgttaccccaaaggaaattcaaggtgctgttatgcaaagggagaactcatgctgcagacgtaaaacaacacgactgccatgtatgatatctaactagaactttctgcccatcac

SEQ ID NO: 229 - DNA, 401 bp, Homo sapiens
tgtgaatcacagaaaaaaaaaatgaactgttagacctcagaaaatgagagggcctaggatagcattcaggcacaaatcaactatctccatcaggacgatctctggacttacccttgtgtaacttctcttaagattcaaattcctcagagagcaggacttgttggccctatttatctccggcccttggccacattgccagtyaattgagaggcccaccaaaactgcatgcagtgccagaagagtgttaccccaaaggaaattcaaggtgctgttatgcaaagggagaactcatgctgcagacgtaaaacaacacgactgccatgtatgatatctaactagaactttctgcccatcactcatcaagtgtacagactagcacttaaagtttcggtgataatagt

SEQ ID NO: 230 - DNA, 401 bp, Homo sapiens
tagacctcagaaaatgagagggcctaggatagcattcaggcacaaatcaactatctccatcaggacgatctctggacttacccttgtgtaacttctcttaagattcaaattcctcagagagcaggacttgttggccctatttatctccggcccttggccacattgccagttaattgagaggcccaccaaaactgcatgcartgccagaagagtgttaccccaaaggaaattcaaggtgctgttatgcaaagggagaactcatgctgcagacgtaaaacaacacgactgccatgtatgatatctaactagaactttctgcccatcactcatcaagtgtacagactagcacttaaagtttcggtgataatagtggtgatggaaaagattacaatctcagacag

SEQ ID NO: 231 - DNA, 401 bp, Homo sapiens
tcaggcacaaatcaactatctccatcaggacgatctctggacttacccttgtgtaacttctcttaagattcaaattcctcagagagcaggacttgttggccctatttatctccggcccttggccacattgccagttaattgagaggcccaccaaaactgcatgcagtgccagaagagtgttaccccaaaggaaattcaagrtgctgttatgcaaagggagaactcatgctgcagacgtaaaacaacacgactgccatgtatgatatctaactagaactttctgcccatcactcatcaagtgtacagactagcacttaaagtttcggtgataatagtggtgatggaaaagattacaatctcagacagccctatcttcctatctagccatcaaaagcacttgg

SEQ ID NO: 232 - DNA, 401 bp, Homo sapiens
tcaatgttgttactgattgtatcaggataaatactaggtctctgattccaaagtaactgcagcaaagtaaacacaccaccacccttaaaaagagacagaagatagaggcaagaaagcaagatagaacacagatgtatttgattactaatcaagcctatttcaacagtgcaaaacatattaatgggaaagagggaggtagaytgaagcccaggcaggccaagctaggcgaaggatctactttcatgtataaatgaataacatggatcattgacagtgaacccaagtgcttttgatggctagataggaagatagggctgtctgagattgtaatcttttccatcaccactattatcaccgaaactttaagtgctagtctgtacacttgatgagtgatgggcaga

SEQ ID NO: 233 - DNA, 401 bp, Homo sapiens
tctagccatcaaaagcacttgggttcactgtcaatgatccatgttattcatttatacatgaaagtagatccttcgcctagcttggcctgcctgggcttcagtctacctccctctttcccattaatatgttttgcactgttgaaataggcttgattagtaatcaaatacatctgtgttctatcttgctttcttgcctctatyttctgtctctttttaagggtggtggtgtgtttactttgctgcagttactttggaatcagagacctagtatttatcctgatacaatcagtaacaacattgaattctctctgcttgctgaactttttatacactatgtgggcttgagcacacgtagtgcataaaagatgtttgtgggtttcctaggtgttcttaatccatca

SEQ ID NO: 234 - DNA, 401 bp, Homo sapiens
aaataggcttgattagtaatcaaatacatctgtgttctatcttgctttcttgcctctatcttctgtctctttttaagggtggtggtgtgtttactttgctgcagttactttggaatcagagacctagtatttatcctgatacaatcagtaacaacattgaattctctctgcttgctgaactttttatacactatgtgggcytgagcacacgtagtgcataaaagatgtttgtgggtttcctaggtgttcttaatccatcagaaaatggattggggtaatcatgatcaattttcctaaggagggaattatagtgaaaaaaagtgaggttaaaaggctcttatctttgccatcactcatccccacctttatttattgctttctctggttttcattagcatgct

SEQ ID NO: 235 - DNA, 401 bp, Homo sapiens
tcaattttcctaaggagggaattatagtgaaaaaaagtgaggttaaaaggctcttatctttgccatcactcatccccacctttatttattgctttctctggttttcattagcatgctgatgaccaaagctacagacattttcatccatattctgactgctgccctgatttcagttccaagttatgaaacctaatccaaacyttagaggggaaactacaaactgtaactttaaatttcagtttacagagtctcccaggatgattccaggaattgagttgggaggggtcctgggaatcagaaaccaaactaccatctccttcctcctctgaaaggacccgaggacatttgaaacataactcataaaagaccattttcacaacatcccagctgagaaaaattgc

SEQ ID NO: 236 - DNA, 401 bp, Homo sapiens
cctacaatttccatatgccctgtgatcttctgtaatccttgggtcagctcagcagtaaggtgataaagatgttccctatggaaccttaaagaagacaattggttcatacccagtttattgtgaaggaaaggttcagtgggaagactagaagacacaagtccatttttatgatggggtgtatgtgagtggtttgtttgaggwgtagttctcagtactttaatcaagaaggtactatgcccattttcaactccaagatgtagtccgaagacaaaaaataaataaataaataaagtcttacagagaatatgaagggcacagggggaattttgtctatcaaaaaaatttaaaaataatgatttcaatctaagtgtctgtttacaaaagctactaaaatcaaaggt

SEQ ID NO: 237 - DNA, 401 bp, Homo sapiens
aagaaaaaaaataggttaaacatttaggcaacatttacagccccaaagtacctaatcactttggtgagttcaggctctgaatgggctagttatcatgtggtccaaacaactgaagaattaagtacatcactaatgaccactagttgtttcagccaagtacagaaatctttgaaatggtttctggctgtgaatgtggctcartatagaaaaaaagtggccaaaagagcctttcagatttctttcctgtagcccttcactcataaccaaggagggctggagaaatagcacttctttctaagagcagagacataatttgagtttgagatattcatgtttagttggagttagtgaaggaatttaaatggtttattaccaccaaggagacacttgcaattggcatt

SEQ ID NO: 238 - DNA, 401 bp, Homo sapiens
atcaaactgctggagactaagaataaagaacaaatcttaaagcagttaagaaaaataatatattacatacagaagagcaaatatttaaatgacagtggattctcatcaggaaccatggaggcgagaagacagtggaacaatatctttaaagtgctgaagataaataaataaatagataaataaaactatcaacgaagacamataaataaatagataaataaaactatcaacctgaaattttgtatctagctaaaatattctcaggaatgaagataaaataaaaatattttcagataaagaaagtctaagaattcattgctagcagacctgtgatacaaaaaattgctgaaggaatttctttagactgtaagaacaagacactagagtaaaacttgcatctt

SEQ ID NO: 239 - DNA, 401 bp, Homo sapiens
ctatcaacgaagacacataaataaatagataaataaaactatcaacctgaaattttgtatctagctaaaatattctcaggaatgaagataaaataaaaatattttcagataaagaaagtctaagaattcattgctagcagacctgtgatacaaaaaattgctgaaggaatttctttagactgtaagaacaagacactagartaaaacttgcatcttcaggaaagaataaagtatcaaaactggtaaatacctggtgtataagtcatggttcaaccagaggagtatttatgccgtcatgcattgtataatgacgttttgctcaatgacagacgcatacatgatggtgctcccacaaaattatgatggagcatatatagaaatctgatatatggcacttgata

SEQ ID NO: 240 - DNA, 401 bp, Homo sapiens
acaagataataacctttttgactaatttaaagtcaaatagtaatgattgggggctttaattgcatctacaaaatttcttcacatcagcacctaaatcaatgtttgactgaataactggaatggtagtttagcctagccaagtttacatatcaaaaagctattacatctgagtaaatataaaacactactttctttcctttyaagctctttaaaatacctatatttgcttacactaaaatctaatattggtggagtttttatgagacatatgtaatataaaatatgttgactatagaataaagggaccaatagtgttgttgggtttacatcagtggtaatattaaattttagttgacgatgacaggtgatagatagaaccattaagatatttataataatgc

SEQ ID NO: 241 - DNA, 401 bp, Homo sapiens
ttgcatctacaaaatttcttcacatcagcacctaaatcaatgtttgactgaataactggaatggtagtttagcctagccaagtttacatatcaaaaagctattacatctgagtaaatataaaacactactttctttccttttaagctctttaaaatacctatatttgcttacactaaaatctaatattggtggagtttttmtgagacatatgtaatataaaatatgttgactatagaataaagggaccaatagtgttgttgggtttacatcagtggtaatattaaattttagttgacgatgacaggtgatagatagaaccattaagatatttataataatgcatagctttaagccaatataaattaaaatagaatactaaacatattcaaataatctaaaa

SEQ ID NO: 242 - DNA, 401 bp, Homo sapiens
ttgttgggtttacatcagtggtaatattaaattttagttgacgatgacaggtgatagatagaaccattaagatatttataataatgcatagctttaagccaatataaattaaaatagaatactaaacatattcaaataatctaaaataaagcaggaaaagggaaacagaggaacaaaaaagaaatcgtaacataataagayagtagatctaaatttaaccatatcagtaattacattaaatgttaatgatctaagcattaaaagatattgtgagagtagatttaaaaaagcaagactcaagcacgttgccaacaagagacatactttaaacacaaagatacatgtagtttgaaagtaagtggataatggtaaagatacaccatgcaaacagtaagcttaag

SEQ ID NO: 243 - DNA, 401 bp, Homo sapiens
ttacagctgaatgtagagttctattataatgtcaatcagatcagggtggttgacagtgatgttcagatcttccgtgtctctactgatattttgtctggttattctatcaattgctgagagaggagtgttaaaatctactaagattatagaggttttttttttctacttctgttgagtatcaggttctgctattagatacawacatatttataattactgtcttctcggtgaattgtcctttttgaaatttttattcctggttatgctctttgtcatgaagtctactcttttaatttgttattaatatagtcactctagtcttcttaagcttactgtttgcatggtgtatctttaccattatccacttactttcaaactacatgtatctttgtgtttaaagt

SEQ ID NO: 244 - DNA, 401 bp, Homo sapiens
catggtacattcaccaagatagaccatatacctgtactgcaaaacacttgtcaataagttcaaaggaacagaaatcatgtaaagtgttttctataatcataagagaattaatcagtaataacatatctaggaaaatccatatctaggaaaattatttaaaagtatgtcatttggaatgtcatttccaattatttggaaatyaaatgacatacttttaaataacttatgcatcacagtcaaaatcacaaggaaaatttaaaaatgttttgaactgaataagggagaaaatacagtatcaaaatttgtaagatgcaacttaagtactgcttaaagggaaacttgcagctttaaatgcaaaagaagaaaggcctaaaaacaacagcctatgattccaccttaag

SEQ ID NO: 245 - DNA, 401 bp, Homo sapiens
catcacagtcaaaatcacaaggaaaatttaaaaatgttttgaactgaataagggagaaaatacagtatcaaaatttgtaagatgcaacttaagtactgcttaaagggaaacttgcagctttaaatgcaaaagaagaaaggcctaaaaacaacagcctatgattccaccttaagaggttcaaaaaagatcaaagtaagcccmaagtaagtagaaggaagaaaatgaagataagagtagaaagaagtcaatgaaattgaaaatagatacctaatagtgaaaattagcaaagccaaaagctggctccttgaaaaaaaaattgacaaactcttagttatactgacaaaaaaaaaaaaaaaaagaaagaaaagaaagagaaagaaaaaaagaatacatcaccaata

SEQ ID NO: 246 - DNA, 401 bp, Homo sapiens
aaagagaaagaaaaaaagaatacatcaccaatatcaggaataaaaaaatatgttatcaccacaaatcctacagattttaaaaggataataagtaagtattggagcaactttatcatattttatagcttagatgaaagggataaatttcttgaaaaatgcaacttactaaagttggcatgaaaagaaacaaaatctgaattrtcattttcgaaatgagaaaatttactgcattatcaaaaagcttccctgaaagaaaaacaatagacaagtggcaaaatatatgaacgtgcaattcacaaaagaagaaacttgaatggttcctaattctatgcaaaaatgtttgacctcagtgatcagaaaaatgtaaataaaaccacatagaaaacatttcacactaagca

SEQ ID NO: 247 - DNA, 401 bp, Homo sapiens
caaaagatggaggcttcagtcataatgaggaatcagacgcccttgctgaatgccctcaactgttttgacttctggaggggcgggccgtgattacagctgcgattaggtgactcaccataattatgttttgttgaaattgaatttattgaagcatgaatcattctgcccaagtcctacctaattgctccaacactgtggatkggaaggatggcggaggagttttccttggaagtcattatagaacgaatgtgcttgcgcacgtgcgtccatccccgcacacagtcggccacactcaccttacagaaagccagcagcgcggccccatcctttgtgatgacatgaggttgccagacaatctttacttgtttagctgtggtttcttcgtctcctaccccctactc

SEQ ID NO: 248 - DNA, 401 bp, Homo sapiens
tgctgaatgccctcaactgttttgacttctggaggggcgggccgtgattacagctgcgattaggtgactcaccataattatgttttgttgaaattgaatttattgaagcatgaatcattctgcccaagtcctacctaattgctccaacactgtggattggaaggatggcggaggagttttccttggaagtcattatagaaygaatgtgcttgcgcacgtgcgtccatccccgcacacagtcggccacactcaccttacagaaagccagcagcgcggccccatcctttgtgatgacatgaggttgccagacaatctttacttgtttagctgtggtttcttcgtctcctaccccctactcttctttgtgagcaactcactatttggccgtttggctccccggg

SEQ ID NO: 249 - DNA, 401 bp, Homo sapiens
gcttgcgcacgtgcgtccatccccgcacacagtcggccacactcaccttacagaaagccagcagcgcggccccatcctttgtgatgacatgaggttgccagacaatctttacttgtttagctgtggtttcttcgtctcctaccccctactcttctttgtgagcaactcactatttggccgtttggctccccgggggtctckgtgcctcagaccgcaggcctctctgagagttgttgatgcctcagagcggctgacttgcagattgtatccgtattcccccagctcagacctcccaccaccactcagcggacaacttagggatgccctcaaagctgcgtgcctgctgcagagctgtaatttaaatcaacttgggagctgtgacgaggcaaagagcaacctca

SEQ ID NO: 250 - DNA, 401 bp, Homo sapiens
tctctactcattaaagtcaactgttaaagagtaaatgctccagggaaaaggtaacattttaaactcatgtggacaaacttttgggggaatgaactgaggggtaactgacaaggacccctgggtatggatgcacaatagggacttgaagagtcaaagaggaaatgaggaataaagcttcactaactcactcactgcccagtygtcccagggacaagtctgggctcctgtccgtcctttgtcttctggtttcttccctctaaaacaccacagactcaaaggtggcagatcagagcaactctagagacccttgcagatatgtcagagtgtcaagggctcaagggccagttcaatcctttcattttatagaccaaaaaacagagacctcaattgctacatgattt

SEQ ID NO: 251 - DNA, 401 bp, Homo sapiens
gacaaggacccctgggtatggatgcacaatagggacttgaagagtcaaagaggaaatgaggaataaagcttcactaactcactcactgcccagttgtcccagggacaagtctgggctcctgtccgtcctttgtcttctggtttcttccctctaaaacaccacagactcaaaggtggcagatcagagcaactctagagaccyttgcagatatgtcagagtgtcaagggctcaagggccagttcaatcctttcattttatagaccaaaaaacagagacctcaattgctacatgatttgtctatggttatacagttaaacggtatgaaaaccaataataaaactcagaaataatggtcttgagaaaaataagcagccctaagccactgtaggaattttcatatg

SEQ ID NO: 252 - DNA, 401 bp, Homo sapiens
tctattcctgttaccttttcctttttccagcctttcagtttggcatcatttcccagcctaacaaatttattgcctattttatcattcagaatggaggtgatggtagcaatcacttttcacctctgagttttacggacactcacctatcacactccagctttggcctctcccagatggacacctattcattggaaagcaacrtgtggaatgagctgtgcggacactggtatgtctaccaaagacctctgggattctatttctctcatgctattcgtcacagtacctgatgccctggtgctcacctccttcttgaaagttatgccatcctaacctccaccattcgtcttttccaattatcctctcttttcactaatgtcctctgacttcttcagttcttgttc

SEQ ID NO: 253 - DNA, 401 bp, Homo sapiens
cctttcagtttggcatcatttcccagcctaacaaatttattgcctattttatcattcagaatggaggtgatggtagcaatcacttttcacctctgagttttacggacactcacctatcacactccagctttggcctctcccagatggacacctattcattggaaagcaacatgtggaatgagctgtgcggacactggtatrtctaccaaagacctctgggattctatttctctcatgctattcgtcacagtacctgatgccctggtgctcacctccttcttgaaagttatgccatcctaacctccaccattcgtcttttccaattatcctctcttttcactaatgtcctctgacttcttcagttcttgttctttcctcaccccttagccacagcatcactt

SEQ ID NO: 254 - DNA, 401 bp, Homo sapiens
ttgcctattttatcattcagaatggaggtgatggtagcaatcacttttcacctctgagttttacggacactcacctatcacactccagctttggcctctcccagatggacacctattcattggaaagcaacatgtggaatgagctgtgcggacactggtatgtctaccaaagacctctgggattctatttctctcatgctrttcgtcacagtacctgatgccctggtgctcacctccttcttgaaagttatgccatcctaacctccaccattcgtcttttccaattatcctctcttttcactaatgtcctctgacttcttcagttcttgttctttcctcaccccttagccacagcatcacttgagtttaatacatatccatctgcattaatttctgtgact

SEQ ID NO: 255 - DNA, 401 bp, Homo sapiens
tgagctgtgcggacactggtatgtctaccaaagacctctgggattctatttctctcatgctattcgtcacagtacctgatgccctggtgctcacctccttcttgaaagttatgccatcctaacctccaccattcgtcttttccaattatcctctcttttcactaatgtcctctgacttcttcagttcttgttctttcctcrccccttagccacagcatcacttgagtttaatacatatccatctgcattaatttctgtgactagaacagtggcaatcccacccccttttcccctatttggagagagacatgggaagaaaacagcttctcattaaggaacttaatcaggtcttatttaagtagtcgtcttaaagctatggctgagcctttggcctacaaagt

SEQ ID NO: 256 - DNA, 401 bp, Homo sapiens
tggtatgtctaccaaagacctctgggattctatttctctcatgctattcgtcacagtacctgatgccctggtgctcacctccttcttgaaagttatgccatcctaacctccaccattcgtcttttccaattatcctctcttttcactaatgtcctctgacttcttcagttcttgttctttcctcaccccttagccacagcrtcacttgagtttaatacatatccatctgcattaatttctgtgactagaacagtggcaatcccacccccttttcccctatttggagagagacatgggaagaaaacagcttctcattaaggaacttaatcaggtcttatttaagtagtcgtcttaaagctatggctgagcctttggcctacaaagtaaagcttagaccttaa

SEQ ID NO: 257 - DNA, 401 bp, Homo sapiens
ccctctatatgtagaagattttatgaaaatctgagggagaagcaggcctcgtataaatgttttgtcattaaattttaaaagaagcatacttttttattaaaattttttaaagaagcatacttcatgtttcagtgaatagatggaagttaaaggaaatcacaagaaacagaagggagtaacattccaagggaggcagaaacrtagagataaaggtagccataaaaatgtaattttctgagattttatctacactaccctaattatagcaagagagttgtactattcttaaaaagcaaaatatagatttaggctacttcatctatttatatgacttaaattacaacaatttttaaaactagtaccaatatttatacaactccaaaataaatctgtggagttga

SEQ ID NO: 258 - DNA, 401 bp, Homo sapiens
tgaccacagtagattcagatgtaatcagtttgttgcttgggatacaccatacttagcctgtcaatccacctgaaaaaggaacaataaattcccgtccttacttttcctcacccaaattagcccaaacaaattagattggtagaaccatttgttcccatctggtttccacaggacatgtagactcagaaaagttttcttttkttgttgttgttgtttgtttgtttttgtttttaacttttattttaggttcgggggtatatgtacaggtttattacataggtaaacttatgtcataggggtttgttgtacagattatttcatcaccccagtataaaacctagtacccattagttatttttcctgatcctctccctcctcccaccctccattcaccctcaatc

SEQ ID NO: 259 - DNA, 401 bp, Homo sapiens
ataatggcttccagctccatccatgtccctgcaaaggacatgatctggttcttttctatggctgcatagtattccatggtgtattaatatatgtaccacaatttcttcatccagtttatcattaatgggcatttaggttgattccatgttgcaaaggttccttaagattccatctttcctttagtttcatagcctattttkcccatttctcccattcctcctttcttccatttctttacccccagatgtccaaagcagcctcctgacttatattcttgttcccctttcttcccctctctttagcctaattcatttagattccagagaaactgagccacattttcaccatttgttggtctcgactaaaatcaaaaatttgtcattaatatgctaccacatgt

SEQ ID NO: 260 - DNA, 401 bp, Homo sapiens
acttatattcttgttcccctttcttcccctctctttagcctaattcatttagattccagagaaactgagccacattttcaccatttgttggtctcgactaaaatcaaaaatttgtcattaatatgctaccacatgtaaacaggcaacaaagtaaatgattttgacctaaatatttgagacattggctcttggtaaatacawaatctccttcatgtcatgaggacatgtatttgaccagacattgggtaaggggttgaggaagctttgtgtcatatttttggggaaagaccactatacctccatcgctacatcatctgagaatttagaataatctgtatcagagactagatggtcaaaaacttaaattgcagaagatataacatcactttctgtgcacctat

SEQ ID NO: 261 - DNA, 401 bp, Homo sapiens
tttgtcattaatatgctaccacatgtaaacaggcaacaaagtaaatgattttgacctaaatatttgagacattggctcttggtaaatacaaaatctccttcatgtcatgaggacatgtatttgaccagacattgggtaaggggttgaggaagctttgtgtcatatttttggggaaagaccactatacctccatcgctacaycatctgagaatttagaataatctgtatcagagactagatggtcaaaaacttaaattgcagaagatataacatcactttctgtgcacctatcagtgtgcatatgtagacaatcacttccagaaagaggggagactgggcagctggctggatgtactgggacatattgaaccattttttgaatgtttggggcttggcaaata

SEQ ID NO: 262 - DNA, 401 bp, Homo sapiens
ctgggcagctggctggatgtactgggacatattgaaccattttttgaatgtttggggcttggcaaataaatgcattgtgcccgaagcacctttttcccttcacttaaaacccatgtgtattggaaaggaaatttaattcttatgtttctgatttgcccacatttaacccatcaaaatccaacttggtgggaaccattggaygcccataagccttctgaaccttgtgtaataaaccacttggagctgtcttttctttggatctcagaaaccacaatgaggaactattttcagtggctttagtcaggggaacaagatgggagcttgattttgccctcagatttcaccacagccaacaaattctaactttgttcagatgcattgtcacgccttgtatttacttc

SEQ ID NO: 263 - DNA, 401 bp, Homo sapiens
gcccgaagcacctttttcccttcacttaaaacccatgtgtattggaaaggaaatttaattcttatgtttctgatttgcccacatttaacccatcaaaatccaacttggtgggaaccattggatgcccataagccttctgaaccttgtgtaataaaccacttggagctgtcttttctttggatctcagaaaccacaatgagraactattttcagtggctttagtcaggggaacaagatgggagcttgattttgccctcagatttcaccacagccaacaaattctaactttgttcagatgcattgtcacgccttgtatttacttcttgccaataacctgaggtctagtaaccactgtttttgtgaaagtcaaacaatatggactgatattcaactccaaaacg

SEQ ID NO: 264 - DNA, 401 bp, Homo sapiens
cattctgacaggctgggaagtgtagcctccagccagaagctggaaaatggcactttggtggtggtcggggtaggcataagacaggaatttatgctgaacagcttggctaagcatacatattcaataaactataggagaaatcatgaatatttatgaaaggagtaatatgcacaggcacaattgagctccatgcctcttcaygggtggcatgttcaaaaaatgatggcattactatttaaaatagtaaggacttggaactaacccaaatgcccatcaatgatagacttgataaagaaaacgtgtcacatgtacaccatgcaatactatgcagctataaaaaagaatgatttcatgtcttttgcagggacatggatgaagctggaagccatcattcttagaaa

SEQ ID NO: 265 - DNA, 401 bp, Homo sapiens
gaggggaacatcacacaccggggcctgttggggggtgaggggcaaggggagggagagcattaggacaaatacctaatgcatgctgagattaaaacctagatgatgggtcgatagatacagcaaaccaccatggcacatgtatacctatgtaataaacctacatgttctgcattatgtatcccagaacttaaagtaaaattwtaaaaaaaaaaagatggcattagaatgatccaaaggtgcagtttttgactttctaatatcaaaaggtaaagcacagaacatgtaaaccctcactgtgcaccctctgttgatgccagaaccactccatggttggtggtctcttattaggaaggaatacattgtaaaactggtgagttatcatgtcaaaactgcaaagagag

SEQ ID NO: 266 - DNA, 401 bp, Homo sapiens
gataattggctaaaggtaataaaggaaggagtcatccctttcttgttttctagagctgatttctgcttactctaggaaagaattctggttaaaggttaataacgaaggggcatactgggatgtgtctgacctcccatcttgtcatggctaggagctcagtttttaaggtttatctggggtccccttggccaagagggagtytattcagttttttgaaggacttaggattttatttttatttctcatgagcaacaaggcttatgatctataaaaacagggaaaataataccttcttcatagggttgtcatgaagattgaatggactatgaatttaaagtatctgttacagagttgaaaggtcacaaatgatagctataaagtatctagcccacaaaccttgc

SEQ ID NO: 267 - DNA, 401 bp, Homo sapiens
gcagtgactgctaaaaggtatggggttccttttggggatgatgaaaatgttctggaattggatgatggtgttggttgtacaattttgtaagtagactaaaaggccccgaattgtatactttaaaatggtgggttttgtattatgggaaattttatgttatataaattacatctcgagtttgagtttctgtaaaaaaaaaamcaaaaaaaaacaaaacaaaaaacaaaaaacaacgccagacgtggtggctcatgcctgtaatcccagtactttgggaggccgaggtgggtggatctcctgaggccaggagttcaaaaccagcctggccaacatggtgaaaccccatctctactaaaaaatacaaaaattagctgagcgcaatggcaggcacctgtaatccc

SEQ ID NO: 268 - DNA, 401 bp, Homo sapiens
gagcaagacttcgtcttaaaaaaaaaaaaaaggcgggggtggtggtggggagtaatggaggttggagtatttcttctcctgcctcttcctttccattttagcccactctggcagcaggcatatatttttttctaaatgtcccactaggcaatttcttcttcatagcttccattttcactagactccagtaacattgtttcytctccttgctgcttggtctagaagtggcaaggttttccaccattgttattctctggtggcctcaacttctttctttccttaacccttttgacaccactgggaataatgtgacttcattaaagtcacaaactattcagtgaattgtttcttgatgggcctctgactgctacacaagtgttcccagctgttagtgaagccat

SEQ ID NO: 269 - DNA, 401 bp, Homo sapiens
tccattttcactagactccagtaacattgtttcctctccttgctgcttggtctagaagtggcaaggttttccaccattgttattctctggtggcctcaacttctttctttccttaacccttttgacaccactgggaataatgtgacttcattaaagtcacaaactattcagtgaattgtttcttgatgggcctctgactgytacacaagtgttcccagctgttagtgaagccatgttctttgaggctctgatgctctggaatgagagctcctatgcaggatgacaatatggcaatagaatttttgaggaatactggtgttttctaatatactactctcaaccccattgtcatctcattctctattctcttgggaaattgcttctcccacaatggcttcagc

SEQ ID NO: 270 - DNA, 401 bp, Homo sapiens
ggtctagaagtggcaaggttttccaccattgttattctctggtggcctcaacttctttctttccttaacccttttgacaccactgggaataatgtgacttcattaaagtcacaaactattcagtgaattgtttcttgatgggcctctgactgctacacaagtgttcccagctgttagtgaagccatgttctttgaggctckgatgctctggaatgagagctcctatgcaggatgacaatatggcaatagaatttttgaggaatactggtgttttctaatatactactctcaaccccattgtcatctcattctctattctcttgggaaattgcttctcccacaatggcttcagctatcacctgtgtacctatgactattgaacttgaggtttttgtctctat

SEQ ID NO: 271 - DNA, 401 bp, Homo sapiens
atctgaaccattgaaatgaaagttttagacattacgacacttcatctgtaaatatttcattttgtatctcctaagaacaaggatattttcctacacacccacaacatcattatcactctcaagaaatttagcattgatgtgacaatattatctagtatatagtaaatattcatattttcccaattatcataatagtgtctyttatggctgtttttctaaatcaaagatcgaatctagtatcataggttacagttatttgttgtatccctttattcttcattaacaattccctcacttatttttttttttttaccttttgtaagagtgagatttttgcagcactcaggccaacttttttgtagccagctctacaaatttggatttttctgattgtttcctca

SEQ ID NO: 272 - DNA, 401 bp, Homo sapiens
tttttaaattgacaaatgaaaactgtgtatgtttattatgtacacttgttgaagtatgtatacattgtggaatgtctaaactgagataattaacatatgaattatctcacgtacttatcacttttgtggtgagaacccttaaaatctactctcttagccattttcaagaatacaatacattattatgaactatagtcaccrtgttgtacaatagatctcctgaacttagtcctcctatctggctgaaatgttgtatcctttgaccaacatctcccaactcctcagccccagcccctggtaaccaccaatctactctctatttctattcattcaacttttttagatactacatgtaagtgagatcatgaagtttgtctcttcctatgcctggcttatttcac

SEQ ID NO: 273 - DNA, 401 bp, Homo sapiens
aacctttcaccagataatttttaaactccgtcatctgcaacattcggctagcattctctgtaaagaactcttcttcaccctcccacctagtcttgttttgcttgtcagtttgtttttgaaatcattagtttttaattcggtgcattaaaaggcattacgatcattattctttttgattctcaatttgtccctaatttgacyagtgagaatttcttcaagctggctcctgtgtttacttgatatgttctcgttagttattgaacattttcttgggacaataaaatattctcccaaaattgcacttttccttcctcagagacctggaatatcttttatggaatgaactaagaaacttaagtgtttaaaccacgagtttattctgaaaccttcaaattgaatac

SEQ ID NO: 274 - DNA, 401 bp, Homo sapiens
tttttgattctcaatttgtccctaatttgactagtgagaatttcttcaagctggctcctgtgtttacttgatatgttctcgttagttattgaacattttcttgggacaataaaatattctcccaaaattgcacttttccttcctcagagacctggaatatcttttatggaatgaactaagaaacttaagtgtttaaaccaygagtttattctgaaaccttcaaattgaatacagcgaactcttcatctccttcccttccatattcacaattcccttccctcatggcggttaacaacatctaatattgacttaattctctttcctataatttgcaaaaagtagtgtgggaattactacaccagtaccataaccaacaacaaacatatagagtaaaatttaaa

SEQ ID NO: 275 - DNA, 401 bp, Homo sapiens
atacagcgaactcttcatctccttcccttccatattcacaattcccttccctcatggcggttaacaacatctaatattgacttaattctctttcctataatttgcaaaaagtagtgtgggaattactacaccagtaccataaccaacaacaaacatatagagtaaaatttaaatttcttttcagttcttttttgcactagrctatatcccacgaaacatattcggtcagggtattgcgttgaaaatcagtttctgtgtgggctgtgttgtcggttgtatagttgggttcatttgtttctctttgtgttcagattagagtattttgtcctttttagtttttttttttaatatgtatgactttaccatggcttaaaagtcaaaactatataaaaagttatata

SEQ ID NO: 276 - DNA, 401 bp, Homo sapiens
acagcgaactcttcatctccttcccttccatattcacaattcccttccctcatggcggttaacaacatctaatattgacttaattctctttcctataatttgcaaaaagtagtgtgggaattactacaccagtaccataaccaacaacaaacatatagagtaaaatttaaatttcttttcagttcttttttgcactaggcyatatcccacgaaacatattcggtcagggtattgcgttgaaaatcagtttctgtgtgggctgtgttgtcggttgtatagttgggttcatttgtttctctttgtgttcagattagagtattttgtcctttttagtttttttttttaatatgtatgactttaccatggcttaaaagtcaaaactatataaaaagttatataaa

SEQ ID NO: 277 - DNA, 401 bp, Homo sapiens
aattctctttcctataatttgcaaaaagtagtgtgggaattactacaccagtaccataaccaacaacaaacatatagagtaaaatttaaatttcttttcagttcttttttgcactaggctatatcccacgaaacatattcggtcagggtattgcgttgaaaatcagtttctgtgtgggctgtgttgtcggttgtatagttrggttcatttgtttctctttgtgttcagattagagtattttgtcctttttagtttttttttttaatatgtatgactttaccatggcttaaaagtcaaaactatataaaaagttatataaaagttcactcctttctcatcccctctaccctattctcattcctatcctatatccattgtgcagacaatcactttattagttt

SEQ ID NO: 278 - DNA, 401 bp, Homo sapiens
acaatcactttattagtttctggtttattcttcccatggttctttttgtaaatataagtgtgtgtggggggggggggcgttctttcttgttttctttctcttttttttacataaaagcctattatatgtactcttttgcactttgcattttttttacttaaaacatatcagtgtaagtaaccctaagcagttaagcaattmataaaaatctttatcattgagacagagtcttgctctgtcgccaggctgaagtgcagtggcgtgatcttggctcactgcaaccgccgtctcccaggttcaagcagttctcccgcctcagccccccgagtagctgggactacaggcgtgcactaccatgcccagctaatttttgtatttttagtagagacagggtttcacca

SEQ ID NO: 279 - DNA, 401 bp, Homo sapiens
aaatataagtgtgtgtggggggggggggcgttctttcttgttttctttctcttttttttacataaaagcctattatatgtactcttttgcactttgcattttttttacttaaaacatatcagtgtaagtaaccctaagcagttaagcaattaataaaaatctttatcattgagacagagtcttgctctgtcgccaggctgragtgcagtggcgtgatcttggctcactgcaaccgccgtctcccaggttcaagcagttctcccgcctcagccccccgagtagctgggactacaggcgtgcactaccatgcccagctaatttttgtatttttagtagagacagggtttcaccatattggccagaatggtctcgatctcttgacctcatgatctgcccacctc

SEQ ID NO: 280 - DNA, 401 bp, Homo sapiens
gttcaagccctaacccccagtgcctcagaatgtgactgtatttggagagaggacctttaaggagataagttgttcttggcctcccagcctccagaactgtgagaaaataaatgcgtgttgtttaagcccctcagtctatggtatcctgttatggcagctgaggcgcactaatgcagcctattttcccacagcttcagcagsagaatgagttgttaaacttgtgaatttttgccaactcaatcggttataaaagtatagttttggtctgtgtaaccgttttaagagtgcagctgagcatcttttcatgtgtttaagggtcaagttcctataatttttgtggaaattcacgatttttgcttttttctcaatttttaaaaatgttttatacattaaggagaggg

SEQ ID NO: 281 - DNA, 401 bp, Homo sapiens
gctgaggcgcactaatgcagcctattttcccacagcttcagcaggagaatgagttgttaaacttgtgaatttttgccaactcaatcggttataaaagtatagttttggtctgtgtaaccgttttaagagtgcagctgagcatcttttcatgtgtttaagggtcaagttcctataatttttgtggaaattcacgatttttgyttttttctcaatttttaaaaatgttttatacattaaggagagggcatttatctctgatgcatgttgcaaatatttttgcttttttagccacacaattctttattaattttatgtagtacaactcctcaatcttttctttcattgtatatggatctttagtcacatttagaaagcctgtcctatattcaagttacaaaata

SEQ ID NO: 282 - DNA, 401 bp, Homo sapiens
gtgaatttttgccaactcaatcggttataaaagtatagttttggtctgtgtaaccgttttaagagtgcagctgagcatcttttcatgtgtttaagggtcaagttcctataatttttgtggaaattcacgatttttgcttttttctcaatttttaaaaatgttttatacattaaggagagggcatttatctctgatgcatgytgcaaatatttttgcttttttagccacacaattctttattaattttatgtagtacaactcctcaatcttttctttcattgtatatggatctttagtcacatttagaaagcctgtcctatattcaagttacaaaatattcattcatgttttcttctagtatttgtatggattgtttattatgtatttttccatttagattt

SEQ ID NO: 283 - DNA, 401 bp, Homo sapiens
caaatatttttgcttttttagccacacaattctttattaattttatgtagtacaactcctcaatcttttctttcattgtatatggatctttagtcacatttagaaagcctgtcctatattcaagttacaaaatattcattcatgttttcttctagtatttgtatggattgtttattatgtatttttccatttagatttctygtgccttcagagtttattatggtatatggtgtaaggtgtggatccagttctgtctttatccaaatagttgtccaactgtcccaataccatttattcagaagtccatacttgcccaatgatttgaaatgccatctatatcaaataatacatttttatgctagcttaaatctatttctggactttctatcttgttcctctga

SEQ ID NO: 284 - DNA, 401 bp, Homo sapiens
ggatccagttctgtctttatccaaatagttgtccaactgtcccaataccatttattcagaagtccatacttgcccaatgatttgaaatgccatctatatcaaataatacatttttatgctagcttaaatctatttctggactttctatcttgttcctctgatctctccattcatgtgccactaccatactgtgttaaccaycaagcagttacagtacgttttagtatccaagaagaccaatctccccttattgcttccctcatcccccctgtgtagggtctcccagccattcttgcatgttcacttttctatataattttagaatcaacttgtttaggtccagaaaacaaaacaaaacaaaagcttgttgacatttttcttgcatttaagttaaattcaca

SEQ ID NO: 285 - DNA, 401 bp, Homo sapiens
tattcagaagtccatacttgcccaatgatttgaaatgccatctatatcaaataatacatttttatgctagcttaaatctatttctggactttctatcttgttcctctgatctctccattcatgtgccactaccatactgtgttaaccaccaagcagttacagtacgttttagtatccaagaagaccaatctccccttattkcttccctcatcccccctgtgtagggtctcccagccattcttgcatgttcacttttctatataattttagaatcaacttgtttaggtccagaaaacaaaacaaaacaaaagcttgttgacatttttcttgcatttaagttaaattcacatgttaacttgagaagagctgacatctgtatgatgttgagtagtttctctcca

SEQ ID NO: 286 - DNA, 401 bp, Homo sapiens
ctccattcatgtgccactaccatactgtgttaaccaccaagcagttacagtacgttttagtatccaagaagaccaatctccccttattgcttccctcatcccccctgtgtagggtctcccagccattcttgcatgttcacttttctatataattttagaatcaacttgtttaggtccagaaaacaaaacaaaacaaaagcwtgttgacatttttcttgcatttaagttaaattcacatgttaacttgagaagagctgacatctgtatgatgttgagtagtttctctccaagaagaacagatgcctttttacttgctcaagtctacttgaatgtctttctgaagtgttttagaattttcctcatttagattttgcatatttcttttcaagtttcacctcttt

SEQ ID NO: 287 - DNA, 401 bp, Homo sapiens
cctcatcccccctgtgtagggtctcccagccattcttgcatgttcacttttctatataattttagaatcaacttgtttaggtccagaaaacaaaacaaaacaaaagcttgttgacatttttcttgcatttaagttaaattcacatgttaacttgagaagagctgacatctgtatgatgttgagtagtttctctccaagaasaacagatgcctttttacttgctcaagtctacttgaatgtctttctgaagtgttttagaattttcctcatttagattttgcatatttcttttcaagtttcacctctttttgttgtaattataaatggatgttttctcttccattacatcttgtaattggttatttttgtatatatgaaagctattgatttttataagttaa

SEQ ID NO: 288 - DNA, 401 bp, Homo sapiens
ttgtaattataaatggatgttttctcttccattacatcttgtaattggttatttttgtatatatgaaagctattgatttttataagttaatttatatcctactgtctcattgaattccttatttaatttcaattagtttaatcatgaattctcatgggttttcaagctttactatcatttcatctgcatagagagtttttkcttctcaaatttttacacctatatctaattgtattgattaacaccgacaagacaatgttaaatagtaaggaagatagcatccttatcttatttctggccttagcaggaatgcctcccaggcttctcaattaaataagatgctaagtttaggacagaggtacacggatatgcataaagcacatatacacatgtacacatat

SEQ ID NO: 289 - DNA, 401 bp, Homo sapiens
ggttatttttgtatatatgaaagctattgatttttataagttaatttatatcctactgtctcattgaattccttatttaatttcaattagtttaatcatgaattctcatgggttttcaagctttactatcatttcatctgcatagagagtttttgcttctcaaatttttacacctatatctaattgtattgattaacaccracaagacaatgttaaatagtaaggaagatagcatccttatcttatttctggccttagcaggaatgcctcccaggcttctcaattaaataagatgctaagtttaggacagaggtacacggatatgcataaagcacatatacacatgtacacatatgtaaactgcgtgaagtctttctttttgagtgcattttttctatttc

SEQ ID NO: 290 - DNA, 401 bp, Homo sapiens
atatctaattgtattgattaacaccgacaagacaatgttaaatagtaaggaagatagcatccttatcttatttctggccttagcaggaatgcctcccaggcttctcaattaaataagatgctaagtttaggacagaggtacacggatatgcataaagcacatatacacatgtacacatatgtaaactgcgtgaagtctttytttttgagtgcattttttctatttctatttaattgagtgcatttttcaggaatgagtgttgaatattactgaaggtcatttcagcacctatggagataattgtttgatttttctccttaaatctattaataaggtgaattatataaattaatttctgcatatttaaagtctgcattcctggaatttgatattgatgtttt

SEQ ID NO: 291 - DNA, 401 bp, Homo sapiens
atctaattgtattgattaacaccgacaagacaatgttaaatagtaaggaagatagcatccttatcttatttctggccttagcaggaatgcctcccaggcttctcaattaaataagatgctaagtttaggacagaggtacacggatatgcataaagcacatatacacatgtacacatatgtaaactgcgtgaagtctttctwtttgagtgcattttttctatttctatttaattgagtgcatttttcaggaatgagtgttgaatattactgaaggtcatttcagcacctatggagataattgtttgatttttctccttaaatctattaataaggtgaattatataaattaatttctgcatatttaaagtctgcattcctggaatttgatattgatgttttat

SEQ ID NO: 292 - DNA, 401 bp, Homo sapiens
tctaattgtattgattaacaccgacaagacaatgttaaatagtaaggaagatagcatccttatcttatttctggccttagcaggaatgcctcccaggcttctcaattaaataagatgctaagtttaggacagaggtacacggatatgcataaagcacatatacacatgtacacatatgtaaactgcgtgaagtctttcttwttgagtgcattttttctatttctatttaattgagtgcatttttcaggaatgagtgttgaatattactgaaggtcatttcagcacctatggagataattgtttgatttttctccttaaatctattaataaggtgaattatataaattaatttctgcatatttaaagtctgcattcctggaatttgatattgatgttttatt

SEQ ID NO: 293 - DNA, 401 bp, Homo sapiens
tgcttatgtccccctaaaattcacattgaattgaaatcctaattcccaaggtggtggcattaggaggtggggcctttgggaggtgatcaggccatcagatatctacccttataagtgggattagtgcccttataaaagagaccccagagagctagctagtccctgtcactatgtgaggacacagcaagaaggtgttgtctrtgaatgaggaggtaggtcttcaacagacaccaaatctgctggcaccttgatcttagacttcttagtctctaaaactgtgagaagtaaacttttgttgtttctaagccacccagtctctgatattctgctatagcagcctagaggaagacagttactcaacatgaggtcaatttctgtgaacattctatttgtgcttaaga

SEQ ID NO: 294 - DNA, 401 bp, Homo sapiens
gtggttttaaatactagtggttttctattgtaatggccttcttctatcaattattttctctctgatctcttttgctttgttccttattttatttttattcccttttttcatggctttcctcactgtttcatattttcagtcaaatctctttttccatgggtactttataacttggcctttatttttaagatgattttgtcktttccttccatatctttcctgagttcactcaattcttatttcgcattttcctgaaaaatagggtttttcacaaaataatagggttttttggtcgtatttcttttataaagcttaaaacttctgatttaaggtttttgttttctatcttcacatgcttgttttaggatattcaattcagtgtgggtgttctgttacagttt

SEQ ID NO: 295 - DNA, 401 bp, Homo sapiens
tatttgatttcggtagaagcattttcatctgctggaatgttttaattcctgttttttttttttaagttattggccttgtatgagtgtcgtctgccattttctattcatttttagatgtttaaatatacagttgtagataggcgcagtcatggcgtttgatgactgaccatgtttcttagttcaagagcaccctcttctgtyggcatgggaaagtgtagtttcttcactgacagcagcttttgtgggggaggggttggtgtgccttctgagctttgagatccacttttgcttggtaggacactctttcttcgttcttcccctccactcagacgcctctgaagccctgctgccaagcggccccctctcctccagaaatagtgccctttccacaggccagccca

SEQ ID NO: 296 - DNA, 401 bp, Homo sapiens
gtgtcgtctgccattttctattcatttttagatgtttaaatatacagttgtagataggcgcagtcatggcgtttgatgactgaccatgtttcttagttcaagagcaccctcttctgttggcatgggaaagtgtagtttcttcactgacagcagcttttgtgggggaggggttggtgtgccttctgagctttgagatccacktttgcttggtaggacactctttcttcgttcttcccctccactcagacgcctctgaagccctgctgccaagcggccccctctcctccagaaatagtgccctttccacaggccagcccagccccatgggcagtcaaaccctccctttcattcgcatcctgaagcagcgccctcaggctacctggcctgtgagttgttcaagc

SEQ ID NO: 297 - DNA, 401 bp, Homo sapiens
tgtgttttagactgttcctctagcattatgactctcttctccatagaatacacacaaaagaaattaacctgcatcttacaactcaggcctcagcatcacaaagaaattaagttctgtgttaactgagtcttggtgtcatgtaaataaatataaacttgtttctctcgctgtttcaaagagaactggggtctctgttcattygattggtagccctctaagggatgggcgcttagttgggagctgccacatgtctgtgttgggttttattttttcacaaggccaaaatgttccctctagaacacacagtcctcaccaagttcagaaagaagccaactgcccttacctcctcccctcttctccatgctcctctgggctcagggtcctgcctctagaccactccc

SEQ ID NO: 298 - DNA, 401 bp, Homo sapiens
caaaagggccctccaaaggtcatcctggcaatgcccttgccttgctttgaaatgactcttgcctgttataaatagatgcaaatattgagtgggtagggagattctcctattcttaaagctgtcaaggtcaggagatgaatctgccccctcagtcacctgctcacaggttcccacgactctgacattcaggaagccaagaaragataaacttcctttatcaggatccaatcttcaacactagtcatttgaaaactatatccaactgtcacttgtagatcctctatctctctgcatctgcatccggaatactggcagcaatgacgacaacaatagcagatcagcataaaaccaaaaacacattcataaacacacacacatacacactacacacacacatacat

SEQ ID NO: 299 - DNA, 401 bp, Homo sapiens
atagcagatcagcataaaaccaaaaacacattcataaacacacacacatacacactacacacacacatacatacatacacacatatacacacaaacacacacatgcacacatgcatacatacatgtacacacacatgcatacacacatacacatgcatacgccatacaaatgcatacacatacacatgcatacacacacayatacaaacatccatgtatacacacacatacacacatgcatatacatatacacatacacactacacacatacacacatacgtgcatacacacatacatgcatgcatacacatacacgcatacacacatccatacatacacacaaacacacatgcatatacacacgcacacacacacaattaggcagagtctcagaggaaag

SEQ ID NO: 300 - DNA, 401 bp, Homo sapiens
ctaagaaacagtgagtgtgctgggctgtaatgaagaatgcatatcgtgaggtggcaggatatattgtcatcagagagtaggctggggccatgtcagggaacacctgaaatgtaaaatttcctgtatccttgaatcacttttttggcttcacttttctttttctttttttttttttttttgagacggagtcttgctttgtcrcccaggctggagtacagtggcacgatctcagctcactgcaacctccgcctctcaggttcaagcaattctcctgtctcagcctcctgagtagctggggattacaggcgcacaccaccacgcctggctaatttttgtatttttttagtagagacagggtttcaccaggttggccaggctggttccgaactcatgacctcgtg

SEQ ID NO: 301 - DNA, 401 bp, Homo sapiens
tggagtgaactgtttctgataacttcttgtggcccaaaatgttatcctgccctaaagaaatactaactggctcagttgaagatgggcatggtagtttccatgtgaggattagaacagcaggaaggtccttgcagaaagagagataatatagccattgcaaaacattgtatccagtctgcaaatgatcatcttagagaagtycttaagcccatttatgaccttagagctaattttaactcaagtaggagagctagtacaacataatcatcaaatcaagtaccaggcaaagaaatggcagaaataagactcaaatccaggtttaacttgggtgcaacctgggcagttagagcacttctccaagtgtccttgtctgtaaattagagatacaaagagtagctaca

SEQ ID NO: 302 - DNA, 401 bp, Homo sapiens
gaacaaacattggggaaagaatcatctcttttattctagtaaaactgaatatctacatgtagaagaatgaaacgagacccctatctctcaccacatacaaaaatcaactcaaaatgggttaaagacttaaatgtaagacccagtgcttttttaaaactactcagcaaacgctggctgttctgagggttcaggtggcttctyaaatctctgtgttcaagggctcatgactcatttatacttcctggaaagtgtcaggaactatttccaaatgggcaactggtgagagtgagatgtcaagggcagtgtggcctcagtctcctagcctgcccctcatggggcgccagattagggtgtcattgcagaaacgtatgttcttggagtttggtttctttttagcatcc

SEQ ID NO: 303 - DNA, 401 bp, Homo sapiens
ggataagctaaaggaaaattctggattggatgggagagtgggtcagtgattctcaaccaggggtggttttgtgtcccagggacatatggcaacatctggagacatgtttgattgttatgaattggggagtgatactgtatctagtgggtacagccaggggtgcagctaaacaccctcacaatgcacaagacagcccctcamaataaaaagtccaacaggcccaaaatgtcaacagtactgaggttgagaaaccatgcattaaatgatcttaagatatcttcaaactccagaactaaattaggggccttaatttttcctctgcatagatgggattccatttagccatccagtgtttctcaaagtagagcagggagaggacaaggatgtcaaaactatctcac

SEQ ID NO: 304 - DNA, 401 bp, Homo sapiens
accaaaaaggaataatttcacctttgttgcaaactatgacttatttctgataatatcaataaagtctgcagattccaatcactacatataaatgctatgtatcaacacatttctgcaggtgtattttgcaggtaaccgagtttttttttaagctacttctgctttttattgtatttaaatgttattgcccattttttaaamcattaagccctttttagtagtgtacatcaactccttatacaggttatcttccttaattatgtatttctgacaattcaacatttaaatataattttatatttaaatccaaaatcattggaagcatattagaattttaaattaaatctaaatttaaagtaattatttaaatatacagaaaatcaaaagtttacatttaaatt

SEQ ID NO: 305 - DNA, 401 bp, Homo sapiens
ttcttacagttatgaaggctgaaaagtacaaagtcaagggttcatatctggtgagagccttcttgatggtggggactctatacagagtcccagggtggcacagggcatcatgtggtgagggggggctgagctcagatctctgtcctgcttataaagccaccagtcccattctcatgataaacctctaatccattaacccaygaatgggttaatctattcttaggagcagagctctcatgacccaatcacttcttaaaagctccacctctcaatattgccacactggggattatgtttcaacatcagttttggaggaaacgaatattcaaaccatagcaaacgtccagttggcaaatgagagttgattcctgtgcaatgtcttttcccaaaggtagctttct

SEQ ID NO: 306 - DNA, 401 bp, Homo sapiens
cagttatgaaggctgaaaagtacaaagtcaagggttcatatctggtgagagccttcttgatggtggggactctatacagagtcccagggtggcacagggcatcatgtggtgagggggggctgagctcagatctctgtcctgcttataaagccaccagtcccattctcatgataaacctctaatccattaacccatgaatgrgttaatctattcttaggagcagagctctcatgacccaatcacttcttaaaagctccacctctcaatattgccacactggggattatgtttcaacatcagttttggaggaaacgaatattcaaaccatagcaaacgtccagttggcaaatgagagttgattcctgtgcaatgtcttttcccaaaggtagctttctctggcc

SEQ ID NO: 307 - DNA, 401 bp, Homo sapiens
ctacagtattgttgtctttcttttgaagctaataaaactgaagcaaagagagattaaatgacgactccaaggacacacagctagtaagtgctagatccaggatttaaaccccaaccactgacgatggctccactgtgccatcttaacattaattgacacccctcttaggcactgtactggttgcctcttaacatttaaccyattttattctctcaagggtcttatgaaagtgttcatgtcatacaatttagagatgagaaatcaggcccagagaaaaccaaaaacagagctagaaactgctggaaatgcagtttgaacccaaattgtctgactccaattttcttttcaatatatagaggcagtattatataatgtatattaattaatatataatgttagta

SEQ ID NO: 308 - DNA, 401 bp, Homo sapiens
taaataagaagacaggatataagagaggtatattgcgctacgaaatgggtgggttctcaagagaaaggtctctctccaaccgtcaggttaaaagcaagtttcttagagctggtatgctcagatttgcattccagatctgcttctcagtgattctccgcacttgggtgggttgcttaacctctctagctgcttctgcaaaaygggattcttgtggtgatgattaaatgggagaatgctcccttcagggagtggatcacaatgaaaaacagtacctgtatgtgccatcggctttgcagttctcaatgcatggccatgaacacagagtgtttataaaaatgtgttttgatgaaataatggctaagtggtctggattttggagattagaaatgatgtaaatttag

SEQ ID NO: 309 - DNA, 401 bp, Homo sapiens
atgatctagttctctcccgaactaagacctctattagaaatgaccctccatcacatcactaacactagcatgtcatacctcccaggctggaagttcttcttggggtctaactttagtgatctctctctctcaatctctccactcccacgttttcttccctcactccctcttacacacacacatacatatctcacatttctyattccagttcagtcctcacagaagctggaatagagataatcaacttcttagaattaaagcatcctctttttgttcagtactggctctttagttactgtttctcttgtcacagtccaggcgagaaaatgtcaagaatgtcggatagagagagagagacagaggagacagagtaagaggtgttttccagaactgagcaccac

SEQ ID NO: 310 - DNA, 401 bp, Homo sapiens
aggcgagaaaatgtcaagaatgtcggatagagagagagagacagaggagacagagtaagaggtgttttccagaactgagcaccacaagtgtggctgctggggtgtcctgcagagaacacagaagcagcatccacagccctgccatcctgaagtctccttcctctcttccaaagactgacgccagcaggctccgtcagaaasgagagccaatggtggagaaagggaaggaactttatctttggaattctgtcttctcagtggtccagaaaattcaaacagggccaggcactccatgtatcttcccagatgggcatcaggtacctgacacaacctggcaccccacattctttctccgatttccctgtcatcaggggcttgacaactctagcagccagggagag

SEQ ID NO: 311 - DNA, 401 bp, Homo sapiens
gccctggagcagggaggagagcaggtgtctcttctagcagcagctggagggttggaggtgatggaaaggtgatattgcaatcaagaatgcaggctgggattgagaaggtcagacctgccattttgccatttctgatctctcaagatagacacaatttcagggttagaatgaaagaggagtagagtgagacagaaagaaagmacagagataaacagtctggcttcattcttttcctggctttggacatgctcttcccttatttttctacctaagaagaggtcttgcagtgtggacagaatcctctggattccgctgccattgggttttgcttcatttctcttaagttaactttttggagcttcattttctccatctgtaaaatgaattgcacaacctcagag

SEQ ID NO: 312 - DNA, 401 bp, Homo sapiens
ggtgatattgcaatcaagaatgcaggctgggattgagaaggtcagacctgccattttgccatttctgatctctcaagatagacacaatttcagggttagaatgaaagaggagtagagtgagacagaaagaaagcacagagataaacagtctggcttcattcttttcctggctttggacatgctcttcccttatttttctayctaagaagaggtcttgcagtgtggacagaatcctctggattccgctgccattgggttttgcttcatttctcttaagttaactttttggagcttcattttctccatctgtaaaatgaattgcacaacctcagagctgtagtaaggattaaataagataacacagatgaaataccctagcaaaatacttgaccaactcaaca

SEQ ID NO: 313 - DNA, 401 bp, Homo sapiens
tttcctggctttggacatgctcttccccttattttttttttttttttttttttttttgagacggagtctcgctctgtcacccaggctggagtgcagtggtgcgatctcagctccctgcaagctccacctcctgggttcacgccattctcctgcctcagcctcccaagtagctgggactacaggcacctgccaccacacccrgctaaattttgtattttttttttagtagagacagggtttcaccatgttagccaggatgatcttgatctcctgaccttgtgatccgcccgcctcagcctcccaaagtgctgggattacaggcgtgagccaccacacccagcccccttattttctacctaagaagaggtcttgcagtgtggacagagccttctggattccac

SEQ ID NO: 314 - DNA, 401 bp, Homo sapiens
agtgcagtggtgcgatctcagctccctgcaagctccacctcctgggttcacgccattctcctgcctcagcctcccaagtagctgggactacaggcacctgccaccacacccggctaaattttgtattttttttttagtagagacagggtttcaccatgttagccaggatgatcttgatctcctgaccttgtgatccgcccrcctcagcctcccaaagtgctgggattacaggcgtgagccaccacacccagcccccttattttctacctaagaagaggtcttgcagtgtggacagagccttctggattccactggcattgggtttctgcttaacttctcttaagttaactttttggaactgccaaaaagttaacaagagaagttaagttaaacaaaatcta

SEQ ID NO: 315 - DNA, 401 bp, Homo sapiens
cgttactctgtgattcttaggaaacatcttctcttgtcctatagttcttaatcagcagacctgtgaccactgggagaggtctgtgattactgagcaaagttgaccacagaaagaatatccatattctcagaacctcatcatgactaatctgggaagcaatcttggaatatccatgtttcctgaaaagagtacagaagccgyggaatcaggacctgagaaggctgtgtagttcctggctttgtgaccaggagactctctggaagtcagtctccttatctgtaagaaaagacaaggaaataattcttatctaatgagttcttgggaaattgggaggagataattgatatgtgtcagcatgttgctcagctcctgggtatggtgccttccatcacgtcagataa

SEQ ID NO: 316 - DNA, 401 bp, Homo sapiens
agagatcttaacaatccagagatattcttttgggattggataatactcacagcttccacaaaagaaccttgcaaaaagttgtggaggctttacaagactagatggagccaggcctccatcagccaattctggagaaacacttgtatgctggaaaactgtccctgggacaaaaaaaaaaaaaaaaaaacaacaacaacaacracaacaacaaaacagcatgacactgataccaacattttaatcacgataattattgttagtgcttggcacaggcaaactcctaatctctgtaacctggtaagggttcttttctaaccaaggactcaaggatcaagttgtctcttcatctcagcatatgcctttcatggttgccccagcaaggtaaagaaagatcgaggtag

SEQ ID NO: 317 - DNA, 401 bp, Homo sapiens
gatcacagctatcggccagaactaatcaaatgggcctaatccaactgcaaagggagcacctgaataataagtgagcgctaaatgtctctaccactgctgaggactcaacagaggggcagtctgatgtctcaccatgctgatagccaggaagctctggatttcctccctctatgttttaatgacttaaccaggggtggtaarggaggcctttagaaaagattctcaaacacaggggcccagggatccctgggaatcatttcttactttgccatcatcttttctccctttttttttttctaatagatagctctcagaggggattaccaaagcaattttatggtctttatagctggttctctccccagtaattcagctgagagtcaaagatcacagaaaacctg

SEQ ID NO: 318 - DNA, 401 bp, Homo sapiens
ttccaggtacagggattttactgtcttcctccaaaagcccagctcatctttagggggccgtaacagtggattttagctcaacataggaaaaaacaccctggagccctcttttccactaactggccttacagtgcccgatggagtgggtcagaacaagccaatcgccccctcgctagagcccttcagatactgaatgagacrtgcctgccctcctgaatctcccctttgacaggcaacccctctccctgcaagtccctgagcctgacatactttctggttgcttctctgcacttgcttccagtcgttctttatcactttgaagcaagggacccacaaacagcccatgtgggcgctctgacttaaaagaggcacaacaggcctgtcacctcccattttctgga

SEQ ID NO: 319 - DNA, 401 bp, Homo sapiens
gcatcaaagcatgatgttgggcttggtgcctctcttccaccattctttctctttttaactccacatgtgtgctggactcttgcttatgatcttccctgtgtagctaccaccacaaaaatgtgtccccggcctttacaaatgaagtgttcagatgcaagccattctcttatagacatggcagtaaagaccaattaaaaaagsgggaaaattggtgctacaatgtcactttgcgggagaatgatgtctgtaaagtgggagcctatgtcttgcaatatgctgatgccctggttgaatagaccaaactattcattccttcctctatggcctggggagatctctctccatcacacctcagtgaagacagctttgatgcatttgactgtggtgccttgggcctccca

SEQ ID NO: 320 - DNA, 401 bp, Homo sapiens
cagagagaggcgtgttagatttccagacaaatattttcgcctagattttgctcaatgaacttgattcactgcagggctttttttcccacaagcgcgtggcagaggtgggctgccacaggagtggccagtgcccgctctctgcagggcactgctatgaatcaccattttcatgaatggaaagaataggaggaggggaaaaakgacaagcagcttgttgctaatgccctcaaacaaggcaaacattttgtgaataatttttagataatatatgacggtttggatctttggttttgttgttttgaatggaatgcatagaaaaaagaacttgtttcaacaaaagtttgatctgatatcctattcatgggctgattctttctcaaatatttggcagctcatccagg

SEQ ID NO: 321 - DNA, 401 bp, Homo sapiens
gctgcccagtagagaggaaagctcaaagtcatatcaattcaacacgcagtactaagtctctcctcttgctccattttctactggcctgtgaccaactacttcctaaagttgaaattgcttccgatttcttttttctctatttcatggataatccatgcagtgtttgctcaacctgtttacactgagatgtgttgaggaacygtcccacggcaagaccacaaggtggggcagtaatgagtaattggaagaaaactgcggtaccttctaaagctcaaatgtggctttctagaacacgatgaagtggagccctacattcattatttgaccttgtttgtttttttttttttttttttttttgagacagaggagtctcactgtctcccagactggagtgcaatggc

SEQ ID NO: 322 - DNA, 401 bp, Homo sapiens
caccatgcctagccctgtgactttttctcaagggcttcaaacagtccctctgcaagttaacctaggcatactcacagagcaagaaggcattcatgataccacaagggaggtgaggtttgtgccatttgctccatgtcactgaggcaggacagccctgtgtggctccattcccacgagagaccgtcttgctccgttaggacygcctcccacctgctaagggtgcttttcttccatggtacagtgatctttaatcatttgtttcaaaaatgtaccaacctcccgggaatgaaactaaccaaacttccctccctccctctctccttcccttccttccttccttccttccttccatccttcctgtttgccttccttccttccttttggattatgttgtttcatta

SEQ ID NO: 323 - DNA, 401 bp, Homo sapiens
ccatccttcctgtttgccttccttccttccttttggattatgttgtttcattaaaatatatttagctgggcatagtggctcacacctgtaatcccagcactctgggaggcagaggtgggcagttcacttaaggtcaggagtttaagaccagcctggccaccatggtgaaaccctgtctctactaaaaatacaaaattagcygcgtgtggtggtggacgcctgtaatcccagctactcgggaggctgaggcaggagaattgcttgaacccaaaaaaagcagaggtttcagtgagctgagatcgtaccactgcactccagcctgggcaacagagtgagattccgtcatatatatattcacatgatttaggaaattctgtatttcagcatcacagtataaagac

SEQ ID NO: 324 - DNA, 401 bp, Homo sapiens
gctgaggcaggagaattgcttgaacccaaaaaaagcagaggtttcagtgagctgagatcgtaccactgcactccagcctgggcaacagagtgagattccgtcatatatatattcacatgatttaggaaattctgtatttcagcatcacagtataaagacttaagttctaagagttgatattgctgtagatgttgggaatgraatcttaggagtaacctttaccggaggcaacttctactgcccttgctacctttggtgtgacctgaaaccccctttcttcattagtaccaagtgacttgtttgccactactcagctgtcccagaatagcttgactccacacatccatgcatttctaggaagaaattaggtgcattataaataacgagtcctctgaaaatgc

SEQ ID NO: 325 - DNA, 401 bp, Homo sapiens
acaaaagctgggtaacttgcagttcccttaccactaaagtggaaacatggatgtctaaagcttttttacagctagaaaagtctatgcttggttagcattgtgctgatgcaattctgttccagataaccgagggagggaatggcggtcctcgcgtgaagactaagtagacgtgctattaccctcctcggttggctccagcartaagcctaatgacctctacaaagctgttctgtaatacagaagtaaacacttactctgcacaatttctccttgcatgtgcttcccactggtagaacattattcatcttgttggtctttccaaagagattcattacttccttgctccttcttattcatccttcataatagaaatgctaaaagtctgaaagacgaggaaggga

SEQ ID NO: 326 - DNA, 401 bp, Homo sapiens
gtcttgtttaccattgaatctgatacctaacatggaatctgacacctgctagacaccctcagaatgtctgtgaaatgatgtaactctacgtgatgtgtgtctactcatagctgtaaaatgagtgagccctcctgtaggtcacagtgtccagcagactggcagggacctgatatgaacatcacagaggaaaaaaacacataygaaacaattaatgcttgccattgggccaaatgctttgtatgtgttatttcatttatatttttatttaatactaagcagacttcatgtaccctgtattttgctaggcagtggagtacatagaaaggaacagaaactgcgtatcttgtcttcccaactgaaaggagacgattaacctacttgaagatgttagaatacaaata

SEQ ID NO: 327 - DNA, 401 bp, Homo sapiens
gtcgtacagctcttaagtagcagacctgggcttaaaaccaggttatcctgggttcaaaatcttaggttctttcacatatcctattcttcctcctggggctaaggtcacgcactatattttgtcactgaaacatacattaatgcacaatctctgagagagtattcctttgtattagcattatattaatatttatattagtgygcatattatatgcagaaaacctcaagtccaaatatataagtaaaaaagtagagataatataaaaattattcctaatttccttcattaatccattggagatacataatttatgaaactctccttaagcctaatttatgttattagcctatgtcctttcttggtataataatttccataattttctacctgttaaataaagt

SEQ ID NO: 328 - DNA, 401 bp, Homo sapiens
ctggacaacatagcaagaccccatctctaaaaaaaaagaaaaaaaaagaatgatagcatttcctttacttgtggagattttctgactataacatgcacactattattataaaataatagactttacaaaatataatggttgtagtttcatttgcactaaataacgataaaccttatttaagtgcaaccttattttatatayatatatatatatatacccattaatttgattgtttttaatgtagccatggacatggagagttcacaaattataatttataaatgtattaagatagaataattgcagaatagaaggatttttaaacaatttaaatagtcaccatttctgccacccattaatcctatatattggttctaggacttattcctggagttatatcc

SEQ ID NO: 329 - DNA, 401 bp, Homo sapiens
actcaaacagtctgctcttagatggtccttattgcagaaagcaggcttatgccctgtaagccgggggtatctccaaatcgtgcaggaggagaggtactgattgctcatttgcttcttctgtgccttaaaatcaccatccttgttaaagaaatgcaagcattgagtagagaagtgtcttgcttgggcttgcttttgagtacygggaattcagctttcctgctttggaaacctgagttgggatgtaggtgtgtagcaaaaggaatgggtggaggcttctaactaatggggaaggcatctctgaaacaagctgagatgtggatctgggattctgggaatcaaataattcttcccagcaccaaactttgatgagagaatactttaatgtctgtggttgcagaata

SEQ ID NO: 330 - DNA, 401 bp, Homo sapiens
tgcttattttatccaagttaaacttccacagaagattcaactataacttatatgggacaatcttcagatcctcagtaacctagtcatcttttgtaatttggatactaagtgtcaggatctcccttaaaataggccatccaatattgaagctagttttaagatacagtctaagcagaaaagagcagagactataactttccrtggcagaagcactccaagactcaagatgctaaagcaatgttgtgactgatttctttttctttgttgtctttaaaaaatatattcagttatgcaagtcagaaatctctacaccctgtcacgacagcatctgggcataattatgccatataagtcaatattgttcacatatggaagaaaagttggatcacaccattcattct

SEQ ID NO: 331 - DNA, 401 bp, Homo sapiens
ttgtgaacagaaatattatctacatataatctacattttggaatccaattgtcctttaaggtttgagttttataattaaatcattttgagatcatggttaacaaatacaagaataatagatgataatctgtagatgtcagctgggcgcagtggctcacacctgtaatcccagcactttgggaggctgaagtgggaggatcycttgagcccacgaagtcaagaccagcctgggcaacatagggaaaacccacctctacaaaaaaatagaaacaattagccaggcatggtgatgcatgcccatagtgccagctacttgaaactgaggtgggaggatcgcttgagctggagaggttgaggctgcagtgatctgaggtcgtgcccctgcactccagtctgggtga

SEQ ID NO: 332 - DNA, 401 bp, Homo sapiens
agtttcgactggtgcttagaggccatggtagagtggacatttttttgtttgtttgtttttgagacaaagtcttgctctgtcacccaggctgaagtgcagtggcacaatctgggctcactgcaacctccgcctcccaggttccagcgattctcctgctgcagcctcctgagtagttgcaattacaagtgcccgcagctaaayccagctagtttttgtattttcagtagagatggggtttcaccatgttggccaggctggtctcaaactcctgtgctcaggtgatccacccgcctcagcctcccaaagtgctgggattacaggcatgagccactgtgcccggcctagagtggacattttattctacatacaatgggaagccatttgagggatttcagcaagga

SEQ ID NO: 333 - DNA, 401 bp, Homo sapiens
ctttaattttaattgtacccatctgggttcttcgaaatggcaagtatttcgtttatgtgcctttctcatcttgggtcaactcagttccatgcactttaaaattattttaggaaagagccacatcttttttttttccttctttctctttctttctttctttctttctttctttctttctttctttctttctttctttctttytctttctttcttttttgtgcatgcatgcgtgcatgcatgtgtgtgtgtgtgtgtgtgtgtgtgtgtgtgtgtgtgtctcactctgctcgcccaggctggagtgcactggtacaatcttggctcactgcagccccgacttcacaagctcaggtgatcttcccacctcaccttagcctcccaagtagctggaattacaggca

SEQ ID NO: 334 - DNA, 401 bp, Homo sapiens
tagtaaataaataaataaataaataaataaataaataaataaatgcacttttttgtatattgagttatatctcaatgaagctgctttttttaaaaaaaaaaaaaacatataacataaaagagattaacagaagagattcaaaagtctagaacaccaaatattaaaggtagcttttcaaatcagtgagaaaataatgatatkacagaaaatggcaaatcatctagaattaaatttctacttcatactatcagcaatataaattgcagactaatctaaattaacaatttaaaagataaaagaaactatagaaaaatacacaattatgtatctgttcttgaatggaaaaagattttccaagcgtaaatacaaagaaaaatttatatagtaaaacaatgattgat

SEQ ID NO: 335 - DNA, 401 bp, Homo sapiens
atcctagcactttgggtggccgagatgggcagattgcctgagctcaggagttcaaaaccagcctgggcaacgtgatgaaaccccatctctactaaaatacaaaaaattagccgggcgtggtggtgtgcacctgtagtcccaactactcgggaggctgaggtgggagactcccttgaacctgggaggtggaggttgcaccgrtctgagattgcatcagtgcactccagcctgggtgacagagcaagactcccgtctcaaaaaaacaaaatggcaaatagcaaaggggataatatttgccacaaatataaaagaaaaggatatatccttgctatataaaaaggtcctatgaaacagggacttagttggatatttctatttttaaaaaggcaaatcatataaac

SEQ ID NO: 336 - DNA, 401 bp, Homo sapiens
agagtttccttgttgggaccccccccccacccgccgggccccaccccgttgatatcctaaatgttctcttgacatcttggatttaccatagagcctagtactgtctgtaaggtcctgggcctgttctaattagtgctgagtatctgttgatctgctacgtgcaaatgggaagataaaactaaatgttagattagggtcaayctttaaaactctttcctagattgtgatggtattcagtggtggttgaccatcatggaattgggagatatttgtgaatgtggctgcaggctgcatgtcctttgtgtctctgactcaagacacacccattgatctcatttgtcttcatttcatcattgggcagaatgcgttctaaaatgaagagactaaaaccttcacagaag

SEQ ID NO: 337 - DNA, 401 bp, Homo sapiens
atgtggcagactgccagttgccttccccagcataacattttcccaatattcattaggaaaagaaccctttaattgtacccaattaaaataccgaatctcccagcctcttctgcagcagatgtggctgtgtgactaagttctgtccaataagatataagtatacatgtttggtgagatttccaggaaggatgctcaagtggrgatgactaagtcagaagaaagttccttttagctttcctgcattcctagaattctgatgcaaaagctggagctacagcagcaatcctggactatgaggtaaccttgggagtactagggaattgggatgctagtaactttctagaagcaccaactcagccccagaatgcctcctgctgacttcttttacgtgggagagtaag

SEQ ID NO: 338 - DNA, 401 bp, Homo sapiens
acaaggacaaactcaggtcatcatacaatctctggcccttcatctttctggcatgacatcatgtgagtcagcacagtcggttttggcatatttctggaagccttttcaacactggaataccttacaataactttactagacatctcactatgccttaaatcaatgtatctttcactcaactttctctcagttcagattccraaaatgttcagggccattccaaacctacttccacaagaggaatattttggacagaaaatcagagtgaaaaaaagactgccatcttagataattgtagtttaattatcttatcctgcatattttacataaatgtgtaagcatgtgaacatgtgactaggactcctttcaagacattggaaaaggccatactagtgaaggac

SEQ ID NO: 339 - DNA, 401 bp, Homo sapiens
tatattagttgttatagacacactcactgtctgtgggactattacaggtttggcccctggaaacatggggattctgatcatttaatttcctgaaattgccattgaaaaagaaaacacaatttggtgaatttagaattgcatacacgaaattatcatgtttattcttgagagaagaaaatttccatttgactagacatccayggaacttagtctcctggttaccttaccacatgtcgaatgcctggtagaactttccacaggctagcttttggctccatacatttcaaaacttgtcccatcaacactcatatgccattggtgcttgttgcacaaggacaaactcaggtcatcatacaatctctggcccttcatctttctggcatgacatcatgtgagtcagc

SEQ ID NO: 340 - DNA, 401 bp, Homo sapiens
ctttaaacattttataattgtggaatttcttttgtcgttttacagaaatattcagttttgtacctcataatttgtatttataattttgtattcttcttcataaaaagggccattgaattgtgtttaactcatgcctcaccaaacctgaaccagcattttcctctgttgaatttctattgcctgcctacagcaaggcccacrtcttcctgtaacagttgcccccagttcccttaactatcaccagggagtcagcagttaatgacatgctcaaccttcctccaattacagatggttaggagagaaaacaaagccataaggccattaaactcatgaggttttgactggtggttcggaggtagagctggccctggctatatctgccgtgtacacaagaaccaaaa

SEQ ID NO: 341 - DNA, 401 bp, Homo sapiens
tgtttatgaggcaccaagagggatacaaatgaccaagattcactcagactaatggactgaaacttcagcagcatgggcagagttaattgcacaccccatgcgcatgtggccttgcacagggcacgtgcaatccttgatgggtctaatgagggcccctctgctgtgatcagagacccgtaattccttccttgtctggaaaayggggcttcaggctcccaatgttctcatgagtatctgtcacatgtggggtgcttttgctgtcttgagaagagttaaatctgtgctgcaatcttttctgatttttcattagatcttccatggcatctgtacaacttctgctttataaattgcattttttcaagtattttgcaaaatcttttgcattgcaatttgagagtgtt

SEQ ID NO: 342 - DNA, 401 bp, Homo sapiens
aacaatagatgcctcatcctcctcactatgttgctgtgagcatctgttgaagtagtggctattcgagtgcattgaaatgcatcagtcagtacacagatacaagggcttctgataactcatacctgaagaatttttgtcaaccctgaagggatttgcttcttggtatacgggtattaaaatactagtataatttctctaacsggagtatgatgtcactagttccagagtcttttcatagatggatgggttttatgataaataactcaccctcagtgttaacttggctttccaactagccaaacaggcctttgtggaacaaggttagcaaatagcatttgggcaaacgatcaaaagggtctccctctcttctatcactctctttatctctcttggtacctctc

SEQ ID NO: 343 - DNA, 401 bp, Homo sapiens
ctttctgtgcctggcttatttcacttaacataatgacatccagttccattcatgttgatgcatataacagatttcattctttttatggctgaatagtaatccattgtatgtatatacacatataacgtctgtatatataatgtgtatataaaatgtatatccataatatatgtatatatacacatatatgtaatatatatwgtgtgtgtgtgtgtgtgtgtgtgtatatatatatatatatatatatatatatatatatatctcacttttttttttgagacagagtcttgctgtgttgcccaggctggagtgcagtggtgcaatctcagctaactgcaacctccacctctcggttcaagggattctcatgcctcagtctcccaaaaagctgggactacagt

SEQ ID NO: 344 - DNA, 401 bp, Homo sapiens
tatcttggctgttgtgaatagtgctgcagtaaacatgggagtacaggtatcattttgatatactgaattcctttcctttgggtaaatatgtcatggtgggtttgctacatgatatgatagttctacttttagttttttgagaaacctccgaactattttctacagtggttgtactaatttacattaccaccagcaatgtayaagagttcctttttcctttttccacatcctcactgacatttattactttctgtctttttgataatatccatccttactgagggaaagagggtatctcactgtggtttcaatttgcatttccctgatgattcgtggtgttgaacttttttttttttcagatacctgctagccaattgtatgtattcttttaagacatatct

SEQ ID NO: 345 - DNA, 401 bp, Homo sapiens
atcaccgtaacactgcactcagagataaccactgttaggacttttatttctttctttctcttctctatacacctgtaacatgcatgtacacctactgcatgcatatgtgtatgatacatgtgcaaacagtgcatgctaacattttctaccctgcttttccatgtgttatttttatcctgtgttccactattttggaaaccrtcagggttgacagagggaaattgatcaagccctttgtcttcattgatgtcaagagtttcagtcaacttggaggccgtggcctcctccttacaggcttcttcagcctttggtttgagtttgggttttttacttttcatttttggaggagccacgttctctctcctttcctattactggagagccgcttcctctcttcactc

SEQ ID NO: 346 - DNA, 401 bp, Homo sapiens
atcttgccacactcaaatctcatcttttttgccatgtttgccccatggcatttctctcagcatcttcttgtgctttttccaaaacatataaaggacaattctactctggaaagtcactgggatactgttctctgagaacaggtattgagaagaagatggtttcctcttggaacttggccaactggctcacagcgaacgtgstgtggactcttaccagctctctctagtatgctaagggcccccaaccagggaccctgaaggcatctcaagagagcagtgtccaggtctgcccttgttggaaggaggatgatgtgaggagatcacctttattaaagctgcaggttgtctgccagattttcaaatttcaatgtaactttttatttaaccaatgctgattaata

SEQ ID NO: 347 - DNA, 401 bp, Homo sapiens
gcaacctgtcatattgggactgtgcagaacttttccatgttttaaaactcaggagaaagaagtaaagatggtaagagtgcacttgagcagaaacaaagggctgtgatcccaaaacaggggacaaatgagctgcaaaggagagacaccaagagcggagagcaagaggcaaagtgatatgaaagactccaaagtgctggttcwgagaaaagaaaaaaaagggacataggtcaggaaaggggagaaggactgacatgaactggcaaatggcaacagatataaaaagccagctttctctggggagtggaaatgtttctaagcatttttgttcatgttagtatgatttgttttttcctaagtaagcttgaaaatcatagagtgaataggagaaaccaaagattagg

SEQ ID NO: 348 - DNA, 401 bp, Homo sapiens
agaaaccaaagattagggaaaatatttttagaacaagctcagggaagtcatataggtttaaaatatatagtctatgcaaataatggcacacatttcagaatggaaacaaaaataagtggcagataatgtgtccaagtaatattggtagcaaatggactgttacgagagcaaatgatattttgcagagctgagagtgtgacytgtcttcaattatttgaatccctatcctacaaaagcattgaaaaaggaagatctggagccaatgactgtgtattacatatattttcattcagtacaagcagacatcatcagtgtgctatatactactttcattattattattatcttaattaaagctgtcttacgagccaggcgcagtggctcacgcctctaatcccaac

SEQ ID NO: 349 - DNA, 401 bp, Homo sapiens
cgagagcaaatgatattttgcagagctgagagtgtgacctgtcttcaattatttgaatccctatcctacaaaagcattgaaaaaggaagatctggagccaatgactgtgtattacatatattttcattcagtacaagcagacatcatcagtgtgctatatactactttcattattattattatcttaattaaagctgtctyacgagccaggcgcagtggctcacgcctctaatcccaacactttgggagaccgaagcaggtggatcacttgaggtcaggagtttgagaccagcctggccaacatggtgaaaccctgtcgctactgaaaatacaaaaattagccaggcatggtggcaggtgcctgtaatcccagctacttgggaggctgagacaggagaatt

SEQ ID NO: 350 - DNA, 401 bp, Homo sapiens
ctttaggaggctgaggcaggtgaatcacctgaggttaggagttcaaggccagcctggccaacatggtgaaaccctgtctctactgaaaatacaaaatttagccgggtgtgctggtgcatgcctgtaatcccagctactcaggaggctgagacaggagaattgcttgaacccgggaggcagaatttgcagcgagccgagatygtgccactgcactccagcctggacaacagagcagaacactgtctcaaaacaaaacagaacaaaaaaagaaaagggatgctttgtggaacagggagattacttgcactggaattgttcaagccaagttctatcaacatttgacacagaaacacatgaccatcgtaaggtctttcccagctcagcattctgtgactgcgtgg

SEQ ID NO: 351 - DNA, 401 bp, Homo sapiens
ttgtggagcttccccagcccgctcaggctgcccccagtcttcccatttttagaaaaattaaaactatggtttgttaaagcttctattctgatggtctctgttacttgtagctgaacgcaatgaattgtgagatacgatttaccacaatttttttctttctttctttcttttttcttcttcttcattttttttttttttttwaaagggttgtgccatgtcacccaggctggagtgcaattgtgcgatcatgactgactgtggtgtcaacctcccaggctcaagcaatcctcccacctcagtctcctaagtagctgtgactataggcatgtgccaccatgccaggctaatttttgtattttttgtagagatggggtcttgctgtgttgcacaggctggtctca

SEQ ID NO: 352 - DNA, 401 bp, Homo sapiens
cctgggaggttgaggttgcagtgagccaagattgcaccactgcactccagcctgggtgacagagcgagatcctgtctcaaaaaaattaaataaatcaatatatggtaatctaatgggatgctatgcagcactaaaagtatattttaaaaaaaacttgagaaatgcttctaagatgaataatacacaattaagtcaattacmtgacttcaaatattattataactgcatgtggacaagaagacaaaaccttcaaggaaaaaaagttttgatattatgagattatgggtaacattgtattcccagccttgaatttctgttattgttactataatatgtgtacaatttttttttagtgaagtaaaacaaacaagtaaaagaaaggttgaaacttcacaatgtag

SEQ ID NO: 353 - DNA, 401 bp, Homo sapiens
ataagtatgcagttatgaggaaaggtgatagaaccacaccatttttatgaataatatactaaaatatattcttaaaaaacttgtcagttgtatatttgataatagcatatttttacttagtccttattaaagatctcgggaaattcaaatctgtttcatctttgatcaggagagaagatgttgcccatccttagcccagarttagaaaggagaaatcatgtgattggccaaggtggtgatgagagtctcttcagaagggaagacttccagttgcaaggtcaggtgacccaatcaactgcctttgaacatctggaaaaatcttctaaaactcttccttctcaagaagtcctgcaactggagctactaatgaaagctctttccagtagcccttacagctaatt

SEQ ID NO: 354 - DNA, 401 bp, Homo sapiens
ttatgaagtgagtattatgatcatttctaatcttatgcatgacaattgagatttaaagagtataactttccaaggtgacaaatttagcaggtgtcagaatccagttctgtcagaagaaataagtagaaaataaaggtgggtttgttggcgactagataatcataataaacattgatataatcgtttataatttttcataamcgtagaaattatacaacctgtgtaaagccaaaatactttggtgatttctgggttcagaagataatgccttttgtagctatggagcttagatcttgtagctatatgtgtttgactgagggaagtggaaattctctaactgggatatcataccaggatgatgaattacctgtgcaaactctcactattgcaaggtcctatct

SEQ ID NO: 355 - DNA, 401 bp, Homo sapiens
agacaataatattataaataaataaataaacaaactgtaaatggtacaaatataaaagagcaatactaacgacaaataataatacatataatgaatgcattgataatgacagtgaaagttagaagtgattcttttagcacagagataagagattagctcatggctcaggccaggcctcacagattatctgtctcctggtgrgactaatgcaagatctgaagtttggtctgtaactttgtcattttggtttgttctatggaccaaactctggagtactgattaacatcattgtctagcagttgaaagctctctgataaccttggggagtgctagatagaatagatacaattttgccttttgattttcctttgcccaaacaagtggttcacatatgaagatgt

SEQ ID NO: 356 - DNA, 401 bp, Homo sapiens
gaagaagaagaagaaaagaaaaactttccctgatatatttaggttgagactagacaccctcttatctgctcccacaatgcccacgtcctccatcactgtactgaccatactctggtaattggctgtttatctgtccaatgccttccctagaccagaagctcctaaaggacagagcctgtattcccattcgtagtaccagcrttcagcacagtagctggacctgactaagcaccaagtaaatatttgttgaattaagaaaattaaaaaaaaagtgagaagtcaacagtctcttaatcaattgtaaagaaacactttactaggatgttctgacagctactccaaagtatcaaatattttaagtgaaattgtgatgtctggtgacattttttcaatattttaga

SEQ ID NO: 357 - DNA, 401 bp, Homo sapiens
ttactttaatatgtccaattataccattatcatctatttatcaaaatagtgccatttaattgccttttgctttaagctgtattttatatgaaagattcctagtgttatttctgtcctttggaatattatccagcagagggaattttaggatctacttgataatacagatttgctaatttccacgttagtacttcagtgttrtttagttacacagtttcgggcctcatgatgtgtttgatgatttgggagtgataaaatgagtcttaagtaaaaatcttttaattaaaacaaacaaaggatcagtttttatgggaaaccagtcagtcagcaagtacatgtgttttaagtccagaaggatgctcaagtggaggggtagagagagagagcccttgggatccagg

SEQ ID NO: 358 - DNA, 401 bp, Homo sapiens
gtatatatccaaaggaatataactgttctaccataaaaacacacgcatgcttatgttcattgcagcactgttcacaatctaaaagacatgaaatcaacctaaatgcccatcaacagtaaacaggataaagaaaatgtggtacatatataccatggaatactatgcagccataaaaagaacaaatttatgtcctttacaggwacatggatggagctggaggccattatccttagcaaactaatgcaggaacagaaaaccaaatactgcatgttctcacttacaaatgggagctaaatgacgagaatacatggacacatagaggcaaacaagagacactggggcctattgagggtggagggtgggaggagggagaggatcagaaaaaataactgttgggtact

SEQ ID NO: 359 - DNA, 401 bp, Homo sapiens
tcccttcccagctctgtctaccactttgtatcacttaagccacgttttatctacgtcttctctgtcactaacctttagtcccttgaaacaggcacaaccaaacaggactgacattttgcaagtcccttgagattttgcctaatccttcacagcaacctacaaagtcaatctggggctggtggaaattctcctctttcagcyttttccttcttttggaatttgggggtgttttcacagagtgatttaccaagaaaacattctaaatcctcaaggatcacttatcccttacctcgtggttgtttgctcgagaccaagcacaactggacatttttgaacttacgtctttctccctaccctctttgttctccttctcccccttcataaaatctgacttcagtttg

SEQ ID NO: 360 - DNA, 401 bp, Homo sapiens
agtgtcatttttacggggtgacaaagcattaagaagatcgctttttcagccttgctgtgcttcattataggtaagtcctcatgtggcaccttttacaagaggaagtattgtcatgctaacaaaatggtttcagatagtatgaaatcagtggtgagaagaatggctataaggaatgggctctacaattactgtcactctcascaaaagagaacgcaaggaatgcatatttcatctcatggcgacctgaggatcgtcctcaattccatatgtaatactttctaaataatggtgacctttttcacgtccccttcctgtatatacaaagtactatgtggataatgcagcctgagtgagcttatgagctaatgcagggatttttattattaacaataatcatagtg

SEQ ID NO: 361 - DNA, 401 bp, Homo sapiens
tggctataaggaatgggctctacaattactgtcactctcagcaaaagagaacgcaaggaatgcatatttcatctcatggcgacctgaggatcgtcctcaattccatatgtaatactttctaaataatggtgacctttttcacgtccccttcctgtatatacaaagtactatgtggataatgcagcctgagtgagcttatgmgctaatgcagggatttttattattaacaataatcatagtgaacaacagtttattcatgtaagatcaagcaactagtaagtggtagaaccaggactgggtcatagggtaggtacatgtgtaccacttttaaatattgccaaactactttgcaaagtattgaattattttacattcccaacagcagtgtctgggaattacaa

SEQ ID NO: 362 - DNA, 401 bp, Homo sapiens
gggaattacaataatccttatcatgcaatgactggcgtattcattctttttaatttttgacattctaatctgtgtgtagtgccatctcattgtagtttgaatttgcatttcgctagtgactaatgatattgagcatcttttcatgagcttatttgcatatgtatgtcttctttgctaaagtgtctatgcaaatcttttgcycatttattaggttgtttgttttcttatattgagttataactttttttttttttacatattctgcaaacatttttaaccagatatgtagtttacaagtgtgttttcccagtctgtttcttacctttacattcccttaccagtatcttttgcaggaaagatgttaaatctgttgaagtgtagtttatcaagttttttctttg

SEQ ID NO: 363 - DNA, 401 bp, Homo sapiens
atgtcttctttgctaaagtgtctatgcaaatcttttgcccatttattaggttgtttgttttcttatattgagttataactttttttttttttacatattctgcaaacatttttaaccagatatgtagtttacaagtgtgttttcccagtctgtttcttacctttacattcccttaccagtatcttttgcaggaaagatgtyaaatctgttgaagtgtagtttatcaagttttttctttggattgtgcttttggtcttgtagctaagaaaactttgcccaaacaagataacaaggattttctcctatttttttctaggcatgttatggttttaggttttacctttatgtctatgatccattttacattaatttttatatatggtgcaagttatggatcaaag

SEQ ID NO: 364 - DNA, 401 bp, Homo sapiens
aaagaccattcataagatgtagtcagtaccagtaaatgagagcacattttcctctaattgggtgtgtggatattggttttcaccctacttggagtttctgactatttgtcctagtgaccatcactatattcatttataatattattaaaatcaatggatgagtcattggttaccatgtgtattccggttctgaaacacacyctcctcttccccagccagcactcacctcagtgatacccagtctattgcaggttttttatgcattttctaaattacatgaaagcaagttgacattctttctttgtcttagtcttccataccattattttggcatgtttaaagtatcatccatctgattatgtttggaaataaaagcatgaatttgttaaagacatgtacct

SEQ ID NO: 365 - DNA, 401 bp, Homo sapiens
ccattattttggcatgtttaaagtatcatccatctgattatgtttggaaataaaagcatgaatttgttaaagacatgtaccttccaaactgcacacttattgtgttttcactttcttatcattgtcccttcattagagaaaaaaatctattctgtgaagtatttggttttttatgcagtctgctctgctctaatatacacraacacctgattctgcaatgagaattagttggtatgtgattgttgagcagggaaatagtactatttcacagaatttcctttagtttatatatggatttttcttggctgaagaagccagattactggagtaaaattataatctccagcaaaaataaaaaaagaaaagtccacagctcagcccctcctctaaagagttgtaca

SEQ ID NO: 366 - DNA, 401 bp, Homo sapiens
gtatcaattatcatttttgtttggactttacaaagttttataggttctaagtgatatatcccttctgcagtttttctcataagttctgtttatacagtatgtgattttgcagaatatgagttttttgagattatatatattgcaatatcatagaaacctttttgtttgataatctttgcttaaaataacctcatttatttygtacgaatgtacaaaaataaaacaatgtagtacttattggctgtttttgtggactgaggtagagatgtcaagaagttcaagccaaaaaaactgaaaatagttaacagaaacaggcaaatctcatatatcttgccctgattattttttcattagagtccctcctattgaggaaggttcattagcatgtgatgaaacacatt

SEQ ID NO: 367 - DNA, 401 bp, Homo sapiens
ttttgattttcatcccaaagtgacttttaaaaaagttttggggttagggggatgtggtggctcacgcctgtaatcctaggactttgggaggccaaggcggggcagagggcggcagatcacctgaggtcaggagttcgaggccagcgtggccaatattacaaaaccccttctctactaaaaatacaaaaactagccaggtgyggtggcaagtgcctgtaatcccagctacttgggaggctgaggtaggagaatcacttgaaccccagggcaaaggtattagtcagcagagattgctccactgcactccagcctgtgcaacagagagagactttgtctcaaaagaaaagttttgggtgaaaagtagaaaaaaagtcataactttgtgtagagacttttccctc

SEQ ID NO: 368 - DNA, 401 bp, Homo sapiens
agcttccttgccttctccccatatcacctctctgcacctcttcacacccatgagggtctttatagcagtgcagtccataaattgttttgaggtaccaataccagtttcttggatattttcttacttaggaacataaatacttaggctgccattgcgttaataaggaaatgctggatagtcagaatcctacattaacccgarcctaaaatctgtagtatcgcttcaaatatctatgtagaaagcatagtttattaaactaaacatctcctgtaatcccagcactttgggaggccaatgtgggtgaatcacttgaggtcaggagtttgagaccagcctggccaacgtggcaaactctgtctctaccaaaaatacaaaaattagccaggtgtggtggtgcaacc

SEQ ID NO: 369 - DNA, 401 bp, Homo sapiens
aagaaaatgtggtacatatacaccatggaatactatgcagccataaaaatgaatgggatcatgtcctttgcaggaacatggatggagctggaggccattatcctaagcggactaacacaggaacagaagagtaaatactgcatattctcactcataagtgggaactaaacaatgagaacctatggatacatggaggggaaygacagacactggggcctacttgagggtggagggtgggaggagggagaagatcagaaaatatacctatagggtactatgcttattacctgggcgatgaaataacctgtacaccaaagccccatgacacgcagcttaccaatataacaaaccagcacatgtacccctgaacctaaaatacaagttaaaaaataaatacataa

SEQ ID NO: 370 - DNA, 401 bp, Homo sapiens
gcgatgaaataacctgtacaccaaagccccatgacacgcagcttaccaatataacaaaccagcacatgtacccctgaacctaaaatacaagttaaaaaataaatacataaaaatttaaaaataaaaaataaaactgcaaacgtgccctgctttctggcagtaccccattccctgttccttagttttctctctggcacttaycatttatctattttattcattgtctgtctcccttcactagaggttcatgagggcaaggattttcatgttctgtccactgctatgtccctaacacttaaaaaaaggctttgcattctatagtcattcgatgaaataggctgaatttagctaaaagggagttcatgtcctttgaagaaacatggatggagctggaggccatt

SEQ ID NO: 371 - DNA, 401 bp, Homo sapiens
cccctgaacctaaaatacaagttaaaaaataaatacataaaaatttaaaaataaaaaataaaactgcaaacgtgccctgctttctggcagtaccccattccctgttccttagttttctctctggcacttatcatttatctattttattcattgtctgtctcccttcactagaggttcatgagggcaaggattttcatgttytgtccactgctatgtccctaacacttaaaaaaaggctttgcattctatagtcattcgatgaaataggctgaatttagctaaaagggagttcatgtcctttgaagaaacatggatggagctggaggccattatccttagcaaatgaacacaggaacagaaaacaaaataccacatgccctcacttatcagtgggagctaaa

SEQ ID NO: 372 - DNA, 401 bp, Homo sapiens
tgcattctatagtcattcgatgaaataggctgaatttagctaaaagggagttcatgtcctttgaagaaacatggatggagctggaggccattatccttagcaaatgaacacaggaacagaaaacaaaataccacatgccctcacttatcagtgggagctaaacaaagaaaacacatggacacaaagagaagaacaacagayactggggtctgctgagggcagaaggtgggaggagggagaagatcagaaaaactacctatcaggtactatgcttcatacctgggttacaaaataatctgtacgtcaaatgcctgtgacacaagggtacctgtataacaaacctgcatatgtacccctgaacctaaaataaaagttgtttgtttggtttttttgtttgtttg

SEQ ID NO: 373 - DNA, 401 bp, Homo sapiens
gtgcagtgggccccctctgattcgtggctttgatttctttggtttcatttatccacagtctaaaaatattaaatggaaaatttaaaaaaaaaacaattcataagtgttaaattgcatgccattctgagtagcgtgatgaaatcttgccctgtcccactctgtcccatgcgggacatgaattcttcccctttgtccagcagwtcctcactgtctatgcctccgacctgttagtcactcagtagccctctcagttattagatcgaccatggcagtatgggggtgcttgtgttgaagtcacccttattttacttcctaatggcctcaaagcacaggagtagcaatgctggagatttggagctgccaaagagaagccctaaagtgcttcctttgagtgaaaactc

SEQ ID NO: 374 - DNA, 401 bp, Homo sapiens
agtgggccccctctgattcgtggctttgatttctttggtttcatttatccacagtctaaaaatattaaatggaaaatttaaaaaaaaaacaattcataagtgttaaattgcatgccattctgagtagcgtgatgaaatcttgccctgtcccactctgtcccatgcgggacatgaattcttcccctttgtccagcagttccwcactgtctatgcctccgacctgttagtcactcagtagccctctcagttattagatcgaccatggcagtatgggggtgcttgtgttgaagtcacccttattttacttcctaatggcctcaaagcacaggagtagcaatgctggagatttggagctgccaaagagaagccctaaagtgcttcctttgagtgaaaactcttaa

SEQ ID NO: 375 - DNA, 401 bp, Homo sapiens
agttcctcactgtctatgcctccgacctgttagtcactcagtagccctctcagttattagatcgaccatggcagtatgggggtgcttgtgttgaagtcacccttattttacttcctaatggcctcaaagcacaggagtagcaatgctggagatttggagctgccaaagagaagccctaaagtgcttcctttgagtgaaaastcttaataaggaaagaaaaacattgaatgctgaagttgttagatcttctatctgttgaactgtgaagaagaaaaaagatatttgtgcatagtatataaagggtttcgtactacccacggtttcaggcatccactgggtgtcatggaatgtatcccctgaggataaggggggattactctatgtattttcgtatccttaca

SEQ ID NO: 376 - DNA, 401 bp, Homo sapiens
ggcatccactgggtgtcatggaatgtatcccctgaggataaggggggattactctatgtattttcgtatccttacatcacttacattatttttcctcagccttgttgaggaaaataaaaacacagaagccaagaaagaaatgagcaagcactggccgggcacagtggctcacacctgtaatcccagcactttgggaggccragacgggtggatcacccaaggtcaggagttcaagaccagcctgggcaagatggtgaaacgccgtctctactaaaaatataaaaattagccaggcacagtggcaggtgcctgtaatcgcagctacttgggaagctgaggcagagaattgcttgaacctggaaggcagaaaagttgcagtgagccgagatcatgccactgca

SEQ ID NO: 377 - DNA, 401 bp, Homo sapiens
acagtaaattctcccttctctcccatccctgtctctagccacccactcctcttctgaggcaagagcagattctgttgccaccttcccaagcaggtagtgcctttaaaaaacaaaaaacaaatgttcacctactatatgcattaccatgcaccttacacttttcatttaataaatataccccagcaatggtgatagcagatyttggaccagcagtggggataaagaaggtcctggtcacctaggtgcctaaagagaagcagatgaagagagaagaccctggagaaatgttttcctgttttgcacctgcaggtgcttgacatgcctctgaaaatgatctcagtgctgtgcagttccacagctgcagtggtacccacaatgccaaaacttgggatgcaaagggg

SEQ ID NO: 378 - DNA, 401 bp, Homo sapiens
tggccaggcttgtcttgaactcctaatgtcaagtgatccacctgccttggccttccaaagtgctgggattacagatgtgagccactgtgcccagccttcatcttcatcttaaaacttcttccatgttcgctgtggctggggacatgatattcctctttccctcccagttgctcctaaggatgagcaaaaagctcttttcaygtcaggggttgtaccgccaatcactaaaaggaacagttgaaatatcaaacatggagaaattgcagtgattacaaaggaatgcaatggggcaccatacagatctgaatttgccagtttgccatatccaggctgtccctgttgcctctggtccaccttaagctaggtgcaaacccagttggaggaaagaaaggaagttatgt

SEQ ID NO: 379 - DNA, 401 bp, Homo sapiens
gaacacaaagcaagtggatacttagtagccataggggaatctgattttgatgtctcctacccaaagtttattcatatttaatattatatatacataagtcttattctttctcttcgtcctttttttttaaaggcaagatctgccttcatcttcggttttctttagtgcttagaagattgccttgctagtgataagtatttktgcttaataagtatatcttgactgtgaaatttgtcaccagttttgaaaggaactctggcagcaaatgaacaaacttaaatccttgtggccagggatcaaaggcaatctcaaaccgaattctataataaagagaagtttggagagagaaagggagtttagtgtaaactcaacttttgaaatccaaaggtatcaggctatca

SEQ ID NO: 380 - DNA, 401 bp, Homo sapiens
atgtataatggcacttcaaaatccaccattaggagtgtctacagtttcctctgcataatttagaggctgaggccatgtacctcaattcagtccagttttctatccaagaggtgcaagctctaaaataaaggaaaggaatattctttatcagtcatcagcttaatctcaaaatattgtgtgctccataattgaccttcattrgggcattatggacaggggcttactatatgctaagctatgtaaatacagatgctcctcagcttataatggggttgtctggataaatccattgtaagttgatccacacataaccctattaaaagtcaaagagcatactgaatgtgtattgcatttgcaccattctaaaatcaatgtgtcccttgtgtaaaatcagttgcatc

SEQ ID NO: 381 - DNA, 401 bp, Homo sapiens
cataagttggggaccatgcgtactctgatagtcagtgttatgtatgaacttgaccaagccgcagggtcccagacatttgtccagccattaatcaggtgtgtctgtgggaatgtttctggatgagatgaacatttgaactggtagattgagaaaagcagattcgtctcccctatgtcagtggacctcatccaatcaagacckgaatagaacaaaaaggctgagtaagagggaactactcctgtctgactgctttgagctgggacatcaatcttctggccttcaagctcaaatgcaaatatcagctcttcttgagtcttgaggctactggttttcagactagaatttataccattggctctcctggttctcaggccttcagatttagactgaaactacactat

SEQ ID NO: 382 - DNA, 401 bp, Homo sapiens
gaatttataccattggctctcctggttctcaggccttcagatttagactgaaactacactatcagcattcctgagtctccaaattgctgactgcagatcttatgactttccagtcattccattccttgtcataactctctttctctttctttctctctctctcctctctctctctctctctctctacatatatatatatayatatatatatatatacatatatatatatatatatatatgaacagatacatcctattggttctgtttctctggagaaccctgactaatatacagttatgtgtcacttaacaataggatatattccaaaaatgtgtcattaggcaatttcatgatttgtgaatatcatagaatattcttacacaaacctggatggtttgctg

SEQ ID NO: 383 - DNA, 401 bp, Homo sapiens
gctctcctggttctcaggccttcagatttagactgaaactacactatcagcattcctgagtctccaaattgctgactgcagatcttatgactttccagtcattccattccttgtcataactctctttctctttctttctctctctctcctctctctctctctctctctctacatatatatatatatatatatatatatatrcatatatatatatatatatatatgaacagatacatcctattggttctgtttctctggagaaccctgactaatatacagttatgtgtcacttaacaataggatatattccaaaaatgtgtcattaggcaatttcatgatttgtgaatatcatagaatattcttacacaaacctggatggtttgctgcacacctaacctgat

SEQ ID NO: 384 - DNA, 401 bp, Homo sapiens
aaaatatctaaacaacttaaatgctaaacaataataggcgattggttaaatgttctatgatgtagttactcagtgaaatactaccttcattacaaaccatgccctcaaggactatttatttacttgggtaatcattcaagctatatagatgagagtaaaaggcctgttacaagcctgaggagagatggtaactaaatataytgtatgctcgatgagaccctgaaattacattttaccatgtttccttaggcttgtagcattccatttttacttctattctgaagcagaaaaaggagattagataaaaactaagaaaaccttaataaaggccgggcgcagtggctcacgcctgtaatctcagcgctgggattacacacatccctgtttttaaaagtgaaatc

SEQ ID NO: 385 - DNA, 401 bp, Homo sapiens
tttttactcaattcattccctacatcacaacctgtggtttttgtatctgatcatagttttcaccctattgaaaatcatggctggccaagtacatgttaacaggaaaattctggacagtccctttgtacacaaaccttactgcttagtttcatctgtgcagattgctgtatactcagccctgcagcagagagaccacactgmagcctttagtaggaaaaggataggaatgacaatacactgcagacactttcgtgagtaagttgctaaagcgtcagtaaaacatcatggtgaggcctctgcaatcagcctgcttagatttaaatgacaattctgtcacttacaaatatataaccatttggatagttatgtttgcactttaagcctcatgttagttatctgga

SEQ ID NO: 386 - DNA, 401 bp, Homo sapiens
catagttttcaccctattgaaaatcatggctggccaagtacatgttaacaggaaaattctggacagtccctttgtacacaaaccttactgcttagtttcatctgtgcagattgctgtatactcagccctgcagcagagagaccacactgcagcctttagtaggaaaaggataggaatgacaatacactgcagacactttcrtgagtaagttgctaaagcgtcagtaaaacatcatggtgaggcctctgcaatcagcctgcttagatttaaatgacaattctgtcacttacaaatatataaccatttggatagttatgtttgcactttaagcctcatgttagttatctggaatagtatataatattacctacctttagtgtcatcataagaattaaatgatt

SEQ ID NO: 387 - DNA, 401 bp, Homo sapiens
gggagggttaatagaatggatatctaaagcagcgattctcgaatataggcttttaaccagcagaatcagcattacttgacccgaaaacttgttagaaatgcaaacacttaagtctcacccctaagctcctgattcagaaaatctagggttagggtttaactgtctgtgttttccttgtttttgtttgtttgtttgttttgycgttgttgtttgttttttgtggttgttttgttttgtttttgagacagggtctggctgtgttgcccaggctagagttcagtggcacaatctcagctcactgcagcctcaacctcctgggctcaagcgatcctcctacaacagcctctgaagtcgttgggaccacaggtgcaccaccacgcttggctaatttttgtattttt

SEQ ID NO: 388 - DNA, 401 bp, Homo sapiens
actagtatcaactgccatacaattatttagtcacccttgggtaactaaacagcataggtatgttgatcaaaatgcccttgagtaattgtcagtggaattttgaactctccccaattcaataagatactgctgcaggtattggtgaggtacagggagcatctaaatggactgtctttcattgtctttcttgagggttctaawtctttggttttctggagaatattataggatagaatgtatatcagaagtgagacttgggagacctgtgtgacctcccaagcatgacaatattcacagatattcagatccatagaacacttcaggaaattgtctttatgttcaaagatctcatagaacatttagttggatacacttcccacacacatctctccatttgatat

SEQ ID NO: 389 - DNA, 401 bp, Homo sapiens
tcaactgccatacaattatttagtcacccttgggtaactaaacagcataggtatgttgatcaaaatgcccttgagtaattgtcagtggaattttgaactctccccaattcaataagatactgctgcaggtattggtgaggtacagggagcatctaaatggactgtctttcattgtctttcttgagggttctaattctttgkttttctggagaatattataggatagaatgtatatcagaagtgagacttgggagacctgtgtgacctcccaagcatgacaatattcacagatattcagatccatagaacacttcaggaaattgtctttatgttcaaagatctcatagaacatttagttggatacacttcccacacacatctctccatttgatatagagtca

SEQ ID NO: 390 - DNA, 401 bp, Homo sapiens
tctttcattgtctttcttgagggttctaattctttggttttctggagaatattataggatagaatgtatatcagaagtgagacttgggagacctgtgtgacctcccaagcatgacaatattcacagatattcagatccatagaacacttcaggaaattgtctttatgttcaaagatctcatagaacatttagttggatacmcttcccacacacatctctccatttgatatagagtcattatcttttcaaactttctttttggtcttttacagaagaacctgttaggatattctcaagatttgttctagtcacacgggggtggagggggagggggggtgtgttatagaagtagctctagatcatttagcttaagagttaaattttttgcaaaaatactttgt

SEQ ID NO: 391 - DNA, 401 bp, Homo sapiens
ttttctggagaatattataggatagaatgtatatcagaagtgagacttgggagacctgtgtgacctcccaagcatgacaatattcacagatattcagatccatagaacacttcaggaaattgtctttatgttcaaagatctcatagaacatttagttggatacacttcccacacacatctctccatttgatatagagtcaytatcttttcaaactttctttttggtcttttacagaagaacctgttaggatattctcaagatttgttctagtcacacgggggtggagggggagggggggtgtgttatagaagtagctctagatcatttagcttaagagttaaattttttgcaaaaatactttgtggtggatttctgatgccagtccgcataggacttttat

SEQ ID NO: 392 - DNA, 401 bp, Homo sapiens
gaacacttcaggaaattgtctttatgttcaaagatctcatagaacatttagttggatacacttcccacacacatctctccatttgatatagagtcattatcttttcaaactttctttttggtcttttacagaagaacctgttaggatattctcaagatttgttctagtcacacgggggtggagggggagggggggtgtgtyatagaagtagctctagatcatttagcttaagagttaaattttttgcaaaaatactttgtggtggatttctgatgccagtccgcataggacttttattattgtaatagtgttttgtctttcttttttaagattaggaaagtatattcaccataatatttgttgttaatcacttattttttatgcaccttttccaaagcaac

SEQ ID NO: 393 - DNA, 401 bp, Homo sapiens
cttttcaaactttctttttggtcttttacagaagaacctgttaggatattctcaagatttgttctagtcacacgggggtggagggggagggggggtgtgttatagaagtagctctagatcatttagcttaagagttaaattttttgcaaaaatactttgtggtggatttctgatgccagtccgcataggacttttattatygtaatagtgttttgtctttcttttttaagattaggaaagtatattcaccataatatttgttgttaatcacttattttttatgcaccttttccaaagcaactatattgtccttatcttgaaaaatgaaaataatagcttccagataaatccttatgatagttttgctatcactagatagtggcagagggaaacagaggaaa

SEQ ID NO: 394 - DNA, 401 bp, Homo sapiens
attaaaaaggtaaagagaaataagtgaagttaattttaataatatattatctttaacctcttttaataatatactctctttaactctttaatatatttaataatatattctcttttatatacatagaacattgtcatttcaacatgtaatcaatataaaaaatatcaattagatattttacattctttttttcacattaartctttgaaatttagcacgtattttacacttacagtacatcttaatttggactagatgcatttcaggttcccaatagccatatgtgtctattggcgttcgtatcatatttatcatggcagttttaaatgtattaaaatgataagtctataaggtatcttcttaagtaaattctcccaagtataacagatatcagaggtaat

SEQ ID NO: 395 - DNA, 401 bp, Homo sapiens
attaccgttccaagacgattttatacaagtgaagcttaagttcagtgtgatgattaacaagctattagagccaagttattgattcctctatctttagagtagctaatgcagtgtgcaccaataggccacagaccccaagctttccttcttcctgtgctcctgttctttgtaatgatgctttccttaggaaaatgaaaatcwcttcgcatacacagtgcaacatctttaaggcaggtgccagcagagaggaaaaacacctaagctgattttgtgtgtttttattattttttctgataataaagattaatgcagatacatgaaaaaaacaaaacataaaaagtaggaacagaaagtcaaatactgcatcttctcacttataaataggagctaaataatgtgta

SEQ ID NO: 396 - DNA, 401 bp, Homo sapiens
tataattgatattgctccattgggtggcctgtggtattcaattcactggaaagtatcaatccctttgccttcagcaataatattttcacagttttgatgtgttttaaaggttattgtctccaggacaggtaattgaggaggcctcacacttcatcagaggccatttcatctttattacacagccacacacaaggcagcatyttctcctttcactgatggattatttcaagatttataaggtctggtctacatgaaagcaaacacatgtttgggtacacagtcaaataattcaaagatatagggtcagcaggatcctaatattaaagtcaggacttccagcctgggcaacaaagtgaaatcttgtctctacaaaaaatacaaaaaattagccgggcatggtg

SEQ ID NO: 397 - DNA, 401 bp, Homo sapiens
tggccaacaagcagatttccatagcagaggagctgcgactggggcagagagccaaagacagcttgtgccaacagccttgtcaatatgccaatcatcttgttacagtttattacatgcgtgacttgattttcttgcatgtgcgttttagaggtaacaacatattggtaaacctgttttcaatccttttgattaccttcaatytgatagctccttcagtaatgaagaaaaaaatgggctattgtgtttgaattacatttatcaaatgattctaagattgttctaacgtgtgcgacaggataagctctcttttagtcaccaattggagaaatatgctcagtgtcaaggaaatggagaaatgcctcattatttatgttgactaatgtataaatacaatatgaaca

SEQ ID NO: 398 - DNA, 401 bp, Homo sapiens
tcataaaacagggaatgttataatatttctctgcttcactgtgtccaaggaagtagagaaatctggtaccatatttatgagcagcaataaaccatgtgttttgctttttcctaaaacaagcctggcaaactgtttaagaacacatgtgcagagcttgggcacagggtgagagtttggtggcacggagtgaggctagaggtrggcgtgtggagagtctgatatcctgatatcaaagcaattccattcagacttaggcctgagctgctcccagtcatccaagtcatttttactaatatctgacaacttgcaaatattgtgttacgttcaacactgaaaatgaggactgtgcatttccttttgtctaaatgatgattaaatggtgaagcagctacagaagtatt

SEQ ID NO: 399 - DNA, 401 bp, Homo sapiens
atgattaaatggtgaagcagctacagaagtattgactaacgacagcatccatttactagaaaatatgtcatttgctactgcaaacatttatggtctgattatacctcatgttacactgcaatggtttgggttttttttttttttttgtatttttggtacatcagaaacacttttcccaatgtttgttagtttaagcattgktcaacatttatttattaagcattgactttataaaataggtaccaggaatatgaaaatgagtaagatatgttccctgacatccagtagctcatagtctagagatgtcaccataataataactagcatttattggacaccacctgtgtctcctgaagcctgttacctcatttaattttgacaaaaccttatgaagtgaatat

SEQ ID NO: 400 - DNA, 401 bp, Homo sapiens
gacccaccttagaaaggaggtcttcatataccctgtctgtgcttattttgtgttttcacaacacaaaatattttaacgatgatttaatttagttgtgtattgatagtctctgtcaatctctcagggttttttttttctgagggaaaaaattacattttgtaattttttaatttagaaacaacagcaataacaacttgagtyggtgacttctgaaccatttttgttctgattatcactgtaaacctaaatgtgtgaacactatcagtagggataatatttaccctatgggactgttaagcaatttaagatgagataagaaatgtcacagggaaaaggtgttactccacacatggtagtcgttattatttattgggttgaagtgttggcctcccaaagggcaa

SEQ ID NO: 401 - DNA, 401 bp, Homo sapiens
aatgccccagaagcccagatcgggacccaccactgggagttacagccctctggagccagcttcagaggtgcacaggtttattctcttccctattcaccagtgaaagaagcccagagcctccattccaggcactctgtgcttcccaaagtaacaattacttaatttgacaagataatttaaagagattgccaaaataacttmcaggagattgccagtataatcagccctcccatatctggggattctacatttccagattcaaccagttgaagagagaaaatatttgaaaaaaaaaatttaaaataatacaaataaaaacaatacaacatagtatttatatcatattgggtattgtaagtaatgtagagaccatgtaaagtatatggaaggatgtgcatagg

SEQ ID NO: 402 - DNA, 401 bp, Homo sapiens
ttcatgactggttttcacaaaaacaagcattagggagagtggaaggaagagcttgcaaaataccttactactgcattatgaatgtggagttgtaattttgaggttcttatccatgaagatggtccttaaaattcagaattttaattaccaacataacaccatcctttcttactagtatcattattttatactatgttttcrgtaggacatatctgatggaaatacatttcttttgatattatttattataactggtgagctacattccacccaatagtaaatgcctctcagcaaaaagatagatacttttgatagttaatatattcagatattatgttgtttgtggaaaccataacttaataaattttaaaacagtgtcttctttaaaagtaattttttat

SEQ ID NO: 403 - DNA, 401 bp, Homo sapiens
tgcccaccctcaccttaaaaccctggcaataattaacattttgtcatttcaagcatgttatataaatggaaccatagagtatataactttcaggtattggttttttccaatcagcataattctttggagattctttacttagcatacttctctggagatttatctaggtgcttgtgtactagtagttcattctttgttatygctgcatagtatttcatgctagggatgttgagccatttacccattgaaagatacttgggttgtttctagtttggggagattataaataaagctgctataaatactcatacacagatttttgtgtaaacatacattttcatttctctgagataaatgcctaggaatgagtgtgatttctagatctgatagtaattgcatgt

SEQ ID NO: 404 - DNA, 401 bp, Homo sapiens
tccacaaggcaagagcttttaattttgataaaaataactttttatacttaattggcaaccaaatacctaaatgtcttttgatagaaaaacttgtcctagataatgtaaaatcattttacatattcacagatccatctccactaatgtcatataagtttaaaaatcacaaagtaatttgaacagattcctaaattataaatyagttaacagttattttttgctctaatctttagttgtacatttacttcaaataaacatttgttaaaaacgtactctgtactgaggaatgaggatacaaaactcataatttgtcatttctttgcttcagggacttacattttactggattacatctaaatttttatttggcagttttattccaggaagatcatgacagtctt

SEQ ID NO: 405 - DNA, 401 bp, Homo sapiens
aatgtcttttgatagaaaaacttgtcctagataatgtaaaatcattttacatattcacagatccatctccactaatgtcatataagtttaaaaatcacaaagtaatttgaacagattcctaaattataaattagttaacagttattttttgctctaatctttagttgtacatttacttcaaataaacatttgttaaaaacrtactctgtactgaggaatgaggatacaaaactcataatttgtcatttctttgcttcagggacttacattttactggattacatctaaatttttatttggcagttttattccaggaagatcatgacagtcttgttttctaaatcttaactgccttttgatgaaaaagaaagataaggctttaatttctctagtgaaacctg

SEQ ID NO: 406 - DNA, 401 bp, Homo sapiens
gtcacacacttttaaaccatcagatctcgagataactcactgtcatgataatagcaagggggaagtctgcccccatcattcaatcatctcccaccaggcacctcccctgacatgtggggactacaattctagatgaggtttggttgtggacacagagtcaaaccatagcacccggctaattttcttttcttttctttttgrtagatatggagtgtcactatgttttgtttccctagctggtcttgaactcctgggctcaagtgatctgcccacctcggcctcccaaaatgctgggattacaggtgtgagccagtacacccagccaagaagtgatttgtataatggcaggattatgaatgattatttctgtgtctttaatttgatcatatttttaaaacagt

SEQ ID NO: 407 - DNA, 401 bp, Homo sapiens
cacacttttaaaccatcagatctcgagataactcactgtcatgataatagcaagggggaagtctgcccccatcattcaatcatctcccaccaggcacctcccctgacatgtggggactacaattctagatgaggtttggttgtggacacagagtcaaaccatagcacccggctaattttcttttcttttctttttggtagmtatggagtgtcactatgttttgtttccctagctggtcttgaactcctgggctcaagtgatctgcccacctcggcctcccaaaatgctgggattacaggtgtgagccagtacacccagccaagaagtgatttgtataatggcaggattatgaatgattatttctgtgtctttaatttgatcatatttttaaaacagtcatt

SEQ ID NO: 408 - DNA, 401 bp, Homo sapiens
gtgatttgtataatggcaggattatgaatgattatttctgtgtctttaatttgatcatatttttaaaacagtcattatttttggtcatcatgctatatactacagagagcagtttttaatttgtgaacgtgtaagtcaacttgatttagctgaacatagcagagtcatcaatcaaactgattgatgactaagggtccgcasggtaacacatcatcatagttgtttccatggtgatgaattatgatgtcacaaactcatcaagactgtgactcagtgaaccatctagacagaggatgaaatgcagtagttcaccaacatgtatgccatctttgtattatatggtattattaattaaaacgaagggctgagtatttctttcattgagattacatgaagatatt

SEQ ID NO: 409 - DNA, 401 bp, Homo sapiens
aaatgcagtagttcaccaacatgtatgccatctttgtattatatggtattattaattaaaacgaagggctgagtatttctttcattgagattacatgaagatattacgtatattcagacttgcctgaacacggctatgctggttttaaattgaaggttttcacaaagcaaaaaattgagaaacagaatttgacatagaatygtgtatttattagaaaagatcttttcttctcatattctgaaaagcacactcattaattacagttaaattcaggcaaatttggcaaattttcgtcagtatacttattcttttgttagttaatagctttgcttttcagttttagatattggtattaaatatataaagagaaaagaccttagtgttctcatgctcagagatta

SEQ ID NO: 410 - DNA, 401 bp, Homo sapiens
tttctgaccttcaactctggtactcgattttctcataaatgtgagaaaatatcttaaccgtatcacagaattgatgtgagaattcaaaataaagcatatttaaagctcctaggttagtgcttcatttatgaaggtcagtgcttcataaatgatagttatttttttccattcaaaatagagataaaaaagactttatctcastaatgtgtcatgcaaataaggattaatcctgttttctattcctgaagcatacagaatgtgatttctttctccatcatttccatctgactatcatctgggagaacacacagctgctccacatctggtgcaacacaggcagatgtagctgtccagagggagaagagtgtttgcttttctccacctaaacactgaccctcttc

SEQ ID NO: 411 - DNA, 401 bp, Homo sapiens
tgtaccagaagtgtccgtatgaacacaaactcctagtaagtgttcttaattgttctgtgggctgatgtgcatttttctcctacaataggaactagcaatctacatattatttaccaagccttcctagatagaagcatttatgattattttttgtttatataaatgtcttgcatttactagaaaatcatgcttttcgagaaygcatttctattatggagtttcttaaagagagaaaaaaaatgtatggctttagaaaaatcctagtgattttttgacagtatgaactgtattgtgtgttcttgtcttggatcaatatcatggcacaatgctttgatagtgtctgaagattaagatatacaatacaatttcctcccttcccttctttcctttttccttccttc

SEQ ID NO: 412 - DNA, 401 bp, Homo sapiens
caaattcctgggctcaagggatcctcccgcctcagcctctcaagcagctgggactacaggtgtgcccctccatgtgcggttaattgtagttttttgtttggggtggttttgtttgtttgtttgtttttgtagagactttgttgcctaggctggtctcaaactcctgggctcaagcagtcctcccactgcaacctcccaaartgctgggattataggtgtgagccaccatgtccagcccaataccattttcttatgtttcccatatatgcataattttacttgcatgcaattctgaatggatgaaacatgttttaatcacctatttttctactttgtagaagagccaaggtaacatgaactaaatcattaaagcagttgacttgttagtggattgtgatagg

SEQ ID NO: 413 - DNA, 401 bp, Homo sapiens
gactgaatcagttcaaagagcctcgatagctttaaatgtctgtgattgatttttctgcagagcaagatcaaacaaaacagtatttcctagctgctgcccttacatgtagtggtgattcacagagaatttggggaggaaagtagccattatgggatgttttgcaaaaggcaataccaagattgatcatcacctgagggcgayacactatagttgttcctctgtatccatgacagattagtcccaggaccccttgcagataccaaaatccacagatgctcatgtcctgcttataaaatggcatagtatttgtacacagccttgtatatcctcctgaatactttaaatcatctctagattacttataatacctagagttgtataaatgctatagaaagtattgt

SEQ ID NO: 414 - DNA, 401 bp, Homo sapiens
ctgtggggtggggatgttcaccgagaagactgctttggtctttgatactagcatttttccttctatatggttaacaaggcacatattttcatttttatatccccagaataaaggcttgaaaggaaacaaaatctcaaataaatggactaatttgttaaattctggactaaacctcagtttattttaacagctttaacaackgtctctggagctcataacacatatagagcactattggtgtcacacaaaaccttgaaatcagaattgccacagaccgaatctgatctgaaatggcatttcattcaactgcagctttgtgtcacaagaacctgtctctgttttcctaacatgggtatgatgcatattcagaagcagccgagtgatttacttcttttttcatt

SEQ ID NO: 415 - DNA, 401 bp, Homo sapiens
gctttggtctttgatactagcatttttccttctatatggttaacaaggcacatattttcatttttatatccccagaataaaggcttgaaaggaaacaaaatctcaaataaatggactaatttgttaaattctggactaaacctcagtttattttaacagctttaacaacggtctctggagctcataacacatatagagcaytattggtgtcacacaaaaccttgaaatcagaattgccacagaccgaatctgatctgaaatggcatttcattcaactgcagctttgtgtcacaagaacctgtctctgttttcctaacatgggtatgatgcatattcagaagcagccgagtgatttacttcttttttcattacgcttgtggatttgcatagattcagaaggg

SEQ ID NO: 416 - DNA, 401 bp, Homo sapiens
caagtgattctccttgcctcagcctcccacgtagctgggattacaggcgcccaccaccacacccggctaattttttttttgtatttttagtagagacggggtttcactatgttggccaggctggtcttgaactcctgacctcgtgatccacctgcatcggcctcccaaagtgctgggattataggcataagccaccttgcytggccctaaaatacttttaataagattgtctcatccttctcttggttttccaatggaaatggtggtatagcaagtccatctgaaacacttctgcaaaataacaatgaagctaatcactgtgggaaacatttacatcatatgtatttgcatacagaggaaattgataatcttgagacatcagcatactagaatagcaagag

SEQ ID NO: 417 - DNA, 401 bp, Homo sapiens
aagtgattctccttgcctcagcctcccacgtagctgggattacaggcgcccaccaccacacccggctaattttttttttgtatttttagtagagacggggtttcactatgttggccaggctggtcttgaactcctgacctcgtgatccacctgcatcggcctcccaaagtgctgggattataggcataagccaccttgctyggccctaaaatacttttaataagattgtctcatccttctcttggttttccaatggaaatggtggtatagcaagtccatctgaaacacttctgcaaaataacaatgaagctaatcactgtgggaaacatttacatcatatgtatttgcatacagaggaaattgataatcttgagacatcagcatactagaatagcaagaga

SEQ ID NO: 418 - DNA, 401 bp, Homo sapiens
ggctggtcttgaactcctgacctcgtgatccacctgcatcggcctcccaaagtgctgggattataggcataagccaccttgcttggccctaaaatacttttaataagattgtctcatccttctcttggttttccaatggaaatggtggtatagcaagtccatctgaaacacttctgcaaaataacaatgaagctaatcacygtgggaaacatttacatcatatgtatttgcatacagaggaaattgataatcttgagacatcagcatactagaatagcaagagacatgttttattctattatattctcttagtcagcaatgtttaattgttgcttaaaggaaactgaataaaacgtgggcgcagtggctcccacctctaatcccagcactttgggaagttg

SEQ ID NO: 419 - DNA, 401 bp, Homo sapiens
tgggattataggcataagccaccttgcttggccctaaaatacttttaataagattgtctcatccttctcttggttttccaatggaaatggtggtatagcaagtccatctgaaacacttctgcaaaataacaatgaagctaatcactgtgggaaacatttacatcatatgtatttgcatacagaggaaattgataatcttgrgacatcagcatactagaatagcaagagacatgttttattctattatattctcttagtcagcaatgtttaattgttgcttaaaggaaactgaataaaacgtgggcgcagtggctcccacctctaatcccagcactttgggaagttgaggagagaggatcgcttcagcccaagagtttgagaccagcataggcaacatagtg

SEQ ID NO: 420 - DNA, 401 bp, Homo sapiens
tctcttggttttccaatggaaatggtggtatagcaagtccatctgaaacacttctgcaaaataacaatgaagctaatcactgtgggaaacatttacatcatatgtatttgcatacagaggaaattgataatcttgagacatcagcatactagaatagcaagagacatgttttattctattatattctcttagtcagcaatktttaattgttgcttaaaggaaactgaataaaacgtgggcgcagtggctcccacctctaatcccagcactttgggaagttgaggagagaggatcgcttcagcccaagagtttgagaccagcataggcaacatagtgagaccccatctctacaaaaaatattttttaaaattagtcgagcatggcggtggctccctgtagtt

SEQ ID NO: 421 - DNA, 401 bp, Homo sapiens
aacatagtgagaccccatctctacaaaaaatattttttaaaattagtcgagcatggcggtggctccctgtagttccagctacatggagggctgaggtgggaggatcacttgaaccagggaggtcaaggctgcaccctggcctgggcaacaaagccaaaccctgtgtcaaaaaaaaaaaaaaaatacgtaaagcaacagttkttctataacggattgcatggtaaaaggaaggatgactttcttactataactccttatgataattttttccaaagtgttaatcttaggggttgaaatttttataggtaatcattaactttagtagaacattattttaaagtttaggtatttcatctgagtatattactctggctttcctaagctagcttagttatatatgt

SEQ ID NO: 422 - DNA, 401 bp, Homo sapiens
ccccatctctacaaaaaatattttttaaaattagtcgagcatggcggtggctccctgtagttccagctacatggagggctgaggtgggaggatcacttgaaccagggaggtcaaggctgcaccctggcctgggcaacaaagccaaaccctgtgtcaaaaaaaaaaaaaaaatacgtaaagcaacagtttttctataacggwttgcatggtaaaaggaaggatgactttcttactataactccttatgataattttttccaaagtgttaatcttaggggttgaaatttttataggtaatcattaactttagtagaacattattttaaagtttaggtatttcatctgagtatattactctggctttcctaagctagcttagttatatatgttcacttcagttt

SEQ ID NO: 423 - DNA, 401 bp, Homo sapiens
attttttaaaattagtcgagcatggcggtggctccctgtagttccagctacatggagggctgaggtgggaggatcacttgaaccagggaggtcaaggctgcaccctggcctgggcaacaaagccaaaccctgtgtcaaaaaaaaaaaaaaaatacgtaaagcaacagtttttctataacggattgcatggtaaaaggaagkatgactttcttactataactccttatgataattttttccaaagtgttaatcttaggggttgaaatttttataggtaatcattaactttagtagaacattattttaaagtttaggtatttcatctgagtatattactctggctttcctaagctagcttagttatatatgttcacttcagtttgagtctccactaaatttta

SEQ ID NO: 424 - DNA, 401 bp, Homo sapiens
tagaacattattttaaagtttaggtatttcatctgagtatattactctggctttcctaagctagcttagttatatatgttcacttcagtttgagtctccactaaattttaacatgaacaaaatattatatgcaacaccatagaagcaattgttaaatcattgttacttttaatatcacctaaatctaacctaagtaaatgygtttatttttattaccaactgatgtagatatacccacatggacagaaaacagcataaaccaccaaaaggacttgaaaattccctcttcaagacttcttttgccaatgactaatgtttaatgctatttgctagagattctggtgccaggaggatttctaaccaggttattttatcaataggaataaaatatcgagggctta

SEQ ID NO: 425 - DNA, 401 bp, Homo sapiens
cacatggacagaaaacagcataaaccaccaaaaggacttgaaaattccctcttcaagacttcttttgccaatgactaatgtttaatgctatttgctagagattctggtgccaggaggatttctaaccaggttattttatcaataggaataaaatatcgagggcttaaattagtgtaatctctcaagatcttgaatttgacrttttcataatttttacttaatcatagctcctggggattggaattactcttttagctggcccgagtctgggaggaggatccaaagtctttgagttgttttcttttatttctttttagcccctaaagcactggaaatggttggccctgaagtgctcctaaggtaaccgcaagatgaggcaaaacaggattgagatgaaaacc

SEQ ID NO: 426 - DNA, 401 bp, Homo sapiens
caagttgaaaaaagagtatggaggaaagcggtgtttaaaattgtccaaagccatcggccgggcatgatggctcacgcctctaatcccaacgatttgagaggctgaggtgggaaaattgcttgagcccaggagtttgagaccagtctgggcaacatggtgaaaccccatctctacaaaaaaagtacaacaattagctgggcrtggtggtgcatgcctgcagtcccagatactcaggaggctgagatgggaggatcacttgagcctaggagtttagggctgcaatgagatgtgagtgcaccaccatactctagcctgggcaacagagcaagaccttgtctcaaataataataataatatataaataaacaaataaataaaattatcctaagtgaaatcagttg

SEQ ID NO: 427 - DNA, 401 bp, Homo sapiens
gcctgggcaacagagcaagaccttgtctcaaataataataataatatataaataaacaaataaataaaattatcctaagtgaaatcagttgttgatagaactgaagtacatcaagcctagtgggactgtggttgtctctccagcctaaaaaaaaaaaagaacaaaaacaacaacaaaaaaacttaactccaaaattgctgytgctccatttatttcaggtgtaagacttctttgagatttaaaaacaatataatcatttacaatgtactttgttggacctaagcataggaaaattcattcattcattcattcattgagtacctgttatatgccaggcaatgcactaggcaccatcaaagatgaataagctatgtgctaaaattcacagtctaacagaagaa

SEQ ID NO: 428 - DNA, 401 bp, Homo sapiens
aattataatttttctcaaacttcccaaaagtggaacattgtgggaaataatatgggagtggacatttgaatttctgtttgactttgcagcagaaatctcttttctaactctctctttcaatctcaccatcagagccatctgattgcttgataacatgattctttttgcccagcccttgttatcctccctcctgccctagtmaccatgacataggctccactcatttctacctaggaacctggatgcactgctttgcactattgcctcctggcaaaacagggcaatgagaaaggaaatggaagcccaaattcagagccctccttcccagcacaagaatgaggagatgccctttttccccggggagggggattcttccctctctcttcagtaattgtctttat

SEQ ID NO: 429 - DNA, 401 bp, Homo sapiens
caaatttcatgttcttctggcagtcaaatcaggaaattcaaggacacaactgctctggcttcgtaaagatggggatcaaattgtttgagttgctgatagcaactctttgcttcttgattctccatgcctcctcaagcttttttggctgaacttttagccttggaacagggtcactggaaaatacttcttaatatccttaawttctaattctaatatttgccttgtttcattttgtttatttggggaggatgaagttcaggtgttcacctatcaaactcacttccttcttttttttagacaaggtcttgctctgtcgcccaggctagagtgcagtggtatgatgttggctcactgcagcctccacctcccaggttcaagtgattctcccacctcagcctccc

SEQ ID NO: 430 - DNA, 401 bp, Homo sapiens
tgctctggcttcgtaaagatggggatcaaattgtttgagttgctgatagcaactctttgcttcttgattctccatgcctcctcaagcttttttggctgaacttttagccttggaacagggtcactggaaaatacttcttaatatccttaatttctaattctaatatttgccttgtttcattttgtttatttggggaggatraagttcaggtgttcacctatcaaactcacttccttcttttttttagacaaggtcttgctctgtcgcccaggctagagtgcagtggtatgatgttggctcactgcagcctccacctcccaggttcaagtgattctcccacctcagcctcccaagtagcagggatcacaggctcgtgctaccacgccccactacttttttgt

SEQ ID NO: 431 - DNA, 401 bp, Homo sapiens
ggcttcgtaaagatggggatcaaattgtttgagttgctgatagcaactctttgcttcttgattctccatgcctcctcaagcttttttggctgaacttttagccttggaacagggtcactggaaaatacttcttaatatccttaatttctaattctaatatttgccttgtttcattttgtttatttggggaggatgaagttyaggtgttcacctatcaaactcacttccttcttttttttagacaaggtcttgctctgtcgcccaggctagagtgcagtggtatgatgttggctcactgcagcctccacctcccaggttcaagtgattctcccacctcagcctcccaagtagcagggatcacaggctcgtgctaccacgccccactacttttttgtattttt

SEQ ID NO: 432 - DNA, 401 bp, Homo sapiens
gcctcctcaagcttttttggctgaacttttagccttggaacagggtcactggaaaatacttcttaatatccttaatttctaattctaatatttgccttgtttcattttgtttatttggggaggatgaagttcaggtgttcacctatcaaactcacttccttcttttttttagacaaggtcttgctctgtcgcccaggctasagtgcagtggtatgatgttggctcactgcagcctccacctcccaggttcaagtgattctcccacctcagcctcccaagtagcagggatcacaggctcgtgctaccacgccccactacttttttgtattttttggtagagacggagtttcatcatgttggccaggctgatcttgaactcctgacctcaagtgatctgcctg

SEQ ID NO: 433 - DNA, 401 bp, Homo sapiens
tctaattctaatatttgccttgtttcattttgtttatttggggaggatgaagttcaggtgttcacctatcaaactcacttccttcttttttttagacaaggtcttgctctgtcgcccaggctagagtgcagtggtatgatgttggctcactgcagcctccacctcccaggttcaagtgattctcccacctcagcctcccawgtagcagggatcacaggctcgtgctaccacgccccactacttttttgtattttttggtagagacggagtttcatcatgttggccaggctgatcttgaactcctgacctcaagtgatctgcctgcctcgacctcccaaagtgctgggattgcaggtgtgagccaccgtgcccagccctaacttccttctgatcaagtcatt

SEQ ID NO: 434 - DNA, 401 bp, Homo sapiens
acatttaattgactgtcaactacaatgataataagacaatgtttgccttaaatgagctctaaatctagttgaagggataacacatggaatggactggtaataaaaagtgggctttgcttggtgctacagttgtgatgtgaactaaagagggagtgttttggactgactttgagttggggaggctttatggaggaggtgacwtttgagttgggccttacgttgtattttatgaaataaaaaaagaggaaagacagtccaagatgagaagacagtataaataaagggacagaggcatggaagggaatttcttggccaaaggatagtaaaaagtacagaaccactaaagtctagagcacataggctagaggctagaataagtgagaaatagctaatcctaggaa

SEQ ID NO: 435 - DNA, 401 bp, Homo sapiens
ttaattgactgtcaactacaatgataataagacaatgtttgccttaaatgagctctaaatctagttgaagggataacacatggaatggactggtaataaaaagtgggctttgcttggtgctacagttgtgatgtgaactaaagagggagtgttttggactgactttgagttggggaggctttatggaggaggtgacatttsagttgggccttacgttgtattttatgaaataaaaaaagaggaaagacagtccaagatgagaagacagtataaataaagggacagaggcatggaagggaatttcttggccaaaggatagtaaaaagtacagaaccactaaagtctagagcacataggctagaggctagaataagtgagaaatagctaatcctaggaagata

SEQ ID NO: 436 - DNA, 401 bp, Homo sapiens
actacaatgataataagacaatgtttgccttaaatgagctctaaatctagttgaagggataacacatggaatggactggtaataaaaagtgggctttgcttggtgctacagttgtgatgtgaactaaagagggagtgttttggactgactttgagttggggaggctttatggaggaggtgacatttgagttgggccttacrttgtattttatgaaataaaaaaagaggaaagacagtccaagatgagaagacagtataaataaagggacagaggcatggaagggaatttcttggccaaaggatagtaaaaagtacagaaccactaaagtctagagcacataggctagaggctagaataagtgagaaatagctaatcctaggaagataaattggggcttaat

SEQ ID NO: 437 - DNA, 401 bp, Homo sapiens
caaatttttgggctcaaatgaacctcctaccttggcctcccaacgtgctgagattacaggcatgagccaccgtgcctggccagctaaggttaatttagtattgaagaaatatttttgttatgaatttagtgtagccaagtgtgcagtgtttagaccatctacagtaatgcacattaatggcctaggccttcacattcactsaccactcactcactgactcacccagagcaatgtctagtcctgcaagctccattcatgggaaaggcctcacacatgtataacatttttttgtcttttattctgtatttttctatgtttagataagtttagatgtataaatacttatccttgtattacggttctctagtgctcagtacagtgacttgctgtacaggtttgta

SEQ ID NO: 438 - DNA, 401 bp, Homo sapiens
tctacagtaatgcacattaatggcctaggccttcacattcactcaccactcactcactgactcacccagagcaatgtctagtcctgcaagctccattcatgggaaaggcctcacacatgtataacatttttttgtcttttattctgtatttttctatgtttagataagtttagatgtataaatacttatccttgtattacrgttctctagtgctcagtacagtgacttgctgtacaggtttgtagcctaggaacgataggttataccatatagccaaggtgtgtagtaagctgtaccatcttggttcgtgtaaggacattcagtgatgaaattgcctactgacacatttctcagaaggtacccccatcgttaagcaaccattactgtattagaaagcagaa

SEQ ID NO: 439 - DNA, 401 bp, Homo sapiens
ccagttttctgcgggaatgaaatgaggtaccatacgtgatcctaccagcataacacctgccaaatagtgcacattcaatactaaaccatcctttttatgcctataaataaaggatttgtactatatcaagagcttctctatgtctgaagttacaatagagtaattccaaattatctttcatggaatcaaatccctgatgaycccttgattttctgagttctgtgaatttcatcacagcccaatttgacccattactgctaattcagtgtttttattagtaaattatatttccaagttgaatcaaaacaacagtatagcaggcacagcttaagacacaagataaataagttgttgcattcaccagtcaaaatatttttaaaaaatcaagaagcaatcactga

SEQ ID NO: 440 - DNA, 401 bp, Homo sapiens
cattcaccagtcaaaatatttttaaaaaatcaagaagcaatcactgaatagttttccttgatttagataccaattaggtttaatgaccaaatgagactgataatcagagctgaggaactcaatcaacaggattgcaacatgctttttttaaacctcttgaaagatcacatcactacagaataaggaaacctatttatttayttaatttgagacagtctctctctgtcacccaagctggagtgcagtggaatgatctcagctcactgcggcctccaccccagttcaagcaatttcatgctcagcctctgagtagctgggactacaggggcacaccaccacgcccagctaatttttttgtatttttagcagagacaggttttcaccatggtggccacgatggt

SEQ ID NO: 441 - DNA, 401 bp, Homo sapiens
cgaattttcattttcataacaagaaaacaaattctttaagtgagaaacagtacattttacacccttccttttcacaaaaatctcacagtattttctgacttatggtcttgataattagcactcctgtttgctcgtgttctctttctttcacaagcaatgtgatcttatttttaaaaaggacctgagacaagtagacaattracccttgaacaacacagatttgaaaagttttggtccacctatacacggattttctcccacctctgccacccatgagagagcaagaccaagccctcctccataccctactccatctgaagatgaccagggtaaagacctttatggggattcacttccacttaattattagtaaatatcttttctcttccttatgattttct

SEQ ID NO: 442 - DNA, 401 bp, Homo sapiens
agcctaagtgacggagtgagactttctcaaaaaaataaataaataaataaataaattatatctcctcttagaagtttgtccctctcttcaactgaaatctatcacagttcgacctacatggtagttgcattgatctgtccatcctggtgccacatatagaacatctactttgtcagatactgttccacatacagtaagcaraacacatgatcccagtccacatgaaggatggtgtccagtcatgtatggcctgattcttttctgtaaagtgctttcacatttgtagttggttcctttcctagagcactcctgtttttgcattcttgtagaaagcaaattatttccaccctgtcactgatttttttctccaattcaaacaatacgaactagaaaagagaact

SEQ ID NO: 443 - DNA, 401 bp, Homo sapiens
aaaacaaggcatagtgaccaggaagatggagtttctagttagtgcatacatatacatgcttatggttacttaatgtgatagaagcaaattattcagatgcctcttctccacagaaggttttggtcttcagcacgtaaagcaacttttttttttttgtttttttgagatagagtctcactctgtcaccaggctggagtgcartggtgcgatctcggctccctgcaaactctgcatccggaattcaagcgattctcctgtctcagcctcctaaatagctgggactacaggcacgcaccaccacgtccagctaatttttgtatttttagtagagacggggtttcactatgttggccaggatggtctcgatctcttgacttcgtgatcctcccgcctcggcctcc

SEQ ID NO: 444 - DNA, 401 bp, Homo sapiens
gagtgcaatggtgcgatctcggctccctgcaaactctgcatccggaattcaagcgattctcctgtctcagcctcctaaatagctgggactacaggcacgcaccaccacgtccagctaatttttgtatttttagtagagacggggtttcactatgttggccaggatggtctcgatctcttgacttcgtgatcctcccgcctyggcctcccaaagtgttgggattacaggcatgagccactgtgcccggcctcatgtagagcactttaaaagtacattggatccatagtaatcacttaactatgaacttgtatatccatatcaaaacttcatgttgtacagcttaaatatagacaataagaataaaataaataaatgaaaatattttaatatgctgagccatg

SEQ ID NO: 445 - DNA, 401 bp, Homo sapiens
ccctttttttatttttattttacgtttgccttttctaacaaattctccttaaaacagccagaatgatctctttgatgtatgatttagccaagtgtggtggctcccactagtaatcccagctactccaggaggctgaggtgggaggattgctgggagttcaagaccagtctgggcaacatagtgagaccccatttctaattwaaaaaaaaaaaaaagaagaaggaaacatgaattagattttgttgctcccttgtttaaactctctattggctttttacttcatttagaataaaatgaaactccctgtcctggcccctatttgatcttatttcctgtcactttcccattgtctgttatacctcacaatcactggactgcagttattgctgttcctccgaact

SEQ ID NO: 446 - DNA, 401 bp, Homo sapiens
acaccatctaaagtgagtcagacaatatgtccagcttcttgtgtgtgctaattaatgacactttgtggactatatatctgtatataactacttcatttcttacagagccttgatagatgaaagtaactagacatttaacttaaaaggtgctaggtcatcttcagttccaggctgtaagcattaagattgactgtacacgamctgttaaaaccattttctgggaaaggctcttaagtacctgtcagagtttacaatttgagagcaaacataaataaacccaaacgcctttcaactttgaacatttgatgcttgaaaatcacctctatttaaatttttgctagtcacatcttctcatcttttggtttcaggacctgattcatcagtttctatagatccattta

SEQ ID NO: 447 - DNA, 401 bp, Homo sapiens
cacaagtaggcaactggaggcttggaaaaaacccagacaacaacaatttgtttttcactggttctgctttcaaacaacttccaagagcacaatacagcaaaaagtgagagttatctatttctcccttcttcatccgaatacaaaatacatcctattttactgctttttttttgtcttttgcctctactttaagacttatcyaagaaataaagaaaagccaaccactcgttctatccacagaagaaaaacacacaccagcatagacaacacaaaagactgtgtctagatgttaagactatatatcctataggactataattttcatagatctaaaaagtgagaaacatatatatgtataaaattatgtgggatgcaagttactgagattttgaatatggggc

SEQ ID NO: 448 - DNA, 401 bp, Homo sapiens
ttccaagagcacaatacagcaaaaagtgagagttatctatttctcccttcttcatccgaatacaaaatacatcctattttactgctttttttttgtcttttgcctctactttaagacttatctaagaaataaagaaaagccaaccactcgttctatccacagaagaaaaacacacaccagcatagacaacacaaaagactrtgtctagatgttaagactatatatcctataggactataattttcatagatctaaaaagtgagaaacatatatatgtataaaattatgtgggatgcaagttactgagattttgaatatggggctctgtcatttaccctaaaatgttcagtggtcttttctattttttctctattctcagtaaaatagaactttctcaacaa

SEQ ID NO: 449 - DNA, 401 bp, Homo sapiens
gtctgaaaacctcctgtgcccttccaccacttaataatctaaccaactattgtaaaattacctattcttctaaaccaccaaatagttcgaatagaatagccttagatatctaataactaaatgaaaccacagtttacctctctaggcttcagtttcttcttcataaaataagaagcttgaactcgaaaagttttagatgcraagttcccaaaaatctagtagaggaagcaaaactctgtttggcaccctgtttccttgccctggacttttagcatttctttcttctggtgtaaagtcaaacaaaaaatagagatgaatctctaaatttacctttttattggggagataagagttgcaatacagggcatacacacagacctgatagtcttctgtatgtcaga

SEQ ID NO: 450 - DNA, 401 bp, Homo sapiens
tcttctaaaccaccaaatagttcgaatagaatagccttagatatctaataactaaatgaaaccacagtttacctctctaggcttcagtttcttcttcataaaataagaagcttgaactcgaaaagttttagatgcgaagttcccaaaaatctagtagaggaagcaaaactctgtttggcaccctgtttccttgccctggayttttagcatttctttcttctggtgtaaagtcaaacaaaaaatagagatgaatctctaaatttacctttttattggggagataagagttgcaatacagggcatacacacagacctgatagtcttctgtatgtcagaagaacaaagagaaggttggaggttttacgaaaaggagaaatgtcttgtcctgaaagaaatttcac

SEQ ID NO: 451 - DNA, 401 bp, Homo sapiens
gggagtggcaacctctgattggtgagtgacaacagtgcagtaggtaaaactaatcttagactcacagtggttgtcttagctgctattagataaaagtgatttcaggttacagcaggcagtttcagcagctaggtttgcagagaattacattcctagaacatgatattttaatatacatatccattgtgaaataaatactayaggcaagcaatttaacatatttatcactttccatagttaacatttttgtaagagcacataaaatctacttattctgcagattttgaatatacaatacaatattatttactataatctttctgttgtacattagatctctagacacactgattctttttttttttttttttttttttttgagacggagtttcagtcttgtc

SEQ ID NO: 452 - DNA, 401 bp, Homo sapiens
ttgatttttttttttttttaagattctacatgtaagtaagatcgtgcagtagttttgtttctatgtctggcttatagttcacttagcaggttcattcatgttgttgcaaatgctagaatctcctttttaggttggataattattccattgtgtatctataccacaatttatttaccgcttcatctgttaatggattcttasgttgttttcatatcttggctattgtgaataatgctgcaatgaacgtcaaagtgcagatatgtccatgaggtgtttatttcatctcttttgggcatacccagaagagggattgctggatcatatggtagcagaatgttttatttggaagggaaacttttatactgttttccataatggctataacaatttgcattccctct

SEQ ID NO: 453 - DNA, 401 bp, Homo sapiens
atctcctttttaggttggataattattccattgtgtatctataccacaatttatttaccgcttcatctgttaatggattcttaggttgttttcatatcttggctattgtgaataatgctgcaatgaacgtcaaagtgcagatatgtccatgaggtgtttatttcatctcttttgggcatacccagaagagggattgctggrtcatatggtagcagaatgttttatttggaagggaaacttttatactgttttccataatggctataacaatttgcattccctctgaaagtggacaagggttccctttattctacactctcaccaacacttgttatctcttgacttttttatacgagtcagtgtaactttaaatccacctggcctgatgctaaattatgaat

SEQ ID NO: 454 - DNA, 401 bp, Homo sapiens
ctataacaatttgcattccctctgaaagtggacaagggttccctttattctacactctcaccaacacttgttatctcttgacttttttatacgagtcagtgtaactttaaatccacctggcctgatgctaaattatgaatgggaaaaattatattaatacttagaatcctgactgggtgcagtggcttatgcctggaaccrtagcactttgggaggctgagatgggaggatcacttgagccaggagttcgagaccagccagggcaacatagtgagacctcatctctctaaaaaataaaatattagcctagtttggtggcacatgcctgtagtcccagctactcaggaggctgaggtgtaaggatcacttgagcccaggaggtcaaggctacggtaagccat

SEQ ID NO: 455 - DNA, 401 bp, Homo sapiens
acacttgttatctcttgacttttttatacgagtcagtgtaactttaaatccacctggcctgatgctaaattatgaatgggaaaaattatattaatacttagaatcctgactgggtgcagtggcttatgcctggaaccatagcactttgggaggctgagatgggaggatcacttgagccaggagttcgagaccagccagggyaacatagtgagacctcatctctctaaaaaataaaatattagcctagtttggtggcacatgcctgtagtcccagctactcaggaggctgaggtgtaaggatcacttgagcccaggaggtcaaggctacggtaagccatgattgcactactgtactctagcctgggtaacaaagcaaagcaagaccatcttcacacacacac

SEQ ID NO: 456 - DNA, 401 bp, Homo sapiens
cctggaaccatagcactttgggaggctgagatgggaggatcacttgagccaggagttcgagaccagccagggcaacatagtgagacctcatctctctaaaaaataaaatattagcctagtttggtggcacatgcctgtagtcccagctactcaggaggctgaggtgtaaggatcacttgagcccaggaggtcaaggctacrgtaagccatgattgcactactgtactctagcctgggtaacaaagcaaagcaagaccatcttcacacacacacacgaaaaaaacttagaattctttttttttttttttttttttttttttttttttttttttgggggagggtctcattctgttgccaggctggagtgcagtggcgatcttggctcactgcaacctctgact

SEQ ID NO: 457 - DNA, 401 bp, Homo sapiens
ttacaggtgcctgccaccatgaccagctaattttttttgtatttttagtagagacggagtttcaccgtgttggccaggatggtctcgatctcctgacctcgtgatcctcccgcctcagtcccccagagtgctgggattacaggcgtgagccatcacgcctggctaaacttagaattctttacattcaaataatgtctggtrtttttttacattattttcatgtatattccctaatttaaacttatccccacccccccccaccccaccatgatgtcgtcaacacagaactgttttctagattaggaaactgaggatagggattttaagtatctcgctcctagatcaggaccgcatccaggttgcagaatcccaatccagcactcttccacatagtcacagca

SEQ ID NO: 458 - DNA, 401 bp, Homo sapiens
ttttcacttagaaaatgtatgagatgttctttctgaaggggttctgtcactaacaggttgcattacttgtacaaccattatatctgaatcttcccagaccgtggtttttttttgtttgtttgttttgttttttattaaatactgtcattaaaccagaactcatggatatctatgtcatttccagctataaaattctagaawcttggggattttacctttttgtttcttattttattttcttttttatcaaaagccttatcacaggtaaaatggtaatctaagcaagaataagctctgtcatgatttatgaagcatggaaattcagctgtggccaggcacagtggctcacatctgtaatcccggcactttgggaggccgaggtgggtggatcacttgaggcc

SEQ ID NO: 459 - DNA, 401 bp, Homo sapiens
tttcttcaaagtaaaatgaagtggttaaacaggattactccccagatgtctccccaaactttacactggtgtaataattgtttgcaaggatcattgggtgattatgtctaaaagcctgcagttattagtcctcatatcaggaatgtgctatccccattcattttattgtagaacctggtttgctgtgggatagttgaaatwtgaagaattcacttggtcagtgtgtttcacggccttgctactttttctgcttggatggactcacttctctatatgattttctctttaaatcagaagtatttgacctgaagcagtaactcctctattcatataatagtgatattctgatacatatgtgaatattataaaaataagtagaatgataatattattactaatat

SEQ ID NO: 460 - DNA, 401 bp, Homo sapiens
atataaacacaaagcaacataggagaaaagtactagttacatgcccatggcaagaaaatatgcctgaaatggttctgtttattttgttgttgttgtttctgcttggaatttcagcagttatttggcaaagtgattagctagctgggaaggaagaaagctcgtctccatcagccttttgtcagagcccgccttttggtcccrgagcccccatgaaagtgtgctgtagaccaggaccactgttgccctgaatgcatgagtatgcagcctttttatcgtaggcttgtcatctcctgagttctgcaatagatgccatgtgaaatccagtatatactcagtctaacacatagctgctgcataagaaaatttctttccatgcttaagcaatttctgcatagagctta

SEQ ID NO: 461 - DNA, 401 bp, Homo sapiens
gcataagaaaatttctttccatgcttaagcaatttctgcatagagcttaattcatagaaatcacttattcattcaaaaagcattcactgagttcctagtaggggccacatattacgctaaagtagatacaaggataaaccaaatgtagatctgtcctttaaagagtataacatgtagggaacaactacactctacatggcratagaaagtcacaagtgggtgagaaccacaggagagaaacagtcagagggtgatgacagccagaactggtatgatgtgaaaggtcaatgacatcagaaacggcctcagggattttatagcctggctccttcaatttcagttcttggtataggccagcttttcaggactgttgattgtccctcttctttgtgtgaaattcc

SEQ ID NO: 462 - DNA, 401 bp, Homo sapiens
aagtcacaagtgggtgagaaccacaggagagaaacagtcagagggtgatgacagccagaactggtatgatgtgaaaggtcaatgacatcagaaacggcctcagggattttatagcctggctccttcaatttcagttcttggtataggccagcttttcaggactgttgattgtccctcttctttgtgtgaaattcctagtgsacagagcttctgcagtgcctcttaggtactttgaaaatcccaacgataaacatctggacccaacacagaagcctccaggaagctgtcttgttggcagtcacctacgagcaggcgataaacacaacacattaaatagtatgtaatgctccagggattgcagtcctctgcctcctaatctctctgtcctttcctcttttatt

SEQ ID NO: 463 - DNA, 401 bp, Homo sapiens
gagcttctgcagtgcctcttaggtactttgaaaatcccaacgataaacatctggacccaacacagaagcctccaggaagctgtcttgttggcagtcacctacgagcaggcgataaacacaacacattaaatagtatgtaatgctccagggattgcagtcctctgcctcctaatctctctgtcctttcctcttttattgacytgtcaggagcatgacagttgtgcacaaccattagcacacaagaaatgactttcctgtgggggtgatggggactggggaacctactctttgagatagaaagttaccctgatgcagaataattacagtccagcttagcctccttataagcgattaatcttacttttcctcttagcttctgtctgtttgtaacctgaggttga

SEQ ID NO: 464 - DNA, 401 bp, Homo sapiens
cttaggtactttgaaaatcccaacgataaacatctggacccaacacagaagcctccaggaagctgtcttgttggcagtcacctacgagcaggcgataaacacaacacattaaatagtatgtaatgctccagggattgcagtcctctgcctcctaatctctctgtcctttcctcttttattgacttgtcaggagcatgacasttgtgcacaaccattagcacacaagaaatgactttcctgtgggggtgatggggactggggaacctactctttgagatagaaagttaccctgatgcagaataattacagtccagcttagcctccttataagcgattaatcttacttttcctcttagcttctgtctgtttgtaacctgaggttgaccgaagttagttctttt

SEQ ID NO: 465 - DNA, 401 bp, Homo sapiens
agatagaaagttaccctgatgcagaataattacagtccagcttagcctccttataagcgattaatcttacttttcctcttagcttctgtctgtttgtaacctgaggttgaccgaagttagttcttttagttggttcttgagaaaaccctttcagtctgtatttccaggaataaatcttaactctaatttaaaaaacaaaarggccctataaatagttacttcaaaccctttaatttatagctgcattctcaatgtcttcatcctcccaaaatcactcacctctacatttttgccctaagtacctaggttagcacaacttttcacaacttctctttaacatttctctgaataacgtgaaccttccttatttccttaaaatataaacttatatatgttcttcc

SEQ ID NO: 466 - DNA, 401 bp, Homo sapiens
ttggaagtgtctcttcttttggaagtgggagaccaagtggaggggaagaacatgaacaagggattgaagccattgagaaagtcatttattcattcgatagatatcttctgagcactgcgctaggacctaaggacagaataatgaaggcagagtttatccttccactagcacagcagtcaaaaccaataggaattaagattrtcagaagtgacaaaactgtacatagacctgttcacataactaacaagggaaacgatggaggtgatgtggaagtctgaacgtggtgcaggaataactcagccaacatccccctcctttgggaaatttagatagatgtaggtagattgagaagtgatataacagtcatgtggcatcattgagagagtcagttttctataatg

SEQ ID NO: 467 - DNA, 401 bp, Homo sapiens
agcagggttattattaaaactctgatcccccaagtccaactgaaggggctccagtcactaccccatcatctcagtggtgagatgtagtcaaggcatctaacctctctgcctcagtttcctcatatgtaaaatagagacaatcattataaagtgtttagcagaatgccagatccacagtaaatatcagtaaaaggaaatttrtaaatttgtaaattgttagggaatggggtgagaccctcaagattctaatcttcaagggaaagaggatgctaaggaaagtcagaggaagcccgagaatcctaagagaggatgatacaagcaaattaattaaggaataagatcaaagaatattgggaaatgtttaatccacttggtgtgggaaaaggacttttttgtttgtt

SEQ ID NO: 468 - DNA, 401 bp, Homo sapiens
ccacagtaaatatcagtaaaaggaaatttataaatttgtaaattgttagggaatggggtgagaccctcaagattctaatcttcaagggaaagaggatgctaaggaaagtcagaggaagcccgagaatcctaagagaggatgatacaagcaaattaattaaggaataagatcaaagaatattgggaaatgtttaatccactyggtgtgggaaaaggacttttttgtttgtttgtttttgagacggagtcttgctctgtcgcccaggctggactgcagtggtgcgttctcggctcactgcaagctccgtctccctggttcaagcgattctcctgcctcagcctcccgagtagctgggattacaggcgccagccacaacgcccacgcccagctaattttttttt

SEQ ID NO: 469 - DNA, 401 bp, Homo sapiens
ggaaatttataaatttgtaaattgttagggaatggggtgagaccctcaagattctaatcttcaagggaaagaggatgctaaggaaagtcagaggaagcccgagaatcctaagagaggatgatacaagcaaattaattaaggaataagatcaaagaatattgggaaatgtttaatccacttggtgtgggaaaaggacttttwtgtttgtttgtttttgagacggagtcttgctctgtcgcccaggctggactgcagtggtgcgttctcggctcactgcaagctccgtctccctggttcaagcgattctcctgcctcagcctcccgagtagctgggattacaggcgccagccacaacgcccacgcccagctaattttttttttttttttttttgtagtagaga

SEQ ID NO: 470 - DNA, 401 bp, Homo sapiens
gagcccggccgaaaaggactcttttttttttttttttacacagagtctcgctctgttgcccaggctggagtgcagtggcgccatctcggctcactgcaagctccgcctcccgggttcacgccattctcctgcctcagcctccagagtagctgggactacaggcgcccgccaccacacccggctaatttatttatttttttmtttttttcttttttgtatttttaatagagacggggtttcaccgtgttagccaggatggtctccgtctcctgacctcatgatccgcctccctcagccttccaaagtactgggattacaggcgtgagccaccgcgcccgaccatgaaaaggactctttttaacctcttctccatgtctaatctgagtttcaacctttttatt

SEQ ID NO: 471 - DNA, 401 bp, Homo sapiens
tttttttttttttacacagagtctcgctctgttgcccaggctggagtgcagtggcgccatctcggctcactgcaagctccgcctcccgggttcacgccattctcctgcctcagcctccagagtagctgggactacaggcgcccgccaccacacccggctaatttatttatttttttctttttttcttttttgtatttttartagagacggggtttcaccgtgttagccaggatggtctccgtctcctgacctcatgatccgcctccctcagccttccaaagtactgggattacaggcgtgagccaccgcgcccgaccatgaaaaggactctttttaacctcttctccatgtctaatctgagtttcaacctttttattcttattctgttataagtacttctc

SEQ ID NO: 472 - DNA, 401 bp, Homo sapiens
tgtgtttgctggtactgaatcctaccctaatacttccgaaatgaacctcattggataactcaactcttcattatgtttttgctaatcttttaggagcctttttgcaacccaccgtctccctaccctcgttgggtctttttttttttcttcttcagtcagggttttgctctgtaacccagtggggaaagtggcatgatgatrgctcactgcagcctcaaattcctggactcaagctattttcccgccttaccttcctgagcagctgggactataggcgcatgccatcatgcgtggctaatttctaaaaaatatatatttttgtagagacatctctctatgttgcatttctggtcttgaactcctaggcgcaagcaatcctcccacctcagcctcccagagtg

SEQ ID NO: 473 - DNA, 401 bp, Homo sapiens
ctgaatcctaccctaatacttccgaaatgaacctcattggataactcaactcttcattatgtttttgctaatcttttaggagcctttttgcaacccaccgtctccctaccctcgttgggtctttttttttttcttcttcagtcagggttttgctctgtaacccagtggggaaagtggcatgatgatggctcactgcagccycaaattcctggactcaagctattttcccgccttaccttcctgagcagctgggactataggcgcatgccatcatgcgtggctaatttctaaaaaatatatatttttgtagagacatctctctatgttgcatttctggtcttgaactcctaggcgcaagcaatcctcccacctcagcctcccagagtgctggggattgcagg

SEQ ID NO: 474 - DNA, 401 bp, Homo sapiens
aagcaatggtgaagtaccctttttaaaatattttatttagaagaatttcttatttcagatacaacagatgatacaagttgaatctagtagggtttagtaagagacaataaacatattaaacaaagccttacgtattttttaaggtgaaaaaaatcccccaagatagagtccgtgcttagaaactacccctgttaaatactkaatatataacatttgtaatgttcaaataccgaaagaaaaaaatgtacttgttcagagtctattcttgagtcaacctaccttctcagctctactgaccccaaacaaggtaaacaagctgatttaagttacaactaacttaaaaattatacatctaactcttttgtaaataactttagggctggtgctcaaaataattggaa

SEQ ID NO: 475 - DNA, 401 bp, Homo sapiens
ccctgggttccagtgattctccagcctcagcctcctgattagctgggattacagacacccgccatgacaccctgctaatttttgtaattttagtagagacagggtttcaacatattggtcaggctggtctcgaactcctgacctcaggtgatccacccaccttggtctctcaaagtgctgggattacaggtgtgagccacygtgcccggcccttggcctttattctttattttccctgatattaatccctaatgaaatgcatttagtacacattcatctttttaaaatgtgcagtttttgtcacagaagtagtgtaaaggacaccagaatggttggaagacgatttttgtggtcttctacaagtcctgttttggtgactctgagcaagtcttaaccttgcc

SEQ ID NO: 476 - DNA, 401 bp, Homo sapiens
ctcagcctcctgattagctgggattacagacacccgccatgacaccctgctaatttttgtaattttagtagagacagggtttcaacatattggtcaggctggtctcgaactcctgacctcaggtgatccacccaccttggtctctcaaagtgctgggattacaggtgtgagccaccgtgcccggcccttggcctttattcyttattttccctgatattaatccctaatgaaatgcatttagtacacattcatctttttaaaatgtgcagtttttgtcacagaagtagtgtaaaggacaccagaatggttggaagacgatttttgtggtcttctacaagtcctgttttggtgactctgagcaagtcttaaccttgcctacatttcctcacatttagggcaag

SEQ ID NO: 477 - DNA, 401 bp, Homo sapiens
accagaatggttggaagacgatttttgtggtcttctacaagtcctgttttggtgactctgagcaagtcttaaccttgcctacatttcctcacatttagggcaagtgtactcttcagggtcaactgcaatatctgtgggagtgctttgtgttttgtagcatgcatcttactagtgcttttgttgttgtttgcgttgtcgtgygggttccccacctcccccataacactgtaaagaaatgctgagactaagtggtagggagtggcagccatctggtgaggatggaaagttaatatctttatttatgtaataaacctccctttagaattctgttactgtcaaatttctcctggaattctaatagtattttcgctatgtgttttgaccttttgctattgggaaga

SEQ ID NO: 478 - DNA, 401 bp, Homo sapiens
tagcatgcatcttactagtgcttttgttgttgtttgcgttgtcgtgcgggttccccacctcccccataacactgtaaagaaatgctgagactaagtggtagggagtggcagccatctggtgaggatggaaagttaatatctttatttatgtaataaacctccctttagaattctgttactgtcaaatttctcctggaattytaatagtattttcgctatgtgttttgaccttttgctattgggaagacacaagtttatgaatgacaaatgggtaggggaatttaggctgcacagttaagtaggaagaatgaagtctttgctagagttagaatccagtttgaccttgaacaagttactcaatgactaagctccatttgcaaaatcgttttgttaaagggatt

SEQ ID NO: 479 - DNA, 401 bp, Homo sapiens
ttatgatttccgcagacttaaatcaaccctgggcttctatccttatactagttttattttatgttttttgtttgtctttagagaaatgatcgtgttctgtcatccaggctggagtgcagggacataatcacagctcactgcagcctcaaactcctgggctccattgatcttcctgcttcaacctgctgagtagctaggacyacaggcacgcaccatcaggcccaactacatttttaaatttttttctagagacgatgtctcgccatgtttcccaagctggtcttgaaccctggcctcaagcaatcctcccacctcagcttccctaagtaccaggattacaggcataagccactgagcccagccccacccttacatgttttctcttggcatcctgatccctg

SEQ ID NO: 480 - DNA, 401 bp, Homo sapiens
gtttcccaagctggtcttgaaccctggcctcaagcaatcctcccacctcagcttccctaagtaccaggattacaggcataagccactgagcccagccccacccttacatgttttctcttggcatcctgatccctggacacaaccccattgagatacccttccatttaatacttttcttagtcatttaccaaatgtgtttayagtaatatattcccatatatttgtggcagagtacagcactcatataaattttgatgaacacagacatgctgtcccatacttgtaggtaaaacaatcatgtgaaattaaaataaatattttgtttagatagatcttgactatcatacacttattctaacaatattgcccattcctcaaaacattctgggaactccttgaaa

SEQ ID NO: 481 - DNA, 401 bp, Homo sapiens
tgctctcagtgatattccctttttttttttttttttggacggagtctcactctgccgccaggctggagggcagtggcaccatcttggctcactgcaacctccgcctcccaggttcaagtgattctcctgcctcagcctcccaagtagctgggactacaggcacgtgccaccacgcctagctaatttttgtgtgtgtgtgtkttttttttagtagaggtggggtttcaccatgttggccgggatggtctcgaactattaaccttgtgatctgcccacctcggcctcccaaagtgctgggattacaggcgtgagccactgtgcccggccgatattcctgtttttatggttgcaattgtatgctatggcaccggtactatgtgggaagaatgatgctaggtact

SEQ ID NO: 482 - DNA, 401 bp, Homo sapiens
gtttatttatgcaaagcacataggatcataggattgctctctaggagaatggagtaattaataccatgaagactcatagacaatgcttcctcccctagaaactcattatttttcgaattttggcactgatctttcctgcctacttcctggcttttactaccttgtgaaaaagccccaagtgttagaagcagcttccccagyttactgccatcccttctaaggtccctggacaccactgcttgtctcctaccttagaaagtgcctacttggccaggcacgctggctcacgcctgtaatcctagaactttgggaggctgaggcaggaggatcacgaggtcaagagatcgagaccatcctggccaacatggtgaaaccccagctctactaaaaatacaaaaatt

SEQ ID NO: 483 - DNA, 401 bp, Homo sapiens
catagacaatgcttcctcccctagaaactcattatttttcgaattttggcactgatctttcctgcctacttcctggcttttactaccttgtgaaaaagccccaagtgttagaagcagcttccccagcttactgccatcccttctaaggtccctggacaccactgcttgtctcctaccttagaaagtgcctacttggccagrcacgctggctcacgcctgtaatcctagaactttgggaggctgaggcaggaggatcacgaggtcaagagatcgagaccatcctggccaacatggtgaaaccccagctctactaaaaatacaaaaattagctgggtgtggtggggcatgcctgtagtctcagctacttgggaggctgaggtaggagattcacttgaagccgg

SEQ ID NO: 484 - DNA, 401 bp, Homo sapiens
gtgttagaagcagcttccccagcttactgccatcccttctaaggtccctggacaccactgcttgtctcctaccttagaaagtgcctacttggccaggcacgctggctcacgcctgtaatcctagaactttgggaggctgaggcaggaggatcacgaggtcaagagatcgagaccatcctggccaacatggtgaaaccccarctctactaaaaatacaaaaattagctgggtgtggtggggcatgcctgtagtctcagctacttgggaggctgaggtaggagattcacttgaagccgggaggtggaggttgcagtgacctgagatcacgccactgcactccagcctggtgacagagagagactctgtctcaaaaaaaaaaaaaaaaagtgcctactcatacc

SEQ ID NO: 485 - DNA, 401 bp, Homo sapiens
gcaataaaaattattaatattgtagaacatgtattgatcctatgtatcaggcttgaattaggtgctttaagtgggttgtctcaattaattcatcactctgattttcaattttctgttctaagaaatgagatcaaatatgcctatcttgccgaagagtagtaaaaattagacaggtagctgcctctggatcctgcagggtasagaaagtattacatatctgcatgtgtggatgtgaaggacacacagatacataaggtaaatgagaatttattataaaggtgtatttgacaaaaattggggaaaaaaggaaaattggagaagatatctttctgtgagattaaagacaaataggggaaaaaagggaccaatgacaagaattagagcaaggtatgtaaagaatt

SEQ ID NO: 486 - DNA, 401 bp, Homo sapiens
gggtagagaaagtattacatatctgcatgtgtggatgtgaaggacacacagatacataaggtaaatgagaatttattataaaggtgtatttgacaaaaattggggaaaaaaggaaaattggagaagatatctttctgtgagattaaagacaaataggggaaaaaagggaccaatgacaagaattagagcaaggtatgtaawgaattccagagctggctctaagagatagagcagctggtcatatatgaaagggatgattcattcttcaggggctatccctcgtgatcctatttccttgcacaagctatcagttaatttattgattcacctcctttatgtgctcataggcatcaagaatacaatggatcacatacaattacagaaaagtttcatttcaggag

SEQ ID NO: 487 - DNA, 401 bp, Homo sapiens
cacacagatacataaggtaaatgagaatttattataaaggtgtatttgacaaaaattggggaaaaaaggaaaattggagaagatatctttctgtgagattaaagacaaataggggaaaaaagggaccaatgacaagaattagagcaaggtatgtaaagaattccagagctggctctaagagatagagcagctggtcatatrtgaaagggatgattcattcttcaggggctatccctcgtgatcctatttccttgcacaagctatcagttaatttattgattcacctcctttatgtgctcataggcatcaagaatacaatggatcacatacaattacagaaaagtttcatttcaggaggtcaaacaggaagtttctttctgacttatattgaagtactcatg

SEQ ID NO: 488 - DNA, 401 bp, Homo sapiens
ttcatttcaggaggtcaaacaggaagtttctttctgacttatattgaagtactcatgatgacaatggagaaaaatgaacataatcttttatgggtggggaccatgtgatctcttttctttcaagctccagagggtttataagacatttccatgttcaatttgattccggaaattatatgttgggtagaacagactcttttyggcaactcatgagaattgccaatgatcccagcatttgctactcttgatagtaattcccttcatttcctccatttttatacatgacctgcagtaaatattgacagttactttgctggaaagaataacacagagacagcttttcttaagctccaaactgaatttcgtggttaattgattggccagctggccctgccgtctct

SEQ ID NO: 489 - DNA, 401 bp, Homo sapiens
aagaatttgtcagggtcccagtccctgtaccttatgggagaattaaattcattaaactgggccagatgtggtggctcacactggtaatctcagcactttgggaggctgaggcaggtggatcacctgaggtcaggagcttgagaccagcctggccaacatggtgaaaccccatctctactaaaaataaaaaaaattagtcargcttggtggtaggtgcctgtaatcccagctagtcaggaggctgaggcaggagagtcgcttgaacctgggaggcagaggttgcagtgagccgagatcgtaccactgcactctagcctgggcgacagaatgagactcagtctccaaaaaataaataaataaattcattaaactgaacaaattgagcccaatttatttgtcat

SEQ ID NO: 490 - DNA, 401 bp, Homo sapiens
ataatttattctcattattttttgtttgttttttgagacagagccctgctctgtcatcagtctggagtacagtggcgcgatctcagctcactgcaatccctgcctcctgggttcaagtgattcccctgcctcagcctcccgagtagctgggactacaggcatgcatcaccatgcccagctaattttttctattttggcagrgatagggtttcaccaggttggccaggatggtctcaatctcctgaccttgtgatgtacccaccttggcctcccaaagcgctgggattacaggtgtgagccaccacgcccagcccattcttgtttcttggtaaaggaaaatccaactatatatatcttctttaggatcacttaaagacccatacagatctgtcaaatagaca

SEQ ID NO: 491 - DNA, 401 bp, Homo sapiens
tctggagtacagtggcgcgatctcagctcactgcaatccctgcctcctgggttcaagtgattcccctgcctcagcctcccgagtagctgggactacaggcatgcatcaccatgcccagctaattttttctattttggcagggatagggtttcaccaggttggccaggatggtctcaatctcctgaccttgtgatgtacccrccttggcctcccaaagcgctgggattacaggtgtgagccaccacgcccagcccattcttgtttcttggtaaaggaaaatccaactatatatatcttctttaggatcacttaaagacccatacagatctgtcaaatagacatccaatattttggtaggctttgaaaggaggtgcaaaaggatgagaaggaatttagggaga

SEQ ID NO: 492 - DNA, 401 bp, Homo sapiens
catgcccagctaattttttctattttggcagggatagggtttcaccaggttggccaggatggtctcaatctcctgaccttgtgatgtacccaccttggcctcccaaagcgctgggattacaggtgtgagccaccacgcccagcccattcttgtttcttggtaaaggaaaatccaactatatatatcttctttaggatcacytaaagacccatacagatctgtcaaatagacatccaatattttggtaggctttgaaaggaggtgcaaaaggatgagaaggaatttagggagatcattatctgaatcattgaacaaatgctccttaagcacctggtgaagagaggctactgagaagcagagcacgtggtcccagcctacgaggtgacaaagtaaaatggcag

SEQ ID NO: 493 - DNA, 401 bp, Homo sapiens
aggtgatggagatgttaattagcttgatttaaccttcctacaatgtatatgtgttagtccgtttttgcgttgctgtaaagaagtatctgaggctaggtaatttgtaaagaaaagtggcttattttggctcatggttctgtagactataaaggtagcgtggtgctagcatctgcttttggtgagggctcagaaagcttccartcatggcagaaggcaaagggggagcccacatatcagatggtgagagggagcaagagttgagggggaggtgccacgctcttttaaacaaccagatctcatatgaactcagagcaagaactcactcattatcgtgagaacaacaccacgctattcacaagggatccacccccatgaccaaaacacctcccactgggccccac

SEQ ID NO: 494 - DNA, 401 bp, Homo sapiens
taaagaagtatctgaggctaggtaatttgtaaagaaaagtggcttattttggctcatggttctgtagactataaaggtagcgtggtgctagcatctgcttttggtgagggctcagaaagcttccaatcatggcagaaggcaaagggggagcccacatatcagatggtgagagggagcaagagttgagggggaggtgccacrctcttttaaacaaccagatctcatatgaactcagagcaagaactcactcattatcgtgagaacaacaccacgctattcacaagggatccacccccatgaccaaaacacctcccactgggccccacttccaacactgcgggttacatttcaacatgagatttaaaaccatgtcagtatacatacatacattaaaatatgca

SEQ ID NO: 495 - DNA, 401 bp, Homo sapiens
catacatacattaaaatatgcatcacattgtgccctatacacatataaattattgtttgtcaattaaagaaagaaataattttaaaataaataggccaggcgcagtggctgatgcctataatccaagcactttgggaggccaaggcaggcagattccttgagcccaggagttcaagagcagcctgggcaacatgatgaaayctcatctctaaaaaaatacaaaaattagctgggtatgtggggggtgcacaggtagtcccagctactcaagaggctgaggtggggggattgcttgagcctggcaggcggaggttgcagtgagattgcaccactgcacaccagcctgggtaacccattgagaacctgtcttaataaataaataaataaataaacaaacaaat

SEQ ID NO: 496 - DNA, 401 bp, Homo sapiens
aacatgatgaaatctcatctctaaaaaaatacaaaaattagctgggtatgtggggggtgcacaggtagtcccagctactcaagaggctgaggtggggggattgcttgagcctggcaggcggaggttgcagtgagattgcaccactgcacaccagcctgggtaacccattgagaacctgtcttaataaataaataaataaayaaacaaacaaataaaggttgtgttagaacaaaaaagaaaaagagtgacaatgcaagccacatagttgaagttgacattaactagcatgtgccaacgagtgacaaaggaaacaagtgcaaagcaagaaaagttagccattatcatggtccatagataatactctcaaccccggctcttgagtgaagcagtattgtgcagtg

SEQ ID NO: 497 - DNA, 401 bp, Homo sapiens
tcccagctactcaagaggctgaggtggggggattgcttgagcctggcaggcggaggttgcagtgagattgcaccactgcacaccagcctgggtaacccattgagaacctgtcttaataaataaataaataaataaacaaacaaataaaggttgtgttagaacaaaaaagaaaaagagtgacaatgcaagccacatagttgragttgacattaactagcatgtgccaacgagtgacaaaggaaacaagtgcaaagcaagaaaagttagccattatcatggtccatagataatactctcaaccccggctcttgagtgaagcagtattgtgcagtggttacatttatggacctaatggtcaaactgcatgagttcaaatcccagctttacagctaatggttgca

SEQ ID NO: 498 - DNA, 401 bp, Homo sapiens
ccagctactcaagaggctgaggtggggggattgcttgagcctggcaggcggaggttgcagtgagattgcaccactgcacaccagcctgggtaacccattgagaacctgtcttaataaataaataaataaataaacaaacaaataaaggttgtgttagaacaaaaaagaaaaagagtgacaatgcaagccacatagttgaarttgacattaactagcatgtgccaacgagtgacaaaggaaacaagtgcaaagcaagaaaagttagccattatcatggtccatagataatactctcaaccccggctcttgagtgaagcagtattgtgcagtggttacatttatggacctaatggtcaaactgcatgagttcaaatcccagctttacagctaatggttgcatt

SEQ ID NO: 499 - DNA, 401 bp, Homo sapiens
aaagcaagaaaagttagccattatcatggtccatagataatactctcaaccccggctcttgagtgaagcagtattgtgcagtggttacatttatggacctaatggtcaaactgcatgagttcaaatcccagctttacagctaatggttgcattatcttgggcaagtcatttaacatctctgccttaattgattcacctctratatagagctgataggagtctgaaatagctgctataaggactaaatacattaatatatagatagcattcataacagtgttttgtggactttccgaaggtgctgggccttccagttgccttaaagaatggattggattagggtggaggagatgaaaaaaggcatcacagtacatcaagtgaatggtgccagtgtaagtaga

SEQ ID NO: 500 - DNA, 401 bp, Homo sapiens
gccttaattgattcacctctaatatagagctgataggagtctgaaatagctgctataaggactaaatacattaatatatagatagcattcataacagtgttttgtggactttccgaaggtgctgggccttccagttgccttaaagaatggattggattagggtggaggagatgaaaaaaggcatcacagtacatcaagtgmatggtgccagtgtaagtagaatacaggaataaggaaatgcccagcttgttggggggttagtatgaagacctctaactgggactgaaagttctagcaacagtaaaataccctggcctccaactactaagtaccctgaatactaagatgagtttatttttcattctatagacaatttccatctctttctttcaaaacaaaca

SEQ ID NO: 501 - DNA, 401 bp, Homo sapiens
aagacctctaactgggactgaaagttctagcaacagtaaaataccctggcctccaactactaagtaccctgaatactaagatgagtttatttttcattctatagacaatttccatctctttctttcaaaacaaacaaatgatgacgctgtatgagtaacagaagatgactgggaacagaaccgtgaacagaattaagcacstgcaatcaaataaaattcacagaaaacttaatttaccacattagtgatatgcataatcgttgttgtgctggtaaccagctcttcaggaaaaaaaaaagaaaattcctgatttgttaaaatactgtcactatggcccactgaaactatcaaaatgaggcatataacaggaaattgggaggagagttatacaatcagctctt

SEQ ID NO: 502 - DNA, 401 bp, Homo sapiens
aatcgttgttgtgctggtaaccagctcttcaggaaaaaaaaaagaaaattcctgatttgttaaaatactgtcactatggcccactgaaactatcaaaatgaggcatataacaggaaattgggaggagagttatacaatcagctcttgctagaaaatgatgatcattgacattacccttatggaaccaatccaaatttgggsgttgttttctttctttctacttttgtgtgtgtgtgtgtgtgtgtgtgtgttctctggtatctgtaaataacatccatatcagtttctccatccattctttgcacatcactggtaatgcctgagatcaaatctgccatgcttttccgggcagatccaggctgggatctcagtatccctgcttccctgaagctgtccaacct

SEQ ID NO: 503 - DNA, 401 bp, Homo sapiens
gtccaacctcactcccttcattgctctgctgtgattaggaaggagccattccaaggactatggggtgctctgtgggataagcccagtccttcttccatacaggaggaacaggggacgggtgctgacaagaggaaatgcatggagcagagagagggaacaggcagatggactcaaggtccccagactctttcgagtgttgartcagaaggactggagctgtgggggaagagggtgggaacagggtagtggaataacatgcagacagatgggctgaactgaaggtccagaggcactcccagccgtcaaccagcagttcttacaccaggttgatgttagggaacaaccccagctttaaaacttatttataccaaagtgccaaatgactcatttgagacaaggct

SEQ ID NO: 504 - DNA, 401 bp, Homo sapiens
tggactcaaggtccccagactctttcgagtgttgaatcagaaggactggagctgtgggggaagagggtgggaacagggtagtggaataacatgcagacagatgggctgaactgaaggtccagaggcactcccagccgtcaaccagcagttcttacaccaggttgatgttagggaacaaccccagctttaaaacttatttayaccaaagtgccaaatgactcatttgagacaaggctttgatctctggggacagggcgttagacagcatgaggcttaggttgccaggtgtcttcccacagttcctgtcttgtgtatgttacacactcaattcatgtcaggatgacactacattggcatgatccaagggaacccataactgacatgaatatgtgtgtgtgtgt

SEQ ID NO: 505 - DNA, 401 bp, Homo sapiens
tggctagagaggacttctcttccttgggggttctagtctattctctgagttgtgacaaacatttccattccctgtaattacccacttgccttgtctctgaagaggcattcacagaataaaccaaaagaattgtagtgttaggaagatgtcaaatcatttgacctctactccatctccggcttttccttctccccccatgaragttatgcatttagttgcctcttgttcattagtctggacttagagcaaagtgactacttcaacttcttgtgctcaacatatgccacagttgcttacatgtgataagtccatttgtcacttgataagcctttgctagaatttccttctctccatctccaaccttataatgatcttgaaggcaatgtcggaggctaggtaag

SEQ ID NO: 506 - DNA, 401 bp, Homo sapiens
ctcctactgtagacttagataggaactcacttttgtcttttttctgctaggccaataaatacgttctcagcttagaaaaggggctttttggtttgcctatttctgtttctttttacagaagttaacacatttatttgtgacctattaaaatcatgaagtattaccactgggttttatgttttaatggaaacatttcatgtytagagaataatgatgcttaaaatttatatgaattaaaattttagcctgtaaaaagagcctggaaatatcttcataccatcatctatccttccacttaaatattaatcacaaagaagatcaggcgtagtgaacatacaccacgcaacctcccattcagagaattcttgtgcaaagaaggattccaaagggtttcagaaata

SEQ ID NO: 507 - DNA, 401 bp, Homo sapiens
ctattaaaatcatgaagtattaccactgggttttatgttttaatggaaacatttcatgtttagagaataatgatgcttaaaatttatatgaattaaaattttagcctgtaaaaagagcctggaaatatcttcataccatcatctatccttccacttaaatattaatcacaaagaagatcaggcgtagtgaacatacaccaygcaacctcccattcagagaattcttgtgcaaagaaggattccaaagggtttcagaaatactaaacttcttgaaattgaaattgaaattgaaatccttcttggcttctctacctgttacttcctctgcagtgctcagtatagtgtctgctctccctcctttctttttctttttttttgagacagattttcactcttgttgc

SEQ ID NO: 508 - DNA, 401 bp, Homo sapiens
attcttgtgcaaagaaggattccaaagggtttcagaaatactaaacttcttgaaattgaaattgaaattgaaatccttcttggcttctctacctgttacttcctctgcagtgctcagtatagtgtctgctctccctcctttctttttctttttttttgagacagattttcactcttgttgcctaggctggagtgtaatggygcaatcttggctcaccgcaacctctgcctcttgggttcaagcgatactcctgcctcagcctcccaaggagctgggattatagtaccacgcccagctaattttgtcttttcagtagagatggggtttctccatgttggtcaggctggtctccaactcctgacctctggtgatcctccctcctcggcctcccaaagtgctgg

SEQ ID NO: 509 - DNA, 401 bp, Homo sapiens
tcaaacccctaaagttatccttgaataaatgagccttgcaaattatatttccccaatttgcacttatttctttcttttttttcttttttctttgagacagagtttcactcttattgcccaggttggagtgcaatggagtgatctcagctcactgcaacctccgcctcctgggtttaagcaattctcctgcctcagcctccyaagtagctgggattacaggcgctccccccctccccatgcctggctaattttttatatttttagtagacagggttttgccgtgttggccaggctggtctcaaactcctgacctcaagggatctgcccacttcggcctcccaaagtgctgggattacagatgtgagccaccgcacccagcctcagaatttcttactgaataa

SEQ ID NO: 510 - DNA, 401 bp, Homo sapiens
caaacccctaaagttatccttgaataaatgagccttgcaaattatatttccccaatttgcacttatttctttcttttttttcttttttctttgagacagagtttcactcttattgcccaggttggagtgcaatggagtgatctcagctcactgcaacctccgcctcctgggtttaagcaattctcctgcctcagcctcccragtagctgggattacaggcgctccccccctccccatgcctggctaattttttatatttttagtagacagggttttgccgtgttggccaggctggtctcaaactcctgacctcaagggatctgcccacttcggcctcccaaagtgctgggattacagatgtgagccaccgcacccagcctcagaatttcttactgaataat

SEQ ID NO: 511 - DNA, 401 bp, Homo sapiens
cccctccccatgcctggctaattttttatatttttagtagacagggttttgccgtgttggccaggctggtctcaaactcctgacctcaagggatctgcccacttcggcctcccaaagtgctgggattacagatgtgagccaccgcacccagcctcagaatttcttactgaataatccttttatagtttgttataactaacwcaaaacagtagtgggagttcttattcagatagctaacaaattccaggcacctcctgtactataagtgtccctcatagcacaaaggggaaagctgccctttaagaatccataaatgcaatgaaactctacttacgccacccaggttgttaggacacaggaatattaatctggagaagcctttgataatagaatatagtcaa

SEQ ID NO: 512 - DNA, 401 bp, Homo sapiens
tagacagggttttgccgtgttggccaggctggtctcaaactcctgacctcaagggatctgcccacttcggcctcccaaagtgctgggattacagatgtgagccaccgcacccagcctcagaatttcttactgaataatccttttatagtttgttataactaacacaaaacagtagtgggagttcttattcagatagctaamaaattccaggcacctcctgtactataagtgtccctcatagcacaaaggggaaagctgccctttaagaatccataaatgcaatgaaactctacttacgccacccaggttgttaggacacaggaatattaatctggagaagcctttgataatagaatatagtcaaagttgacattgtgctagaatatttgaattttttattt

SEQ ID NO: 513 - DNA, 401 bp, Homo sapiens
aggacacaggaatattaatctggagaagcctttgataatagaatatagtcaaagttgacattgtgctagaatatttgaattttttattttccagaaattattttcactctttgctacatttactgtttgacctcaaaagaatgtaaaagcaagtttttaaatacaccattatttcctcaagaggctacagaattgctggawttttttttttttttggaatggagtcttgctctgccacccaggctggagtgcagtggcatgaccttggcacactgcaacctccacctcctgggttcaagcagttctcttacctcagcctcctgagtagctgagattacaggtgtgtgctcccatgcctggctaatttttgtatttttagtagaggtggggtttcgccatgt

SEQ ID NO: 514 - DNA, 401 bp, Homo sapiens
taatctggagaagcctttgataatagaatatagtcaaagttgacattgtgctagaatatttgaattttttattttccagaaattattttcactctttgctacatttactgtttgacctcaaaagaatgtaaaagcaagtttttaaatacaccattatttcctcaagaggctacagaattgctggatttttttttttttttkgaatggagtcttgctctgccacccaggctggagtgcagtggcatgaccttggcacactgcaacctccacctcctgggttcaagcagttctcttacctcagcctcctgagtagctgagattacaggtgtgtgctcccatgcctggctaatttttgtatttttagtagaggtggggtttcgccatgttggccaggctggtct

SEQ ID NO: 515 - DNA, 401 bp, Homo sapiens
ggaggagaccaggagtcaccaagccctgccttacaagcaagaattaaaaaaaaaaaccaacctccaaaaatgtatcttttctgatcagtttctctctctgtaagacttaagaagtcaagtgaatccaagttgcatttcgtttccctttaccctcatggtgtagaaattgagcaataggtggggtcatgggcacatttgagrcagtattgcagcttttagtcagaactcgtttttacccaacttctgctttttttgttttttgtggtttttttctttcttttgagtttgaaaatccaacactgttttacgttattttttttttttttatcaatagtggaaactcagaaatttaaaagatgagattttgctttgccaattaggttcatttgacaaaaatgtac

SEQ ID NO: 516 - DNA, 401 bp, Homo sapiens
aatgaacctaattggcaaagcaaaatctcatcttttaaatttctgagtttccactattgataaaaaaaaaaaaaataacgtaaaacagtgttggattttcaaactcaaaagaaagaaaaaaaccacaaaaaacaaaaaaagcagaagttgggtaaaaacgagttctgactaaaagctgcaatactgtctcaaatgtgcccrtgaccccacctattgctcaatttctacaccatgagggtaaagggaaacgaaatgcaacttggattcacttgacttcttaagtcttacagagagagaaactgatcagaaaagatacatttttggaggttggtttttttttttaattcttgcttgtaaggcagggcttggtgactcctggtctcctccaatagcctaatttt

SEQ ID NO: 517 - DNA, 401 bp, Homo sapiens
aattcttgcttgtaaggcagggcttggtgactcctggtctcctccaatagcctaattttcctcccaaaagtaagcttttgattccactagggtgggcaagtatctttgtactctgccatttcagagataagagccaactgattttagaaatccaagagcaaatgaaatgtgcaacattttactcttggattgcttgagacygcatctgtatgcctattccctggagtgcagactgtcttgtgagcatttggtgccactgaactctgaaaatcagggcatgcagcatctgctggggaacacaatctggtttctgtggatcagaagcatttctctacacgttagagtcttaaggatttttaaagagagcaggtctactcaggcaactcgttaccgtccctttc

SEQ ID NO: 518 - DNA, 401 bp, Homo sapiens
gttgggctccattagaaggggtctttctggagactgtggtttcttagagcaccgcagtctacccctgaatgaaaggcatcctgctggctaattaacttgcagcactttcttctctattgtgccttgattttttgttttgtttggttttaatctcagaccttgtttctagtaggactgatggagtcttccattatttcgccyaaatattttatgaagaacagagtttccaatcactcactcactctgatttcattacttttagcatacttttcgttcttataatctgcaataaagaggaagactattaagattcaagcttgtttgagcaagtacactgccagagcaaaatcagaggaattcatggaagactgggaaggtgcaagtaatctttctcttagaat

SEQ ID NO: 519 - DNA, 401 bp, Homo sapiens
cagcttgggcaacatggcaaaaccccatctctacccaaacaaacaaacaaacaaacaaacaaactagccaggcatggtggcaactgcttgtagtctagctacagcttccatgaattgttcagtgatgctctcttaactccatgatctatggaacaggcaggcagaaatggagagacagtgggtggaggggaggagcctcaratggcacagaaatctgtagcatctagatcagagtgacacctcagagtttcgagtaacgacaggcacctggcccatggctgggattgttaccagtggtgaatctgtgcaggtctgcagcaacctcaattcttccctctttaggataaaaacttcattgaggggcataaagcagaagagactgaagcaagttttagagaggg

SEQ ID NO: 520 - DNA, 401 bp, Homo sapiens
tccatgatctatggaacaggcaggcagaaatggagagacagtgggtggaggggaggagcctcagatggcacagaaatctgtagcatctagatcagagtgacacctcagagtttcgagtaacgacaggcacctggcccatggctgggattgttaccagtggtgaatctgtgcaggtctgcagcaacctcaattcttccctcyttaggataaaaacttcattgaggggcataaagcagaagagactgaagcaagttttagagagggagtgaaagtttattaaaaagctttagagcaggaatgaaaagaaagtaaaatacacttggaagagggccaagcagacatcttggaggtcaagcacagggatttgaccttctgacttgggggtgtatatgctggcctat

SEQ ID NO: 521 - DNA, 401 bp, Homo sapiens
gaatgaaaagaaagtaaaatacacttggaagagggccaagcagacatcttggaggtcaagcacagggatttgaccttctgacttgggggtgtatatgctggcctatttccagcacttcacatcccttttcccttgattcttcctttaggatgggctgccgcatgtgtggtggcctgctagtgcttgggaagtgagcatgcrcagtgtgtttactggcgttgtacacatgctcacctgaggcgttcttcgggtgaatgcccccagaaagtcatgtaccagttaaactctgccatttggcctcttagtgcatatgtgtgagccccctgcccaactcctgggaccttatcgggaagctgataatcaccagtttcagatttttcctatttataaaaagactgcct

SEQ ID NO: 522 - DNA, 401 bp, Homo sapiens
ttcccttgattcttcctttaggatgggctgccgcatgtgtggtggcctgctagtgcttgggaagtgagcatgcgcagtgtgtttactggcgttgtacacatgctcacctgaggcgttcttcgggtgaatgcccccagaaagtcatgtaccagttaaactctgccatttggcctcttagtgcatatgtgtgagccccctgcscaactcctgggaccttatcgggaagctgataatcaccagtttcagatttttcctatttataaaaagactgcctttccctggagctggctgcaaccaattattttagagagacagttaccaaccacctgttcatcacttgatggtggcctgacattcctggttagggcggggggcactgtcctgctctgctcatgtctgcc

SEQ ID NO: 523 - DNA, 401 bp, Homo sapiens
ctgtcctgctctgctcatgtctgcctacctactgtagcaggaccacaggcagacttttcttaaggagactcccagctttcttcctggtgaaggacccagagtttgtgagcccaggactgcagataatcaaaaatattaaattatggaagctaaatatttaaaataataactcataatggctctccaaatgtctgtaaaagsatttcacttaacaatagctggcaaatattatgtcagtggaaaatacagccaagttttggctcatccaaaatgttgtcatcaccgccactttaatatttaagagattaatgtttcaaggctcttccagaacattgtatttggagtctgattgaaagactcctcggtcctccctcattattctttatgtgtgactttggatg

SEQ ID NO: 524 - DNA, 401 bp, Homo sapiens
tgtctgcctacctactgtagcaggaccacaggcagacttttcttaaggagactcccagctttcttcctggtgaaggacccagagtttgtgagcccaggactgcagataatcaaaaatattaaattatggaagctaaatatttaaaataataactcataatggctctccaaatgtctgtaaaagcatttcacttaacaatasctggcaaatattatgtcagtggaaaatacagccaagttttggctcatccaaaatgttgtcatcaccgccactttaatatttaagagattaatgtttcaaggctcttccagaacattgtatttggagtctgattgaaagactcctcggtcctccctcattattctttatgtgtgactttggatgctctcaatacacttaaa

SEQ ID NO: 525 - DNA, 401 bp, Homo sapiens
ataataactcataatggctctccaaatgtctgtaaaagcatttcacttaacaatagctggcaaatattatgtcagtggaaaatacagccaagttttggctcatccaaaatgttgtcatcaccgccactttaatatttaagagattaatgtttcaaggctcttccagaacattgtatttggagtctgattgaaagactcctyggtcctccctcattattctttatgtgtgactttggatgctctcaatacacttaaattgtatgtccctgtggggaatggttctcaaagtgtagtatctggagcagcagtatcagcttcacctgggaacttgttaaaaacataaattcttgcagctggatgtggtggcacatgcctataatcccaatgggagaccaaggtag

SEQ ID NO: 526 - DNA, 401 bp, Homo sapiens
attatttctttactaaaattgcccattcacccctttgttttatttttcacacctaaaaaagtagccaagtttgaaaaagaaagagtgcttgccaccctttctgtccttaaagtgaaggccttgtctatctcattctgatctctgcccatccatcaccattcagcactcctgttgtgacttcgccccatctccaatcttacrttttattttcttggtacttttggtcatggctgtgttgatggtttcagaagaatacatagtatattaaggggtaacgtttacaaatgttcttgagagaactaatttttcccccttcatctcaaggtgtcactgcttaattattacggttcctaaataatctctatgatgtggttaactatccctaccttgcagaaatcctg

SEQ ID NO: 527 - DNA, 401 bp, Homo sapiens
cccatccatcaccattcagcactcctgttgtgacttcgccccatctccaatcttacattttattttcttggtacttttggtcatggctgtgttgatggtttcagaagaatacatagtatattaaggggtaacgtttacaaatgttcttgagagaactaatttttcccccttcatctcaaggtgtcactgcttaattattayggttcctaaataatctctatgatgtggttaactatccctaccttgcagaaatcctggaagatgagacggacctagcatgttgtgtgagcaacatctcttagagataatcaatttgatatcagcacaattattgtggctactattgaaatttctcttagtttcttagattagtactattggaactaatcgtaaataaccaa

SEQ ID NO: 528 - DNA, 401 bp, Homo sapiens
cttaattattacggttcctaaataatctctatgatgtggttaactatccctaccttgcagaaatcctggaagatgagacggacctagcatgttgtgtgagcaacatctcttagagataatcaatttgatatcagcacaattattgtggctactattgaaatttctcttagtttcttagattagtactattggaactaatcrtaaataaccaatttacaagaaaatgagtctcattatattatagcacgaagaggatatattcaaggagatacacctttgaatctattatttcaatatgcttataaaaattactattttaataaatttcccagtcaatcctattgtgtagaaaaagatgcttcactgcttacatattttaagatgagactgggaacaagcag

SEQ ID NO: 529 - DNA, 401 bp, Homo sapiens
tgtgtgagcaacatctcttagagataatcaatttgatatcagcacaattattgtggctactattgaaatttctcttagtttcttagattagtactattggaactaatcgtaaataaccaatttacaagaaaatgagtctcattatattatagcacgaagaggatatattcaaggagatacacctttgaatctattatttcratatgcttataaaaattactattttaataaatttcccagtcaatcctattgtgtagaaaaagatgcttcactgcttacatattttaagatgagactgggaacaagcagattaatttggttgtgagactgggagtttatgagcaaaatgttcttctgtgcaaacgaagtagaagcttcatttcccttcccttcagttaacc

SEQ ID NO: 530 - DNA, 401 bp, Homo sapiens
aaaactagactaaattctgtattgaaaatgtcaagtcaaatatgctatataaccaagaaatatcagtatagaatatcatgtattctacacaaagctacattttcaatataatccccgtaattatttcactcatcaaagaaatgtatccagctgagacataaaaccttagtcaatacattaagaaaaaaagaaaaaaaaaamcatttttttcccagtcatggggaccagattgtgtctattattcatcagggcctagcacaatgttgagcacatgctcaaggtggccaataaatccttgtggaattgagttatatgggagtcaattattcggtcacagcactggattaggaggttgagttcttgatagatttactttcagctgacctgtgtgacatttggta

SEQ ID NO: 531 - DNA, 401 bp, Homo sapiens
catttggtatttggttttgcaactttgttttcagttttttccatgcaccaatactcctagaattttgcttttgcctcttttcttccctttcatcttttttttttttctgtagaaagcatacatccatatagaagacctctctcaatacattccagatctgagttgcagtgcagcacttagcttagcttgtgaaacactccraagacttgattaatatcatcctatggagagatgtccttaagtaacttagaatggatcctttatttttccagtaaataaactctaaaatatgaggatttccaaggtgaaaagaaagagtacgaacccaagaaatattttaaatgaatcttgttttccgtccctgtgttctgagtaacatctgtattcacccctgcctttcc

SEQ ID NO: 532 - DNA, 401 bp, Homo sapiens
attttgagtgtgtgtctggtggggaggggaggctggggggttaaattcatgtatttttaggcaagagtattaacaagaattgtttgaaatatttctgaaaccatagtgtcataatgaaaataaaaataaggtggagactggattgtaattaacacagagcttttccatccaagacagagcttaaaggcctctgtttgttgsgtcactttaaaaacaaaccctactgccagggatttccaatggcaactttaccgtgcttacccaaatggaggtaattaacaacccttctgtgtgttaaattaggtatccacagaattgggaagcttcagatatttattgtaaaccagagcctcttttcttgttaatctcctaagacagcagactagtgactcgcacccatt

SEQ ID NO: 533 - DNA, 401 bp, Homo sapiens
atatttgtcgttgaattatttcttctttgtttaaaattaaataatgcaagtgatttttttgctttagaaaggaggattacctcttcctagacatccagttgaaaaaccaactggtatagaagggaaatgcatctcaaacagatacaatgacattcttataattgcctcatttgaaatggcagcatttccccgtgctaattrtcatccttcagctaatttggtacattaaaggagattgttattaaaaacaaaattaaatccattgccagcatctttattctctaccagaagaaaaattttaaataaatgcaaatacgccagttttgctcttaatgttgcctgaagttgaaaggatgaactttgacttgaaagataatgagttccccctgttggaggatttg

SEQ ID NO: 534 - DNA, 401 bp, Homo sapiens
ttgtcatccttcagctaatttggtacattaaaggagattgttattaaaaacaaaattaaatccattgccagcatctttattctctaccagaagaaaaattttaaataaatgcaaatacgccagttttgctcttaatgttgcctgaagttgaaaggatgaactttgacttgaaagataatgagttccccctgttggaggatytgaggtttgggttggataatcccagatgatgttggaatagacagaatttaaggttccttctaaccctgaaattctatggttataaaatctatccttttcttgaaagaagaaatattaaaggtgttttcaaggaaatggtagcataatgaaaaaggaaacacaaaaatgatttttttttccttaataagcagtcacctcac

SEQ ID NO: 535 - DNA, 401 bp, Homo sapiens
tttttcttcttaattgcttgtttgtttgtaacaaatgcggtggtgtaatcaagtggcttttacaataataaaacacttctctcaattaaactgtactgcaaggaattcaaagatgaacagaagtgttccttcttttaagatgctgatttataagcatccctttaaaatttacttctctttctgatgataaaagtaatacaygtttattgtagaacatttggaaactaccagggactataaataacccatagttgccccaccagagaatattcttaatatttttggcatatttcttccatatatctattcatattcacattcatactaatttgtcatattctaatttaaccctctctttaatgtttataatgtgattatactactttttaacacatgaattg

SEQ ID NO: 536 - DNA, 401 bp, Homo sapiens
ggagacagacaacaaaaactaatataaaactaaaaatgtgaaaattcagcatttaatgaaaatgagagcttagaaaaactagcatccagatttattcagactgaatttctgccctgggaaattaatgaatcatccttcagttcccttttgtgtctgtgcatccagcctcattgtccttggtagtaaattatgtacaccccstgtttccaaatcttcaaatacggtatgcgtcataaagtcacaaaatgtgtaatttgggtcctgtatacaaattctagccagggaatggcttgtagtcatgctaaatattctttacaagttgtgtaacaaagacaaagaaaactacaaaaaaatacagagaaacataaaaaatttcccattccttctgtgattctgatgta

SEQ ID NO: 537 - DNA, 401 bp, Homo sapiens
gagacagacaacaaaaactaatataaaactaaaaatgtgaaaattcagcatttaatgaaaatgagagcttagaaaaactagcatccagatttattcagactgaatttctgccctgggaaattaatgaatcatccttcagttcccttttgtgtctgtgcatccagcctcattgtccttggtagtaaattatgtacacccccygtttccaaatcttcaaatacggtatgcgtcataaagtcacaaaatgtgtaatttgggtcctgtatacaaattctagccagggaatggcttgtagtcatgctaaatattctttacaagttgtgtaacaaagacaaagaaaactacaaaaaaatacagagaaacataaaaaatttcccattccttctgtgattctgatgtat

SEQ ID NO: 538 - DNA, 401 bp, Homo sapiens
aaagattcctccaggacttttacaaagacagatgatattaaactgaacaaatagtgctaggtaagaagtaaattgttcaattttttcacagaagccctgtagattttagcaaaaccccccagacacaatgtcaaatttctcagcaggtttcttggttttagctcttccccttaagcagtagttaacagaatattagatgtkagtaaagttatttctttgttattctacctgggttgactaccagagaaaatccctgaacattttacaaattgctcaacagattttcatctaaaactagaaaagattggccagttatggtggctcacgcaggaacattgctttagcccaggagctgaaaaccagcctgggcaatagagtgggacccccatttctaaaaaata

SEQ ID NO: 539 - DNA, 401 bp, Homo sapiens
tgggttgactaccagagaaaatccctgaacattttacaaattgctcaacagattttcatctaaaactagaaaagattggccagttatggtggctcacgcaggaacattgctttagcccaggagctgaaaaccagcctgggcaatagagtgggacccccatttctaaaaaataatatcttttaaaaaattaactgggcatgkcggtgcgtgcctgtagtctcacctacttgggaaactaaggagggaggattgcttaagcccaggatgtcaagattgcggtgagctatgattgagccactgcactccagcctggaggacagagtaagaccctgtctcaaaataaaataaaataaaattagaaaggataggtgtgtttgggagggagactggttaaccagggt

SEQ ID NO: 540 - DNA, 401 bp, Homo sapiens
atcttttaaaaaattaactgggcatggcggtgcgtgcctgtagtctcacctacttgggaaactaaggagggaggattgcttaagcccaggatgtcaagattgcggtgagctatgattgagccactgcactccagcctggaggacagagtaagaccctgtctcaaaataaaataaaataaaattagaaaggataggtgtgtktgggagggagactggttaaccagggtgcatgggattatgtagggcttgtctttcatttgagttcttctctgtctttttaaaatgactgattatatatattaggtcccaatggcttcattatcactattattttaattcataggttaaattttataaacatgaaaaataattgacaagcctagaatctcttttctgtctat

SEQ ID NO: 541 - DNA, 401 bp, Homo sapiens
tgcatgggattatgtagggcttgtctttcatttgagttcttctctgtctttttaaaatgactgattatatatattaggtcccaatggcttcattatcactattattttaattcataggttaaattttataaacatgaaaaataattgacaagcctagaatctcttttctgtctatattagaaattaaattatatttagtakttaagcaatacatgcatacatctcatgtaatccttactgtaattaaggatgaaattatgtaacataggaaaagcagatgtaagttctcattctccttttacacataaggaaattaattcagacttttagtaagttgcccagaatcacgcagctagtaagattcagaactaaaaatataatacctgtcttctaattcaaat

SEQ ID NO: 542 - DNA, 401 bp, Homo sapiens
atctctaaaatgatacctaaaatgataaacaatttaaatttagcataattatcattttttaacttacagattgacatatttctgcaagtgttttcagaggcattggggccacagaagtccaagtcaaggcaatttttggtactaaacagttgacttttctctggtccacatcaaagctgccactgtgcagagaactaacaygtgtacgtgactagtctagggctggaggaacatgcgaggggactgttggaaaggaaaaacttttgctcttcctctaggttcagtaaattagggactgtaaacaataataatgaaagattaataagaggcacacaattttattttattttattttatttacttatttttttgagacagagtttcactcttgttgcccaagc

SEQ ID NO: 543 - DNA, 401 bp, Homo sapiens
aaaatgatacctaaaatgataaacaatttaaatttagcataattatcattttttaacttacagattgacatatttctgcaagtgttttcagaggcattggggccacagaagtccaagtcaaggcaatttttggtactaaacagttgacttttctctggtccacatcaaagctgccactgtgcagagaactaacatgtgtaygtgactagtctagggctggaggaacatgcgaggggactgttggaaaggaaaaacttttgctcttcctctaggttcagtaaattagggactgtaaacaataataatgaaagattaataagaggcacacaattttattttattttattttatttacttatttttttgagacagagtttcactcttgttgcccaagctggggt

SEQ ID NO: 544 - DNA, 401 bp, Homo sapiens
gtgcagagaactaacatgtgtacgtgactagtctagggctggaggaacatgcgaggggactgttggaaaggaaaaacttttgctcttcctctaggttcagtaaattagggactgtaaacaataataatgaaagattaataagaggcacacaattttattttattttattttatttacttatttttttgagacagagtttcrctcttgttgcccaagctggggtgcaatggcacgatctcggctcaccacaacctccccttcccgggttcaagtgattctcctgtctcagcctaccaagtagctgggattacagacatgcatgaccatgcccggctaattttgtagttttagtagagactgggtttctccatgttggtcagcctagtctcgaactcctgacc

SEQ ID NO: 545 - DNA, 401 bp, Homo sapiens
cctccccttcccgggttcaagtgattctcctgtctcagcctaccaagtagctgggattacagacatgcatgaccatgcccggctaattttgtagttttagtagagactgggtttctccatgttggtcagcctagtctcgaactcctgacctcaggtcatccactcacctcggcctcccaaagtactgggattacaggtgtragccactgtgcccggccccacaattttattaatatttatatgcaaaagagcttgtagaaaatcagtgaaaatcaaagaagtggttagactagggggtttataaacccttcttaaccaaggaaagagggtttgggcatcaatgagtggcgaattgtggagaagtgactagaaaatgtatgagagaaactaatggaaggtat

SEQ ID NO: 546 - DNA, 401 bp, Homo sapiens
actgggattacaggtgtgagccactgtgcccggccccacaattttattaatatttatatgcaaaagagcttgtagaaaatcagtgaaaatcaaagaagtggttagactagggggtttataaacccttcttaaccaaggaaagagggtttgggcatcaatgagtggcgaattgtggagaagtgactagaaaatgtatgagasaaactaatggaaggtatattagttcattctcaagctgctataaggacatgcccaagactgggtaatttataaatgaaaaaggtttgactcacagttctgcagggcgtggaggcctcaggaaacttacaatcatggcagaaggagaagcaaacacatccttcttcacatggcggctgcaaggagaagtgctgagcaaaaag

SEQ ID NO: 547 - DNA, 401 bp, Homo sapiens
caagctgctataaggacatgcccaagactgggtaatttataaatgaaaaaggtttgactcacagttctgcagggcgtggaggcctcaggaaacttacaatcatggcagaaggagaagcaaacacatccttcttcacatggcggctgcaaggagaagtgctgagcaaaaaggggaaaagccccttataaaaccatcagatcytgtgagaactcactcactatcgtgagaactcacttactatcatgagaacagcatgagggtaacagcccccatcgttcaattacctccgaccaggtcccttccatgacacatggggattatggggactacaattcaagatgagatttgggtggggacacagccaaaccatatcagaaggtaaggcctattttagtaagttt

SEQ ID NO: 548 - DNA, 401 bp, Homo sapiens
gaacttttaataaggactctcagaatagacttttaaaagctcttgagagtagtaagccaagccaagaactagccattagactttatctgtaatacctatagattggggtgaatttcccttttcttgaggactctaaaacattctgagtttcccgggcctgccagaaagttaccttccttacccacctgtaaggctaggaaygctataaaccagatacgagtcttacttttccaagggagctttgtaagcgttggtgccatatagtcaaccttagttccttaaaactattgggttatatgtgattccacacacattattttcaaacatgacactgcagtcaaagccttagtaggacagccaatgtttccaattgtgttctgttccaaggagaatagattctt

SEQ ID NO: 549 - DNA, 401 bp, Homo sapiens
agcaatgtcagcagaagcctgtactccagagtacctgccaaggttttgttcatgaatctctaatatgctctttctttgctgaagatgaggcatgctggcctacagctgactgcgtgagccttcagaaaagcatcggcataaaacaataattatctgtgaatgacaatgacttaaaatggccatacctaaagatctgatgaragttcattataagtcaattgataaagaaatttggttgtttctctgacatacaggattttaagataataatgtgataccaagacatctcatgaacagcaagggcattgacagatttctaggaattttatatgatttctgaaaaatcaaaatcattatatccatacaaatataacccaggaatggttaaatatcttctcttt

SEQ ID NO: 550 - DNA, 401 bp, Homo sapiens
gcaatgtcagcagaagcctgtactccagagtacctgccaaggttttgttcatgaatctctaatatgctctttctttgctgaagatgaggcatgctggcctacagctgactgcgtgagccttcagaaaagcatcggcataaaacaataattatctgtgaatgacaatgacttaaaatggccatacctaaagatctgatgaawgttcattataagtcaattgataaagaaatttggttgtttctctgacatacaggattttaagataataatgtgataccaagacatctcatgaacagcaagggcattgacagatttctaggaattttatatgatttctgaaaaatcaaaatcattatatccatacaaatataacccaggaatggttaaatatcttctcttta

SEQ ID NO: 551 - DNA, 401 bp, Homo sapiens
gccaaggttttgttcatgaatctctaatatgctctttctttgctgaagatgaggcatgctggcctacagctgactgcgtgagccttcagaaaagcatcggcataaaacaataattatctgtgaatgacaatgacttaaaatggccatacctaaagatctgatgaaagttcattataagtcaattgataaagaaatttggtygtttctctgacatacaggattttaagataataatgtgataccaagacatctcatgaacagcaagggcattgacagatttctaggaattttatatgatttctgaaaaatcaaaatcattatatccatacaaatataacccaggaatggttaaatatcttctctttatttgacagtgcttcccatgcaatttagcatattaa

SEQ ID NO: 552 - DNA, 401 bp, Homo sapiens
ctgactgcgtgagccttcagaaaagcatcggcataaaacaataattatctgtgaatgacaatgacttaaaatggccatacctaaagatctgatgaaagttcattataagtcaattgataaagaaatttggttgtttctctgacatacaggattttaagataataatgtgataccaagacatctcatgaacagcaagggcaytgacagatttctaggaattttatatgatttctgaaaaatcaaaatcattatatccatacaaatataacccaggaatggttaaatatcttctctttatttgacagtgcttcccatgcaatttagcatattaaataagctcatctctctttttacaaactgagagaacaaatcctttgagagttttccaggggccctctggg

SEQ ID NO: 553 - DNA, 401 bp, Homo sapiens
agttcaagaccagcctaggcaatatagtgagaccctgtctctctaagaacaaaaaatagattaataaaccaagaaaacttgctcattttaaaagtagttttgcatcaatacactactgtattagtccattcttgcattgctgtaaagaaatacctgagattgggtaatttacaaagaaaagaggttgactcacagctctgyaggctatacaagaagcatagcagcttctgcttcttgggaggcatcgggaagcctccaatcatggcagaaggtgaaggtggagtaggcgtcttacatggcaggagcaggagcaacagagggtaaggggtcaggtgccacacaattttaaatgaccagatcttacgagaactcggtcactatcatgagtacagtaccaagag

SEQ ID NO: 554 - DNA, 401 bp, Homo sapiens
aaaccaagaaaacttgctcattttaaaagtagttttgcatcaatacactactgtattagtccattcttgcattgctgtaaagaaatacctgagattgggtaatttacaaagaaaagaggttgactcacagctctgtaggctatacaagaagcatagcagcttctgcttcttgggaggcatcgggaagcctccaatcatggyagaaggtgaaggtggagtaggcgtcttacatggcaggagcaggagcaacagagggtaaggggtcaggtgccacacaattttaaatgaccagatcttacgagaactcggtcactatcatgagtacagtaccaagagggatggtgctaaaacgttcttgagaaatctgcccccatgattacaatcacctcccaccagagccc

SEQ ID NO: 555 - DNA, 401 bp, Homo sapiens
cagcgtggccacacaatgtaagattctctctctcttctgttctctctctctctctttcactgacctttacaactttctatatcaactcacatttttgtcctttattttcgtctttcttatttaaatgacctctaaactactctaggataaaagtcactgtcatttttctttaacaaaaacacatctcactctccttgtackctttgcctgcagagttttgtttcttcatcctattattactagtagttcatttacacatatttattagatttcttaacccttagtaacattaactttcagtgaaaatgaagaaataaacaatcataaactgtcagttacatacatgcattctgtgctacattagcacaggtataaatacattctctcataatttctagagg

SEQ ID NO: 556 - DNA, 401 bp, Homo sapiens
tcactgacctttacaactttctatatcaactcacatttttgtcctttattttcgtctttcttatttaaatgacctctaaactactctaggataaaagtcactgtcatttttctttaacaaaaacacatctcactctccttgtactctttgcctgcagagttttgtttcttcatcctattattactagtagttcatttacayatatttattagatttcttaacccttagtaacattaactttcagtgaaaatgaagaaataaacaatcataaactgtcagttacatacatgcattctgtgctacattagcacaggtataaatacattctctcataatttctagaggcatgtttcctaacagtacaacttctcagtgtggcaaaagagcaggtttattaacag

SEQ ID NO: 557 - DNA, 401 bp, Homo sapiens
aaaaatacaaaaaaaattagctgggcatggtggcgggcgcctgtagtcccagctactcaggaggctgaggcagaagaatggcgtgaaccagggaggcggagcttgcagtaagccgagatggtgccactgcactccagcctgggctacagagcgagactctgtctcagaaaaaaaaaaaaaaaaaaaaaaaaaaaaggaaayaaacttaaatagataaactttagtaattagtattttggtattttattttctttgaaattatctacctatttcatgaatatccgtcatttaacttagtataactttaagatttcaagtaaccaaaaagatgtcggaaactatttttaagtcactgtactataaaacagttattgttgaaataaagtttgtcagagtaatga

SEQ ID NO: 558 - DNA, 401 bp, Homo sapiens
catgcttgagcccacttacccaactcctgcgatcttagccggaagctgctgatcagcagtttcaggctcttcctatctgttgggagcctgcctttccctggtgccagctgcaaccaattattattttagagagacagttaaggactgcctgtccatcacctgatggtcgtctgacattcctggttgtggggaaagggaacmctctcctgccctgctcatgtctgactagctacctgctgtaacaaaactagttttatttaccaaagattatcttatatcagataaacttgaaaacatgttggttatatttaaccattaaataatatatttggttatatttctgagtgttttagcaatacttataagcgtttttttaagccaacttagaatagaactcattt

SEQ ID NO: 559 - DNA, 401 bp, Homo sapiens
tcgtctgacattcctggttgtggggaaagggaaccctctcctgccctgctcatgtctgactagctacctgctgtaacaaaactagttttatttaccaaagattatcttatatcagataaacttgaaaacatgttggttatatttaaccattaaataatatatttggttatatttctgagtgttttagcaatacttataagygtttttttaagccaacttagaatagaactcatttattaatttggtaataccatcatgaagtgaggaaatgtcacataggtacaacgtacatacatatatacacacataaacatataaagatttatttattttaaaatttaatttatttaaagacagaaacagatagagattattttaagattttagccacaggtcaggta

SEQ ID NO: 560 - DNA, 401 bp, Homo sapiens
ccttgagaagacagttttgggtggtagaatattttacagctgaaggaggaagagaaaggatgtaaactttcaagtagtccctttaaaagggagttttcgggcatttttgtctgttacagggttgctgcgtatcattgttgttattatcaattgttattttctgggacaaacagtaaattcttctggtagcactaagctttrtcaagcaggcattttaaaagtggggttggggggatgatttttgatgggtgtgaattttaagttcttgtctaaatctaatttatgcttgttaattttgttaagggagctatccagtctaaccatgatacacttgtttccttctttcaacttgatcctcccataggtagcaatacgatgtttatttagggtgagagctctct

SEQ ID NO: 561 - DNA, 401 bp, Homo sapiens
agactctgccctcatgatggaaagtcattcccaaaaatagccaaagagaaagcgagctgagaccctcatggtcacaggcagcaaaaatggtgtttgtgaaaatggtatctccagcgttacacaaaagtgaggcacctgcaatcacagacttgtggacctgtgacacttgggaggcaatacggagatgggactttttcagcmctaataagacaatgaggaaggacaacaagtgccttgtatgagctgagacactttatttagacaaattccccagattgctgacatagccaggtgggaaattttgctagtgttttttagggtccttgactgacagcgagcacctccagactcaggcccaaccacctgtgcttcccagtgggcaaaaacacagacagaggtga

SEQ ID NO: 562 - DNA, 401 bp, Homo sapiens
cgtagctaaaccagctaacacttttcaaccattaacaaagaatggtagagtggaatgtggtggcacatgcctatagtcctaactactcaggaagctgaggtaggaggactccttaagcccaggagtttgaggtcaacctgggcaatatacaatatagtgaggccctgtagagatctataatatgttctgctgtaaacatawgtccacaatataagggccatttttttttttttgagacagagtttcacactgtcgcccgggctggagtgtagtggtgcgatcgctgctcactgcaacctccacctcccaggttcaagcaattctcctgcctcagcctcccgagtagctgggattacaggtacctgccaccatacccagctagtttttttttttttgtattt

SEQ ID NO: 563 - DNA, 401 bp, Homo sapiens
gttttttgtttgtttgtttgtgtgagacagagtctcactccgtcactcaggctggagtgcagtggggcaatcttgggtcactgcaacctctacctcccaggttcaagcaattctcaggcctcagcttcctgagtagctggactataggcacacaccaccatgcccaaataatttttatattcttagtagagatggagtctyaccatgttggcaacgctagtctcgaactcctgacctcaaacgatttacccacttcagcctcccaaagtgctgggatcacaggtatgagccaccgtgcctggcctatacttgtgtatactttgaattgtttcttttttcttttcctattttattttattttattttatattttattttattttattttttgcgacagggtc

SEQ ID NO: 564 - DNA, 401 bp, Homo sapiens
atggagtctcaccatgttggcaacgctagtctcgaactcctgacctcaaacgatttacccacttcagcctcccaaagtgctgggatcacaggtatgagccaccgtgcctggcctatacttgtgtatactttgaattgtttcttttttcttttcctattttattttattttattttatattttattttattttattttttgygacagggtctggctctgtcacccaggctggagtacagtggtgcaatcatgactcactgcagcctcaagctcctgtgctcaggtgatcagcctcctaggtagctgggactacaggtgaccaccatcacacccagctaatttttgtattttttcatagagatgaggtttcaccatgttgcccaggctggtctcaaattccca

SEQ ID NO: 565 - DNA, 401 bp, Homo sapiens
gaaagacatttctgcttagatactaatacagacgtacatatttggacaatatacatgtagaataaaaattatttgaattacttcaacaattatgcctacttttcaaaaggctttgacatcagctatataatttagccatttggcaatattaatcataacttactaaactaaatttgacattgggataacagaatggtaaamtgcaaactgaaatcatagctttatgatcacatgaatttccttcaacagctgttcactgttattactacttatacctttgttagcattccaagaggatctgataatcatagctagcattgattgagcaattattatataccagacattcttctaagtgccattatctcatttaccctacataacaaccttatatggtaagt

SEQ ID NO: 566 - DNA, 401 bp, Homo sapiens
aaaagtcacgtttcttagttaatagaagtcatagtacataagaaggtaagagtgtcaaattattactagttgtgaatgaattattactagttgtgaaagaatagcaattcatggttttgtttggtcagcaactactgcaccctagtgatgatgaattctctagcagattggaaagatacttagggagggaaaaaagatgamtttattaaggatctctggaaataggaaaatatttaagaaattctataagtaaaaatgagtaattttatgtcagaataaaataaaatttggctgaatttatagttctccatctgcctgcttgcacctatttggggcttattttacctttgtggttaccctgagaagcatggtatggctggagcctaagtgagttctttgtt

SEQ ID NO: 567 - DNA, 401 bp, Homo sapiens
aaagtcacgtttcttagttaatagaagtcatagtacataagaaggtaagagtgtcaaattattactagttgtgaatgaattattactagttgtgaaagaatagcaattcatggttttgtttggtcagcaactactgcaccctagtgatgatgaattctctagcagattggaaagatacttagggagggaaaaaagatgacwttattaaggatctctggaaataggaaaatatttaagaaattctataagtaaaaatgagtaattttatgtcagaataaaataaaatttggctgaatttatagttctccatctgcctgcttgcacctatttggggcttattttacctttgtggttaccctgagaagcatggtatggctggagcctaagtgagttctttgttt

SEQ ID NO: 568 - DNA, 401 bp, Homo sapiens
aagtcacgtttcttagttaatagaagtcatagtacataagaaggtaagagtgtcaaattattactagttgtgaatgaattattactagttgtgaaagaatagcaattcatggttttgtttggtcagcaactactgcaccctagtgatgatgaattctctagcagattggaaagatacttagggagggaaaaaagatgactwtattaaggatctctggaaataggaaaatatttaagaaattctataagtaaaaatgagtaattttatgtcagaataaaataaaatttggctgaatttatagttctccatctgcctgcttgcacctatttggggcttattttacctttgtggttaccctgagaagcatggtatggctggagcctaagtgagttctttgtttt

SEQ ID NO: 569 - DNA, 401 bp, Homo sapiens
agtcacgtttcttagttaatagaagtcatagtacataagaaggtaagagtgtcaaattattactagttgtgaatgaattattactagttgtgaaagaatagcaattcatggttttgtttggtcagcaactactgcaccctagtgatgatgaattctctagcagattggaaagatacttagggagggaaaaaagatgacttkattaaggatctctggaaataggaaaatatttaagaaattctataagtaaaaatgagtaattttatgtcagaataaaataaaatttggctgaatttatagttctccatctgcctgcttgcacctatttggggcttattttacctttgtggttaccctgagaagcatggtatggctggagcctaagtgagttctttgttttc

SEQ ID NO: 570 - DNA, 401 bp, Homo sapiens
cacgtttcttagttaatagaagtcatagtacataagaaggtaagagtgtcaaattattactagttgtgaatgaattattactagttgtgaaagaatagcaattcatggttttgtttggtcagcaactactgcaccctagtgatgatgaattctctagcagattggaaagatacttagggagggaaaaaagatgactttatkaaggatctctggaaataggaaaatatttaagaaattctataagtaaaaatgagtaattttatgtcagaataaaataaaatttggctgaatttatagttctccatctgcctgcttgcacctatttggggcttattttacctttgtggttaccctgagaagcatggtatggctggagcctaagtgagttctttgttttccaa

SEQ ID NO: 571 - DNA, 401 bp, Homo sapiens
atgtaaacacaaattggtttaaatgttctctagcaaagatcaaatctaggacagagaaacaggagaagaaaacaggaaaatggggctcagagcaggggaatgctgtgttgtttaaaagccctggatgtcaagatctaaggaaaagataaaaaaaatagaaccaccaccaaatataaatatttatttccatatctcacaatkttttcctaggccattgctggtgacagactttccagtccttttcagaacttactcatcacatccttttcatgcttgttattttgatcctgaacatccttgcaattcaaagagcatacagaggcagagtgaaattagcataatgtcacctattgctatgatgctcgttacctctacctccagaacatgccaaggaggcttcc

SEQ ID NO: 572 - DNA, 401 bp, Homo sapiens
aggcttccaggagccccagaaacacagaacagcaaatcttcaaaagcccagtgcccagagcagatagagaagaaaattctattttgaaatccataaagaatctcataattccttgcaatatctagtcccaattgtgtgggtgacaaactgacctatggcagaattaggaatgtgaataggaatgaggcttggagttccccrttcctgctgccctaactcccactggaatgtaatttgtacaaagagtgacgacatgtgaagatgtaaaaaagaggactagagatgtcctcctgagctgcacaaggaagaaagctttttatgaccatccaattctgcaaaggacctagtttcccagtgcttattaccataaggatgcagaaagaaaaaagttcacagacata

SEQ ID NO: 573 - DNA, 401 bp, Homo sapiens
atgtgcagaaaagaagatgatactctacaagctcggcttttcggcgacaaatctgaagtccatctccattgtgagtatttcttgtgtggcttggacttcctccattccagagaaagaatcagtcctttgggaatccatgaagtgtgtcctactgtgccaggcctcagccactaacacaggtccctagagagccacgctgtyttgctccatgacctccaatgagaaccccttcttactgccctttcattgattaaaacgagacatttgctgggacctcattgtgggctaagcacattgcagtcagtttccacataatgttgctcattggatcattattttaatttttctctctttacctcccccacagctgtctttccttatctgcaatgttgcatgcacaa

SEQ ID NO: 574 - DNA, 401 bp, Homo sapiens
ttattgttaagtttctcaaagaaatgctcaatgcctgggtcaaggacctctacagatcttgagtctccttaggacatccctttccaactactcatctatttgatgccagattttctacatgtacttcaaccaaaacaacatattgcaacagattgaatgcagaagcagatatgagaatctgctgccttctatgaagcaagwcattaaagagatttgcaaaaatgtaaaccagtgctctcttcttttcaaaattatttttattagggaaaatatatttttcataaaaactaggttatttatattactatttaatggagttagcgttgctatttttaaaagagtttatttaaaaagtcttaacttctttagtggtaatttctagtacggtaaacactggtaga

SEQ ID NO: 575 - DNA, 401 bp, Homo sapiens
caaaaattagttgggcatggtggcacgtgcctgtaatcccagctacttgagaggctgaggcaggagaatcacttgaacctaggaggcagaagtgagcgagatcatgccactgcactccagcctgggagacagagtgaggctctgtctctgaataactaactaactaactaactaaataaataaataaaaataggagttgcraacagtaggtctatatccgtgattgcttttcctcaccactgttgcctcctttttttaaatctctgaccaaagaactggctccacttggtgtcctgtatcagatttcttgggccggccatggtggctcattcctgtaattccagcactttgagaggctgaggcaggaggattgtttgagctcaggagttcaagaccagccc

SEQ ID NO: 576 - DNA, 401 bp, Homo sapiens
caaaattttttctagatccaaagcctttctcacagtgtcaagaaaaaaacattccagttgccagatgaacagcctgtttcagtatcatgggtctaaaaccagacaccggtttttcctccagcaattcttctggttaacttgatgcctctgtctatcatggaaaccgaatttcctaagtcatgcggatttaaaatgctagawaatttcttactcctgtcaccactacatctaatgaatcagatgttctgtttgttcttgtgtcatcccagtccactccctgacaaagcatatctgcaagcttatcatcataaacctaggctatttcaatgctcacgtattaaccgctcatcacatgaatagaatttcatttgcctgatttaccctacacagcccttcaaatt

SEQ ID NO: 577 - DNA, 401 bp, Homo sapiens
ctcacagtgtcaagaaaaaaacattccagttgccagatgaacagcctgtttcagtatcatgggtctaaaaccagacaccggtttttcctccagcaattcttctggttaacttgatgcctctgtctatcatggaaaccgaatttcctaagtcatgcggatttaaaatgctagaaaatttcttactcctgtcaccactacatmtaatgaatcagatgttctgtttgttcttgtgtcatcccagtccactccctgacaaagcatatctgcaagcttatcatcataaacctaggctatttcaatgctcacgtattaaccgctcatcacatgaatagaatttcatttgcctgatttaccctacacagcccttcaaatttatcaacgaaatcactggttttcaactt

SEQ ID NO: 578 - DNA, 401 bp, Homo sapiens
tttttaagtaggaaaagcttttccgttaagtttttgcaaacaaatataatgcaatccaatatttgccctccagagctatgccgatttaccgctgaaaccttgagcaaagaaatactcaaagcttcttctaggttgtgtatgtatcacaacagaggaaggaaattatgagatgggcacttgggagactgttttcttggttgycctctccttgtgaaaggtagggctacaagcccccatttgagccaaagctaaggagtccggtgcccatgaaaaagacgcaagcaatgctgtcctccttgatggaggctgcagaggccagaaccacacctgctcagcctggccctgaggctgcccctgtggcactgctgaaagtcagtcttttgtcacaaggaaggcagttt

SEQ ID NO: 579 - DNA, 401 bp, Homo sapiens
ccaaagctaaggagtccggtgcccatgaaaaagacgcaagcaatgctgtcctccttgatggaggctgcagaggccagaaccacacctgctcagcctggccctgaggctgcccctgtggcactgctgaaagtcagtcttttgtcacaaggaaggcagtttttctagacaagattattctcatcttaagcatgaggtaggggkgtgtgtgtgtgtgtgtgtgtgtgtgtgtgtgtgtgtagtgtctaagtctacttgcctgctttctgggaactggtaagagataggaagatagaagtcatcatcattccaaatgtaggctttcacttaatttctctattttcagtctgagaccacacatttacacgtctctcagcctgcatccccaagctcaaaatatttct

SEQ ID NO: 580 - DNA, 401 bp, Homo sapiens
ctcacaaggctgcagtgaggtgctggccaagactgggatctcatgggaaggctcaaaaggaaaagcatctgcttgtatgctcctggagttacaggcaggttttagttcctgatgagctgatgaactgacagtcttgttctttagtgactctgggccagaggtcaccctcatcccttgccacatggctctctctctatagcrgtttgtttaatcaaagctagcaatggtgagaatctgccaacaaaatagaagtcactattttgtgtgacataatcacagaaatgacatctcatcatgtttgccacattctattggctaaaagcaagtcaccaggcccgcccatactcaaagtgaggagattatacaaggcgtaaataccaagaggcagattagaggctatt

SEQ ID NO: 581 - DNA, 401 bp, Homo sapiens
catactcaaacaaacaaccagtagcaactgacatattctcttcctgaggcattttatttctttggatttggggatccctcacctccttgtcttttcccttcatctcttgtctctgactcacaggctcctttgttcattctcatttttcttccttttggagggctaaatgctagaggtctacagggctccatttgtagcccytcactcttctgtaattgtacctaagaaatctagtctcatgacttcatagtccatgtatatattgatgactcttacatttatatctccagctccaacctttcccctatgccccagatagtacccaattggcatctgtacttggactcaaagacattgcaaacttaatttctcaaagaattaaaaatagaactaccattcga

SEQ ID NO: 582 - DNA, 401 bp, Homo sapiens
tggactcaaagacattgcaaacttaatttctcaaagaattaaaaatagaactaccattcgatccagcaatcccactacttggcatctactcaaaggaaaagaaatcattatatcaaaaagatacccccacttatatgtttattgcagcactactcacaaagggcaaaaacatggaatcaacctaagtgtccatcaatggaytggataaagaaaacgtaatatatatacatacacatacaacacacacagacacacaccgtggaatactactaagccataaaaaataaaatcatgtcttttgcagcaacatggatggaactggaggccattatcttaagtgagataacccagaaacagaaagtcaaatattacatgttctctacttataagtgggagctaaa

SEQ ID NO: 583 - DNA, 401 bp, Homo sapiens
ttgcagcactactcacaaagggcaaaaacatggaatcaacctaagtgtccatcaatggattggataaagaaaacgtaatatatatacatacacatacaacacacacagacacacaccgtggaatactactaagccataaaaaataaaatcatgtcttttgcagcaacatggatggaactggaggccattatcttaagtgarataacccagaaacagaaagtcaaatattacatgttctctacttataagtgggagctaaatatgtgtaccatggatatagaaattggaataatagacattggagactcataaagttgggagggtgggaagggttcaagggatgagaaattacttaatgggtcaatatacactaaaagctcagatttcatcgctattcagta

SEQ ID NO: 584 - DNA, 401 bp, Homo sapiens
cttctcactcagcatatcctgacagcgccaccttcagaatatgtccattatccaaccgcttctccccagttctcctgcaatcacccaggcgtggtcatggttgtctcttgcctccccttgactggtacatgtgccgttcccctttgcatcaagcttacatctgctccaggatgtttatatttgcagttctctccacctgtrattcttatccttctgatttgcatgtaactcactcctttttatcatccaggtctctccttaaataccacctctttggaaagccttccttgatcactctgtgtaaaatagccaagcctccgttattcactatcttcttttgctcttttatttttcatatatgactacgtgaaagtactgtctgttgttatatgtctagttac

SEQ ID NO: 585 - DNA, 401 bp, Homo sapiens
ctctgtgtaaaatagccaagcctccgttattcactatcttcttttgctcttttatttttcatatatgactacgtgaaagtactgtctgttgttatatgtctagttacttgttatgtgagtataagcttaatcaaggtgatgctttattgcacttagcgctgtttccctgcaatccagtacaaagcccagtatgcaagttayaggcatgtggtaagtgttggatatgtgcaccagttgttggacaggattgaacttgcgaggtcagctttctcaggtgacctcaaaggcaagcatctagtttattgagtagtacaacaataggtatgttgcatgtacatatatgtataacagaagtttatttttaagcctgaattagtgatagcaactctgttcatgagtga

SEQ ID NO: 586 - DNA, 401 bp, Homo sapiens
catctataaaatgagaaagcgcaaaataatacttctacctcatagggttatcgtgaaagttaaatgacagagggataaatgaaatatttagtatagtgcctaccacaaaagccaaagctaaataagtgatagccaagaacaaacacagaaaacttcctaaataaaaatatgactagaataactgcaatgtatggaccaacwtaagcatgtttaagagtgaacacaggtgctattagtaattacacctggaccaaaagtataacccaggatgctccctgacatactagaatttatggtttctctatctataatctattaacatagcaggcagtaaaccttctcttttgtggagaccacacatgcaccagacaagcagagcctggattcaatagtgtgccagt

SEQ ID NO: 587 - DNA, 401 bp, Homo sapiens
tgggattatggattaatcagagcttgtgtttaccaaagtaaagttcaccaatgattcttcaaatatgttcacaagctaacttggcttatatccatttacttataaatcagaagattccccaagattttactgtctgcctgattaaagctagtatgtgctataagtgttttcaaaatacttttttccaaaactgtccttgcrgttaaggtgctgttaattcaaagtacctatgacttcctatcagaaatgaaaatgacacttaatgagaacaaaacatgttttcacttgaaatgcaattaaaccaaattagagaaacagtgtttttctaactaagataaaaacagctgtgtttatgaagctgctctaatggtattagaatttctcttatggaaacttttgct

SEQ ID NO: 588 - DNA, 401 bp, Homo sapiens
gtctaatcaaatatgagttctggtgccacagaacggtgatgttgaaatacttggcttttattggctatttatggcttttattggcatattaatgtcttttactgatgatctcaagttctgtctatagaaacactgatgtctgattgtaacagtttaatcaatgtataaaccaatctggatggttcagatctcaattccaaygtttcctttagaattcaggctcaatttatctaaattgggctcattatattattattaattttactttggctgtgggctgagttaaagcacactatgtttttgagcattctaagattgttccaggatttaaaaatttaagattactttggtttcttggaattatttaatacacactgcaattttccctgaaaaacacatgt

SEQ ID NO: 589 - DNA, 401 bp, Homo sapiens
taaagcacactatgtttttgagcattctaagattgttccaggatttaaaaatttaagattactttggtttcttggaattatttaatacacactgcaattttccctgaaaaacacatgtaagctaaaatggctctatggaaatgttttatgttttaatgaggttgggaaacagagataaattcacctggtcataatcatatrgtaaatattgatgaaaggaacctttttctaaactgcatagctataattctattccagctgattttgtagatttaaaatattttaccctgtttttgtgtctcttaaggagtttctgtttgggttatttgtacagatcaacagataaaaacttatgtcctagaacaaaaacagcacacttaaacacatcttttctttcaggc

SEQ ID NO: 590 - DNA, 401 bp, Homo sapiens
actttttgtgcctggaatgcaccttggagatagccttgcccattgaccagctcttcatttttcagagggagaagaaagtgaggtctgggaaagtaaaatgcctgcccaaatccacagagctaagaagtagcagagcagagatcattaccacacattccaaactttcttcctatttatcatttttctccttgagccccttcytgtctgagacactacatcaattaaaattattttataactgcaacatcgtacaattgccatgtaacacattgcccaatgcaattcaatcttagcatctggaaacaatactcatttattaactcacactttgtaggtcagaagtctgagcacagcatagcaaagtcctgttcttagggtctcaccaggctgaaatcaaggtg

SEQ ID NO: 591 - DNA, 401 bp, Homo sapiens
ctgatgaggtcagacccactcaagacaatctccctcttgattagctcaaagccaaccgactaacaactagtcaagagtggtatcccatcatagtcgtaattctcacttgtgctcaaaggggaggggatcatacagcatgtgtgtaccaggggacaggagtcttgggagtcatcttagaattctgtttgttgggccgagcgyggtggctcatgcctgtaatcccagcactttgggaggccgaggcaggtggatcacgagatcagaagatcgagaccatcctggctaacacggtgaaaccccttctctactaaaaaaatacaaaaaattagccgggtgtggtggtgggtgcctgtagtcccagctactcgggaggctgaggcagaagaatggtgtgaacccgg

SEQ ID NO: 592 - DNA, 401 bp, Homo sapiens
cagtgctttttcaaggacaggcttctccttctcaggttttggttactcttgttttcttttttcttttttgagacagagtctcgctctgttgcccaggctggagtgcaggggcgagatctcagttcactgcaacctccgcctcctgggttcaagtgattctcctccctcagcctccggagtagctgggataacaagcacgcrccaccatgaccacctaatttttgtatttttagtagagacagggtttcatcatgttggtcaggctgggctggaactcctgacctcaagtgatccacctgcctcggcctcctaaaatgctgggattacaggtgtgagccactgcgctcagctgagacagtgaccctctgagggtctcagcatatttaggcagagattggggg

SEQ ID NO: 593 - DNA, 401 bp, Homo sapiens
ttcaaggacaggcttctccttctcaggttttggttactcttgttttcttttttcttttttgagacagagtctcgctctgttgcccaggctggagtgcaggggcgagatctcagttcactgcaacctccgcctcctgggttcaagtgattctcctccctcagcctccggagtagctgggataacaagcacgcgccaccatgmccacctaatttttgtatttttagtagagacagggtttcatcatgttggtcaggctgggctggaactcctgacctcaagtgatccacctgcctcggcctcctaaaatgctgggattacaggtgtgagccactgcgctcagctgagacagtgaccctctgagggtctcagcatatttaggcagagattgggggatggcggag

SEQ ID NO: 594 - DNA, 401 bp, Homo sapiens
ggctgggctggaactcctgacctcaagtgatccacctgcctcggcctcctaaaatgctgggattacaggtgtgagccactgcgctcagctgagacagtgaccctctgagggtctcagcatatttaggcagagattgggggatggcggagggagctactgagtgacaggcaccctctgagagggagcctatctcaagcaatmaaaaaatatcaactatatgtagggactctttctcccttcaagagttacaattaaacttttcagaaatcaagatcaaggctatagatgataaaaattttattcctaaaggaatttatagacaagaaattgattgaaccttataataccccatgatggttcatgagaaacacgtgttcatatgattataccatgtttttaaa

SEQ ID NO: 595 - DNA, 401 bp, Homo sapiens
caggatcagaaactaggcatcatccttcatttttctctttacatcttccttttcttctcacctgcctccagccacctggtgcccccaattctacagtgttcacctctgaagcagcctttattctcatcgcctgtcttgcttcttccctgacctgggtcctcttcctcattctttgactgggcttgtaaattctcttcttaygcagcgtcagtttcagttcacacccccagccatcccagctcctgctaccagagttaatcttctaaaatagagcgattatcttgtcattccttcgtgtaacgtctctattagattcctgctgctataaaatagaaacttctccttagagggcagccaggtcctttccaatctcattgctgcataaacaatctcttttctat

SEQ ID NO: 596 - DNA, 401 bp, Homo sapiens
gcccatggtccaatatttaaaactgttttcttcttaactacatgataattctataaaaacatcatttacttgccactacctcaaatgttgataattcgtaaatgtttatctacagctgtaagttgttttctaaactgcaagcctgtatttccaagtacctgaaactaaacatgcccaaagacaattcattaatctccatcycttccccgcaaaaaaagccagacagagctcaggatcagaaactaggcatcatccttcatttttctctttacatcttccttttcttctcacctgcctccagccacctggtgcccccaattctacagtgttcacctctgaagcagcctttattctcatcgcctgtcttgcttcttccctgacctgggtcctcttcctcattc

SEQ ID NO: 597 - DNA, 401 bp, Homo sapiens
ccttgaactgataaacaaaaaaaatctgagagtatttggtagtgaaagtaaaggaagggtcaaaagaaaggttttgttgttgttgttgttgttgttatggtttcttcgatagaaaggggaaagataatggttaattggggtaacccagcagtagagattggcagggcaggtaaaccggcaggataaaggaaggagagtatsgattagattttaataaaagtttcttagggaagaggggaagaaggaaaaaaaatgagttgagaaaatatatattaaattctttacattgtgcttctgagacagagctatatttagcacccataataaatatatctgataagaatcctagtatgttgctgactctcagcactctgcttattattatgaaaataaaaatgact

SEQ ID NO: 598 - DNA, 401 bp, Homo sapiens
ggataaaggaaggagagtatggattagattttaataaaagtttcttagggaagaggggaagaaggaaaaaaaatgagttgagaaaatatatattaaattctttacattgtgcttctgagacagagctatatttagcacccataataaatatatctgataagaatcctagtatgttgctgactctcagcactctgcttattrttatgaaaataaaaatgacttccatgtcattctgtcagactatcttttggaaaaaacaacgacaacaaaaataaaaatgtcattcaatccgaaagcaagagaaaagacaccttagtaaaagtaactttcttcttccacctacaaaaccagaacttgaagacacaaatctcaggtgtactgaaataatttttttgtgcctg

SEQ ID NO: 599 - DNA, 401 bp, Homo sapiens
catcactcatgacattcaatttatattttaacatcattataaatttaacagggttgaatctttcaaaataacaaagaaagtgccttttgaaaggaaagaagataggaacgttgaagaggttcttgtgggatcactcttaaaagccatcaaagcatctgaatctctctcttggctgtataataaatattcacagaaaatgascgccaagtatgtatggagaaatgccttggcatttgctgtatgaatatggatgatgggaatatagtaaaatattagagagattaaatcgaaactcaaagatagttctaaggtgtttactgggtgtcacttgatgaaatgactctatggggatttatcactaatctgtgaagtgaaaaataatcttaatgacgagaggaagc

SEQ ID NO: 600 - DNA, 401 bp, Homo sapiens
gaaaaataatcttaatgacgagaggaagcttacctacattatgcctaggacaagaagtttttaattcccctctaacaatgcagtgtttgcaggttttcatccattatcactttgttagagtatctttgctaaaacccaactattttataggctcctctgacacctcacaagaatccaagggaggaaatgaagccaatgaaygtaatctacaggtgaaatttgcagagcaaattcaggaaaaaaaagtaattatgcttcttcgcattttatcattttataacctcaccttttagagaaggtattgagagattatactgattagtccagatttttattttacatatttgttcatggaaaggcttattttaacagaagttatatcaaggtgtcatgcccagtac

SEQ ID NO: 601 - DNA, 401 bp, Homo sapiens
aaaaataatcttaatgacgagaggaagcttacctacattatgcctaggacaagaagtttttaattcccctctaacaatgcagtgtttgcaggttttcatccattatcactttgttagagtatctttgctaaaacccaactattttataggctcctctgacacctcacaagaatccaagggaggaaatgaagccaatgaatrtaatctacaggtgaaatttgcagagcaaattcaggaaaaaaaagtaattatgcttcttcgcattttatcattttataacctcaccttttagagaaggtattgagagattatactgattagtccagatttttattttacatatttgttcatggaaaggcttattttaacagaagttatatcaaggtgtcatgcccagtacc

SEQ ID NO: 602 - DNA, 401 bp, Homo sapiens
tgaagccaatgaatgtaatctacaggtgaaatttgcagagcaaattcaggaaaaaaaagtaattatgcttcttcgcattttatcattttataacctcaccttttagagaaggtattgagagattatactgattagtccagatttttattttacatatttgttcatggaaaggcttattttaacagaagttatatcaaggtrtcatgcccagtaccatcatcatcatcatcaaaaatttgactgaataaacctaagtacacacacacttctgtatgtatagtcttacatatgaaagtaatatttattcattcaccaaaaagtgtaaagagaggttaaaacaggatgggatgaaacagtgtacaaaggtaaggaaaagcttttccaagcaactgaatttccct

SEQ ID NO: 603 - DNA, 401 bp, Homo sapiens
ttctgtatgtatagtcttacatatgaaagtaatatttattcattcaccaaaaagtgtaaagagaggttaaaacaggatgggatgaaacagtgtacaaaggtaaggaaaagcttttccaagcaactgaatttcccttgaagcctgaatgttaagtggaattaggcaaagtgtggtggttgtgggaaaattcaggcagaggayatagctagtgcaaaggctctaaggctgcaaaggatgcaagctatgcaggcatggtgcaaggaggcctttgtgcaggaagcaaagggagataaggctggggtgacccacagcctccttcttcaagggcactggatagactcctgggaagcaaaaagtgactggattaaatttgcatttcaaaaagactcttctgactacag

SEQ ID NO: 604 - DNA, 401 bp, Homo sapiens
aagaagactgaagtcaaaatgacaggacttggggatgggtgggccacagaaagaaggtgtcaaaatgattgtcacagtaggtaattagtcattgattgatagaaaatggtaatcactgaactaggctttttatcttcattctgttctatttaggttcattctatccaaagtatttagtgcttaccagatgcaaagtacagytaggaattctaatggatagaaatataaaataaatggatacaaggttatataatctctgccttttataggcttatattcaaatacagacaatgctttccatttccagtaaagggaatgagaaattatatacccagatacatatatattgacacacaaataaacagatgtatacacatgtatgtctacatatgtatatatgt

SEQ ID NO: 605 - DNA, 401 bp, Homo sapiens
agtgcttaccagatgcaaagtacagttaggaattctaatggatagaaatataaaataaatggatacaaggttatataatctctgccttttataggcttatattcaaatacagacaatgctttccatttccagtaaagggaatgagaaattatatacccagatacatatatattgacacacaaataaacagatgtatacacrtgtatgtctacatatgtatatatgtatgtacacatatgcatatataagtatatatatatttgcatcatatacacatctaaattaagggtgcattgccaggggactgaggaggataaataatgcagaggtcagaaaaaataactgaaaagttgtgtgggatggtattcttcttataagaaattcaaatgttttcaaaaatg

SEQ ID NO: 606 - DNA, 401 bp, Homo sapiens
taattttatcataaaactgtctttacagcagacaatgactaattagtaattctattaaatatttgccaaatggagggtaccacttctagttttgccagagtgtcttgctggtcagaagtgctttgcctaataaactcaacatgcatgttgatgctaagagtctaactgaacacacaggacaatcgcaaatctgtgaattaktaagcctcctttcacctcaatttcctccctcataggatggtctttgcttatctcagttttgtgtgtattccttttgattgcttgttaatgtctcctatttatagtgatattatgtaacagtattatttaatctccaaaatagtatacaattgtcaagtaccattgctgtaattatggtgcagatgttggaatatgcttgg

SEQ ID NO: 607 - DNA, 401 bp, Homo sapiens
atttgccaaatggagggtaccacttctagttttgccagagtgtcttgctggtcagaagtgctttgcctaataaactcaacatgcatgttgatgctaagagtctaactgaacacacaggacaatcgcaaatctgtgaattattaagcctcctttcacctcaatttcctccctcataggatggtctttgcttatctcagtttygtgtgtattccttttgattgcttgttaatgtctcctatttatagtgatattatgtaacagtattatttaatctccaaaatagtatacaattgtcaagtaccattgctgtaattatggtgcagatgttggaatatgcttggaatcactaaaagtgaatttaccataagctaaggaagcttactcttcagggactctgactt

SEQ ID NO: 608 - DNA, 401 bp, Homo sapiens
gagacagggtctcactgtcacccaggctggagtgcagtggcacatttatggcttactgcagcctcaacctcccagggctcaggtgatcctcccacctcaacctcccagtagctgggactacaggggagcaccactacacctggctaatttttgtatcttttttttttttttttttttttttgtagagacaggatttcctcwtgttgcccaggctggtcttgaactcctgggctcaagcaatccactcatctcagcctctgaaagtgtaagaattacaggcatgagccaccatgcccggcctgtctatcacttagttcagtaaatgcttggtgaatgaatgaatcatatacatttagtggttgttttgaagttgactctgctgagtgagattttctttgcat

SEQ ID NO: 609 - DNA, 401 bp, Homo sapiens
tgttgtcatgcaattgccatttcatcatctttccttaaagtcccttcaatatttatcattgcaatttttgtcctgtcttttcccctccatcacatcaaaccaaggaaaaacaattattaaatgctcatttatactcacctccctcccttcatattcattcattcctcagccctaaaattcctgtaggaatcctgcttttaygttttatcttaaagccaccatagtatctgtaatctcttttgtagtcatgtatgcttaattttggaagactgtcaactacctcttcaggttcattcaaatgaaaatgtatcacaaaatcgtaggctcttcccctatctcactgaaaaattactctaaaaatgaccagtaagctgttttgttgaagcaaaacatccagaccc

SEQ ID NO: 610 - DNA, 401 bp, Homo sapiens
gcttaattttggaagactgtcaactacctcttcaggttcattcaaatgaaaatgtatcacaaaatcgtaggctcttcccctatctcactgaaaaattactctaaaaatgaccagtaagctgttttgttgaagcaaaacatccagaccctaaacaaaagtagcagaattacaacatttccatagaaatggattttgagcttyttttttttaattggggaaaatcttattttttaaaaaagaatcccagaatacatctttgctttcagtccttcaattctaacataatgccatgttagagaatatgtggtagtgtatactaagcccttatgctgtctcacatccttgggacaaaatatctgttttgaaaggacttctctgaattttgctatttcctgcttact

SEQ ID NO: 611 - DNA, 401 bp, Homo sapiens
ttaaattgcggccatcagaaaataccgtgagaaggtagtgtgtaggtaatggttcagttagcattgaccccagaaaatggaaaaccaagagcaagtcacttttaaagtgaagcaatagattttgaatatggattgttccaactcaccacttcccgaccacaacacaagagtatttcactgtaaattaagagtctaagttarccacagctgtggtataactctgagaatgttttctctacatgtagacaattctggatccttccgtggtgcccgttaccctggtctttaacttttgtcctttgcaggggaggggtcaatgccttcttatttccttgctctttagaattctccaccagagggaggacaagggaaggagtaggtttcacgcgcagttctgagta

SEQ ID NO: 612 - DNA, 401 bp, Homo sapiens
tgcccgttaccctggtctttaacttttgtcctttgcaggggaggggtcaatgccttcttatttccttgctctttagaattctccaccagagggaggacaagggaaggagtaggtttcacgcgcagttctgagtaaagtttgaatctgagaaaccccacattcacgaattaaggatctccagttctgtgtctttattctacycctccctgcctgcttttccactcggcagaactgagtgactttcagcggctgcagtaattttaattattctgagtcgtgctccgaactatgttttttatcagttcctctgaaaattaagttgtagtaagagacgtagtttaagggcacccccttagaagaggccaggttattttttaaggaaaagctgaagatttctaaca

SEQ ID NO: 613 - DNA, 401 bp, Homo sapiens
aactcttctgggaaagccaccaacgttccccccgcacccctcccagggttcctgaccacggagactctgcttggggcacaggtgtgggagtcgcaaacttttctctgcgccgtccttttccgcgtggaatgggacggagcagccctcccaggcgctgcctggctgcggaggggagcgggcagcgagagcctcgggtctcckcctgggttcccgggtctccggggcgctggcctcggtctccgcgcagcgtccagcgacccctgtcgggggttcccggcagccgcgccgccaccccccgcccggccagcgcgggaggaaaaggggctgcgcccgggagcgccgagcccaggctcctcccggtggcgtgtccgcgcctcggggtgggggtgtggtggggaaga

SEQ ID NO: 614 - DNA, 401 bp, Homo sapiens
gggcagaggccaggacgcgagccgccagcggtgggacccatcgacgacttcccggggcgacaggagcagccccgagagccagggcgagcgcccgttccaggtggccggaccgcccgccgcgtccgcgccgcgctccctgcaggcaacgggagacgcccccgcgcagcgcgagcgcctcagcgcggccgctcgctctccccmtcgagggacaaacttttcccaaacccgatccgagcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccaggtgg

SEQ ID NO: 615 - DNA, 401 bp, Homo sapiens
gcccttggaccaaactcgcctgcgccgagagccgtccgcgtagagcgctccgtctccggcgagatgtccgagcgcaaagaaggcagaggcaaagggaagggcaagaagaaggagcgaggctccggcaagaagccggagtccgcggcgggcagccagagcccaggtgggtgcgcagcgcggcccgggccccacgatcctccycctgctcctcctactcctcctcctcctcggatgccgtggcctctccctccccctctccctcgcccgtcctcttcgccctgcgctctgagcgcccgttgagtcgcgcggtgcttcccctcctgggggccgccgctcacctgggcgccgagtcctaccgggcgcctacgcccagagctcagggcaagggacagcagtcccgg

SEQ ID NO: 616 - DNA, 401 bp, Homo sapiens
gtccgagcgcaaagaaggcagaggcaaagggaagggcaagaagaaggagcgaggctccggcaagaagccggagtccgcggcgggcagccagagcccaggtgggtgcgcagcgcggcccgggccccacgatcctcctcctgctcctcctactcctcctcctcctcggatgccgtggcctctccctccccctctccctcgccsgtcctcttcgccctgcgctctgagcgcccgttgagtcgcgcggtgcttcccctcctgggggccgccgctcacctgggcgccgagtcctaccgggcgcctacgcccagagctcagggcaagggacagcagtcccggccgcaccctcccagagtcccgggagcgcttcgctccctggcacggcccctccccagcgccttagc

SEQ ID NO: 617 - DNA, 401 bp, Homo sapiens
ctcctcctcggatgccgtggcctctccctccccctctccctcgcccgtcctcttcgccctgcgctctgagcgcccgttgagtcgcgcggtgcttcccctcctgggggccgccgctcacctgggcgccgagtcctaccgggcgcctacgcccagagctcagggcaagggacagcagtcccggccgcaccctcccagagtccygggagcgcttcgctccctggcacggcccctccccagcgccttagcggctgagcccagcccgggagtgggacctgggctataggagtcgaggctgcgtgcgcgcgtgccccgcgccataagcgctttgcacgggggccgtgtgccctctagcgggaaacgctggaatgggccgcctggagggagagccggtcccctcggtg

SEQ ID NO: 618 - DNA, 401 bp, Homo sapiens
ggacagcagtcccggccgcaccctcccagagtcccgggagcgcttcgctccctggcacggcccctccccagcgccttagcggctgagcccagcccgggagtgggacctgggctataggagtcgaggctgcgtgcgcgcgtgccccgcgccataagcgctttgcacgggggccgtgtgccctctagcgggaaacgctggaaygggccgcctggagggagagccggtcccctcggtgtgcctggcagcgcagaagtgggtggtcgagcaagaggccgcgtgggaagttagcttcggcgttttggggcacagggcaagcgatgtagagtgcgcgccggttcatcttgattcagtcctgtgctacggagactcaagagcagcggcagggattcctaaaggtcagc

SEQ ID NO: 619 - DNA, 401 bp, Homo sapiens
ccctcggtgtgcctggcagcgcagaagtgggtggtcgagcaagaggccgcgtgggaagttagcttcggcgttttggggcacagggcaagcgatgtagagtgcgcgccggttcatcttgattcagtcctgtgctacggagactcaagagcagcggcagggattcctaaaggtcagcgaagcagatgaaatctagtccagtgycaggaatcagctctgccccaaacaagttctacaaacttttggcttaacagttctatttttcaagacctctgtgataaatatggataatttactttcccaagagatgtaagaaaacaaccgtttatctgaatttttgaacccaattgtttgctggttatctatataataaaatggcaattgaagctttctagaaacaaatc

SEQ ID NO: 620 - DNA, 401 bp, Homo sapiens
gaagcgctgatgctttggacattaccttggacaaaagaggcatgtcgaataaatacttgtttatgagtgattggtaggtagctgataaaagaggggaggaaggagatgggggagaatttattttttgcctctgcaattcccaattattaattgatccagatgccttaagtcgcttcgagcgtacccccaacacacatttcwtgaagcacttctgcagaattaattcactagtacttgataatactgtggctacagtaatactgtagaagtatttgtcccttgctggaagaaaacaaaccacatttgcttggtcttcttactggtatcactgatagcatttcagaacctgggttattggcagagaccttcagcagagttttgagcaaaacagaaattgaaca

SEQ ID NO: 621 - DNA, 401 bp, Homo sapiens
ggaagaaaacaaaccacatttgcttggtcttcttactggtatcactgatagcatttcagaacctgggttattggcagagaccttcagcagagttttgagcaaaacagaaattgaacagagagtaattgtttcaccactggccattcttgtgcttgcttctttaataagatcctgctgatcatattcacttcaattactgaycattgcccctcatgagggtgtattgaattttcccttatttttaccctatattttggggagagaccatagcactttattcaacaagataggaaattggggagctggattagttacctccctgcgtgactttgtctactgtcctttctaaggtcatcatagtacattacggagatttcagacctagctcaaatcgttgtaaa

SEQ ID NO: 622 - DNA, 401 bp, Homo sapiens
atggtaagtgccagtgtagttttccttgtcttaggagaattcatacctgatgactataatggtttgtgagaaaaacccacactctaggtttcaggcattcccaaaggaacgacctatagatgccataactctagtattcacacttgccgtgccagcccaaattaggacttaggaggacaagtttctcttgtattgctcccrtcttagtgcctgtcagttccaagtttcactcctttcttaaaacagccactgcaactgtctgtccttgatgatatgtatgcaatagccaagcctctcttcagctgttttaacattgcctcatgacagtcaggggagagataatggggtgggatctcagccactcagccccgctgtgtgctatattgtacgcataggcataa

SEQ ID NO: 623 - DNA, 401 bp, Homo sapiens
tcctgtctcccttcactcctggtaggaggggcatttgtgagtctgggtgatggaaacattgagaaacctagtatcccaaataggtagaagcaggctgtgtgtttccccaatgacatcacccacctgtttccctcactaagtggctctgcaggctgaagtgcttgtaaacattcttgcgtataaagtaccctgaccctaacrgggcagcctgtgtttacagaacaagaggaatgtccagtgatctgtttcaacctggttacttgtaaccacctagcctatgtaattattaaaaacattaaagattattcacagaatgaaataggacttattttgcagaattaaaatagcaggaagagcggaagaaagttgtatattcagacatgaatacttctcatacccat

SEQ ID NO: 624 - DNA, 401 bp, Homo sapiens
cggggcagcctgtgtttacagaacaagaggaatgtccagtgatctgtttcaacctggttacttgtaaccacctagcctatgtaattattaaaaacattaaagattattcacagaatgaaataggacttattttgcagaattaaaatagcaggaagagcggaagaaagttgtatattcagacatgaatacttctcatacccrtgaaatggtcagataacactaatgaaaaagaggagaaaaaatatatatagagaaccaaaaaatattagacaaacatgaacactgaatacctactacatgccaggcactgtgctggacacttttatgtttattaatgagggacctgctatttattgaattcttaccatgttccaggcagtaaaacagatgtatattgtgta

SEQ ID NO: 625 - DNA, 401 bp, Homo sapiens
cagatgtatattgtgtattttgtcatccgtcctttcgaaaataagaaaattccttgctattaggcagcaagatgccagatggcctagtccttagctcttcagccagattgcctgggtttaaatcctaattaggctgttccttaaatatgtagtcatgggaaagttacttaatctttctgtgcctaaatttcctttttctgyaatatggggataataatactattattattatcatgagggtttcatgaagcttaaaggaaataatccatgaaaagcactctgaacatcaaccaacatgtaataagtgcccagtaatattagctgtctttataaaactacttaagaaatgaattatctgtgtctttgcaaagatatttgatataatttccaaaatttacctt

SEQ ID NO: 626 - DNA, 401 bp, Homo sapiens
tggatattttttaccataaaaggtaaattttggaaattatatcaaatatctttgcaaagacacagataattcatttcttaagtagttttataaagacagctaatattactgggcacttattacatgttggttgatgttcagagtgcttttcatggattatttcctttaagcttcatgaaaccctcatgataataataatastattattatccccatattgcagaaaaaggaaatttaggcacagaaagattaagtaactttcccatgactacatatttaaggaacagcctaattaggatttaaacccaggcaatctggctgaagagctaaggactaggccatctggcatcttgctgcctaatagcaaggaattttcttattttcgaaaggacggatgacaa

SEQ ID NO: 627 - DNA, 401 bp, Homo sapiens
ccagcaatgttcatagaattgagtgcaggtgtttctaaatataaacaaaataaggaatatattaaattcacatttctagttttttatttcctcctcagccttcctagaatggctataggtagtgtttaaagttctggaagtgtgatagattcttaccaaatcaatgttctgttagcattctccaagtaacaagttaaattsgatttgaacagtactgatagtttatcatgttctgttaggatagatgaaaacaaaccaaatgccagtattttgggtgagtcactttatttgctcaaagacgcaaatgaatgaacttctggtagtttagttaaccctggagctccccctcttgacaaatagaaaaaacaaaaaaataaatcagatagtattagcttgaaaac

SEQ ID NO: 628 - DNA, 401 bp, Homo sapiens
aaatcaatgttctgttagcattctccaagtaacaagttaaattggatttgaacagtactgatagtttatcatgttctgttaggatagatgaaaacaaaccaaatgccagtattttgggtgagtcactttatttgctcaaagacgcaaatgaatgaacttctggtagtttagttaaccctggagctccccctcttgacaaayagaaaaaacaaaaaaataaatcagatagtattagcttgaaaacggatttcttttacccagataaaaaatatgccactaaggtacatacaatgaagttttcttgaaggaactaatgatattttggatttctcctaattttgtgcatgtctgcacttgtgtgtgtccttgtgtatgttttaatgggttaaaaaaaaaagcga

SEQ ID NO: 629 - DNA, 401 bp, Homo sapiens
cagtgtttgtcgctttagaaaaagtgaggtgttagaaaaaaagaagaaatggacagctttatttatttttggttttactaaacataaagctggctacttttcaacaattgactaataattcactgtatataaccctgtttacaaaagtacattttattaaggaaaatattccatgttctcatctttcttctaaattttttwaaaaaagcctctgcagtaaattcccaaaagaattcagaatgtaggcagaaaatatgttaattttttaaaattaggaaacaaaattatttgagaatgtcgttaacacattagatttcttttgcatttttaaactagaaattagatatgcactttcttatgttgccctgattatgactgataaagtgcatttttcagtccaa

SEQ ID NO: 630 - DNA, 401 bp, Homo sapiens
gggactctgttcttccagtagattgcccagaatactgcagtaacttaaaaaaaaaaagagagagagagagaagaaaaaagcagtctttggggaatagcagtgtgatgctcctggagcaccctctggaggccagaaaaaggtgacatggggcataggaagaatgaagagaattctgatcatctctgctcttatgtagaggcmgaactccacagatctcacttgacttggactgaaaaatgcactttatcagtcataatcagggcaacataagaaagtgcatatctaatttctagtttaaaaatgcaaaagaaatctaatgtgttaacgacattctcaaataattttgtttcctaattttaaaaaattaacatattttctgcctacattctgaattcttttgg

SEQ ID NO: 631 - DNA, 401 bp, Homo sapiens
atgtggtccagctggaaaacaaactccagttgaggcagctaacatgtatggaatcgggactctgttcttccagtagattgcccagaatactgcagtaacttaaaaaaaaaaagagagagagagagaagaaaaaagcagtctttggggaatagcagtgtgatgctcctggagcaccctctggaggccagaaaaaggtgacayggggcataggaagaatgaagagaattctgatcatctctgctcttatgtagaggcagaactccacagatctcacttgacttggactgaaaaatgcactttatcagtcataatcagggcaacataagaaagtgcatatctaatttctagtttaaaaatgcaaaagaaatctaatgtgttaacgacattctcaaataattttg

SEQ ID NO: 632 - DNA, 401 bp, Homo sapiens
aggtgaaagcatcaaagttggacaacagatctttactaacacatttgttttcatctcttcctatctctctgtttcctcaacccaggggcaacaaatgtttaggagccccactttgattgagtgcacatttttgaacacaggtttgagtgactaattcttgtggatcatgggctgattttatttatgtactttttttgagayggagtctcactgtgtcacccaggctggagtgcagtggtgccatctcgactcactgcaagctccgcctcccgggttcacgccattctcctgcctcagcctcccgagtagctgggactacaggcgcccaccaccacgcccggctgattttttgtatttctagtagagacggggtttcaccgtgttagccaggatggtctcca

SEQ ID NO: 633 - DNA, 401 bp, Homo sapiens
catcaaagttggacaacagatctttactaacacatttgttttcatctcttcctatctctctgtttcctcaacccaggggcaacaaatgtttaggagccccactttgattgagtgcacatttttgaacacaggtttgagtgactaattcttgtggatcatgggctgattttatttatgtactttttttgagacggagtctcrctgtgtcacccaggctggagtgcagtggtgccatctcgactcactgcaagctccgcctcccgggttcacgccattctcctgcctcagcctcccgagtagctgggactacaggcgcccaccaccacgcccggctgattttttgtatttctagtagagacggggtttcaccgtgttagccaggatggtctccatctcctgac

SEQ ID NO: 634 - DNA, 401 bp, Homo sapiens
cccgggttcacgccattctcctgcctcagcctcccgagtagctgggactacaggcgcccaccaccacgcccggctgattttttgtatttctagtagagacggggtttcaccgtgttagccaggatggtctccatctcctgacctcgtgatccgcccgccttggcctcccaaagtgctgggattacaggcgtgagccaccgsgcccggcggggctgatattttaatatggtcacacaggccttgttatttatggtttacctgcttatgttaattccaccctgtacttgttatctttgacatgagttactctcaggatattactccccttccctttttaaagtgtaatattgttcccattgtttcttgctccaagaaatctgtcgttgatgggaatagtcttt

SEQ ID NO: 635 - DNA, 401 bp, Homo sapiens
tcacgccattctcctgcctcagcctcccgagtagctgggactacaggcgcccaccaccacgcccggctgattttttgtatttctagtagagacggggtttcaccgtgttagccaggatggtctccatctcctgacctcgtgatccgcccgccttggcctcccaaagtgctgggattacaggcgtgagccaccgcgcccggyggggctgatattttaatatggtcacacaggccttgttatttatggtttacctgcttatgttaattccaccctgtacttgttatctttgacatgagttactctcaggatattactccccttccctttttaaagtgtaatattgttcccattgtttcttgctccaagaaatctgtcgttgatgggaatagtctttgactgcc

SEQ ID NO: 636 - DNA, 401 bp, Homo sapiens
ctcccgagtagctgggactacaggcgcccaccaccacgcccggctgattttttgtatttctagtagagacggggtttcaccgtgttagccaggatggtctccatctcctgacctcgtgatccgcccgccttggcctcccaaagtgctgggattacaggcgtgagccaccgcgcccggcggggctgatattttaatatggtyacacaggccttgttatttatggtttacctgcttatgttaattccaccctgtacttgttatctttgacatgagttactctcaggatattactccccttccctttttaaagtgtaatattgttcccattgtttcttgctccaagaaatctgtcgttgatgggaatagtctttgactgccacagtgccccgaacctttcattc

SEQ ID NO: 637 - DNA, 401 bp, Homo sapiens
attttttacaaccaataaatcagctaaaatcatatccatattaaatgaatttagcataaaattagaatgacagagcaactagcattttcccttggtgagaaaagtagtaagtttccaccataaaatatgactcatttttatttgacctattagcagatagttgttattgcaccttatcttaaccaccattatttctacagmgagattacagagcaatgagtaggaaatatggaagattgctgaataatcctggctttagttatggagtaatatgaaaaaaggaaaagaaaaccttttgttcactttcacagtactacatcaatgcaagctttttagtagttttaatgtatgaattttatcatcaggtgaaaatgcatctcattattagatggcctatttta

SEQ ID NO: 638 - DNA, 401 bp, Homo sapiens
aaatgaatttagcataaaattagaatgacagagcaactagcattttcccttggtgagaaaagtagtaagtttccaccataaaatatgactcatttttatttgacctattagcagatagttgttattgcaccttatcttaaccaccattatttctacagagagattacagagcaatgagtaggaaatatggaagattgctgrataatcctggctttagttatggagtaatatgaaaaaaggaaaagaaaaccttttgttcactttcacagtactacatcaatgcaagctttttagtagttttaatgtatgaattttatcatcaggtgaaaatgcatctcattattagatggcctattttataaataaagttcaaatttcgatatagccagtttttaaaactg

SEQ ID NO: 639 - DNA, 401 bp, Homo sapiens
tttttatttgacctattagcagatagttgttattgcaccttatcttaaccaccattatttctacagagagattacagagcaatgagtaggaaatatggaagattgctgaataatcctggctttagttatggagtaatatgaaaaaaggaaaagaaaaccttttgttcactttcacagtactacatcaatgcaagctttttrgtagttttaatgtatgaattttatcatcaggtgaaaatgcatctcattattagatggcctattttataaataaagttcaaatttcgatatagccagtttttaaaactgtatatgtttataactcatccctttgttgcatataatctcccaattttagagacttacattgcatttttaaaggatagcacagtctctttagc

SEQ ID NO: 640 - DNA, 401 bp, Homo sapiens
cactttcacagtactacatcaatgcaagctttttagtagttttaatgtatgaattttatcatcaggtgaaaatgcatctcattattagatggcctattttataaataaagttcaaatttcgatatagccagtttttaaaactgtatatgtttataactcatccctttgttgcatataatctcccaattttagagacttacwttgcatttttaaaggatagcacagtctctttagcaatcccacagggaatctaaaaccagcctttacaaggagatgtcaaaattccaatttgcagtgatatttttgtcaaaacaagtaacgtgtgttattacgtatagtaagttaacacaatttttcatttgtgtatcaagtaggcattggtttttgttttttggtttttg

SEQ ID NO: 641 - DNA, 401 bp, Homo sapiens
ttccaatttgcagtgatatttttgtcaaaacaagtaacgtgtgttattacgtatagtaagttaacacaatttttcatttgtgtatcaagtaggcattggtttttgttttttggtttttggttttttttgagactaagtcttgctctgtcacccaggctggagtgcagtggtgcgatctcagctcactgcaagctccccccrctgggttcatgccattctcctgcctcagcctcccaagtagctgagactacaggcgcctgccaccacacccagctaatttttttcgtatttttagtagagacggtgtttcactgtgttagccaggatggtctcgatctcctgaccttgtgattaacccacctcggcctcccaaagtgctgggattacaggcatgagccact

SEQ ID NO: 642 - DNA, 401 bp, Homo sapiens
agaggaaaccaataatttgaatactgagttgaatggaaaatgaaatacatgattttccatatattaaattgaatagaaatgggagacttatgtcttgagggtttttcttttctttttttgaaacatgttgatgatattgtgtgttcccaagtgccctcaacctgacaggaaggagaaatgtcaatagaaatagttacttaytatattttgatatttagaaaggaacagtggagttttaaatgttgtgcatatcatttatgccccatccaaaaggatagcgatccacttaaaagaaggcagaaaagacaaggaccgctatcgttctccaggaatcaacagaagctaagggagagcgtagagtatagtccctgtggcctctacggagtcatcgccttctcaga

SEQ ID NO: 643 - DNA, 401 bp, Homo sapiens
caatagaaatagttacttattatattttgatatttagaaaggaacagtggagttttaaatgttgtgcatatcatttatgccccatccaaaaggatagcgatccacttaaaagaaggcagaaaagacaaggaccgctatcgttctccaggaatcaacagaagctaagggagagcgtagagtatagtccctgtggcctctacrgagtcatcgccttctcagactgaatccacacaaggccgccttgactgaaagagcctctacattggactgtgcatggcgtggggaaatgtgattacttgcaatatgctactgattacagaataaatattatgcattcggttttcactaaacttaaaaagcccagtgaaaaccccacttctgcatgtggtgaatgtggttat

SEQ ID NO: 644 - DNA, 401 bp, Homo sapiens
ggacctgtgtgtttgggagagccgcaaagtggctgtgcctctgatatcacaggactctatgaaagaggcaaccaaagaagacctgaggctcttcttgtcattttagatatgtgatttcataaagttactcttgaccttaatgcagatggccatttgggatttgtgtctgtgcatttcatccacagctggatggcatagttrctgagtaatcctgttgctctggagcaatgtaagttacagctgtggccaggcacgtggctcatgcctgtaatcccaacactttgggaagccaaagtgggagatctcttgaactcagggattcaagaccagcctgggcaacacaggagtcctcatctctactaaaaatccaaaaagttagccgggcaaggtggcatgtgcct

SEQ ID NO: 645 - DNA, 401 bp, Homo sapiens
tactcttgaccttaatgcagatggccatttgggatttgtgtctgtgcatttcatccacagctggatggcatagttgctgagtaatcctgttgctctggagcaatgtaagttacagctgtggccaggcacgtggctcatgcctgtaatcccaacactttgggaagccaaagtgggagatctcttgaactcagggattcaagwccagcctgggcaacacaggagtcctcatctctactaaaaatccaaaaagttagccgggcaaggtggcatgtgcctgtggtccagctgttaaggagggtgagatgggaggatcacttgagcccaggaggttgaggctgtggtgggcaattatgatacaccactgcactccagtttaggtgacagtgtaagacttgtctcag

SEQ ID NO: 646 - DNA, 401 bp, Homo sapiens
taatcccaacactttgggaagccaaagtgggagatctcttgaactcagggattcaagaccagcctgggcaacacaggagtcctcatctctactaaaaatccaaaaagttagccgggcaaggtggcatgtgcctgtggtccagctgttaaggagggtgagatgggaggatcacttgagcccaggaggttgaggctgtggtgrgcaattatgatacaccactgcactccagtttaggtgacagtgtaagacttgtctcagcgaaaaaaaaaaaaaaaaaaaaagttaacaaccatatcaaaagaacaatttattcaccccccaaattgtcttggaattctcacattaatttacagagagaaataaccctttggaaaattcacaatgttttgctagtattagta

SEQ ID NO: 647 - DNA, 401 bp, Homo sapiens
aagtaccaaatttgcagattgcctgaaaatgttcttatgtactttttatggatacaggctttcttttcaattagattcaaaaagtatcaatacattattatactttaaattgactagagctaccttttaaatggtcagataggttttctttttaaagaattacttttgtgattactagccacctgaaatggtgtttcaagkccatgcatggtgcctcatacttgtattcccagcactttgaggggctgaggtgggagcatcgcttgagctcgagttcaagaccagcctgggcaacatggcaaaaatctgtctctacaaaaaaatacaaaaaaaaaaaagaaagaaaaagaaaaaccattagccaggcatggtgggatgcacctgtagtcccatctacttgg

SEQ ID NO: 648 - DNA, 401 bp, Homo sapiens
ataggttttctttttaaagaattacttttgtgattactagccacctgaaatggtgtttcaaggccatgcatggtgcctcatacttgtattcccagcactttgaggggctgaggtgggagcatcgcttgagctcgagttcaagaccagcctgggcaacatggcaaaaatctgtctctacaaaaaaatacaaaaaaaaaaaaraaagaaaaagaaaaaccattagccaggcatggtgggatgcacctgtagtcccatctacttgggaggctgaggcagaaggatcacttgagctctcagaggttgcagtgagctggaatcgtgacaccacactccaggctgggtgacagagtgagaccctgtgtcaaaataaaataaaataagatataaatgaaatagtattt

SEQ ID NO: 649 - DNA, 401 bp, Homo sapiens
cccagcactttgaggggctgaggtgggagcatcgcttgagctcgagttcaagaccagcctgggcaacatggcaaaaatctgtctctacaaaaaaatacaaaaaaaaaaaagaaagaaaaagaaaaaccattagccaggcatggtgggatgcacctgtagtcccatctacttgggaggctgaggcagaaggatcacttgagytctcagaggttgcagtgagctggaatcgtgacaccacactccaggctgggtgacagagtgagaccctgtgtcaaaataaaataaaataagatataaatgaaatagtattttcaagacttggtataaatgcatagctgttaagctgtaaggacatcaataaatgttcataaataggtaactttctacattctatcacctta

SEQ ID NO: 650 - DNA, 401 bp, Homo sapiens
catggtgggatgcacctgtagtcccatctacttgggaggctgaggcagaaggatcacttgagctctcagaggttgcagtgagctggaatcgtgacaccacactccaggctgggtgacagagtgagaccctgtgtcaaaataaaataaaataagatataaatgaaatagtattttcaagacttggtataaatgcatagctgytaagctgtaaggacatcaataaatgttcataaataggtaactttctacattctatcaccttattttctataatacagaaaatatactttaaattattttatttatctgagtccttttattctttttgcttttttttttgtttgcaagaccaaagtcagagcaatttcaattctattttgagcattttattttggcactca

SEQ ID NO: 651 - DNA, 401 bp, Homo sapiens
ctttttgttccttcttgcagtatcttgtacccaagtagcttcaataaattttgactgacaagatgaccataaaataagtagtcctaatgtcataacatggaaaaggagggtaatgggtgtgtgtgtgaatgtgaattagtatagtatagtaatgagctgcctcagcgctcttaaactttttttagcaccaatgcctttatrcaggaaactctgaaaacaagcaagagatctttgtgggctctctgtaaattatgtgcatgcaaaaaagcatcctcagtgcttgtcccttgacagctgctctgagaccactgagtcactcgcttgcatctgtgagccactgaccagtcactgattgcagtgacagtttatcactaacaacctgaattatctttgacccagag

SEQ ID NO: 652 - DNA, 401 bp, Homo sapiens
ttgactgacaagatgaccataaaataagtagtcctaatgtcataacatggaaaaggagggtaatgggtgtgtgtgtgaatgtgaattagtatagtatagtaatgagctgcctcagcgctcttaaactttttttagcaccaatgcctttatgcaggaaactctgaaaacaagcaagagatctttgtgggctctctgtaaatkatgtgcatgcaaaaaagcatcctcagtgcttgtcccttgacagctgctctgagaccactgagtcactcgcttgcatctgtgagccactgaccagtcactgattgcagtgacagtttatcactaacaacctgaattatctttgacccagagacccaggggtcaaaggcctcctagtccattattaaatttccagggcagta

SEQ ID NO: 653 - DNA, 401 bp, Homo sapiens
ctcaggaagacacagatcctttgagggtggtagagactcctaaagtctgctgattcatacccagctggccccattaacaagagaattaaagagtggctagaaagggactgaagagttcactgggggcccatccaaaaagctgggggtacagacaactgctgattcccagccagtcatggattttattttgagacaggtctygtttggtcgcccaggctggagtgcaatagtgtgatcatagctcactgtagcctgggactcctgggctctagtgatcctcccaccttaacctcccaagtagctgggactacaggcatataccaccatgctcggttattttttttttaattttgtagagatggggtcttgctatgctgcccaagctggtctagaacttctgg

SEQ ID NO: 654 - DNA, 401 bp, Homo sapiens
cggcatggtttcagtgtgtgagtgtgcatgtatgtacgtgtgtgtgagaatgtgggtaagtgtgtgagcctgtaaaagtgtatcatgagtagctgtctcttgggaggagagggaatcaggaaccttggtcaaataacaccatgtactggcatggaaaggcactgttctgggcagcagtcaagaagacagaggaggactaamtgtatatgatttgggcaatcatttaaagtatctgggatttatttccttgtctaataattagcaaggttgatgtagatgaggtataacaactttaggggtagaagaaagctatgattcaaagtcggcattttattagatcagtggtctctgatttggtgtgatgtgttcctggtatgttttcgcttagaacaaggttgttt

SEQ ID NO: 655 - DNA, 401 bp, Homo sapiens
gaacaaggttgtttaacccatggcccatgggcttcatgcatcctaggacagctttgaatgtggcccaacacaaatttgtaaactttcttaaaacattatgagcttttttgtgattattttttccagttcatcagctgtcattagtgttagtgtattttatatgtggcccaagataattcttcttccaatgtggcccagggragccaaaatattggctcacttagagtctttagtagtgactatgtttttagaaagcctaaggtatttgaagaataatagtcacataaagtctaggcaaatagcaaatttgttttatgtaggaaacagcttatcttctatagaataacttttgatgggtgaaattgtagtgttaatttatcttagtgaacacctagaagatt

SEQ ID NO: 656 - DNA, 401 bp, Homo sapiens
cctaaggtatttgaagaataatagtcacataaagtctaggcaaatagcaaatttgttttatgtaggaaacagcttatcttctatagaataacttttgatgggtgaaattgtagtgttaatttatcttagtgaacacctagaagatttgtttaggtatgtgaattgtttagtgaaaatgtagaagatggtaagtatatttgkgaattactagtgcagaagccttttattaaattctcaatagaaactgggtactggttatgaattgtgatctctcagatacctttaacttagtcgtctctcatgagtatgtgatacaattgaagttgtgggatttaacctctgctgagcagcagaaagatatgaagcagtggaaaatacaggctaaacagagcctttcagaa

SEQ ID NO: 657 - DNA, 401 bp, Homo sapiens
ctagaagatttgtttaggtatgtgaattgtttagtgaaaatgtagaagatggtaagtatatttgggaattactagtgcagaagccttttattaaattctcaatagaaactgggtactggttatgaattgtgatctctcagatacctttaacttagtcgtctctcatgagtatgtgatacaattgaagttgtgggatttaayctctgctgagcagcagaaagatatgaagcagtggaaaatacaggctaaacagagcctttcagaataatttttcctcagtggtttatgttcttcacattcagtttttattcttctccatccttaactttctatagctgatacttccattttggatataggctctgtttcatttccagaggtatctctttccctaaactcat

SEQ ID NO: 658 - DNA, 401 bp, Homo sapiens
ttaacttagtcgtctctcatgagtatgtgatacaattgaagttgtgggatttaacctctgctgagcagcagaaagatatgaagcagtggaaaatacaggctaaacagagcctttcagaataatttttcctcagtggtttatgttcttcacattcagtttttattcttctccatccttaactttctatagctgatacttccrttttggatataggctctgtttcatttccagaggtatctctttccctaaactcatcaagtactttttcaaccttgttgatcatcttaacagaagaatcgtaacattaagttgatttatgacaccatatctctgtagtaactctgaattgtcatcaagctattcattgaaaggatacctcttaaactttctattactaagat

SEQ ID NO: 659 - DNA, 401 bp, Homo sapiens
gaataatttttcctcagtggtttatgttcttcacattcagtttttattcttctccatccttaactttctatagctgatacttccattttggatataggctctgtttcatttccagaggtatctctttccctaaactcatcaagtactttttcaaccttgttgatcatcttaacagaagaatcgtaacattaagttgatttrtgacaccatatctctgtagtaactctgaattgtcatcaagctattcattgaaaggatacctcttaaactttctattactaagatccccaaatgtatctattctcctctcgataaaatgagtattgtcttagacaagcagcagactgctttttaagtgaagtggattaagctgctgtgatcgtccctgtgaatgtgtatga

SEQ ID NO: 660 - DNA, 401 bp, Homo sapiens
aaaaacttaatctaaaagaagaaatattgagcaagcattgggtgcttcatgaatggcctaaaaatatatttaagtctatcacctggaggttcgttcatttattgccaggaaagaatcaatcccacttttttttctctttccctgtggctcaactccaggaaggtgttttaattgattggtgggaagattttaacacagacraaacttataagctaagcatcattatttctagtacagtggcttcaccacattatagggtaataaagttaaaagctgccgattgtttcagaattattttcagaaatttacatcctctttattcctattaccttgagaaagagttcagagaatgggcttagtcagcatgcaattatggtattagataaagcatacagtttggt

SEQ ID NO: 661 - DNA, 401 bp, Homo sapiens
acttttttttctctttccctgtggctcaactccaggaaggtgttttaattgattggtgggaagattttaacacagacaaaacttataagctaagcatcattatttctagtacagtggcttcaccacattatagggtaataaagttaaaagctgccgattgtttcagaattattttcagaaatttacatcctctttattccyattaccttgagaaagagttcagagaatgggcttagtcagcatgcaattatggtattagataaagcatacagtttggtctaaaaccaagatgcagctagtctcatcagggtactttaatcaatcaagggttgttaagaaggtaggaatgtcagtctaacttcattcctttgaaggtgatcagcctttcttctctggaggct

SEQ ID NO: 662 - DNA, 401 bp, Homo sapiens
ggctcaactccaggaaggtgttttaattgattggtgggaagattttaacacagacaaaacttataagctaagcatcattatttctagtacagtggcttcaccacattatagggtaataaagttaaaagctgccgattgtttcagaattattttcagaaatttacatcctctttattcctattaccttgagaaagagttcakagaatgggcttagtcagcatgcaattatggtattagataaagcatacagtttggtctaaaaccaagatgcagctagtctcatcagggtactttaatcaatcaagggttgttaagaaggtaggaatgtcagtctaacttcattcctttgaaggtgatcagcctttcttctctggaggctttgaagattttgtcctatggct

SEQ ID NO: 663 - DNA, 401 bp, Homo sapiens
cactttgggaggctgaggcaggaggattacttgagctcaggagtttgaaaccagcctgggcaacatagaaaaacaccatcttcctctccccactaccaaaaaaaaaagttagccagttataatggtgcacatgtgtagtaccagctacttgggaggctgaggtgggaggattgctggagcccaggagtttgagggtacagygaaccatgatcatatcactgcactgcagccagggtgacagagaaagacccagtctcaaaaaaaaattgttctccaacaaatcttttttgaagttatggcatttaaccctgcccaccattcccattcccctgtcagtttcactatcatgcattcctttttctttatcccactcaggaccattgggcttgtcaaacatataa

SEQ ID NO: 664 - DNA, 401 bp, Homo sapiens
cccctgtcagtttcactatcatgcattcctttttctttatcccactcaggaccattgggcttgtcaaacatataaattggctttcaggaaaattcttctccatatctcttaaaaatttcctgtatcccattctcttccctctttttggaattttctttttctataatttaaagatagagtcttgctgttttctaggctgckctcaaactcctgacctcaagcgatccttctactttggcctcccagtgttgggattataggtgtgaaccattatgcccggtctagaactttaactaaaagtacgttggaccttctttctctgaacactggttctttttacttccctcacgtttcccatcattttgtttcactctcccgctgtatctcttttgaagttatct

SEQ ID NO: 665 - DNA, 401 bp, Homo sapiens
ccctgtcagtttcactatcatgcattcctttttctttatcccactcaggaccattgggcttgtcaaacatataaattggctttcaggaaaattcttctccatatctcttaaaaatttcctgtatcccattctcttccctctttttggaattttctttttctataatttaaagatagagtcttgctgttttctaggctgctstcaaactcctgacctcaagcgatccttctactttggcctcccagtgttgggattataggtgtgaaccattatgcccggtctagaactttaactaaaagtacgttggaccttctttctctgaacactggttctttttacttccctcacgtttcccatcattttgtttcactctcccgctgtatctcttttgaagttatctt

SEQ ID NO: 666 - DNA, 401 bp, Homo sapiens
tttttctttatcccactcaggaccattgggcttgtcaaacatataaattggctttcaggaaaattcttctccatatctcttaaaaatttcctgtatcccattctcttccctctttttggaattttctttttctataatttaaagatagagtcttgctgttttctaggctgctctcaaactcctgacctcaagcgatccttytactttggcctcccagtgttgggattataggtgtgaaccattatgcccggtctagaactttaactaaaagtacgttggaccttctttctctgaacactggttctttttacttccctcacgtttcccatcattttgtttcactctcccgctgtatctcttttgaagttatcttccccttcactcattgactcttcagctat

SEQ ID NO: 667 - DNA, 401 bp, Homo sapiens
tgcttgatggttggtctctgaccctgtgttggccattgctctgtaaaaattatctgtgtcattcctcaaggcatgggatagaagcacattcctgccgagaaggtaagtgttactgctgggtacttggaaacttaccgagttcgtggcttgaggttctgtggcctaccagcctgtgcacacctaggcatgtagaaacctgtyggtagactcagcttttccctctggctgaactcaggatcaagacaatgtgtctggccggacacggtggctcatgcctgtaatcccagcactttgggaggccaaggagacgggtggatcacctgaggtcaggagttcgtgaccagcctgaccaacatggcgaaaccccatctctactaaaaatacaaaaattagccgggcct

SEQ ID NO: 668 - DNA, 401 bp, Homo sapiens
gcttgatggttggtctctgaccctgtgttggccattgctctgtaaaaattatctgtgtcattcctcaaggcatgggatagaagcacattcctgccgagaaggtaagtgttactgctgggtacttggaaacttaccgagttcgtggcttgaggttctgtggcctaccagcctgtgcacacctaggcatgtagaaacctgtckgtagactcagcttttccctctggctgaactcaggatcaagacaatgtgtctggccggacacggtggctcatgcctgtaatcccagcactttgggaggccaaggagacgggtggatcacctgaggtcaggagttcgtgaccagcctgaccaacatggcgaaaccccatctctactaaaaatacaaaaattagccgggcctg

SEQ ID NO: 669 - DNA, 401 bp, Homo sapiens
ggtaagtgttactgctgggtacttggaaacttaccgagttcgtggcttgaggttctgtggcctaccagcctgtgcacacctaggcatgtagaaacctgtcggtagactcagcttttccctctggctgaactcaggatcaagacaatgtgtctggccggacacggtggctcatgcctgtaatcccagcactttgggaggccraggagacgggtggatcacctgaggtcaggagttcgtgaccagcctgaccaacatggcgaaaccccatctctactaaaaatacaaaaattagccgggcctggtggtgggtgccaataatcccagctacttgggaggctgaggcaagagaattgcttgaacctgggagggggaggttgcagtgagccgagatcacaccactt

SEQ ID NO: 670 - DNA, 401 bp, Homo sapiens
ttactgctgggtacttggaaacttaccgagttcgtggcttgaggttctgtggcctaccagcctgtgcacacctaggcatgtagaaacctgtcggtagactcagcttttccctctggctgaactcaggatcaagacaatgtgtctggccggacacggtggctcatgcctgtaatcccagcactttgggaggccaaggagacrggtggatcacctgaggtcaggagttcgtgaccagcctgaccaacatggcgaaaccccatctctactaaaaatacaaaaattagccgggcctggtggtgggtgccaataatcccagctacttgggaggctgaggcaagagaattgcttgaacctgggagggggaggttgcagtgagccgagatcacaccacttcactccag

SEQ ID NO: 671 - DNA, 401 bp, Homo sapiens
atctctactaaaaatacaaaaattagccgggcctggtggtgggtgccaataatcccagctacttgggaggctgaggcaagagaattgcttgaacctgggagggggaggttgcagtgagccgagatcacaccacttcactccagccacagaacatctcaaaaaaaaaaaaaaaaagacagcatgtctttagttctctggggytaaatgggtatgtttagatttggtgtagcttttttggggttctttactccttttggggtcccagcttaatgtgaagagggtctactctaagactccccacttcaggaagatcctggagcctctatccctttctccaggatgagcaaatgcacttgaagaaaaagtagttgctttgtccaggtattttgcgtctatctcta

SEQ ID NO: 672 - DNA, 401 bp, Homo sapiens
tctttagttctctggggctaaatgggtatgtttagatttggtgtagcttttttggggttctttactccttttggggtcccagcttaatgtgaagagggtctactctaagactccccacttcaggaagatcctggagcctctatccctttctccaggatgagcaaatgcacttgaagaaaaagtagttgctttgtccaggtrttttgcgtctatctctaaggtttgagatttgccttagaatttgtccaaaaaatgatcttttttgtcaaattcttttaaggtgatgtttattatatttcattcagcacttttaattgtttccagccaggaggttcttgtgagaaacttaccttccaggaatggaagaggaatcattagtcattaaggagctaaaaatgtta

SEQ ID NO: 673 - DNA, 401 bp, Homo sapiens
ttggggtcccagcttaatgtgaagagggtctactctaagactccccacttcaggaagatcctggagcctctatccctttctccaggatgagcaaatgcacttgaagaaaaagtagttgctttgtccaggtattttgcgtctatctctaaggtttgagatttgccttagaatttgtccaaaaaatgatcttttttgtcaaaktcttttaaggtgatgtttattatatttcattcagcacttttaattgtttccagccaggaggttcttgtgagaaacttaccttccaggaatggaagaggaatcattagtcattaaggagctaaaaatgttaggaaaatattaatgcttcagaaacaatttgttgatctgaagagtacctaagttagggattttcagaaagt

SEQ ID NO: 674 - DNA, 401 bp, Homo sapiens
cttgtgagaaacttaccttccaggaatggaagaggaatcattagtcattaaggagctaaaaatgttaggaaaatattaatgcttcagaaacaatttgttgatctgaagagtacctaagttagggattttcagaaagtagcattttgttttgttttgttttgttttgtttgagacatggtcttgctctgtggcccaggctgraatgcagtggcgcgatctcagctcactacaacctccacaaacgattctgttgcctcagcctcctgagtagctgggattataagcaggcaccaccacacccggctaatttttgtatttttcgtagagacggggtttcaccatattggccaggctggtcttgaactcctggcctcaagtgcccacctcggcctcccaaagtg

SEQ ID NO: 675 - DNA, 401 bp, Homo sapiens
tacaggcatgagccactgcacctggccagtagcattctttggatgaattgactctggttttgttagccttcagaatctaaggaagtttatatctggtcaattttgagaaagaagatctctaagaaatgcctcaagtggggaagggatggactttataaaccaaaatgtcaatgtgttaatcctaaatatctcaatgaaatrgggcaaaatatggctaagaaaaatttgacaaggtgtaaatctcactgaatgcagttgctgtggttgctaccaagtttacaagagaagaaaccaggttttgccaatgcatcaaaaattagattaaaaaagaaatttctctggcctagtctgatgcttagccttcaagagtaaaatgatgtgttgttagatgacttgaccaa

SEQ ID NO: 676 - DNA, 401 bp, Homo sapiens
caaaaattagattaaaaaagaaatttctctggcctagtctgatgcttagccttcaagagtaaaatgatgtgttgttagatgacttgaccaaagtcccatagccaaggcagggagagtgttgcagcctggtggtccattatctgatgccactggatctatgtagatgagtttggaacttcactttctgccttgtctgggacrtaaaaactttgggtgcccctcacttcttttttatataaactgttgtgttttattgccctggcactcttaagttgctgtttcaccctagcagtgcctgcttcaagccatcaccaatgaaagtgcctttgaagtgtcttaaggcgtgtgcattgcctagacaaatagttggcagaagaggtcaacacaagcgtctttgcttt

SEQ ID NO: 677 - DNA, 401 bp, Homo sapiens
ccattccctgggctagaaagtagtgttaaaatcaactccaggtgtttccgttattcttaaggcctaagtattattaaagcagaaaataatatggttaggatatgaatgggaggggattggtcattcgctgcttgggaatcacttatttgcaatagagctcattctctcttgacagttgctcctacatgtctaaaatacacsataaatctcgaagtgctcgtttcaatattgaagtttcctccaagtgagctgttatcacttttgtcttcaagagaatgctaactcactgaatctctttgagctttttatgctttacactgtggattttggagtttgctttggtttttccactagttctttaagttaaatttaggtaaagaaatttaaaatcatcaggctta

SEQ ID NO: 678 - DNA, 401 bp, Homo sapiens
aaatctcgaagtgctcgtttcaatattgaagtttcctccaagtgagctgttatcacttttgtcttcaagagaatgctaactcactgaatctctttgagctttttatgctttacactgtggattttggagtttgctttggtttttccactagttctttaagttaaatttaggtaaagaaatttaaaatcatcaggcttattyttaaaaactcaagtattatcaaatgatggatgatagagatatgctttgggaatatatccaagtacttcaaatgatagaagttagaacaccaggagacatttagtaagagattcttctgctgggtgaaatcaacatcaacatcacctggaaactcgtgggtagtgcaaatcccctggcctcaccccagagctcctggaatg

SEQ ID NO: 679 - DNA, 401 bp, Homo sapiens
ttatcaaatgatggatgatagagatatgctttgggaatatatccaagtacttcaaatgatagaagttagaacaccaggagacatttagtaagagattcttctgctgggtgaaatcaacatcaacatcacctggaaactcgtgggtagtgcaaatcccctggcctcaccccagagctcctggaatgaagctgtccgggatakggcccggcatcctgtgttttaataagctgtttgtgggattctaatgttcgccaaagttttaggatcatgcctgcagagaatcctgctcactatgaaagtttgcatgcctgagtaagtcaaaagagatctttgaaaatagtatctaggtttgagagacttgctgtgattgtataattttagcaaatgtgtcaaaacaaatt

SEQ ID NO: 680 - DNA, 401 bp, Homo sapiens
ttttgagacagcatctcgctctgtcactcaggctggagtgcagtggtacgattttgactcattgcaacctctgcctcccaaattcaagcaattctcctgcctcaggctcccaagtagctaagattacaggcgcacaccaccacacctggctaatttttgaattttttgcagagaggggttttgccatgttggccaggctgktcttgagctcttgacttcaactgatccgcccaccttggcctcccaaagtgctgggattacaggcatgagccaccgtgcctggccaggccaagatgattcttgaagtgtgtgtgctgtgcgtactgtgtgtgcatgtatacgtacatttctgatggttcactttagtgatactgtagttcagtttatctgggattgcctca

SEQ ID NO: 681 - DNA, 401 bp, Homo sapiens
caggctcccaagtagctaagattacaggcgcacaccaccacacctggctaatttttgaattttttgcagagaggggttttgccatgttggccaggctggtcttgagctcttgacttcaactgatccgcccaccttggcctcccaaagtgctgggattacaggcatgagccaccgtgcctggccaggccaagatgattcttraagtgtgtgtgctgtgcgtactgtgtgtgcatgtatacgtacatttctgatggttcactttagtgatactgtagttcagtttatctgggattgcctcagtagaaagctatactcttacttgtttaactttctgtctattttctcctttaaaaaagaaaagattatatagtagacttatacttggtatctgtctgaaaaaa

SEQ ID NO: 682 - DNA, 401 bp, Homo sapiens
atcagaaatacaggcttgtatagaaatcaccattttttaaataaaattttatttttattaaattattgtgagctcattgttttttaaaacaatcatacacaagtaaaacatttaccttgggggtgggcatcagttccttttcccatccgttttcatctctagttttctttcctaaaaacaagccctctcaacaattctagktaattctttttctgatagttatcttttttttttttttttttttgagatggagtcttgctctgtctcccaggctggagtgcagtggcgcgatcttggctcactgcaacctctgcctcctgggttcaaccagttctcctgcctcagcctcctgagtagctgggattacaggcacacaccatcacgcctggctaatttttgta

SEQ ID NO: 683 - DNA, 401 bp, Homo sapiens
aagccctctcaacaattctagttaattctttttctgatagttatcttttttttttttttttttttgagatggagtcttgctctgtctcccaggctggagtgcagtggcgcgatcttggctcactgcaacctctgcctcctgggttcaaccagttctcctgcctcagcctcctgagtagctgggattacaggcacacaccaycacgcctggctaatttttgtatttttagtagagacgggatttttaccatgttggccaggctggtctcggactcctgacctcaggtgatccgcccaccttggcctcccaaagtgctgggattacaggcgtgagccactgctcctggcctctgatatttatctttatattttctatataacatgcatgttctgctgtttctt

SEQ ID NO: 684 - DNA, 401 bp, Homo sapiens
aaagggcctgcttttaaatattttttttggtgttgtccactacttgaacttcatcaagttcatcagaaaaaatttaaatggtatttctaaataagtggtttgctccactctatcattaagcttgttaagatgaataaaaatcagaatattttcaaatgtatgtaaatacatacattcagtgtatttcccagcttcagaaastcctaatttctgaaagtccttgttatcaactcttgttttgaaaggaccctttctttaagataaagtgtcacatacattattttgtattgatacaaattattgatgattatggatattttaggtaatcaatactaagataattatgttaatatgaattatgattatagggtattggtaacagacaggagtgtaggtgaatg

SEQ ID NO: 685 - DNA, 401 bp, Homo sapiens
tatgaaagaagagcatagtttatgtgtgaatatatgggcaacatgtatatagatgtccacatgaactgcgcctggttaggtcctagaaaagcctatggtgggaaaggtgggttaagaggattgaagttaggagtgaacttgttatagtcttcataaatatttttatagccagtaacataaaacaataaagacagtgctccraattaaagatacagcaatgacatgacatctacatttgaaactagtaataatggatgccgggccctattactaatttctgcttagcattccttactcaccagaaatgttttaggatcttagttcctttgcagcttgggatgcttgtaaattcacttgttcttttataaataatgtgcatacaaggagattttcaacagcat

SEQ ID NO: 686 - DNA, 401 bp, Homo sapiens
gataggacataaaagtgaacataaaaggacaaaaagctagcaataaactgtgtataaaaattacatggggtttggaatcagtttacggattgagttccagctcagtctctgactatttttatggccatgtgtgtatatttgtaacttaaaatgagcatatatggtcactgcctgtatgtaattatttactgagttgtcttwccatgggaatttttctacctattccttttgagtaactttgaaaattctgcttctgttgtaccttaattgcaagtcactcaatttactcctattggtagtaacaagaaaaagatgaaacagagttttgaaatagacatgaagcaaaagtaatggcaagaggaatggaggtgtttgcaaataaagcttgaagaaagatgtga

SEQ ID NO: 687 - DNA, 401 bp, Homo sapiens
tttctttgaaaacaggaaatgagaataataaaagtggatgagaaaaaaacacttgtggtaaaaagtcagaagacaggcagattttaaaatgtgggcaacttaggaatatagagaagataggacataaaagtgaacataaaaggacaaaaagctagcaataaactgtgtataaaaattacatggggtttggaatcagtttayggattgagttccagctcagtctctgactatttttatggccatgtgtgtatatttgtaacttaaaatgagcatatatggtcactgcctgtatgtaattatttactgagttgtcttaccatgggaatttttctacctattccttttgagtaactttgaaaattctgcttctgttgtaccttaattgcaagtcactcaattta

SEQ ID NO: 688 - DNA, 401 bp, Homo sapiens
acttttatgtcctatcttctctatattcctaagttgcccacattttaaaatctgcctgtcttctgactttttaccacaagtgtttttttctcatccacttttattattctcatttcctgttttcaaagaaacaattgcaacacattttagaaatgcttacgacataagtggcacactcctgaagtctttccaaaatctatyttcctccatgtcttaatttttgaaatttatatacttagcttctttctgctaattcccacagaaaaactcagcacaattttataagcaatgtgaatgatcttagagccctcataggaataaattgcacagaatgttatcaatatatcataagtgaggtacaggattaattttctaagatatcatttaaaatttcttctatt

SEQ ID NO: 689 - DNA, 401 bp, Homo sapiens
attctcatttcctgttttcaaagaaacaattgcaacacattttagaaatgcttacgacataagtggcacactcctgaagtctttccaaaatctattttcctccatgtcttaatttttgaaatttatatacttagcttctttctgctaattcccacagaaaaactcagcacaattttataagcaatgtgaatgatcttagakccctcataggaataaattgcacagaatgttatcaatatatcataagtgaggtacaggattaattttctaagatatcatttaaaatttcttctattgttactatacacatttgatttattaagccaaaacttcattcctctaagaataataatcccgtcacagttctaatcctagcatagtgtcgtcttaactgcaatgta

SEQ ID NO: 690 - DNA, 401 bp, Homo sapiens
ctttataactatcaatgatttttagctactgtataataaagaaagctgaaataagatgtaattgaaagttaggatagtacattgcagttaagacgacactatgctaggattagaactgtgacgggattattattcttagaggaatgaagttttggcttaataaatcaaatgtgtatagtaacaatagaagaaattttaaaygatatcttagaaaattaatcctgtacctcacttatgatatattgataacattctgtgcaatttattcctatgagggctctaagatcattcacattgcttataaaattgtgctgagtttttctgtgggaattagcagaaagaagctaagtatataaatttcaaaaattaagacatggaggaaaatagattttggaaagact

SEQ ID NO: 691 - DNA, 401 bp, Homo sapiens
ctaagatatacttacgataaatgttgccattactcggttcaggcaaaacattgtttgcaagtggggataggatatacagattaggaaaaatggtggcgaaattaacctcaaagtcagaaaataaatatgtcgcaaatttgtagattaatatattatttgatatttatttatttggtgtgtataattttatttgatgatgayttccttctttgaaaaaatgagatgacagattcacccaaactgtgactttgtagaaacccttgggtgaggacagttatcagacagaggaaatgaggcctgtagaatgtaaaattagagacataattatggaatgtctcaaccacttggaatctgaataattgcatctacccaacagaggaaaatgcctctgttgctgtatt

SEQ ID NO: 692 - DNA, 401 bp, Homo sapiens
gaagctaagatatacttacgataaatgttgccattactcggttcaggcaaaacattgtttgcaagtggggataggatatacagattaggaaaaatggtggcgaaattaacctcaaagtcagaaaataaatatgtcgcaaatttgtagattaatatattatttgatatttatttatttggtgtgtataattttatttgatgrtgatttccttctttgaaaaaatgagatgacagattcacccaaactgtgactttgtagaaacccttgggtgaggacagttatcagacagaggaaatgaggcctgtagaatgtaaaattagagacataattatggaatgtctcaaccacttggaatctgaataattgcatctacccaacagaggaaaatgcctctgttgctg

SEQ ID NO: 693 - DNA, 401 bp, Homo sapiens
atcatcaaataaaattatacacaccaaataaataaatatcaaataatatattaatctacaaatttgcgacatatttattttctgactttgaggttaatttcgccaccatttttcctaatctgtatatcctatccccacttgcaaacaatgttttgcctgaaccgagtaatggcaacatttatcgtaagtatatcttagctkcttctccttatctgaatggggttctagttcccttagcattctcagataacattctgaatattctactctgtatatccagtttgttcatagacctcaaaatgcagaccctgcatggattattcccattctgctgtctgtctttatgaaacctgccaatacttattgctagatgaaattctacagatgttccatgatgactt

SEQ ID NO: 694 - DNA, 401 bp, Homo sapiens
cataaattgtctgtggctacttttgtgctacaactgcagagttgggtctctacagaaatgtttgcagatccctgttatcgagtcaataagtcatcatggaacatctgtagaatttcatctagcaataagtattggcaggtttcataaagacagacagcagaatgggaataatccatgcagggtctgcattttgaggtctahgaacaaactggatatacagagtagaatattcagaatgttatctgagaatgctaagggaactagaaccccattcagataaggagaagaagctaagatatacttacgataaatgttgccattactcggttcaggcaaaacattgtttgcaagtggggataggatatacagattaggaaaaatggtggcgaaattaacctcaa

SEQ ID NO: 695 - DNA, 401 bp, Homo sapiens
aatctgtatatcctatccccacttgcaaacaatgttttgcctgaaccgagtaatggcaacatttatcgtaagtatatcttagcttcttctccttatctgaatggggttctagttcccttagcattctcagataacattctgaatattctactctgtatatccagtttgttcatagacctcaaaatgcagaccctgcatggrttattcccattctgctgtctgtctttatgaaacctgccaatacttattgctagatgaaattctacagatgttccatgatgacttattgactcgataacagggatctgcaaacatttctgtagagacccaactctgcagttgtagcacaaaagtagccacagacaatttatgagcaaatgaatgcaagtgtgctccaataa

SEQ ID NO: 696 - DNA, 401 bp, Homo sapiens
taagccaaatcctgcctaccatctgtttttgtaaataaagttttattggagcacacttgcattcatttgctcataaattgtctgtggctacttttgtgctacaactgcagagttgggtctctacagaaatgtttgcagatccctgttatcgagtcaataagtcatcatggaacatctgtagaatttcatctagcaataagwattggcaggtttcataaagacagacagcagaatgggaataatccatgcagggtctgcattttgaggtctatgaacaaactggatatacagagtagaatattcagaatgttatctgagaatgctaagggaactagaaccccattcagataaggagaagaagctaagatatacttacgataaatgttgccattactcggttc

SEQ ID NO: 697 - DNA, 401 bp, Homo sapiens
gaaaaattgaatattgctacagcaaatggtaaggatatatattttatgtcaggatattgggataatagtatctcttcaaggaatattatagagcagaggtcagcaaaatacggctcataagccaaatcctgcctaccatctgtttttgtaaataaagttttattggagcacacttgcattcatttgctcataaattgtctrtggctacttttgtgctacaactgcagagttgggtctctacagaaatgtttgcagatccctgttatcgagtcaataagtcatcatggaacatctgtagaatttcatctagcaataagtattggcaggtttcataaagacagacagcagaatgggaataatccatgcagggtctgcattttgaggtctatgaacaaactgga

SEQ ID NO: 698 - DNA, 401 bp, Homo sapiens
ggaatggtgctaaaccattcatgagaaatccacccccatgatccagtcacctcctaccagaccccacccccaatattggagattacaattcaacatgagatttgggtggggaaaaatatacaaactataccacacagtgatctgatcaagcactgaatttgagatttcttagaattatatatttgggtaagtttattaaargccttttgaagacctgatgaaatgagagagggaaatgaggcctggagaatgtaaaattagagacataattacggaatgtctcacccacttggaatttgaataattgcatctacccaaccagattagtaatgtataaaatcttctatcctgtttaaatttgagaggttacagcattatgctcctaagatctttaaaaggtc

SEQ ID NO: 699 - DNA, 401 bp, Homo sapiens
ctatgtttggctttagatttgtctagggtttacttctgttcctgagtcatctggagttccctgaaagcagagaactccatgctttcaaggttgaagcataactgattctctcacaatctggcattctgaacaggattgaaggttcagaaattctatgtactaacataggagagttaggtggcaaagctgtagtgtaaactstatgaaacaaagattacacattgtgttaggccattcttgcattgctgtaaaaaataactgagactgggtaatttatcaagaaaagaggtttaactggcccacagttctacaggctttacaagaagcctgatgctggcatctgccaggcttctggtgaagcctcagggagctttcaatcatgacggaaggcaaaggagtag

SEQ ID NO: 700 - DNA, 401 bp, Homo sapiens
cccatccccaacccttccaggaagggaacttttatagtattaggcaataagcagagccgatgaactagtaattagtcaactataaagcaaccacaaaagtaggaaagaagagaaagaagaagtaggccaaccacaaggcaattgaacatgaagtgagctaaaacatgcatcctagaaaaagaggaagtagccaagaatgamatatcatgaagaatggcaatgcctatcatgaatgtctatgccagaggggtatgtccaaccagtatggacactaagacaaaggtcaattgtgaaacctggggaaaaatctgaagctggagttagaagaagaggtattcctagggagcaaggaatgggatttatgatctgctgttcagctttattctatgtttggctttaga

SEQ ID NO: 701 - DNA, 401 bp, Homo sapiens
actacaaagtttagttaattttactaccacccacccccactttcccatccccaacccttccaggaagggaacttttatagtattaggcaataagcagagccgatgaactagtaattagtcaactataaagcaaccacaaaagtaggaaagaagagaaagaagaagtaggccaaccacaaggcaattgaacatgaagtgagmtaaaacatgcatcctagaaaaagaggaagtagccaagaatgacatatcatgaagaatggcaatgcctatcatgaatgtctatgccagaggggtatgtccaaccagtatggacactaagacaaaggtcaattgtgaaacctggggaaaaatctgaagctggagttagaagaagaggtattcctagggagcaaggaatggga

SEQ ID NO: 702 - DNA, 401 bp, Homo sapiens
aatgcccacagggatattattatctccagtgtagatctctactttaagatccagactttgttaatgttgcttgtcttaaaaagtataaatatccatgcagttgagcagtcctaaaatacaacttttcctttactcatgccctatctccagcccactacaaagtttagttaattttactaccacccacccccactttcccayccccaacccttccaggaagggaacttttatagtattaggcaataagcagagccgatgaactagtaattagtcaactataaagcaaccacaaaagtaggaaagaagagaaagaagaagtaggccaaccacaaggcaattgaacatgaagtgagctaaaacatgcatcctagaaaaagaggaagtagccaagaatgacatat

SEQ ID NO: 703 - DNA, 401 bp, Homo sapiens
ttcttggctacttcctctttttctaggatgcatgttttagctcacttcatgttcaattgccttgtggttggcctacttcttctttctcttctttcctacttttgtggttgctttatagttgactaattactagttcatcggctctgcttattgcctaatactataaaagttcccttcctggaagggttggggatgggaaartgggggtgggtggtagtaaaattaactaaactttgtagtgggctggagatagggcatgagtaaaggaaaagttgtattttaggactgctcaactgcatggatatttatactttttaagacaagcaacattaacaaagtctggatcttaaagtagagatctacactggagataataatatccctgtgggcatttaatgctc

SEQ ID NO: 704 - DNA, 401 bp, Homo sapiens
tttaaatgaccagatctaaaaaataacagttaagaattataaaaggtgtaagaactgccattcccagaccaagtttcacccttattagaacattcaagccttacattggtgtcaccatttaatctttacaagaatactctctcaaaatttcctactttatacttattttctaggtaaccatgagcattaaatgcccacagrgatattattatctccagtgtagatctctactttaagatccagactttgttaatgttgcttgtcttaaaaagtataaatatccatgcagttgagcagtcctaaaatacaacttttcctttactcatgccctatctccagcccactacaaagtttagttaattttactaccacccacccccactttcccatccccaaccctt

SEQ ID NO: 705 - DNA, 401 bp, Homo sapiens
ggtggtagtaaaattaactaaactttgtagtgggctggagatagggcatgagtaaaggaaaagttgtattttaggactgctcaactgcatggatatttatactttttaagacaagcaacattaacaaagtctggatcttaaagtagagatctacactggagataataatatccctgtgggcatttaatgctcatggttacytagaaaataagtataaagtaggaaattttgagagagtattcttgtaaagattaaatggtgacaccaatgtaaggcttgaatgttctaataagggtgaaacttggtctgggaatggcagttcttacaccttttataattcttaactgttattttttagatctggtcatttaaatctgaggacccagatatgctagtcttat

SEQ ID NO: 706 - DNA, 401 bp, Homo sapiens
taaaaataaattttaaaaaataaaacacatattaatcaggaacacatttattttggagaattcctatgaaggttgcttgaagatacttttttcctaaactttagagctatgtaagacaaagaacccattttaaatttgtgctttctataagactagcatatctgggtcctcagatttaaatgaccagatctaaaaaataayagttaagaattataaaaggtgtaagaactgccattcccagaccaagtttcacccttattagaacattcaagccttacattggtgtcaccatttaatctttacaagaatactctctcaaaatttcctactttatacttattttctaggtaaccatgagcattaaatgcccacagggatattattatctccagtgtagatct

SEQ ID NO: 707 - DNA, 401 bp, Homo sapiens
aatacagggtttaaaaattattttttaaaaaacaaaaaaagaagagaaaatgagggaaagagaatcaaatattccaaaaagtttctcctgagctagacacctccttccatttttatgtgtcatgtgagatggactattctctaagggctgaaagtaatttcataatatttaaacaactgatttaaaataattacattattkgaaaattgctaagagagtagattttaaatgtcctcaccacaaacaaatggtatgtgagatgatgacacgtatgctaaatagcttgctttagccattccacagtgtatgcatatttcaaaacatcacgttgtataccataaatacatacaattttgtcaatttaaaaattaattaaaaataaattttaaaaaataaaacac

SEQ ID NO: 708 - DNA, 401 bp, Homo sapiens
ttaatttttaaattgacaaaattgtatgtatttatggtatacaacgtgatgttttgaaatatgcatacactgtggaatggctaaagcaagctatttagcatacgtgtcatcatctcacataccatttgtttgtggtgaggacatttaaaatctactctcttagcaattttcaaataatgtaattattttaaatcagttgtktaaatattatgaaattactttcagcccttagagaatagtccatctcacatgacacataaaaatggaaggaggtgtctagctcaggagaaactttttggaatatttgattctctttccctcattttctcttctttttttgttttttaaaaaataatttttaaaccctgtattgactcctggctattattttgaatatgagt

SEQ ID NO: 709 - DNA, 401 bp, Homo sapiens
aatgttttgttttgtttttttaagaaaaaatgtttaagatttattgccctgggtaaattatagggaattgagacttaatttcacttctgaaaaaattcagaatcttgtagaaagagacaaatgaaaattatggttcaattggcattgggatagttatctacaaggctcaaattgttagcactcatattcaaaataatagcyaggagtcaatacagggtttaaaaattattttttaaaaaacaaaaaaagaagagaaaatgagggaaagagaatcaaatattccaaaaagtttctcctgagctagacacctccttccatttttatgtgtcatgtgagatggactattctctaagggctgaaagtaatttcataatatttaaacaactgatttaaaataatta

SEQ ID NO: 710 - DNA, 401 bp, Homo sapiens
ataaatataaatataaaatataagtcgaaattatttttttaaaaagagaaaaatatagggaaaaaagaatctgccttttaaacaaaagcatcgcaggcgattctcatacaggtggttagtatgggcacaactttctcataaatattacactgtgccaggggcagccctctccaaaagttatatggcatttacagagttaawgtccaccccataaagtctttccagtaaaaatgttttgttttgtttttttaagaaaaaatgtttaagatttattgccctgggtaaattatagggaattgagacttaatttcacttctgaaaaaattcagaatcttgtagaaagagacaaatgaaaattatggttcaattggcattgggatagttatctacaaggctcaaat

SEQ ID NO: 711 - DNA, 401 bp, Homo sapiens
taatatttatgagaaagttgtgcccatactaaccacctgtatgagaatcgcctgcgatgcttttgtttaaaaggcagattcttttttccctatatttttctctttttaaaaaaataatttcgacttatattttatatttatatttatattattttaaaaaataatttcaacttatattttatattcaacctacgtatgcargtttgtcatatgagtgatattgcgtggtgctgaggtttagggtacgaatgatcccatcacccagatactgagcatagtacccaatagttggtttttcaactcctgttccctccttccctccctgctctagtagtccccagtgtctgtttttgccatctttatgtctatgagtatttgatgtttagctcctacttataagt

SEQ ID NO: 712 - DNA, 401 bp, Homo sapiens
aaagactctgattcagtaattcagagaaaagcctaacaatatgctcttttacatggaatcaacctaggtgcccatcagtggtggactgcattgaggaaatgtagtatatacacactgtggaatatcatgtagccataaaaaggaatgaaactgcatcctgtgtagcaacagggatgcagctggaggccattatcctaagcraattaatgcaggaacagaaaaccaaatactccatgctctcacttataagtaggagctaaacatcaaatactcatagacataaagatggcaaaaacagacactggggactactagagcagggagggaaggagggaacaggagttgaaaaaccaactattgggtactatgctcagtatctgggtgatgggatcattcgtacc

SEQ ID NO: 713 - DNA, 401 bp, Homo sapiens
aacctctccctcccaggttcaagcgattctctagcctcagcctcctgagtagctgggattacaggcacctgccaccacgcccggataattttttgtatttttagtagagacaagatttcagtatgttggccaggctggtctggaatgcctgacctcgtgatccacccgcctcagccttccaaagtgctgagatcacaggcrtgagccaccgtgcccggccagatctttttaaataagatcttcatgtgttataatactaaggttgtctctttaatcacaaaaagccagtcctttattgcttcaaattgttcatgtgctgatatcatatgtctattgaggtctgacttactacattaacacacgctggcagaaccactgcagacattctctgtcctcctttt

SEQ ID NO: 714 - DNA, 401 bp, Homo sapiens
atatctttctttagagctagagtttagacaggaagtatgtcatagaactatggtgtgtccttcccaagtgagggatttcattagaccttgacttagaattgaatgtaatgttaatctgtaaaggcttgttgtgagggttttgcttaaatttttaggtttattaaatttttaggtttattaaaattcctaaatgatgatatwtttatcagcaaagacaaaaatactgctacttaagtagtagtcactagaaaaataatttattttgttttgttaccttaactgtcagatttaagcacattcctaccaatatttgggtttgggctctgaaacccaaaacttttgaaagctttggaaaattttgtagaagttctttcttctgtctttttttttttttttttttt

SEQ ID NO: 715 - DNA, 401 bp, Homo sapiens
ataatttattttgttttgttaccttaactgtcagatttaagcacattcctaccaatatttgggtttgggctctgaaacccaaaacttttgaaagctttggaaaattttgtagaagttctttcttctgtcttttttttttttttttttttttggatactattgccactgggatgcaagcattcataaattatgtttaaatayaaaaattaggtcggtaactctctgctcgtggattcctgaaacacaatgtacaaaaacccagaatagatcttcgtggctcactttagagcgtaacagcactgggaggtgatctgacttttcattttacacgcaagaaactgaggctggagagaaagaagggttgtctgggtcacaactgttgaggtcagcctctgggattg

SEQ ID NO: 716 - DNA, 401 bp, Homo sapiens
aaagctttggaaaattttgtagaagttctttcttctgtcttttttttttttttttttttttggatactattgccactgggatgcaagcattcataaattatgtttaaatataaaaattaggtcggtaactctctgctcgtggattcctgaaacacaatgtacaaaaacccagaatagatcttcgtggctcactttagagcrtaacagcactgggaggtgatctgacttttcattttacacgcaagaaactgaggctggagagaaagaagggttgtctgggtcacaactgttgaggtcagcctctgggattgaacttcatttcctgaattcctcatggttttccacctccattcctcagcttctgtgatccctttggtatggggctgtttcttgaaccttga

SEQ ID NO: 717 - DNA, 401 bp, Homo sapiens
tgccaccacacctgggtaatttttgtattttttgtaaagatgatgttttaccatgttgcccaggctggtctcaaactcctgggctcaagtgatctgcctacctcagcctcccaaagggctgggattacaggcgtgagacacggcgcccagcctgagctctctatattttagtttcctttaataacatgatcagttccaccyatgaatttaattaattccttatgagtttgctattaaaataaaatgagcggttattgatgcttattgtgtgtgtgacagtatcctattcaaagtatgggatacagttctttgattctctcactccatcctgtggaaggtaaacttaccttgcagatgaggatgaagttcacctaggtaatagatggacttggccaagccat

SEQ ID NO: 718 - DNA, 401 bp, Homo sapiens
tgtgtgtgtgacagtatcctattcaaagtatgggatacagttctttgattctctcactccatcctgtggaaggtaaacttaccttgcagatgaggatgaagttcacctaggtaatagatggacttggccaagccatgagactttaggtgggaaaactggaatcagaacctgagccaacctgacttcaaagcccaagttttytcctttgtgttgtctcctgagtcattttcctgtatagccccgctgaataagcattttccaaattatattttgtattgtgcaaaacttcacttccttttttattcaaagactatgtatttcatgcttcaaggttttctgagaactgcagtattctgcaaattctcaaaaaatgcttttgctctttctaagatttcatgact

SEQ ID NO: 719 - DNA, 401 bp, Homo sapiens
aggtttcttactgtcaacaatctgtttaatttgattttggttattttccacctcttcagcaaattcatgtcactgggaaccctaagataaggaatttgccttccctgatcttcctgtggggtagaagtaaaatgataagaattcaatcgttaggttgtccatggacctgcttaagagtgcaaggaggttttaaaggtcctyagaaagtcaaatgtggaccctgctgaagaagggatgcagcgttgcttacttaaaactgtagtgaacagagaagagagaaatttaactttgatattgtaggcctcaacgagtaaagaaggctttcaatcctggcaggtactaaggttttagtatcattctctttaataagtcatctgtggacaaaaatgaaaaaatcagaa

SEQ ID NO: 720 - DNA, 401 bp, Homo sapiens
acacattgaacaaattacacaggaaacctgcaaaatgttatcaagtaacaatgagctttcagctatgatcccagctaagtcttccctgcataaatgtgtaaaaagcccaccatcacatatgtgcagatcccacagtgacaaccggtgactgacaattatgttagctaatgaaagccacacgaataccattaatcagtcctytatgcaggatgtccttttgttaatagtcatgttagaagtagataaattactatggagagtctccaaaactcccccagtcatatttctctctccttgtactatctcacgaagttgattacagtcctgcaacagagtggataaactttgtgctcttccatctggtcttctcttctgagctgcactcacaaatttggctataa

SEQ ID NO: 721 - DNA, 401 bp, Homo sapiens
ttataattctacagtcttgtaaaattacacacaaacacacacacacacacacacctatatatacccatgcacttcttaaagttcttagttttcaagtttaactcagaaaaaagctaactgaccatcatatggacagcaagcccctgccaaaactagtccatttgacttggtctgtagcttatcacaaaatctcagataaaytgataaaactgtatgcattctttcttgtttgcctactcagctacttataagtcccatttatctagcagtagaaaaataaatgagagaagatataaagtattctaatatatctatgcctacatctgatctgtttgtaaatataattaacatttgaaaatcctccgtaaagcttcatccaaaaggggcttctccacgacagt

SEQ ID NO: 722 - DNA, 401 bp, Homo sapiens
ggttgctcagccagtgaactccgaccacgctgggtgaggctacatttgtaatctgttctggaaaacgggaaccatgctccattttcaacctgcactcacccactcctatttctttctagggtgtctctgtttttttgcttttaaccttgggtttgactttttccagtggacatttctaactaggaaacttgatggtttccrcttagattcccttattctccaacttaataaacatcaatacatttagaatccttcgctttccaagactctaactgatttgtggccacagtgatctttaaaattcattagactttcgtcattccattttctttattaaggcatccatatgagctgccatgtgtttgctctctttcatatatctgaatttactgattgttgtc

SEQ ID NO: 723 - DNA, 401 bp, Homo sapiens
gctttttttcccacaagcgcgtggcagaggtgggctgccacaggagtggccagtgcccgctctctgcagggcactgctatgaatcaccattttcatgaatggaaagaataggaggaggggaaaaaggacaagcagcttgttgctaatgccctcaaacaaggcaaacattttgtgaataatttttagataatatatgacggkttggatctttggttttgttgttttgaatggaatgcatagaaaaaagaacttgtttcaacaaaagtttgatctgatatcctattcatgggctgattctttctcaaatatttggcagctcatccagggcacagaaaaggcacagtgttgaatcatgttgctttcaagctctgtatcagtggaaggcactggaagggattatc

SEQ ID NO: 724 - DNA, 401 bp, Homo sapiens
ggaagaaaactgcggtaccttctaaagctcaaatgtggctttctagaacacgatgaagtggagccctacattcattatttgaccttgtttgtttttttttttttttttttttttgagacagaggagtctcactgtctcccagactggagtgcaatggcatgatctcggctcactgcatcctccgcctcctgggctcaagcrattctccagcctcagcctcctgagtagctgggactacaggcgcaagccaccatgcccagctaattttttttttttttttttttttttagtagagacgggatttcactatgttggccaggatggtctgaaactccggacctcaagtgctccacctgcctcggcctcccaaagtgctgggattacaggcatgagccaccatg

SEQ ID NO: 725 - DNA, 401 bp, Homo sapiens
tctagaacacgatgaagtggagccctacattcattatttgaccttgtttgtttttttttttttttttttttttgagacagaggagtctcactgtctcccagactggagtgcaatggcatgatctcggctcactgcatcctccgcctcctgggctcaagcgattctccagcctcagcctcctgagtagctgggactacaggygcaagccaccatgcccagctaattttttttttttttttttttttttagtagagacgggatttcactatgttggccaggatggtctgaaactccggacctcaagtgctccacctgcctcggcctcccaaagtgctgggattacaggcatgagccaccatgcctagccctgtgactttttctcaagggcttcaaacagtccc

SEQ ID NO: 726 - DNA, 401 bp, Homo sapiens
gaaattagatctcctggccgggcgtagactcacgtctatcatcccagcactttgggaggccgaggagggtggatcacctgagatcatgagttcgagaccagcctggccaacatggtgaaaccccaactctactaaaaatacaaaaattagctgggcgtggtggtgggtgcctgtagtcccagctactcaagaggttgaggsaggagaatcacttgaaccctggaggcagagattgcagtgagatgagattgtgccattgcactccagcctaagtgacggagtgagactttctcaaaaaaataaataaataaataaataaattatatctcctcttagaagtttgtccctctcttcaactgaaatctatcacagttcgacctacatggtagttgcattgatct

SEQ ID NO: 727 - DNA, 401 bp, Homo sapiens
aatctacttattctgcagattttgaatatacaatacaatattatttactataatctttctgttgtacattagatctctagacacactgattctttttttttttttttttttttttttgagacggagtttcagtcttgtcactcaggctggagttcagtggcgcaatctcggctcactgcaacttctgcctcctgggttcaratgattctcctgcctcagcctgccgagtagctgggattacaggcatgcaccaccatgcccggctaacttttatattttgggtagagacagggtttcaccatgttggccaggctggtctcaaactcctggcctcaggtgatccgcctgccttggcctcccaaagttctgggattacaggagtgagccaccatgcctggcct

SEQ ID NO: 728 - DNA, 401 bp, Homo sapiens
ttttttttttgtagtagagacggggtttcaccatgttggccagattggtcttgatctcctgacttcatgtgatccgcccgcctcggcctcccgaagtgctgggattacaggcgtgagccactgagcccggccgaaaaggactcttttttttttttttttacacagagtctcgctctgttgcccaggctggagtgcagtggygccatctcggctcactgcaagctccgcctcccgggttcacgccattctcctgcctcagcctccagagtagctgggactacaggcgcccgccaccacacccggctaatttatttatttttttctttttttcttttttgtatttttaatagagacggggtttcaccgtgttagccaggatggtctccgtctcctgacctcat

SEQ ID NO: 729 - DNA, 401 bp, Homo sapiens
gtatattagttcattctcaagctgctataaggacatgcccaagactgggtaatttataaatgaaaaaggtttgactcacagttctgcagggcgtggaggcctcaggaaacttacaatcatggcagaaggagaagcaaacacatccttcttcacatggcggctgcaaggagaagtgctgagcaaaaaggggaaaagcccctyataaaaccatcagatcctgtgagaactcactcactatcgtgagaactcacttactatcatgagaacagcatgagggtaacagcccccatcgttcaattacctccgaccaggtcccttccatgacacatggggattatggggactacaattcaagatgagatttgggtggggacacagccaaaccatatcagaaggtaagg

SEQ ID NO: 730 - DNA, 401 bp, Homo sapiens
gtgctacattagcacaggtataaatacattctctcataatttctagaggcatgtttcctaacagtacaacttctcagtgtggcaaaagagcaggtttattaacagatccaaatacctttagtctctgccaaattaagaagtcaaaagtagccgggcgcagtggctcacgcctgtaatcccagcacttcgggaggctgagaygggcggatcatgaggtcaggagatggagaccatcctggctaacacaatgaaaccccatctctactaaaaatacaaaaaaaattagctgggcatggtggcgggcgcctgtagtcccagctactcaggaggctgaggcagaagaatggcgtgaaccagggaggcggagcttgcagtaagccgagatggtgccactgcactcc

SEQ ID NO: 731 - DNA, 401 bp, Homo sapiens
attgtcatatttaatttgtttttatgcctttctcacaatattttaggttcatgagatcagagacagtattttatttgtttctctactgcctagcttattatttatttatttatttatttatttatttatttatttatttattgttatgagacagggtctcactgtcacccaggctggagtgcagtggcacatttatggctyactgcagcctcaacctcccagggctcaggtgatcctcccacctcaacctcccagtagctgggactacaggggagcaccactacacctggctaatttttgtatcttttttttttttttttttttttttgtagagacaggatttcctcatgttgcccaggctggtcttgaactcctgggctcaagcaatccactcatctcag

SEQ ID NO: 732 - DNA, 401 bp, Homo sapiens
gtgcgcagcgcggcccgggccccacgatcctcctcctgctcctcctactcctcctcctcctcggatgccgtggcctctccctccccctctccctcgcccgtcctcttcgccctgcgctctgagcgcccgttgagtcgcgcggtgcttcccctcctgggggccgccgctcacctgggcgccgagtcctaccgggcgcctacrcccagagctcagggcaagggacagcagtcccggccgcaccctcccagagtcccgggagcgcttcgctccctggcacggcccctccccagcgccttagcggctgagcccagcccgggagtgggacctgggctataggagtcgaggctgcgtgcgcgcgtgccccgcgccataagcgctttgcacgggggccgtgtgccctc

SEQ ID NO: 733 - DNA, 401 bp, Homo sapiens
ccctgtaacagcctgggtctccttggtgctggacctcccatatccagcctcttcatactgattcagctcaacagactttttctcttgaagcctgaattacaaaaccagagcttttttttttttttttttttgacggagtctcgctctgtcgccaggctggagtgcagtgggtgcaatctcggctcactgcaacctctgccwcctgggttcaagtgattcccctgcctcagacttttgagtagctgggactacaggtgtgcgccaccatgcccagctaatttttgtatttttagtagagacagggtttcaccatgttggccaggatggtgtcgatctcttgacctcgcaatccacatgcctcggcctcccaaagtgctgggattacgggcatgagccacggc

SEQ ID NO: 734 - DNA, 401 bp, Homo sapiens
agtgcagtggtgccatctcgactcactgcaagctccgcctcccgggttcacgccattctcctgcctcagcctcccgagtagctgggactacaggcgcccaccaccacgcccggctgattttttgtatttctagtagagacggggtttcaccgtgttagccaggatggtctccatctcctgacctcgtgatccgcccgcctyggcctcccaaagtgctgggattacaggcgtgagccaccgcgcccggcggggctgatattttaatatggtcacacaggccttgttatttatggtttacctgcttatgttaattccaccctgtacttgttatctttgacatgagttactctcaggatattactccccttccctttttaaagtgtaatattgttcccattgtt

SEQ ID NO: 735 - DNA, 401 bp, Homo sapiens
ctcctgggctctagtgatcctcccaccttaacctcccaagtagctgggactacaggcatataccaccatgctcggttattttttttttaattttgtagagatggggtcttgctatgctgcccaagctggtctagaacttctgggctcaaggagtccttccaccttggcctcccaaagtgctgggattataggcatgaaccrctgtgcctggcttgccagtgatggattcaaagtcgcctctgggttgtaaccaggaagcatgcctgggccagactgagagggctgaaggatgacggcatggtttcagtgtgtgagtgtgcatgtatgtacgtgtgtgtgagaatgtgggtaagtgtgtgagcctgtaaaagtgtatcatgagtagctgtctcttgggagga

SEQ ID NO: 736 - DNA, 401 bp, Homo sapiens
ttttggatataggctctgtttcatttccagaggtatctctttccctaaactcatcaagtactttttcaaccttgttgatcatcttaacagaagaatcgtaacattaagttgatttatgacaccatatctctgtagtaactctgaattgtcatcaagctattcattgaaaggatacctcttaaactttctattactaagatycccaaatgtatctattctcctctcgataaaatgagtattgtcttagacaagcagcagactgctttttaagtgaagtggattaagctgctgtgatcgtccctgtgaatgtgtatgataaatcattttgatggcctttttaaatcagagtcaccgagtcccacacagagaaacagagaatgctgtctgtcattattacaata

SEQ ID NO: 737 - DNA, 401 bp, Homo sapiens
cctgtcggtagactcagcttttccctctggctgaactcaggatcaagacaatgtgtctggccggacacggtggctcatgcctgtaatcccagcactttgggaggccaaggagacgggtggatcacctgaggtcaggagttcgtgaccagcctgaccaacatggcgaaaccccatctctactaaaaatacaaaaattagccrggcctggtggtgggtgccaataatcccagctacttgggaggctgaggcaagagaattgcttgaacctgggagggggaggttgcagtgagccgagatcacaccacttcactccagccacagaacatctcaaaaaaaaaaaaaaaaagacagcatgtctttagttctctggggctaaatgggtatgtttagatttggtgtag

SEQ ID NO: 738 - DNA, 401 bp, Homo sapiens
tcatgcctgtaatcccagcactttgggaggccaaggagacgggtggatcacctgaggtcaggagttcgtgaccagcctgaccaacatggcgaaaccccatctctactaaaaatacaaaaattagccgggcctggtggtgggtgccaataatcccagctacttgggaggctgaggcaagagaattgcttgaacctgggaggsggaggttgcagtgagccgagatcacaccacttcactccagccacagaacatctcaaaaaaaaaaaaaaaaagacagcatgtctttagttctctggggctaaatgggtatgtttagatttggtgtagcttttttggggttctttactccttttggggtcccagcttaatgtgaagagggtctactctaagactccccactt

SEQ ID NO: 739 - DNA, 401 bp, Homo sapiens
cattctggttagggggcaagagatagagactttattaaccaattaaatttctttcacaagcaatttatttgtaaaataaatccttcctctttacaatctgagagcacaaaaaattgttaaagtagctatatcctttccacttaaattccacataactccttttctatttaaattgaggctttgagaactgatcactaaatyagtatataaccaactaatattaatcagttgaacactgctcaatgggtactatgtcatttagaaagtgtttattcctaataacaacttattttccttactaaaacacttatttattaaaaattactaagatattatattctcagtactgcattacactatgttacaattttaccacttcagagctacatataaacagtgca

SEQ ID NO: 740 - DNA, 401 bp, Homo sapiens
ctgggtatatacccaaaggattataaatcatgctgctataaagacacatgcaaatgtatgtttattgtggcactattcacaatagcaaagacttggaaccaacccaaatgtccatcaatgatagactggattaagaaaatgtggcacatatagaccatggaatactatacagccacaaaaaaggatgaattcatgtccttkgtaggtacatggatgaagctggaaaccatcattctgagcaaactatcgcaaggacagaaaaccaaacactgcatgttctgactcattggtgggaactgaacaatgagaacacttggacacaggttgggaaacaccacacaccggggcctgtcatgggggtgaggggagcagggagggatagcctgaggagaaatacctaa

SEQ ID NO: 741 - DNA, 401 bp, Homo sapiens
cccaaatgctccatatagcccatttgcagtgtcccgcattgaaggtctggtgtgcagatgtgtctctttagataagggtgctgctcttttgatatctgttaaatcacaatgacttggactgtctcatgtttagaagactcagaactttgtagaaaagatgagaggaaaagcgaagagaggaaaaagagtaaggaagagcayggaaaaagaaaagcagttaaaacaaacatctaaatagaagtctctgtgctttcaaagctttgatttctcaagcaaaacatttcttaagtgaaaaagccaccacttattcattctttatttttacctttctagtgtttggtgtaaagtaggagcataaatcatgtcccattaattcattcaagagtcctaatgctaattct

SEQ ID NO: 742 - DNA, 401 bp, Homo sapiens
actggtcagccacagggctatggagagggtgagcaggattggggctggcaagaatgataagaaggaacaaacctggaaccatgattactcattcatggtcttccagtttacactctactgtgatgaccatgaaagagaaaaagaattagtaaaaaagaaaaaaatacatagacagactgccctcccagacacagaaaaatkattttcttacccataagaaaattccatcaaacatgaagattaaatagggacctaaaatctgatacctaccagtatctggacacacctggatcctgtggaggtggctctgggcagacagacaagttggaggggcagatggaattagtcaagtagggaaagtcagagtgaataagtcaggaaagccctcccacaggaagaat

SEQ ID NO: 743 - DNA, 401 bp, Homo sapiens
attgagtacaaggggacattgggagccaagtcagagatccatcccagcatctctggaggtctgtaagaagctgtggtctctctcttctctcaaggagaattgcaggagctctgatgcctcagaatttcccatgcaggcagaccaaggggagaaggagagatgctttctccaaagagctcaagaggagtcaggagaaggtgrgattagcagccagcaggaaaaggcatggacctctggtaaagcctttagagatatcttcctttctcctgcggtgggtggggggaggttgtgatggatataaagaggcctgatggaaagtctggatttacatcaagtgtgaggggaaggactctgggaaggacggcagtacaggaaagtcaacacgtcactggccttcattg

SEQ ID NO: 744 - DNA, 401 bp, Homo sapiens
ggggaaggactctgggaaggacggcagtacaggaaagtcaacacgtcactggccttcattgtattggccacccaggactgagttccggcatgaggcttgtccatgttgttttttgtttgccctatacccacagcacagatggcccatggtgtggaggaggctcatttcaaatccatcatcagtcatctaaagcgcactaargttcaacaaatgatttcaagttcatcttcagtcaacttcagtggacttttaaaattcaacaaatgatttttcaatgtctatctcaagccaggcacaggtttgttactggaaagcaacacaagatgttaacagaatggatgtttctgtcctagaagaaatgtctgcattctacaaagaaaaatggcagccaggcaccatgg

SEQ ID NO: 745 - DNA, 401 bp, Homo sapiens
ggcagtacaggaaagtcaacacgtcactggccttcattgtattggccacccaggactgagttccggcatgaggcttgtccatgttgttttttgtttgccctatacccacagcacagatggcccatggtgtggaggaggctcatttcaaatccatcatcagtcatctaaagcgcactaaagttcaacaaatgatttcaagtycatcttcagtcaacttcagtggacttttaaaattcaacaaatgatttttcaatgtctatctcaagccaggcacaggtttgttactggaaagcaacacaagatgttaacagaatggatgtttctgtcctagaagaaatgtctgcattctacaaagaaaaatggcagccaggcaccatggctcatgcctgtaatcccaacag

SEQ ID NO: 746 - DNA, 401 bp, Homo sapiens
caggactgagttccggcatgaggcttgtccatgttgttttttgtttgccctatacccacagcacagatggcccatggtgtggaggaggctcatttcaaatccatcatcagtcatctaaagcgcactaaagttcaacaaatgatttcaagttcatcttcagtcaacttcagtggacttttaaaattcaacaaatgatttttyaatgtctatctcaagccaggcacaggtttgttactggaaagcaacacaagatgttaacagaatggatgtttctgtcctagaagaaatgtctgcattctacaaagaaaaatggcagccaggcaccatggctcatgcctgtaatcccaacagtttaggaggcaaagtcaggaggatcattttaaggccaggagttcaagacc

SEQ ID NO: 747 - DNA, 401 bp, Homo sapiens
aacacaagatgttaacagaatggatgtttctgtcctagaagaaatgtctgcattctacaaagaaaaatggcagccaggcaccatggctcatgcctgtaatcccaacagtttaggaggcaaagtcaggaggatcattttaaggccaggagttcaagaccatcctgggcaacatagcaaaacctgacttctacaaaaaaaatktaaaacatagccaggcatggtggcttgcacctgtcatcccagctactcaggaggctgaagcaggacgattgcttaagcccaggagttggaggttaccatgagctatgattgcaccactgcactccagcctaaagctaggcaacagagtgagactctgtctttaaaaaaataataataattttttaaaaaaggaaaaatgg

SEQ ID NO: 748 - DNA, 401 bp, Homo sapiens
gatgttaacagaatggatgtttctgtcctagaagaaatgtctgcattctacaaagaaaaatggcagccaggcaccatggctcatgcctgtaatcccaacagtttaggaggcaaagtcaggaggatcattttaaggccaggagttcaagaccatcctgggcaacatagcaaaacctgacttctacaaaaaaaatgtaaaacrtagccaggcatggtggcttgcacctgtcatcccagctactcaggaggctgaagcaggacgattgcttaagcccaggagttggaggttaccatgagctatgattgcaccactgcactccagcctaaagctaggcaacagagtgagactctgtctttaaaaaaataataataattttttaaaaaaggaaaaatggaaactaa

SEQ ID NO: 749 - DNA, 401 bp, Homo sapiens
gaaatgtctgcattctacaaagaaaaatggcagccaggcaccatggctcatgcctgtaatcccaacagtttaggaggcaaagtcaggaggatcattttaaggccaggagttcaagaccatcctgggcaacatagcaaaacctgacttctacaaaaaaaatgtaaaacatagccaggcatggtggcttgcacctgtcatccyagctactcaggaggctgaagcaggacgattgcttaagcccaggagttggaggttaccatgagctatgattgcaccactgcactccagcctaaagctaggcaacagagtgagactctgtctttaaaaaaataataataattttttaaaaaaggaaaaatggaaactaaacaagtaaacaaaccggcataatcatttagttg

SEQ ID NO: 750 - DNA, 401 bp, Homo sapiens
caaagaaaaatggcagccaggcaccatggctcatgcctgtaatcccaacagtttaggaggcaaagtcaggaggatcattttaaggccaggagttcaagaccatcctgggcaacatagcaaaacctgacttctacaaaaaaaatgtaaaacatagccaggcatggtggcttgcacctgtcatcccagctactcaggaggctraagcaggacgattgcttaagcccaggagttggaggttaccatgagctatgattgcaccactgcactccagcctaaagctaggcaacagagtgagactctgtctttaaaaaaataataataattttttaaaaaaggaaaaatggaaactaaacaagtaaacaaaccggcataatcatttagttgctgctaaatactctgaa

SEQ ID NO: 751 - DNA, 401 bp, Homo sapiens
tggaggttaccatgagctatgattgcaccactgcactccagcctaaagctaggcaacagagtgagactctgtctttaaaaaaataataataattttttaaaaaaggaaaaatggaaactaaacaagtaaacaaaccggcataatcatttagttgctgctaaatactctgaagaagataaaatatccaggccagttcaggcrtggtggctcacgcctgtaatcccagcactttgggaggccaaggatggtggatcacaaggtcaggagttcgagaccatcctggccaacatggtgaaaccccatctctactaaaaatacaaaaattagctgggcatggtggcgggcgcctgtagtcctagctactcaggaggctgaggcagcagaatcgcttgaaaccagga

SEQ ID NO: 752 - DNA, 401 bp, Homo sapiens
gtctttaaaaaaataataataattttttaaaaaaggaaaaatggaaactaaacaagtaaacaaaccggcataatcatttagttgctgctaaatactctgaagaagataaaatatccaggccagttcaggcatggtggctcacgcctgtaatcccagcactttgggaggccaaggatggtggatcacaaggtcaggagttcragaccatcctggccaacatggtgaaaccccatctctactaaaaatacaaaaattagctgggcatggtggcgggcgcctgtagtcctagctactcaggaggctgaggcagcagaatcgcttgaaaccaggaggcagaggttgcaatgagccgagatggcgccactgcactccagcctggcaacagaactagattccatctg

SEQ ID NO: 753 - DNA, 401 bp, Homo sapiens
gaagaaagaattcagggcaagtccatacagtaaagggaaagcaagtttaagtaagtagaagaatcaaagaatggctactccatacacagagcagccctgaaggctgctagctgccattttatggttatttattgattatatgctaaacagaaggtggattattcttgcctcccatttttagaccatatagggtgacttccygacgttgccatggcatctgtgaagtgtcatggtgctggtgggagtgtagcaatgaggatgaccagcggtcactctcgtcaccatattgattttagtgggttttagccagctcctttactgcagcctgttttatttatttatttatgacccgtattttgtgccgacctcctatatatcctgtgacttagaatgcctaaccc

SEQ ID NO: 754 - DNA, 401 bp, Homo sapiens
aagggaaagcaagtttaagtaagtagaagaatcaaagaatggctactccatacacagagcagccctgaaggctgctagctgccattttatggttatttattgattatatgctaaacagaaggtggattattcttgcctcccatttttagaccatatagggtgacttcctgacgttgccatggcatctgtgaagtgtcatgrtgctggtgggagtgtagcaatgaggatgaccagcggtcactctcgtcaccatattgattttagtgggttttagccagctcctttactgcagcctgttttatttatttatttatgacccgtattttgtgccgacctcctatatatcctgtgacttagaatgcctaaccctctggcaatgcagcccagtgggtcttagcctt

SEQ ID NO: 755 - DNA, 401 bp, Homo sapiens
ctcctatatatcctgtgacttagaatgcctaaccctctggcaatgcagcccagtgggtcttagccttattttacccagttcctatttaagatggagttgctgtggttcaaatgcctctaacactggggcttataaaaagaaaattagtaagatattcaatgttcggagtgagggctgtggataaaatgtcacacccacagrcagaagagaggaaagaaaagtaagctattgaatgtttaagtgggacttaacagaggccaagatagagagagagagagacaaaaagaaatgggcagcgggagagcagcatgtctcctatgtgtgtgtgtctgtgtgtctgtgtgtgtgtgtgtgtctgtgtgtctgtgtgcgtgtgtgtgtgcatgcatgcaactctcata

SEQ ID NO: 756 - DNA, 401 bp, Homo sapiens
aacactggggcttataaaaagaaaattagtaagatattcaatgttcggagtgagggctgtggataaaatgtcacacccacagacagaagagaggaaagaaaagtaagctattgaatgtttaagtgggacttaacagaggccaagatagagagagagagagacaaaaagaaatgggcagcgggagagcagcatgtctcctaygtgtgtgtgtctgtgtgtctgtgtgtgtgtgtgtgtctgtgtgtctgtgtgcgtgtgtgtgtgcatgcatgcaactctcatagttaattggcttgtgcccatggagtcggtaagagtgcggccactaagctcacgcctgaacactgctatagggggctgggcagaggctgaatctgtctccaggaaagtaatttacagga

SEQ ID NO: 757 - DNA, 401 bp, Homo sapiens
tgcatgcaactctcatagttaattggcttgtgcccatggagtcggtaagagtgcggccactaagctcacgcctgaacactgctatagggggctgggcagaggctgaatctgtctccaggaaagtaatttacaggagtggagagaaaagctctgtgagccagcgcctggggcctctccactctctgggacagatcgctgccygtattttcagggtctgtcttcccacaagatggtcaaagcctcaggcacagggtgctcagtacgtctacaacagccacatcaagcaaaagcacagattttctttgcaccctcaacctttattattgtttcaagtcaaggaaatagggagatggctgacactgaacactggactaatttccaagatttgtgggttccaggcc

SEQ ID NO: 758 - DNA, 401 bp, Homo sapiens
aggcaaccaatgtgaccagtttcttgtgtatccttctagagatatttgattcatttctctgtgtgtgtgtgtgtgtgtgtgtgtgtgtgtgtgtgtgtctgatatggtttggctgtgtcccacctaaatctcatcttgaattgtagttcccataatccccatgtgtcatggaaaggaccaggtgagaggtaattgaatcaygggggcggttaccctcctgctgttctcatgatagtgagtgagttctcacataattggatggtttcataaggggcttctccacctcctttgctctcattcttcttcttcctgctgccatgtgaagaaggacatgtttgcctccccttccaccatgattgtaagtttcctgaggcctcccaagccctatggaattgtgtatc

SEQ ID NO: 759 - DNA, 401 bp, Homo sapiens
tcttaatttatatttcccttgacaaacaatggcttgacttacagagaaaaggcgtctcaggaacttggacaaacttgagaggagaactaaattagaggttaaataaaattcagccttcccacttcagccaggttctgccatctctagttactataaacaattgggattctgaaagtgttgaaaaaaatacattgacaatcycccccaaaggagaaaggaattataagaccattttgagcctctctgcttctcagcagtaatatggttacatgggacaaatttagcccagagaaagacacctagctactgttccactaaacccttggaaaggggagtaaatggtcccaacctttgtcatttgtctgcaaagtcctcttctaagacatgcatggaatccataa

SEQ ID NO: 760 - DNA, 401 bp, Homo sapiens
aacttttttagatactacatgtaagtgagatcatgaagtttgtctcttcctatgcctggcttatttcacttaacataatgttgtctaggttcatctatgtcgttacacgtgacaggatttcattcatatttaaggctattcctttgtgcatctatacatattttctttatccaatcacctgtcgatgggcacttaggttgrttccataacttggctattgtgaacagtgttgcaatgaacattggagtgcagatatctcttcaaataatgatcttgtgtcctttggatgtatactcagtagtgagattgctggatcatatgatagttctatttttaagtttttgaggaagttctacactgttttccataataactgtattgttttctcatatttaaattct

SEQ ID NO: 761 - DNA, 401 bp, Homo sapiens
tactagattattctaatgtaaatatagctttctcttttgcaatcagtaagtcacttgtgaggttaaactttatatgactatcttgtgacccaacaacctttcaccagataatttttaaactccgtcatctgcaacattcggctagcattctctgtaaagaactcttcttcaccctcccacctagtcttgttttgcttgtcrgtttgtttttgaaatcattagtttttaattcggtgcattaaaaggcattacgatcattattctttttgattctcaatttgtccctaatttgactagtgagaatttcttcaagctggctcctgtgtttacttgatatgttctcgttagttattgaacattttcttgggacaataaaatattctcccaaaattgcacttttc

SEQ ID NO: 762 - DNA, 401 bp, Homo sapiens
aagtgaaagacctctacaatgaaactataaaatattgatgaaaaaaaaggacacggccaggcacggtggctcacgcctgtaatcccagcactttgggaggccgaggcgggcggatcatgaggtcaggagatcaagatcatcctggctaacacagtgaaaacctgtatctactaaaaatacaaaaaaattagccaggcgtgrtggcaggcacctgcagtcccagctactcaggaggctgaggcaggagaatggcatgaacccgggaggtggagcttgcagtgagctgagattgcgccactgcactccagcctgggcgacagagcgaggctccatctccaaaaaaaaaaaaaaagaaaaagaaaaaaaggacacaaataaatagaaagatatcctgtgttcat

SEQ ID NO: 763 - DNA, 401 bp, Homo sapiens
catgttgctcagctcctgggtatggtgccttccatcacgtcagataatcaccatcttgcaagaacaagggcagggccatgaagcaggccaaatgccagaagactgtctggacctttgcaggtaccaaacacatccttgttgaatgaatcaatgcaatcatatctatggtttgtggctagggcagccagaagattggtagaytggcatcagtaagagctatctggacatataggaagtgctttccaattgttgagtcgcagagatcttaacaatccagagatattcttttgggattggataatactcacagcttccacaaaagaaccttgcaaaaagttgtggaggctttacaagactagatggagccaggcctccatcagccaattctggagaaacacttg

SEQ ID NO: 764 - DNA, 401 bp, Homo sapiens
tggcatcagtaagagctatctggacatataggaagtgctttccaattgttgagtcgcagagatcttaacaatccagagatattcttttgggattggataatactcacagcttccacaaaagaaccttgcaaaaagttgtggaggctttacaagactagatggagccaggcctccatcagccaattctggagaaacacttgyatgctggaaaactgtccctgggacaaaaaaaaaaaaaaaaaaacaacaacaacaacaacaacaacaaaacagcatgacactgataccaacattttaatcacgataattattgttagtgcttggcacaggcaaactcctaatctctgtaacctggtaagggttcttttctaaccaaggactcaaggatcaagttgtctctt

SEQ ID NO: 765 - DNA, 401 bp, Homo sapiens
gttgagtcgcagagatcttaacaatccagagatattcttttgggattggataatactcacagcttccacaaaagaaccttgcaaaaagttgtggaggctttacaagactagatggagccaggcctccatcagccaattctggagaaacacttgtatgctggaaaactgtccctgggacaaaaaaaaaaaaaaaaaaacaamaacaacaacaacaacaacaaaacagcatgacactgataccaacattttaatcacgataattattgttagtgcttggcacaggcaaactcctaatctctgtaacctggtaagggttcttttctaaccaaggactcaaggatcaagttgtctcttcatctcagcatatgcctttcatggttgccccagcaaggtaaagaaag

SEQ ID NO: 766 - DNA, 401 bp, Homo sapiens; identical to SEQ ID NO: 135

SEQ ID NO: 767 - DNA, 401 bp, Homo sapiens; identical to SEQ ID NO: 136

SEQ ID NO: 768 - DNA, 401 bp, Homo sapiens; identical to SEQ ID NO: 194

SEQ ID NO: 769 - DNA, 401 bp, Homo sapiens; identical to SEQ ID NO: 445

SEQ ID NO: 770 - DNA, 401 bp, Homo sapiens; identical to SEQ ID NO: 610

SEQ ID NO: 771 - DNA, 401 bp, Homo sapiens; identical to SEQ ID NO: 664

(19) United States
(12) Patent Application Publication
(10) Pub. No.: US 2013/0273543 A1
(43) Pub. Date: Oct. 17, 2013

(54) GENETIC VARIANTS USEFUL FOR RISK ASSESSMENT OF THYROID CANCER

(71) Applicants: Julius Gudmundsson, Reykjavik (IS); Patrick Sulem, Reykjavik (IS)

(72) Inventors: Julius Gudmundsson, Reykjavik (IS); Patrick Sulem, Reykjavik (IS)

(73) Assignee: deCODE Genetics ehf., Reykjavik (IS)

(21) Appl. No.: 13/997,037

(22) PCT Filed: Dec. 20, 2011
(86) PCT No.: PCT/IS2011/050015

§ 371 Date: Jun. 21, 2013

(30) Foreign Application Priority Data
Dec. 21, 2010 (IS) 050003

(51) Int. Cl. C12Q 1/68; G06F 19/22

(52) U.S. Cl. CPC C12Q 1/6886; G06F 19/22
USPC 435/6.11; 536/24.31; 702/20

(57) ABSTRACT

The invention discloses genetic variants that have been determined to be susceptibility variants of thyroid cancer. Methods of disease management, including methods of determining susceptibility to thyroid cancer, methods of predicting response to therapy and methods of predicting prognosis of thyroid cancer using such variants are described. The invention further relates to kits useful in the methods of the invention.

### INTRODUCTION

Thyroid carcinoma is the most common classical endocrine malignancy, and its incidence has been rising rapidly in the US as well as other industrialized countries over the past few decades. Thyroid cancers are classified histologically into four groups: papillary, follicular, medullary, and undifferentiated or anaplastic thyroid carcinomas (DeLellis, R. A., J Surg Oncol, 94, 662 (2006)). In 2008, it is expected that over 37,000 new cases will be diagnosed in the US, about 75% of them being females (the ratio of males to females is 1:3.2) (Jemal, A., et al., Cancer statistics, 2008. CA Cancer J Clin, 58: 71-96, (2008)). If diagnosed at an early stage, thyroid cancer is a well manageable disease with a 5-year survival rate of 97% among all patients, yet about 1,600 individuals were expected to die from this disease in 2008 in the US (Jemal, A., et al., Cancer statistics, 2008. CA Cancer J Clin, 58: 71-96, (2008)). Survival rate is poorer (˜40%) among individuals that are diagnosed with a more advanced disease; i.e. individuals with large, invasive tumors and/or distant metastases have a 5-year survival rate of ≈40% (Sherman, S. I., et al., 3rd, Cancer, 83, 1012 (1998), Kondo, T., Ezzat, S., and Asa, S. L., Nat Rev Cancer, 6, 292 (2006)). For radioiodine-resistant metastatic disease there is no effective treatment and the 10-year survival rate among these patients is less than 15% (Durante, C., et al., J Clin Endocrinol Metab, 91, 2892 (2006)).

Although relatively rare (1% of all malignancies in the US), the incidence of thyroid cancer more than doubled between 1984 and 2004 in the US (SEER web report; Ries L, Melbert D, Krapcho M et al (2007) SEER cancer statistics review, 1975-2004. National Cancer Institute, Bethesda, Md., http://seer.cancer.gov/csr/1975_{—}2004/, based on November 2006 SEER data submission). Between 1995 and 2004, thyroid cancer was the third fastest growing cancer diagnosis, behind only peritoneum, omentum, and mesentery cancers and “other” digestive cancers [SEER web report]. Similarly dramatic increases in thyroid cancer incidence have also been observed in Canada, Australia, Israel, and several European countries (Liu, S., et al., Br J Cancer, 85, 1335 (2001), Burgess, J. R., Thyroid, 12, 141 (2002), Lubina, A., et al., Thyroid, 16, 1033 (2006), Colonna, M., et al., Eur J Cancer, 38, 1762 (2002), Leenhardt, L., et al., Thyroid, 14, 1056 (2004), Reynolds, R. M., et al., Clin Endocrinol (Oxf), 62, 156 (2005), Smailyte, G., et al., BMC Cancer, 6, 284 (2006)).

Thus, there is a need for better understanding of the molecular causes of thyroid cancer progression to develop new diagnostic tools and better treatment options. The present invention provides thyroid cancer susceptibility variants and their use in various diagnostic applications.

### SUMMARY OF THE INVENTION

The present invention relates to methods of risk management of thyroid cancer, based on the discovery that certain genetic variants are correlated with risk of thyroid cancer. Thus, the invention includes methods of determining an increased susceptibility or increased risk of thyroid cancer, as well as methods of determining a decreased susceptibility of thyroid cancer, through evaluation of certain markers that have been found to be correlated with susceptibility of thyroid cancer in humans. Other aspects of the invention relate to methods of assessing prognosis of individuals diagnosed with thyroid cancer, methods of assessing the probability of response to a therapeutic agents or therapy for thyroid cancer, as well as methods of monitoring progress of treatment of individuals diagnosed with thyroid cancer.

In one aspect, the invention relates to a method of determining a susceptibility to Thyroid Cancer, the method comprising analyzing nucleic acid sequence data from a human individual for at least one polymorphic marker selected from the group consisting of rs7005606 and rs966423, and correlated markers in linkage disequilibrium therewith, wherein different alleles of the at least one polymorphic marker are associated with different susceptibilities to Thyroid Cancer in humans, and determining a susceptibility to Thyroid Cancer from the nucleic acid sequence data.

In another aspect, the invention relates to a method of determining a susceptibility to thyroid cancer in a human individual, the method comprising determining the presence or absence of at least one allele of at least one polymorphic marker selected from the group consisting of the markers rs7005606 and rs966423, and markers in linkage disequilibrium therewith, in a nucleic acid sample obtained from the individual, wherein the presence of the at least one allele is indicative of a susceptibility to thyroid cancer.

The invention also relates to a method of determining a susceptibility to thyroid cancer, the method comprising determining the presence or absence of at least one allele of at least one polymorphic marker selected from the group consisting of the markers rs7005606 and rs966423, and markers in linkage disequilibrium therewith, wherein the determination of the presence of the at least one allele is indicative of a susceptibility to thyroid cancer.

In another aspect the invention further relates to a method for determining a susceptibility to thyroid cancer in a human individual, comprising determining whether at least one allele of at least one polymorphic marker is present in a nucleic acid sample obtained from the individual, or in a genotype dataset derived from the individual, wherein the at least one polymorphic marker is selected from the group consisting of markers rs7005606 and rs966423, and markers in linkage disequilibrium therewith, and wherein the presence of the at least one allele is indicative of a susceptibility to thyroid cancer for the individual.

The invention also provides a method of identification of a marker for use in assessing susceptibility to Thyroid Cancer in human individuals, the method comprising (i) identifying at least one polymorphic marker in linkage disequilibrium with rs7005606 or rs966423; (ii) obtaining sequence information about the at least one polymorphic marker in a group of individuals diagnosed with Thyroid Cancer; and (iii) obtaining sequence information about the at least one polymorphic marker in a group of control individuals; wherein determination of a significant difference in frequency of at least one allele in the at least one polymorphism in individuals diagnosed with Thyroid Cancer as compared with the frequency of the at least one allele in the control group is indicative of the at least one polymorphism being useful for assessing susceptibility to Thyroid Cancer.

Further provided are prognostic methods and methods of assessing probability to treatment. Thus, a further aspect of the invention relates to a method of predicting prognosis of an individual diagnosed with Thyroid Cancer, the method comprising obtaining sequence data about a human individual about at least one polymorphic marker selected from the group consisting of rs7005606 or rs966423, and markers in linkage disequilibrium therewith, wherein different alleles of the at least one polymorphic marker are associated with different susceptibilities to Thyroid Cancer in humans, and predicting prognosis of the Thyroid Cancer from the sequence data. Also provided is a method of assessing probability of response of a human individual to a therapeutic agent for preventing, treating and/or ameliorating symptoms associated with Thyroid Cancer, comprising obtaining sequence data about a human individual identifying at least one allele of at least one polymorphic marker selected from the group consisting of rs7005606 or rs966423, and markers in linkage disequilibrium therewith, wherein different alleles of the at least one polymorphic marker are associated with different probabilities of response to the therapeutic agent in humans, and determining the probability of a positive response to the therapeutic agent from the sequence data.

The invention also provides kits. In one such aspect, the invention relates to a kit for assessing susceptibility to Thyroid Cancer in human individuals, the kit comprising reagents for selectively detecting at least one at-risk variant for Thyroid Cancer in the individual, wherein the at least one at-risk variant is selected from the group consisting of rs7005606 or rs966423, and markers in linkage disequilibrium therewith, and a collection of data comprising correlation data between the at least one at-risk variant and susceptibility to Thyroid Cancer.

Further provided is the use of an oligonucleotide probe in the manufacture of a diagnostic reagent for diagnosing and/or assessing a susceptibility to Thyroid Cancer, wherein the probe is capable of hybridizing to a nucleic acid segment with sequence as set forth in any one of SEQ ID NO:1-771, and wherein the nucleic acid segment is 15-400 nucleotides in length.

The invention also provides computer-implemented applications. In one such application, the invention relates to an apparatus for determining a susceptibility to Thyroid Cancer in a human individual, comprising a processor and a computer readable memory having computer executable instructions adapted to be executed on the processor to analyze information for at least one human individual with respect to at least one marker selected from the group consisting of rs7005606 or rs966423, and markers in linkage disequilibrium therewith, and generate an output based on the marker or amino acid information, wherein the output comprises at least one measure of susceptibility to Thyroid Cancer for the human individual.

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein.

### BRIEF DESCRIPTION OF THE DRAWINGS

The foregoing and other objects, features and advantages of the invention will be apparent from the following more particular description of preferred embodiments of the invention.

FIG. 1 provides a diagram illustrating a computer-implemented system utilizing risk variants as described herein.

FIG. 2 provides another diagram illustrating a computer-implemented system utilizing risk variants as described herein.

FIG. 3 shows an exemplary system for determining risk of thyroid cancer as described further herein.

FIG. 4 shows a system for selecting a treatment protocol for a subject diagnosed with thyroid cancer.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.

Sequence conucleotide ambiguity as described herein, including sequence listing, is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A, C, G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles. Haplotypes are described herein in the context of the marker name and the allele of the marker in that haplotype, e.g., “3 rs7005606” refers to the 3 allele of marker rs7005606 being in the haplotype, and is equivalent to “rs7005606 allele 3”. Furthermore, allelic codes in haplotypes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers and/or haplotypes of the invention as described herein may be characteristic of increased susceptibility (i.e., increased risk) of thyroid cancer, as characterized by a relative risk (RR) or odds ratio (OR) of greater than one for the particular allele or haplotype. Alternatively, the markers and/or haplotypes of the invention are characteristic of decreased susceptibility (i.e., decreased risk) of thyroid cancer, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or they can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “thyroid cancer therapeutic agent” refers to an agent that can be used to ameliorate or prevent symptoms associated with thyroid cancer.

The term “thyroid cancer-associated nucleic acid”, as described herein, refers to a nucleic acid that has been found to be associated to thyroid cancer. This includes, but is not limited to, the markers and haplotypes described herein and markers and haplotypes in strong linkage disequilibrium (LD) therewith. In one embodiment, a thyroid cancer-associated nucleic acid refers to a genomic region, such as an LD-block, found to be associated with risk of thyroid cancer through at least one polymorphic marker located within the region or LD block.

### Variants Associated with Risk of Thyroid Cancer

The present inventors have identified genomic regions that contain markers that correlate with risk of thyroid cancer. On chromosome 2q35, a region exemplified by markers rs966423, rs12990503 and rs737308 has been found to correlate with risk of thyroid cancer. Further, a region on chromosome 8p12, exemplified by markers rs7005606 and rs2439302, has been found to associate with risk of thyroid cancer. Markers in these regions are useful for assessing genetic risk of thyroid cancer in human individuals.

As a consequence, the present invention in one aspect provides a method of determining a susceptibility to Thyroid Cancer, the method comprising analyzing nucleic acid sequence data from a human individual for at least one polymorphic marker selected from the group consisting of rs7005606 and rs966423, and correlated markers in linkage disequilibrium therewith, wherein different alleles of the at least one polymorphic marker are associated with different susceptibilities to Thyroid Cancer in humans, and determining a susceptibility to Thyroid Cancer from the nucleic acid sequence data.

In one preferred embodiment, suitable markers are selected from the group consisting of markers in linkage disequilibrium with rs7005606 characterized by values of the linkage disequilibrium measure r² of greater than 0.2. In another preferred embodiment, suitable markers are selected from the group consisting of markers in linkage disequilibrium with rs966423 characterized by values of the linkage disequilibrium measure r² of greater than 0.2. In certain other preferred embodiment, suitable polymorphic markers are selected from markers that are in linkage disequilibrium with rs7005606 and/or rs966423 characterized by values of the linkage disequilibrium measure r² of greater than 0.8.

Certain alleles of risk variants of thyroid cancer are predictive of increased risk (increased susceptibility) of thyroid cancer. Thus, the G allele of rs7005606, the C allele of rs966423, the G allele of rs737308, the C allele of rs12990503 and the C allele of rs2439302 (G allele of rs2439302 on the complementary strand) are all alleles indicative of increased risk of thyroid cancer. Other exemplary risk alleles of thyroid cancer are listed in the Tables herein. For example, Tables 1 and 8 list markers on chromosome 2q35 that are predictive of thyroid cancer, and the risk allele predictive of increased risk of thyroid cancer for each marker. Further, Tables 2 and 7 list markers on chromosome 8p12 that are predictive of thyroid cancer, and the risk allele of each marker that is predictive of increased risk of thyroid cancer. Any of the markers listed in these tables are thus informative of predicting risk of thyroid cancer, and are therefore within scope of the present invention. The markers on chromosome 2q35 are furthermore all correlated, which means that they are indicative of the same underlying genetic predisposition. Likewise, the markers on chromosome 8p12 are all correlated and thus also indicative of the same genetic predisposition.

In certain embodiments, determination of the presence of at least one allele selected from the group consisting of the G allele of rs7005606, the C allele of rs966423, the G allele of rs737308, the C allele of rs12990503 and the C allele of rs2439302 is indicative of increased risk of thyroid cancer for the individual. In another embodiment, the G allele of rs57481445, the T allele of rs16857609, the T allele of rs16857611, the C allele of rs12990503, the A allele of rs13388294, the T allele of rs3821098, the C allele of rs11693806 and the C allele of rs11680689 are indicative of increased risk of thyroid cancer.

Determination of the absence of risk alleles is indicative that the individual does not have the increased risk conferred by the allele. In certain embodiments, alleles indicative of increased risk of thyroid cancer are selected from the group consisting of the marker alleles listed in Table 7 and Table 8 having a risk (odds ratio) of greater than one. In certain other embodiments, alleles indicative of risk of thyroid cancer are selected from the group consisting of the marker alleles listed in Table 1 that are correlated with the at-risk C allele of rs966423. In certain other embodiments, alleles indicative of risk of thyroid cancer are selected from the group consisting of the marker alleles listed in Table 2 that are correlated with the at-risk G allele of rs7005606.

As will be described in more detail in the below, the skilled person will appreciate that marker alleles in linkage disequilibrium with any one of these at-risk alleles of thyroid cancer are also predictive of increased risk of thyroid cancer, and may thus also be suitably selected for use in the methods of the invention.

The allele that is detected can suitably be the allele of the complementary strand of DNA, such that the nucleic acid sequence data includes the identification of at least one allele which is complementary to any of the alleles of the polymorphic markers referenced above. For example, the allele that is detected may be the complementary C allele of the at-risk G allele of rs7005606. The allele that is detected may also be the complementary G allele of the at-risk C allele of rs966423.

In certain embodiments, the nucleic acid sequence data is obtained from a biological sample containing nucleic acid from the human individual. The nucleic acids sequence may suitably be obtained using a method that comprises at least one procedure selected from (i) amplification of nucleic acid from the biological sample; (ii) hybridization assay using a nucleic acid probe and nucleic acid from the biological sample; (iii) hybridization assay using a nucleic acid probe and nucleic acid obtained by amplification of the biological sample, and (iv) nucleic acid sequencing, in particular high-throughput sequencing. The nucleic acid sequence data may also be obtained from a preexisting record. For example, the preexisting record may comprise a genotype dataset for at least one polymorphic marker. In certain embodiments, the determining comprises comparing the sequence data to a database containing correlation data between the at least one polymorphic marker and susceptibility to the condition.

In another aspect, a method is provided that comprises (1) obtaining a sample containing nucleic acid from a human individual; (2) obtaining nucleic acid sequence data about at least one polymorphic marker in the sample, wherein different alleles of the at least one marker are associated with different susceptibilities of thyroid cancer in humans; (3) analyzing the nucleic acid sequence data about the at least one marker; and (4) determining a risk of thyroid cancer from the nucleic acid sequence data. In certain embodiments, the analyzing comprises determining the presence or absence of at least one allele of the at least one polymorphic marker.

It is contemplated that in certain embodiments of the invention, it may be convenient to prepare a report of results of risk assessment. Thus, certain embodiments of the methods of the invention comprise a further step of preparing a report containing results from the determination, wherein said report is written in a computer readable medium, printed on paper, or displayed on a visual display. In certain embodiments, it may be convenient to report results of susceptibility to at least one entity selected from the group consisting of the individual, a guardian of the individual, a genetic service provider, a physician, a medical organization, and a medical insurer.

In another aspect, the invention relates to a method of determining a susceptibility to thyroid cancer in a human individual, comprising determining whether at least one at-risk allele in at least one polymorphic marker is present in a genotype dataset derived from the individual, wherein the at least one polymorphic marker is selected from the group consisting of the markers rs7005606 and rs966423, and markers in linkage disequilibrium therewith, and wherein determination of the presence of the at least one at-risk allele is indicative of increased susceptibility to thyroid cancer in the individual.

A genotype dataset derived from an individual is in the present context a collection of genotype data that is indicative of the genetic status of the individual for particular genetic markers. The dataset is derived from the individual in the sense that the dataset has been generated using genetic material from the individual, or by other methods available for determining genotypes at particular genetic markers (e.g., imputation methods). The genotype dataset comprises in one embodiment information about marker identity and the allelic status of the individual for at least one allele of a marker, i.e. information about the identity of at least one allele of the marker in the individual. The genotype dataset may comprise allelic information (information about allelic status) about one or more marker, including two or more markers, three or more markers, five or more markers, ten or more markers, one hundred or more markers, and so on. In some embodiments, the genotype dataset comprises genotype information from a whole-genome assessment of the individual, which may include hundreds of thousands of markers, or even one million or more markers spanning the entire genome of the individual.

Another aspect of the invention relates to a method of determining a susceptibility to thyroid cancer in a human individual, the method comprising obtaining nucleic acid sequence data about a human individual identifying at least one allele of at least one polymorphic marker selected from the group consisting of the markers rs7005606 and rs966423, and markers in linkage disequilibrium therewith, wherein different alleles of the at least one polymorphic marker are associated with different susceptibilities to thyroid cancer in humans, and determining a susceptibility to thyroid cancer from the nucleic acid sequence data.

In certain embodiments, the sequence data is analyzed using a computer processor to determine a susceptibility to thyroid cancer from the sequence data. Alternatively, the sequence data is transformed into a risk measure of thyroid cancer for the individual.

Obtaining nucleic acid sequence data may comprise steps of obtaining a biological sample from the human individual and transforming the sample to analyze sequence of the at least one polymorphic marker in the sample. Alternatively, sequence data obtained from a dataset may be transformed. Any suitable method known to the skilled artisan for obtaining a biological sample may be used, for example using the methods described herein. Likewise, transforming the sample to analyze sequence may be performed using any method known to the skilled artisan, including the methods described herein for determining disease risk.

### Assessment of Other Biomarkers for Thyroid Cancer

Certain embodiments of the invention further comprise assessing the quantitative levels of a biomarker for thyroid cancer. For example, the levels of a biomarker may be determined in concert with analysis of particular genetic markers. Alternatively, biomarker levels are determined at a different point in time, but results of such determination are used together with results from sequencing analysis for particular polymorphic markers. The biomarker may in some embodiments be assessed in a biological sample from the individual. In some embodiments, the sample is a blood sample. The blood sample is in some embodiments a serum sample. In preferred embodiments, the biomarker is selected from the group consisting of thyroid stimulating hormone (TSH), thyroxine (T4) and thriiodothyronine (T3). In certain embodiments, determination of an abnormal level of the biomarker is indicative of an abnormal thyroid function in the individual, which may in turn be indicative of an increased risk of thyroid cancer in the individual. The abnormal level can be an increased level or the abnormal level can be a decreased level. In certain embodiments, the determination of an abnormal level is determined based on determination of a deviation from the average levels of the biomarker in the population. In one embodiment, abnormal levels of TSH are measurements of less than 0.2 mIU/L and/or greater than 10 mIU/L. In another embodiment, abnormal levels of TSH are measurements of less than 0.3 mIU/L and/or greater than 3.0 mIU/L. In another embodiment, abnormal levels of T₃ (free T₃) are less than 70 ng/dL and/or greater than 205 ng/dL. In another embodiment, abnormal levels of T₄ (free T₄) are less than 0.8 ng/dL and/or greater than 2.7 ng/d L.

The markers conferring risk of thyroid cancer, as described herein, can be combined with other genetic markers for thyroid cancer. Such markers are typically not in linkage disequilibrium with rs7005606 or rs966423, or other markers in linkage disequilibrium with those markers. Any of the methods described herein can be practiced by combining the genetic risk factors described herein with additional genetic risk factors for thyroid cancer.

Thus, in certain embodiments, a further step is included, comprising determining whether at least one at-risk allele of at least one at-risk variant for thyroid cancer not in linkage disequilibrium with any one of the markers rs7005606 or rs966423, or markers in linkage disequilibrium therewith, is present in a sample comprising genomic DNA from a human individual or a genotype dataset derived from a human individual. In other words, genetic markers in other locations in the genome can be useful in combination with the markers of the present invention, so as to determine overall risk of thyroid cancer based on multiple genetic variants. Selection of markers that are not in linkage disequilibrium (not in LD) can be based on a suitable measure for linkage disequilibrium, as described further herein. In certain embodiments, markers that are not in linkage disequilibrium have values of the LD measure r² correlating the markers of less than 0.2. In certain other embodiments, markers that are not in LD have values for r² correlating the markers of less than 0.15, including less than 0.10, less than 0.05, less than 0.02 and less than 0.01. Other suitable numerical values for establishing that markers are not in LD are contemplated, including values bridging any of the above-mentioned values.

In one embodiment, assessment of one or more of the markers described herein is combined with assessment of at least one marker selected from the group consisting of marker rs965513 on chromosome 9q22 and marker rs944289 on chromosome 14q13, or a marker in linkage disequilibrium therewith, to establish overall risk. In certain such embodiments, determination of the presence of the A allele of rs965513 and/or the T allele of rs944289 is indicative of increased risk of thyroid cancer. In one embodiment, the A allele of rs965513 is an at-risk allele of thyroid cancer, and the T allele of rs944289 is an at-risk allele of thyroid cancer.

In certain embodiments, multiple markers as described herein are determined to determine overall risk of thyroid cancer. Thus, in certain embodiments, an additional step is included, the step comprising determining whether at least one allele in each of at least two polymorphic markers is present in a sample comprising genomic DNA from a human individual or a genotype dataset derived from a human individual, wherein the presence of the at least one allele in the at least two polymorphic markers is indicative of an increased susceptibility to thyroid cancer.

The genetic markers of the invention can also be combined with non-genetic information to establish overall risk for an individual. Thus, in certain embodiments, a further step is included, comprising analyzing non-genetic information to make risk assessment, diagnosis, or prognosis of the individual. The non-genetic information can be any information pertaining to the disease status of the individual or other information that can influence the estimate of overall risk of thyroid cancer for the individual. In one embodiment, the non-genetic information is selected from age, gender, ethnicity, socioeconomic status, previous disease diagnosis, medical history of subject, family history of thyroid cancer, biochemical measurements, and clinical measurements.

### Assays

The invention also provides assays for determining susceptibility to thyroid cancer. In one such aspect, the invention provides an assay for determining a susceptibility to thyroid cancer in a human subject, the assay comprising steps of (i) obtaining a nucleic acid sample from the human subject; (ii) assaying the nucleic acid sample to determine the presence or absence of at least one allele of at least one polymorphic marker conferring increased susceptibility to thyroid cancer in humans, and (iii) determining a susceptibility to thyroid cancer for the human subject from the presence or absence of the at least one allele; wherein the at least one polymorphic marker is selected from the group consisting of rs7005606 and rs966423, and markers correlated therewith, and wherein determination of the presence of the at least one allele is indicative of an increased susceptibility to thyroid cancer for the subject.

Correlated markers useful in the assays may include any of the surrogate markers described in the above as useful in the methods described herein. Thus, in certain embodiments, useful surrogate markers correlated with rs7005606 are selected from the group consisting of the markers set forth in Table 2 and Table 7 herein. Further, in certain embodiments, useful surrogate markers correlated with rs966423 are selected from the group consisting of the markers set forth in Table 1 and Table 8 herein.

### Obtaining Nucleic Acid Sequence Data

Sequence data can be nucleic acid sequence data, which may be obtained by means known in the art. Sequence data is suitably obtained from a biological sample of genomic DNA, RNA, or cDNA (a “test sample”) from an individual (“test subject). For example, nucleic acid sequence data may be obtained through direct analysis of the sequence of the polymorphic position (allele) of a polymorphic marker. Suitable methods, some of which are described herein, include, for instance, whole genome sequencing methods, whole genome analysis using SNP chips (e.g., Infinium HD BeadChip), cloning for polymorphisms, non-radioactive PCR-single strand conformation polymorphism analysis, denaturing high pressure liquid chromatography (DHPLC), DNA hybridization, computational analysis, single-stranded conformational polymorphism (SSCP), restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis; heteroduplex analysis, chemical mismatch cleavage (CMC), RNase protection assays, use of polypeptides that recognize nucleotide mismatches, such as E. coli mutS protein, allele-specific PCR, and direct manual and automated sequencing. These and other methods are described in the art (see, for instance, Li et al., Nucleic Acids Research, 28(2): e1 (i-v) (2000); Liu et al., Biochem Cell Bio 80:17-22 (2000); and Burczak et al., Polymorphism Detection and Analysis, Eaton Publishing, 2000; Sheffield et al., Proc. Natl. Acad. Sci. USA, 86:232-236 (1989); Orita et al., Proc. Natl. Acad. Sci. USA, 86:2766-2770 (1989); Flavell et al., Cell, 15:25-41 (1978); Geever et al., Proc. Natl. Acad. Sci. USA, 78:5081-5085 (1981); Cotton et al., Proc. Natl. Acad. Sci. USA, 85:4397-4401 (1985); Myers et al., Science 230:1242-1246 (1985); Church and Gilbert, Proc. Natl. Acad. Sci. USA, 81:1991-1995 (1988); Sanger et al., Proc. Natl. Acad. Sci. USA, 74:5463-5467 (1977); and Beavis et al., U.S. Pat. No. 5,288,644).

Recent technological advances have resulted in technologies that allow massive parallel sequencing to be performed in relatively condensed format. These technologies share sequencing-by-synthesis principle for generating sequence information, with different technological solutions implemented for extending, tagging and detecting sequences. Exemplary technologies include 454 pyrosequencing technology (Nyren, P. et al. Anal Biochem 208:171-75 (1993); http://www.454.com), Illumina Solexa sequencing technology (Bentley, D. R. Curr Opin Genet Dev 16:545-52 (2006); http://www.illumina.com), and the SOLID technology developed by Applied Biosystems (ABI) (http://www.appliedbiosystems.com; see also Strausberg, R. L., et al. Drug Disc Today 13:569-77 (2008)). Other sequencing technologies include those developed by Pacific Biosciences (http://www.pacificbiosciences.com), Complete Genomics (http://www.completegenomics.com), Intelligen Bio-Systems (http://www.intelligentbiosystems.com), Genome Corp (http://www.genomecorp.com), ION Torrent Systems (http://www.iontorrent.com) and Helicos Biosciences (http://www.helicosbio.som). It is contemplated that sequence data useful for performing the present invention may be obtained by any such sequencing method, or other sequencing methods that are developed or made available. Thus, any sequence method that provides the allelic identity at particular polymorphic sites (e.g., the absence or presence of particular alleles at particular polymorphic sites) is useful in the methods described and claimed herein.

Alternatively, hybridization methods may be used (see Current Protocols in Molecular Biology, Ausubel et al., eds., John Wiley & Sons, including all supplements). For example, a biological sample of genomic DNA, RNA, or cDNA (a “test sample”) may be obtained from a test subject.

The subject can be an adult, child, or fetus. The DNA, RNA, or cDNA sample is then examined. The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to Thyroid Cancer, a hybridization sample can be formed by contacting the test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 10, 15, 30, 50, 100, 250 or 500 nucleotides in length that is sufficient to specifically hybridize under stringent conditions to appropriate mRNA or genomic DNA. In certain embodiments, the nucleic acid probe is capable of hybridizing to a nucleic acid with sequence as set forth in any one of SEQ ID NO:1-771. Hybridization can be performed by methods well known to the person skilled in the art (see, e.g., Current Protocols in Molecular Biology, Ausubel et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen et al., Bioconjug. Chem. 5:3-7 (1994)). The PNA probe can be designed to specifically hybridize to a molecule in a sample suspected of containing one or more of the marker alleles that are associated with risk of thyroid cancer.

In one embodiment of the invention, a test sample containing genomic DNA obtained from the subject is collected and the polymerase chain reaction (PCR) is used to amplify a fragment comprising one or more polymorphic marker. As described herein, identification of particular marker alleles can be accomplished using a variety of methods. In another embodiment, determination of a susceptibility is accomplished by expression analysis, for example using quantitative PCR (kinetic thermal cycling). This technique can, for example, utilize commercially available technologies, such as TaqMan® (Applied Biosystems, Foster City, Calif.). The technique can for example assess the presence of an alteration in the expression or composition of a polypeptide or splicing variant(s) that is encoded by a nucleic acid associated described herein. Alternatively, this technique may assess expression levels of genes or particular splice variants of genes, that are affected by one or more of the variants described herein. Further, the expression of the variant(s) can be quantified as physically or functionally different.

Allele-specific oligonucleotides can also be used to detect the presence of a particular allele in a nucleic acid. An “allele-specific oligonucleotide” (also referred to herein as an “allele-specific oligonucleotide probe”) is an oligonucleotide of any suitable size, for example an oligonucleotide of approximately 10-50 base pairs or approximately 15-30 base pairs, that specifically hybridizes to a nucleic acid which contains a specific allele at a polymorphic site (e.g., a polymorphic marker). An allele-specific oligonucleotide probe that is specific for one or more particular alleles at polymorphic markers can be prepared using standard methods (see, e.g., Current Protocols in Molecular Biology, supra). PCR can be used to amplify the desired region. Specific hybridization of an allele-specific oligonucleotide probe to DNA from a subject is indicative of the presence of a specific allele at a polymorphic site (see, e.g., Gibbs et al., Nucleic Acids Res. 17:2437-2448 (1989) and WO 93/22456).

With the addition of analogs such as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tm) of 64° C. and 74° C. when in complex with complementary DNA or RNA, respectively, as opposed to 28° C. for both DNA and RNA for the corresponding DNA nonamer. Substantial increases in Tm are also obtained when LNA monomers are used in combination with standard DNA or RNA monomers. For primers and probes, depending on where the LNA monomers are included (e.g., the 3′ end, the 5′ end, or in the middle), the Tm could be increased considerably. It is therefore contemplated that in certain embodiments, LNAs are used to detect particular alleles at polymorphic sites associated with particular vascular conditions, as described herein.

In certain embodiments, arrays of oligonucleotide probes that are complementary to target nucleic acid sequence segments from a subject can be used to identify polymorphisms in a nucleic acid. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier et al., Adv Biochem Eng Biotechnol 109:433-53 (2008); Hoheisel, Nat Rev Genet. 7:200-10 (2006); Fan et al., Methods Enzymol 410:57-73 (2006); Raqoussis & Elvidge, Expert Rev Mol Diagn 6:145-52 (2006); Mockler et al., Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Also, standard techniques for genotyping can be used to detect particular marker alleles, such as fluorescence-based techniques (e.g., Chen et al., Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave).

Suitable biological sample in the methods described herein can be any sample containing nucleic acid (e.g., genomic DNA) and/or protein from the human individual. For example, the biological sample can be a blood sample, a serum sample, a leukapheresis sample, an amniotic fluid sample, a cerbrospinal fluid sample, a hair sample, a tissue sample from skin, muscle, buccal, or conjuctival mucosa, placenta, gastrointestinal tract, or other organs, a semen sample, a urine sample, a saliva sample, a nail sample, a tooth sample, and the like. Preferably, the sample is a blood sample, a salive sample or a buccal swab.

### Protein Analysis

Missense nucleic acid variations may lead to an altered amino acid sequence, as compared to the non-variant (e.g., wild-type) protein, due to one or more amino acid substitutions, deletions, or insertions, or truncation (due to, e.g., splice variation). In such instances, detection of the amino acid substitution of the variant protein may be useful. This way, nucleic acid sequence data may be obtained through indirect analysis of the nucleic acid sequence of the allele of the polymorphic marker, i.e. by detecting a protein variation. Methods of detecting variant proteins are known in the art. For example, direct amino acid sequencing of the variant protein followed by comparison to a reference amino acid sequence can be used. Alternatively, SDS-PAGE followed by gel staining can be used to detect variant proteins of different molecular weights. Also, Immunoassays, e.g., immunofluorescent immunoassays, immunoprecipitations, radioimmunoasays, ELISA, and Western blotting, in which an antibody specific for an epitope comprising the variant sequence among the variant protein and non-variant or wild-type protein can be used. In certain embodiments of the present invention, the R721W substitution is detected in a protein sample. The detection may be suitably performed using any of the methods described in the above.

In some cases, a variant protein has altered (e.g., upregulated or downregulated) biological activity, in comparison to the non-variant or wild-type protein. The biological activity can be, for example, a binding activity or enzymatic activity. In this instance, altered biological activity may be used to detect a variation in protein encoded by a nucleic acid sequence variation. Methods of detecting binding activity and enzymatic activity are known in the art and include, for instance, ELISA, competitive binding assays, quantitative binding assays using instruments such as, for example, a Biacore® 3000 instrument, chromatographic assays, e.g., HPLC and TLC.

Alternatively or additionally, a protein variation encoded by a genetic variation could lead to an altered expression level, e.g., an increased expression level of an mRNA or protein, a decreased expression level of an mRNA or protein. In such instances, nucleic acid sequence data about the allele of the polymorphic marker, or protein sequence data about the protein variation, can be obtained through detection of the altered expression level. Methods of detecting expression levels are known in the art. For example, ELISA, radioimmunoassays, immunofluorescence, and Western blotting can be used to compare the expression of protein levels. Alternatively, Northern blotting can be used to compare the levels of mRNA. These processes are described in Sambrook et al., Molecular Cloning: A Laboratory Manual, 3^{rd} ed. Cold Spring Harbor Laboratory Press, Cold Spring Harbor, N.Y. (2001).

Any of these methods may be performed using a nucleic acid (e.g., DNA, mRNA) or protein of a biological sample obtained from the human individual for which a susceptibility is being determined. The biological sample can be any nucleic acid or protein containing sample obtained from the human individual. For example, the biological sample can be any of the biological samples described herein.

### Number of Polymorphic Markers/Genes Analyzed

With regard to the methods of determining a susceptibility described herein, the methods can comprise obtaining sequence data about any number of polymorphic markers and/or about any number of genes. For example, the method can comprise obtaining sequence data for about at least 1, 2, 3, 4, 5, 6, 7, 8, 9, 10, 15, 20, 25, 30, 40, 50, 100, 500, 1000, 10,000 or more polymorphic markers. In certain embodiments, the sequence data is obtained from a microarray comprising probes for detecting a plurality of markers. The markers can be independent of rs7005606 and rs966423 and/or the markers may be in linkage disequilibrium with rs7005606 and/or rs966423. The polymorphic markers can be the ones of the group specified herein or they can be different polymorphic markers that are not listed herein. In a specific embodiment, the method comprises obtaining sequence data about at least two polymorphic markers. In certain embodiments, each of the markers may be associated with a different gene. For example, in some instances, if the method comprises obtaining nucleic acid data about a human individual identifying at least one allele of a polymorphic marker, then the method comprises identifying at least one allele of at least one polymorphic marker. Also, for example, the method can comprise obtaining sequence data about a human individual identifying alleles of multiple, independent markers, which are not in linkage disequilibrium.

### Linkage Disequilibrium

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots.

For the methods described herein, a significant r² value can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one specific embodiment of invention, the significant r² value can be at least 0.2. In another specific embodiment of invention, the significant r² value can be at least 0.5. In one specific embodiment of invention, the significant r² value can be at least 0.8. Alternatively, linkage disequilibrium as described herein, refers to linkage disequilibrium characterized by values of r² of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). Linkage disequilibrium can be determined in a single human population, as defined herein, or it can be determined in a collection of samples comprising individuals from more than one human population. In one embodiment of the invention, LD is determined in a sample from one or more of the HapMap populations. These include samples from the Yoruba people of Ibadan, Nigeria (YRI), samples from individuals from the Tokyo area in Japan (JPT), samples from individuals Beijing, China (CHB), and samples from U.S. residents with northern and western European ancestry (CEU), as described (The International HapMap Consortium, Nature 426:789-796 (2003)). In one such embodiment, LD is determined in the Caucasian CEU population of the HapMap samples. In another embodiment, LD is determined in the African YRI population. In yet another embodiment, LD is determined in samples from the Icelandic population.

If all polymorphisms in the genome were independent at the population level (i.e., no LD between polymorphisms), then every single one of them would need to be investigated in association studies, to assess all different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273:1516-1517 (1996); Maniatis, N., et al., Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33: 382-387 (2003)).

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the invention.

By way of example, the markers rs7005606 and rs966423 may be detected directly to determine risk of Thyroid Cancer. Alternatively, any marker in linkage disequilibrium with rs7005606 and rs966423 may be detected to determine risk.

The present invention thus refers to the rs7005606 and rs966423 markers used for detecting association to Thyroid Cancer, as well as markers in linkage disequilibrium with these markers. Thus, in certain embodiments of the invention, markers that are in LD with these markers, e.g., markers as described herein, may be used as surrogate markers.

Suitable surrogate markers may be selected using public information, such as from the International HapMap Consortium (http://www.hapmap.org) and the International 1000 genomes Consortium (http://www.1000genomes.org). The stronger the linkage disequilibrium to the anchor marker, the better the surrogate, and thus the mores similar the association detected by the surrogate is expected to be to the association detected by the anchor marker. Markers with values of r² equal to 1 are perfect surrogates for the at-risk variants, i.e. genotypes for one marker perfectly predicts genotypes for the other. In other words, the surrogate will, by necessity, give exactly the same association data to any particular disease as the anchor marker. Markers with smaller values of r² than 1 can also be surrogates for the at-risk anchor variant.

The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and select appropriate surrogate markers.

In certain embodiments, suitable surrogate markers of rs7005606 are selected from the group consisting of the markers set forth in Table 1. In certain embodiments, suitable surrogate markers of rs966423 are selected from the group consisting of the markers set forth in Table 2.

TABLE 1
Surrogate markers of anchor marker rs966423 on Chromosome 2. Markers were
selected using data from Caucasian HapMap dataset or the publically available 1000 Genomes
project (http://www.1000genomes.org). Markers that have not been assigned rs names are
identified by their position in NCBI Build 36 of the human genome assembly. Shown are the
marker names and position in NCBI Build 36, risk alleles for the surrogate markers, i.e. alleles
that are correlated with the at-risk C allele of rs966423 and the other allele for that marker.
Linkage disequilibrium measures D′ and r², and corresponding p-value, are also shown. The
last column refers to the sequence listing number, identifying the particular SNP.

| | | | | | | | Seq |
|---|---|---|---|---|---|---|---|
| | Pos. In | CORRELATED | OTHER | | | | ID |
| SNP | NCBI B36 | ALLELE | ALLELE | D′ | r² | P-value | No: |
| rs12151423 | 217945526 | A | G | 0.65 | 0.31 | 1.80E−05 | 1 |
| rs12151670 | 217945682 | G | A | 0.65 | 0.31 | 1.80E−05 | 2 |
| rs12614420 | 217946991 | T | A | 0.61 | 0.3 | 3.10E−05 | 3 |
| rs12620884 | 217947126 | G | A | 0.69 | 0.33 | 4.80E−06 | 4 |
| s.217951552 | 217951552 | G | A | 0.58 | 0.26 | 0.00024 | 5 |
| rs7575155 | 217952389 | G | A | 0.66 | 0.28 | 7.10E−05 | 6 |
| rs2373058 | 217958794 | C | G | 1 | 0.31 | 1.20E−10 | 7 |
| rs6706673 | 217959947 | A | G | 0.94 | 0.55 | 1.50E−10 | 8 |
| s.217961378 | 217961378 | C | T | 1 | 0.22 | 2.70E−09 | 9 |
| rs34587525 | 217961934 | A | G | 1 | 0.31 | 1.20E−10 | 10 |
| s.217962214 | 217962214 | C | T | 1 | 0.52 | 4.10E−17 | 11 |
| s.217963774 | 217963774 | C | T | 0.95 | 0.73 | 2.50E−15 | 12 |
| rs4674161 | 217964254 | C | T | 1 | 0.86 | 2.40E−28 | 13 |
| rs6723847 | 217964734 | T | C | 0.94 | 0.55 | 1.50E−10 | 14 |
| rs12232972 | 217965517 | T | C | 1 | 0.31 | 1.20E−10 | 15 |
| rs10932715 | 217968028 | C | T | 1 | 0.33 | 3.20E−11 | 16 |
| rs58933889 | 217970178 | A | G | 1 | 0.27 | 1.60E−09 | 17 |
| rs17191752 | 217970985 | G | A | 0.92 | 0.79 | 4.10E−16 | 18 |
| s.217971087 | 217971087 | C | T | 0.92 | 0.79 | 4.10E−16 | 19 |
| s.217971103 | 217971103 | A | G | 1 | 0.25 | 5.80E−09 | 20 |
| rs17804901 | 217971121 | C | G | 0.92 | 0.79 | 4.10E−16 | 21 |
| s.217972044 | 217972044 | C | T | 0.89 | 0.27 | 0.0002 | 22 |
| s.217972052 | 217972052 | G | A | 0.89 | 0.24 | 0.00015 | 23 |
| s.217972365 | 217972365 | T | G | 1 | 0.21 | 6.80E−08 | 24 |
| rs12989997 | 217974601 | C | T | 1 | 0.86 | 2.40E−28 | 25 |
| rs55806820 | 217974990 | C | T | 1 | 0.89 | 1.10E−29 | 26 |
| rs1351163 | 217976237 | G | A | 1 | 0.31 | 1.20E−10 | 27 |
| rs9752576 | 217977690 | A | G | 1 | 0.29 | 4.50E−10 | 28 |
| rs6759952 | 217979964 | T | C | 0.88 | 0.75 | 5.10E−15 | 29 |
| rs1351164 | 217980143 | C | T | 0.8 | 0.25 | 0.00024 | 30 |
| rs57004880 | 217980826 | C | A | 0.8 | 0.25 | 0.00024 | 31 |
| rs73079697 | 217980999 | T | G | 1 | 0.31 | 1.20E−10 | 32 |
| s.217981194 | 217981194 | T | C | 1 | 0.31 | 1.20E−10 | 33 |
| rs6720623 | 217981325 | A | G | 0.84 | 0.5 | 4.80E−09 | 34 |
| s.217981456 | 217981456 | A | G | 0.88 | 0.75 | 5.10E−15 | 35 |
| rs6720977 | 217981620 | A | G | 0.84 | 0.5 | 4.80E−09 | 36 |
| rs6721000 | 217981698 | A | G | 0.84 | 0.5 | 4.80E−09 | 37 |
| rs1382430 | 217982533 | T | C | 0.84 | 0.5 | 4.80E−09 | 38 |
| rs1382431 | 217982668 | T | C | 0.84 | 0.5 | 4.80E−09 | 39 |
| rs4674163 | 217982725 | G | A | 0.84 | 0.5 | 4.80E−09 | 40 |
| rs10932716 | 217982906 | G | A | 0.84 | 0.5 | 4.80E−09 | 41 |
| rs11674838 | 217982945 | T | C | 0.84 | 0.5 | 4.80E−09 | 42 |
| rs4674164 | 217983142 | T | C | 0.84 | 0.5 | 4.80E−09 | 43 |
| rs4674165 | 217983188 | T | C | 0.84 | 0.5 | 4.80E−09 | 44 |
| s.217983484 | 217983484 | G | A | 1 | 0.28 | 4.70E−11 | 45 |
| rs4674167 | 217983615 | T | C | 0.84 | 0.5 | 4.80E−09 | 46 |
| rs981938 | 217984063 | G | A | 0.84 | 0.5 | 4.80E−09 | 47 |
| rs4674168 | 217984406 | T | C | 0.88 | 0.75 | 5.10E−15 | 48 |
| rs4674169 | 217984485 | T | C | 0.84 | 0.5 | 4.80E−09 | 49 |
| s.217985081 | 217985081 | G | A | 0.84 | 0.5 | 4.80E−09 | 50 |
| s.217985297 | 217985297 | C | T | 1 | 0.37 | 8.30E−14 | 51 |
| rs6736742 | 217985394 | A | G | 1 | 0.6 | 2.00E−19 | 52 |
| rs1478575 | 217986800 | T | A | 0.84 | 0.5 | 4.80E−09 | 53 |
| rs2113832 | 217986937 | A | G | 0.84 | 0.5 | 4.80E−09 | 54 |
| rs2162001 | 217987015 | T | C | 0.88 | 0.75 | 5.10E−15 | 55 |
| rs1600210 | 217987026 | C | A | 0.84 | 0.5 | 4.80E−09 | 56 |
| rs1600211 | 217987248 | A | G | 0.84 | 0.5 | 4.80E−09 | 57 |
| rs1600212 | 217987355 | T | C | 0.84 | 0.5 | 4.80E−09 | 58 |
| rs10191791 | 217987492 | A | G | 0.84 | 0.5 | 4.80E−09 | 59 |
| rs34413965 | 217987716 | C | T | 0.84 | 0.5 | 4.80E−09 | 60 |
| rs34756249 | 217987731 | T | C | 0.84 | 0.5 | 4.80E−09 | 61 |
| rs7567847 | 217987818 | C | A | 0.84 | 0.5 | 4.80E−09 | 62 |
| s.217987934 | 217987934 | C | T | 0.84 | 0.5 | 4.80E−09 | 63 |
| rs10183694 | 217988168 | A | T | 0.8 | 0.25 | 0.00024 | 64 |
| rs7584902 | 217988183 | T | G | 1 | 0.6 | 2.00E−19 | 65 |
| rs1118149 | 217988491 | A | G | 1 | 0.6 | 2.00E−19 | 66 |
| rs1118150 | 217988513 | C | A | 1 | 0.6 | 2.00E−19 | 67 |
| rs1118151 | 217988663 | T | G | 1 | 0.6 | 2.00E−19 | 68 |
| rs13388148 | 217989745 | G | T | 1 | 0.31 | 1.20E−10 | 69 |
| rs13406698 | 217991330 | G | A | 1 | 0.31 | 1.20E−10 | 70 |
| rs13395110 | 217991548 | G | T | 0.84 | 0.5 | 4.80E−09 | 71 |
| rs13432615 | 217991684 | T | C | 0.84 | 0.5 | 4.80E−09 | 72 |
| rs994532 | 217992455 | G | A | 0.84 | 0.5 | 4.80E−09 | 73 |
| rs994533 | 217992523 | C | G | 0.84 | 0.5 | 4.80E−09 | 74 |
| rs10490762 | 217992642 | A | T | 0.84 | 0.5 | 4.80E−09 | 75 |
| rs1478576 | 217992769 | C | T | 0.84 | 0.5 | 4.80E−09 | 76 |
| s.217992813 | 217992813 | A | G | 0.96 | 0.83 | 2.00E−17 | 77 |
| s.217993044 | 217993044 | A | G | 0.96 | 0.83 | 2.00E−17 | 78 |
| rs13401747 | 217993059 | C | T | 0.84 | 0.5 | 4.80E−09 | 79 |
| s.217993346 | 217993346 | T | A | 0.88 | 0.23 | 0.00062 | 80 |
| s.217993357 | 217993357 | G | C | 0.88 | 0.75 | 5.10E−15 | 81 |
| rs11676600 | 217993634 | A | C | 1 | 0.6 | 2.00E−19 | 82 |
| s.217994344 | 217994344 | C | T | 1 | 0.6 | 2.00E−19 | 83 |
| rs7603771 | 217995359 | T | A | 0.84 | 0.5 | 4.80E−09 | 84 |
| rs7577615 | 217995426 | T | C | 0.84 | 0.5 | 4.80E−09 | 85 |
| rs11890853 | 217996436 | T | C | 1 | 0.6 | 2.00E−19 | 86 |
| s.217996462 | 217996462 | A | G | 1 | 0.31 | 1.20E−10 | 87 |
| rs11890939 | 217996470 | T | G | 1 | 0.6 | 2.00E−19 | 88 |
| s.217996825 | 217996825 | G | A | 1 | 0.31 | 1.20E−10 | 89 |
| s.217997076 | 217997076 | A | T | 0.96 | 0.83 | 2.00E−17 | 90 |
| s.217997492 | 217997492 | G | T | 0.84 | 0.5 | 4.80E−09 | 91 |
| rs12694415 | 217997602 | G | A | 0.88 | 0.75 | 5.10E−15 | 92 |
| rs12694416 | 217997742 | A | C | 0.88 | 0.75 | 5.10E−15 | 93 |
| s.217998287 | 217998287 | C | T | 0.88 | 0.75 | 5.10E−15 | 94 |
| s.217998293 | 217998293 | C | T | 0.84 | 0.5 | 4.80E−09 | 95 |
| s.217998603 | 217998603 | T | C | 0.84 | 0.5 | 4.80E−09 | 96 |
| rs12624106 | 217998690 | G | A | 1 | 0.31 | 1.20E−10 | 97 |
| s.217998914 | 217998914 | C | T | 0.84 | 0.5 | 4.80E−09 | 98 |
| rs2194736 | 217999216 | T | C | 0.84 | 0.5 | 4.80E−09 | 99 |
| rs3732009 | 217999638 | A | G | 1 | 0.31 | 1.20E−10 | 100 |
| rs1478579 | 217999769 | T | C | 1 | 0.6 | 2.00E−19 | 101 |
| rs1478580 | 217999894 | T | C | 1 | 0.31 | 1.20E−10 | 102 |
| s.218000386 | 218000386 | T | C | 1 | 0.52 | 4.10E−17 | 103 |
| s.218000403 | 218000403 | C | G | 1 | 0.52 | 4.10E−17 | 104 |
| rs1478581 | 218000897 | A | G | 1 | 0.31 | 1.20E−10 | 105 |
| s.218001450 | 218001450 | G | A | 0.82 | 0.42 | 3.70E−07 | 106 |
| rs6745321 | 218001479 | T | C | 0.96 | 0.83 | 2.00E−17 | 107 |
| rs7594625 | 218001809 | G | T | 0.84 | 0.5 | 4.80E−09 | 108 |
| s.218002336 | 218002336 | T | A | 0.75 | 0.43 | 2.50E−07 | 109 |
| rs12990503 | 218002462 | C | G | 1 | 0.52 | 4.10E−17 | 110 |
| rs6734808 | 218002816 | T | C | 1 | 0.31 | 1.20E−10 | 111 |
| rs10181989 | 218003160 | C | T | 0.8 | 0.25 | 0.00024 | 112 |
| rs13388294 | 218003651 | A | G | 0.93 | 0.48 | 1.30E−08 | 113 |
| rs1382435 | 218004248 | T | C | 0.79 | 0.46 | 3.90E−08 | 114 |
| rs13004333 | 218004386 | C | G | 0.79 | 0.46 | 3.90E−08 | 115 |
| rs57481445 | 218004619 | G | A | 1 | 0.52 | 4.10E−17 | 116 |
| rs16857609 | 218004753 | T | C | 1 | 0.52 | 4.10E−17 | 117 |
| rs16857611 | 218004977 | T | C | 1 | 0.52 | 4.10E−17 | 118 |
| rs11680689 | 218005945 | C | G | 1 | 0.57 | 1.20E−18 | 119 |
| rs1233081 | 218008489 | T | C | 1 | 0.62 | 3.00E−20 | 120 |
| rs16857630 | 218008775 | G | T | 1 | 0.21 | 6.80E−08 | 121 |
| rs12478966 | 218008808 | A | G | 1 | 0.71 | 6.40E−23 | 122 |
| rs12473807 | 218008967 | A | T | 1 | 0.71 | 6.40E−23 | 123 |
| rs4674176 | 218009364 | G | C | 1 | 0.6 | 2.00E−19 | 124 |
| rs13002451 | 218009586 | G | A | 1 | 0.6 | 2.00E−19 | 125 |
| rs2618146 | 218010258 | G | A | 0.84 | 0.5 | 9.60E−09 | 126 |
| rs2618147 | 218010383 | A | C | 1 | 0.6 | 2.00E−19 | 127 |
| rs12617808 | 218010462 | T | C | 1 | 0.87 | 4.50E−29 | 128 |
| rs2568176 | 218012203 | A | G | 1 | 0.57 | 1.20E−18 | 129 |
| rs2618148 | 218012351 | T | C | 1 | 0.65 | 4.20E−21 | 130 |
| s.218012693 | 218012693 | C | T | 1 | 0.37 | 2.00E−12 | 131 |
| rs2568175 | 218012753 | A | T | 1 | 0.71 | 6.40E−23 | 132 |
| rs6715218 | 218013309 | C | T | 1 | 0.52 | 4.10E−17 | 133 |
| rs6729012 | 218013638 | C | A | 1 | 0.52 | 4.10E−17 | 134 |
| s.218013951 | 218013951 | C | A | 1 | 0.29 | 4.50E−10 | 135 |
| s.218013960 | 218013960 | T | C | 1 | 0.52 | 4.10E−17 | 136 |
| s.218013975 | 218013975 | C | T | 1 | 0.37 | 2.00E−12 | 137 |
| s.218014108 | 218014108 | G | A | 1 | 0.37 | 8.30E−14 | 138 |
| rs73069129 | 218014146 | C | A | 1 | 0.62 | 3.00E−20 | 139 |
| s.218014260 | 218014260 | G | A | 1 | 0.83 | 5.10E−28 | 140 |
| rs12694417 | 218014334 | T | C | 1 | 0.52 | 4.10E−17 | 141 |
| rs12988242 | 218014439 | A | G | 1 | 0.52 | 4.10E−17 | 142 |
| s.218014948 | 218014948 | T | C | 1 | 0.29 | 4.50E−10 | 143 |
| rs10084346 | 218014981 | T | C | 1 | 0.31 | 1.20E−10 | 144 |
| s.218015468 | 218015468 | C | T | 1 | 0.52 | 4.10E−17 | 145 |
| s.218015572 | 218015572 | G | T | 1 | 0.23 | 2.00E−08 | 146 |
| rs2045933 | 218015701 | A | T | 1 | 0.55 | 7.40E−18 | 147 |
| rs1318847 | 218015940 | T | C | 1 | 0.6 | 2.00E−19 | 148 |
| s.218016001 | 218016001 | G | A | 1 | 0.27 | 1.60E−09 | 149 |
| rs974405 | 218016155 | C | T | 1 | 0.52 | 4.10E−17 | 150 |
| rs974406 | 218016283 | C | G | 1 | 0.55 | 7.40E−18 | 151 |
| rs6712801 | 218016746 | A | G | 1 | 0.52 | 4.10E−17 | 152 |
| rs2618149 | 218017255 | G | T | 1 | 0.23 | 2.00E−08 | 153 |
| s.218017265 | 218017265 | A | G | 1 | 0.6 | 2.00E−19 | 154 |
| s.218017466 | 218017466 | G | C | 1 | 0.6 | 2.00E−19 | 155 |
| s.218017473 | 218017473 | A | C | 1 | 0.6 | 2.00E−19 | 156 |
| rs4674178 | 218017503 | C | T | 1 | 0.6 | 2.00E−19 | 157 |
| s.218017512 | 218017512 | A | T | 1 | 0.6 | 2.00E−19 | 158 |
| rs4142171 | 218017985 | G | T | 1 | 0.52 | 4.10E−17 | 159 |
| rs1478595 | 218018144 | G | T | 1 | 0.55 | 7.40E−18 | 160 |
| rs1478596 | 218018181 | C | G | 1 | 0.6 | 2.00E−19 | 161 |
| rs966423 | 218018585 | C | T | 1 | 1 | — | 162 |
| rs4674179 | 218018931 | A | C | 1 | 0.31 | 1.20E−10 | 163 |
| rs2618150 | 218019691 | G | A | 1 | 0.52 | 4.10E−17 | 164 |
| s.218020843 | 218020843 | G | C | 0.93 | 0.43 | 9.80E−09 | 165 |
| rs13418112 | 218022274 | A | G | 0.94 | 0.55 | 1.50E−10 | 166 |
| s.218022292 | 218022292 | A | G | 1 | 0.23 | 2.00E−08 | 167 |
| rs13418037 | 218022386 | T | C | 1 | 0.31 | 1.20E−10 | 168 |
| rs7569925 | 218023281 | T | G | 1 | 0.27 | 1.60E−09 | 169 |
| rs2568173 | 218023698 | A | G | 0.48 | 0.23 | 0.00064 | 170 |
| rs1871231 | 218024089 | G | A | 1 | 0.25 | 5.80E−09 | 171 |
| rs12622350 | 218024426 | A | G | 1 | 0.25 | 5.80E−09 | 172 |
| rs2618154 | 218024572 | A | T | 1 | 0.23 | 2.00E−08 | 173 |
| rs967047 | 218025930 | A | C | 1 | 0.25 | 5.80E−09 | 174 |
| rs2568172 | 218027406 | G | C | 1 | 0.25 | 5.80E−09 | 175 |
| rs1564241 | 218029002 | T | C | 1 | 0.25 | 5.80E−09 | 176 |
| rs1564242 | 218029051 | A | G | 1 | 0.25 | 5.80E−09 | 177 |
| rs1478601 | 218029446 | C | T | 1 | 0.25 | 5.80E−09 | 178 |
| rs2568170 | 218029678 | A | C | 1 | 0.25 | 5.80E−09 | 179 |
| s.218030382 | 218030382 | G | C | 0.86 | 0.55 | 2.50E−09 | 180 |
| rs4619585 | 218030483 | A | T | 1 | 0.23 | 2.00E−08 | 181 |
| rs12614421 | 218031559 | G | T | 1 | 0.25 | 5.80E−09 | 182 |
| rs2568169 | 218032611 | G | C | 1 | 0.25 | 5.80E−09 | 183 |
| rs2568168 | 218032726 | G | A | 1 | 0.25 | 5.80E−09 | 184 |
| rs2618138 | 218033005 | C | T | 1 | 0.25 | 5.80E−09 | 185 |
| rs2618139 | 218035046 | A | G | 0.74 | 0.4 | 3.30E−07 | 186 |
| rs2568167 | 218035363 | T | C | 1 | 0.25 | 5.80E−09 | 187 |
| rs2568166 | 218035412 | C | T | 1 | 0.25 | 5.80E−09 | 188 |
| rs57662183 | 218036387 | G | A | 1 | 0.25 | 5.80E−09 | 189 |
| s.218038408 | 218038408 | T | C | 1 | 0.21 | 6.80E−08 | 190 |
| s.218038630 | 218038630 | T | A | 1 | 0.21 | 6.80E−08 | 191 |
| rs1478583 | 218038886 | C | T | 1 | 0.25 | 5.80E−09 | 192 |
| s.218040446 | 218040446 | T | C | 1 | 0.25 | 5.80E−09 | 193 |
| s.218040448 | 218040448 | T | C | 1 | 0.25 | 5.80E−09 | 194 |
| rs1478584 | 218041443 | A | G | 1 | 0.25 | 5.80E−09 | 195 |
| rs2568160 | 218042708 | A | C | 0.93 | 0.48 | 6.60E−09 | 196 |
| rs2568159 | 218042748 | T | C | 0.93 | 0.48 | 6.60E−09 | 197 |
| rs2568158 | 218042779 | T | C | 0.93 | 0.48 | 6.60E−09 | 198 |
| s.218042836 | 218042836 | T | C | 1 | 0.21 | 6.80E−08 | 199 |
| rs1478585 | 218043029 | A | G | 0.93 | 0.48 | 6.60E−09 | 200 |
| rs1478586 | 218043137 | A | G | 0.93 | 0.48 | 6.60E−09 | 201 |
| rs1478587 | 218043227 | T | C | 0.93 | 0.48 | 6.60E−09 | 202 |
| rs2568156 | 218043685 | C | T | 0.93 | 0.45 | 3.40E−08 | 203 |
| s.218043796 | 218043796 | C | A | 1 | 0.23 | 2.00E−08 | 204 |
| rs2568155 | 218044150 | A | G | 0.93 | 0.48 | 6.60E−09 | 205 |
| rs2568154 | 218044299 | G | A | 0.93 | 0.48 | 6.60E−09 | 206 |
| rs1382436 | 218044568 | A | G | 0.93 | 0.48 | 6.60E−09 | 207 |
| rs2618142 | 218044915 | G | A | 0.93 | 0.48 | 6.60E−09 | 208 |
| rs2618143 | 218044931 | T | C | 0.93 | 0.48 | 6.60E−09 | 209 |
| rs1478588 | 218046394 | A | G | 1 | 0.21 | 6.80E−08 | 210 |
| rs1478590 | 218046715 | T | C | 1 | 0.25 | 5.80E−09 | 211 |
| rs1382438 | 218047074 | C | A | 0.93 | 0.48 | 6.60E−09 | 212 |
| rs1382439 | 218047110 | A | G | 1 | 0.25 | 5.80E−09 | 213 |
| rs1382440 | 218047213 | A | G | 0.93 | 0.48 | 6.60E−09 | 214 |
| rs2618144 | 218047592 | C | T | 1 | 0.25 | 5.80E−09 | 215 |
| rs2568153 | 218047749 | A | C | 0.93 | 0.48 | 6.60E−09 | 216 |
| rs1963252 | 218050313 | A | G | 0.77 | 0.38 | 1.30E−06 | 217 |
| s.218050347 | 218050347 | A | T | 0.71 | 0.35 | 7.00E−06 | 218 |
| rs10490763 | 218051350 | C | T | 1 | 0.25 | 5.80E−09 | 219 |
| rs768434 | 218052016 | G | C | 1 | 0.25 | 5.80E−09 | 220 |
| rs768435 | 218052123 | T | C | 0.93 | 0.48 | 6.60E−09 | 221 |
| s.218052600 | 218052600 | T | G | 1 | 0.21 | 6.80E−08 | 222 |
| rs10804261 | 218052675 | G | C | 1 | 0.25 | 5.80E−09 | 223 |
| s.218052731 | 218052731 | A | G | 1 | 0.21 | 6.80E−08 | 224 |
| s.218052803 | 218052803 | A | G | 1 | 0.21 | 6.80E−08 | 225 |
| rs12989540 | 218053068 | A | T | 1 | 0.21 | 6.80E−08 | 226 |
| rs13013662 | 218053325 | G | C | 1 | 0.21 | 6.80E−08 | 227 |
| rs1478591 | 218054307 | T | A | 1 | 0.21 | 6.80E−08 | 228 |
| rs1478592 | 218054352 | C | T | 0.93 | 0.48 | 6.60E−09 | 229 |
| rs1478593 | 218054382 | A | G | 1 | 0.21 | 6.80E−08 | 230 |
| rs1478594 | 218054417 | A | G | 1 | 0.21 | 6.80E−08 | 231 |
| rs2068972 | 218054696 | A | G | 0.93 | 0.48 | 6.60E−09 | 232 |
| rs2618145 | 218054796 | T | C | 1 | 0.25 | 5.80E−09 | 233 |
| rs9677520 | 218054937 | C | T | 1 | 0.25 | 5.80E−09 | 234 |
| rs4479407 | 218055221 | T | C | 1 | 0.21 | 6.80E−08 | 235 |
| rs66838277 | 218056427 | T | A | 1 | 0.23 | 2.00E−08 | 236 |
| rs12990931 | 218056993 | G | A | 1 | 0.21 | 6.80E−08 | 237 |
| rs35856653 | 218058451 | A | C | 0.93 | 0.48 | 6.60E−09 | 238 |
| s.218058636 | 218058636 | A | G | 1 | 0.25 | 5.80E−09 | 239 |
| rs7420802 | 218059559 | C | T | 1 | 0.21 | 6.80E−08 | 240 |
| rs17807893 | 218059618 | C | A | 1 | 0.21 | 6.80E−08 | 241 |
| rs2373065 | 218059873 | T | C | 1 | 0.21 | 6.80E−08 | 242 |
| rs1072086 | 218060195 | T | A | 0.93 | 0.45 | 3.40E−08 | 243 |
| rs2373066 | 218060619 | T | C | 0.93 | 0.48 | 6.60E−09 | 244 |
| rs4555323 | 218060847 | A | C | 1 | 0.21 | 6.80E−08 | 245 |
| s.218061215 | 218061215 | A | G | 1 | 0.21 | 6.80E−08 | 246 |
| rs874839 | 218062332 | G | T | 0.93 | 0.48 | 6.60E−09 | 247 |
| rs874840 | 218062375 | T | C | 0.93 | 0.48 | 6.60E−09 | 248 |
| s.218062582 | 218062582 | G | T | 1 | 0.21 | 6.80E−08 | 249 |
| rs13404164 | 218064006 | C | T | 1 | 0.21 | 6.80E−08 | 250 |
| rs10490764 | 218064112 | T | C | 1 | 0.21 | 6.80E−08 | 251 |
| rs6754157 | 218064973 | G | A | 1 | 0.21 | 6.80E−08 | 252 |
| rs6725886 | 218065003 | A | G | 1 | 0.21 | 6.80E−08 | 253 |
| rs6754268 | 218065042 | G | A | 0.93 | 0.48 | 6.60E−09 | 254 |
| rs6754393 | 218065181 | G | A | 0.93 | 0.48 | 6.60E−09 | 255 |
| rs6754399 | 218065197 | G | A | 0.93 | 0.48 | 6.60E−09 | 256 |
| rs12475467 | 218065627 | G | A | 0.93 | 0.48 | 6.60E−09 | 257 |
| rs10191880 | 218066205 | G | T | 1 | 0.21 | 6.80E−08 | 258 |
| rs6731141 | 218066868 | T | G | 1 | 0.25 | 5.80E−09 | 259 |
| rs6705050 | 218067133 | T | A | 1 | 0.25 | 5.80E−09 | 260 |
| rs12473831 | 218067243 | C | T | 1 | 0.21 | 6.80E−08 | 261 |
| rs7598065 | 218067576 | C | T | 1 | 0.21 | 6.80E−08 | 262 |
| rs7584377 | 218067654 | A | G | 1 | 0.21 | 6.80E−08 | 263 |
| rs10184642 | 218069676 | C | T | 1 | 0.27 | 1.60E−09 | 264 |
| s.218070159 | 218070159 | A | T | 1 | 0.27 | 1.60E−09 | 265 |
| rs13393933 | 218070584 | T | C | 1 | 0.27 | 1.60E−09 | 266 |
| s.218073127 | 218073127 | C | A | 1 | 0.23 | 2.00E−08 | 267 |
| s.218073626 | 218073626 | T | C | 1 | 0.21 | 6.80E−08 | 268 |
| rs4372880 | 218073793 | T | C | 1 | 0.27 | 1.60E−09 | 269 |
| s.218073841 | 218073841 | G | T | 1 | 0.21 | 6.80E−08 | 270 |
| s.218075720 | 218075720 | C | T | 1 | 0.21 | 6.80E−08 | 271 |
| rs7597620 | 218076132 | A | G | 1 | 0.27 | 1.60E−09 | 272 |
| rs13432053 | 218077072 | C | T | 1 | 0.27 | 1.60E−09 | 273 |
| rs13418746 | 218077241 | T | C | 1 | 0.27 | 1.60E−09 | 274 |
| s.218077469 | 218077469 | A | G | 1 | 0.27 | 1.60E−09 | 275 |
| s.218077471 | 218077471 | C | T | 1 | 0.27 | 1.60E−09 | 276 |
| rs9989863 | 218077552 | A | G | 1 | 0.27 | 1.60E−09 | 277 |
| rs61349367 | 218077934 | C | A | 1 | 0.27 | 1.60E−09 | 278 |
| rs12328323 | 218077983 | G | A | 0.59 | 0.23 | 0.00045 | 279 |
| rs13423066 | 218078783 | C | G | 1 | 0.27 | 1.60E−09 | 280 |
| rs58054018 | 218078939 | T | C | 1 | 0.27 | 1.60E−09 | 281 |
| rs56871250 | 218079003 | C | T | 1 | 0.27 | 1.60E−09 | 282 |
| rs66476209 | 218079206 | T | C | 1 | 0.27 | 1.60E−09 | 283 |
| rs9989823 | 218079446 | T | C | 1 | 0.27 | 1.60E−09 | 284 |
| rs9989824 | 218079498 | T | G | 1 | 0.25 | 5.80E−09 | 285 |
| s.218079610 | 218079610 | A | T | 1 | 0.27 | 1.60E−09 | 286 |
| rs7589686 | 218079703 | C | G | 1 | 0.27 | 1.60E−09 | 287 |
| s.218080014 | 218080014 | T | G | 0.5 | 0.22 | 0.00035 | 288 |
| rs7592756 | 218080060 | A | G | 1 | 0.27 | 1.60E−09 | 289 |
| s.218080235 | 218080235 | T | C | 1 | 0.21 | 6.80E−08 | 290 |
| s.218080237 | 218080237 | A | T | 1 | 0.23 | 2.00E−08 | 291 |
| s.218080238 | 218080238 | A | T | 1 | 0.21 | 6.80E−08 | 292 |
| s.218082009 | 218082009 | G | A | 1 | 0.21 | 6.80E−08 | 293 |
| rs59862963 | 218083502 | G | T | 1 | 0.27 | 1.60E−09 | 294 |
| rs6717678 | 218083915 | C | T | 0.5 | 0.22 | 0.00035 | 295 |
| rs9288528 | 218083998 | G | T | 1 | 0.27 | 1.60E−09 | 296 |
| rs7558156 | 218084937 | T | C | 1 | 0.27 | 1.60E−09 | 297 |
| rs13429028 | 218085570 | A | G | 1 | 0.27 | 1.60E−09 | 298 |
| s.218085899 | 218085899 | C | T | 1 | 0.29 | 4.50E−10 | 299 |
| rs66829776 | 218086791 | A | G | 1 | 0.27 | 1.60E−09 | 300 |
| rs2888485 | 218087463 | T | C | 1 | 0.27 | 1.60E−09 | 301 |
| rs13390257 | 218090481 | C | T | 0.5 | 0.22 | 0.00035 | 302 |
| rs10209831 | 218091398 | A | C | 1 | 0.27 | 1.60E−09 | 303 |
| rs2373077 | 218093085 | A | C | 1 | 0.27 | 1.60E−09 | 304 |
| rs9288529 | 218094817 | C | T | 0.53 | 0.25 | 0.00017 | 305 |
| rs10173367 | 218094823 | A | G | 1 | 0.27 | 1.60E−09 | 306 |
| rs60483917 | 218095422 | T | C | 1 | 0.27 | 1.60E−09 | 307 |
| rs13008340 | 218098259 | C | T | 0.56 | 0.29 | 4.00E−05 | 308 |
| rs12621646 | 218098946 | T | C | 0.88 | 0.24 | 7.50E−05 | 309 |
| rs12694419 | 218099262 | C | G | 0.57 | 0.32 | 1.30E−05 | 310 |
| rs750365 | 218099708 | A | C | 0.49 | 0.22 | 0.00084 | 311 |
| rs2011862 | 218099775 | T | C | 1 | 0.27 | 1.60E−09 | 312 |
| rs6729351 | 218101545 | A | G | 0.6 | 0.24 | 0.00066 | 313 |
| s.218101634 | 218101634 | G | A | 0.8 | 0.37 | 2.00E−06 | 314 |
| rs11889534 | 218102220 | C | T | 0.54 | 0.27 | 0.00012 | 315 |
| s.218102832 | 218102832 | G | A | 1 | 0.23 | 2.00E−08 | 316 |
| s.218103282 | 218103282 | G | A | 1 | 0.27 | 1.60E−09 | 317 |
| rs749386 | 218434270 | A | G | 0.66 | 0.21 | 0.002 | 318 |

TABLE 2
Surrogate markers of anchor marker rs7005606 on Chromosome 8. Markers were
selected using data from Caucasian HapMap dataset or the publically available 1000 Genomes
project (http://www.1000genomes.org). Markers that have not been assigned rs names are
identified by their position in NCBI Build 36 of the human genome assembly. Shown are the
marker names and position in NCBI Build 36, predicted risk alleles for the surrogate markers,
i.e. alleles that are correlated with the at-risk G allele of rs7005606 and the other allele for that
marker. Linkage disequilibrium measures D′ and r², and corresponding p-value, are also
shown. The last column refers to the sequence listing number identifying the particular SNP.

| | Pos. In | CORRELATED | OTHER | | | | Seq ID |
|---|---|---|---|---|---|---|---|
| SNP | NCBI B36 | ALLELE | ALLELE | D′ | R² | P-value | No: |
| s.32285834 | 32285834 | C | G | 0.52 | 0.24 | 5.20E−05 | 319 |
| s.32287197 | 32287197 | T | G | 0.59 | 0.25 | 2.80E−05 | 320 |
| rs35110336 | 32289082 | C | T | 0.51 | 0.22 | 9.00E−05 | 321 |
| s.32289719 | 32289719 | C | T | 0.51 | 0.22 | 9.00E−05 | 322 |
| rs11989384 | 32290067 | C | T | 0.68 | 0.26 | 7.10E−05 | 323 |
| rs4317533 | 32290309 | G | A | 0.68 | 0.26 | 7.10E−05 | 324 |
| rs10503907 | 32291552 | G | A | 0.52 | 0.21 | 8.20E−05 | 325 |
| rs1545961 | 32292898 | C | T | 0.66 | 0.22 | 0.00024 | 326 |
| rs1386441 | 32296926 | A | G | 0.63 | 0.33 | 4.30E−06 | 327 |
| s.32299420 | 32299420 | T | C | 0.74 | 0.21 | 0.0001 | 328 |
| rs17631978 | 32301490 | T | C | 0.69 | 0.31 | 9.00E−07 | 329 |
| rs1948098 | 32304474 | C | T | 0.69 | 0.31 | 9.00E−07 | 330 |
| rs1487157 | 32306264 | G | A | 0.73 | 0.27 | 1.60E−05 | 331 |
| rs7013878 | 32313826 | T | C | 0.69 | 0.31 | 9.00E−07 | 332 |
| s.32321440 | 32321440 | T | C | 0.69 | 0.26 | 1.30E−05 | 333 |
| rs7001724 | 32331968 | G | T | 0.68 | 0.29 | 1.60E−06 | 334 |
| rs11783991 | 32332462 | G | A | 0.7 | 0.34 | 2.50E−07 | 335 |
| rs17633955 | 32333715 | C | T | 0.8 | 0.21 | 0.00014 | 336 |
| rs1623372 | 32335144 | G | A | 0.67 | 0.34 | 1.60E−07 | 337 |
| rs1487152 | 32336767 | T | C | 0.53 | 0.24 | 1.50E−05 | 338 |
| rs1487151 | 32337096 | A | G | 0.67 | 0.34 | 1.60E−07 | 339 |
| rs7838052 | 32337813 | A | G | 0.8 | 0.21 | 0.00014 | 340 |
| rs1487150 | 32339961 | A | G | 0.67 | 0.34 | 1.60E−07 | 341 |
| rs55986591 | 32340530 | C | G | 0.67 | 0.34 | 1.60E−07 | 342 |
| s.32343712 | 32343712 | T | A | 0.79 | 0.3 | 7.20E−06 | 343 |
| s.32344321 | 32344321 | C | T | 0.8 | 0.22 | 9.80E−05 | 344 |
| rs7817155 | 32346134 | A | G | 0.56 | 0.3 | 2.20E−06 | 345 |
| rs6468099 | 32346583 | C | G | 0.83 | 0.28 | 1.30E−05 | 346 |
| s.32347084 | 32347084 | T | A | 0.8 | 0.22 | 9.80E−05 | 347 |
| rs6992907 | 32347468 | C | T | 0.56 | 0.3 | 2.20E−06 | 348 |
| rs5006809 | 32347630 | T | C | 0.56 | 0.3 | 2.20E−06 | 349 |
| rs4733317 | 32348224 | T | C | 0.56 | 0.3 | 2.20E−06 | 350 |
| rs2881648 | 32349067 | A | T | 0.65 | 0.28 | 4.50E−06 | 351 |
| s.32350958 | 32350958 | C | A | 0.61 | 0.37 | 1.20E−07 | 352 |
| rs2347504 | 32352599 | A | G | 0.66 | 0.31 | 5.80E−07 | 353 |
| rs4733323 | 32359391 | C | A | 0.52 | 0.24 | 2.70E−05 | 354 |
| s.32361498 | 32361498 | A | G | 0.82 | 0.24 | 3.30E−05 | 355 |
| rs11989773 | 32367878 | A | G | 0.73 | 0.28 | 3.60E−05 | 356 |
| rs67950512 | 32369780 | A | G | 0.71 | 0.25 | 0.00012 | 357 |
| s.32370642 | 32370642 | T | A | 0.71 | 0.25 | 0.00012 | 358 |
| rs11784074 | 32371612 | T | C | 0.6 | 0.21 | 0.00065 | 359 |
| rs10101959 | 32372076 | C | G | 0.6 | 0.21 | 0.00065 | 360 |
| s.32372236 | 32372236 | C | A | 0.8 | 0.21 | 7.40E−05 | 361 |
| rs2347512 | 32372626 | T | C | 0.52 | 0.24 | 2.70E−05 | 362 |
| rs6993762 | 32372788 | C | T | 0.68 | 0.21 | 0.00054 | 363 |
| rs28366800 | 32374794 | T | C | 0.66 | 0.24 | 0.00017 | 364 |
| s.32375113 | 32375113 | G | A | 0.66 | 0.24 | 0.00017 | 365 |
| rs11779244 | 32376065 | C | T | 0.65 | 0.22 | 0.00027 | 366 |
| s.32376625 | 32376625 | C | T | 0.65 | 0.22 | 0.00027 | 367 |
| rs4276645 | 32377215 | G | A | 0.75 | 0.23 | 0.00016 | 368 |
| rs10954838 | 32378177 | C | T | 0.5 | 0.24 | 8.70E−05 | 369 |
| rs9297190 | 32378468 | C | T | 0.65 | 0.22 | 0.00027 | 370 |
| rs9886497 | 32378538 | T | C | 0.65 | 0.22 | 0.00027 | 371 |
| s.32378777 | 32378777 | C | T | 0.75 | 0.23 | 0.00016 | 372 |
| s.32379600 | 32379600 | A | T | 0.65 | 0.22 | 0.00027 | 373 |
| s.32379604 | 32379604 | A | T | 0.65 | 0.22 | 0.00027 | 374 |
| rs67790398 | 32379798 | G | C | 0.65 | 0.22 | 0.00027 | 375 |
| s.32380123 | 32380123 | G | A | 0.65 | 0.22 | 0.00027 | 376 |
| rs17713685 | 32381124 | C | T | 0.65 | 0.22 | 0.00027 | 377 |
| rs11775675 | 32382047 | T | C | 0.65 | 0.22 | 0.00027 | 378 |
| rs17635931 | 32383853 | G | T | 0.64 | 0.21 | 0.00041 | 379 |
| rs28635357 | 32388654 | G | A | 0.64 | 0.21 | 0.00041 | 380 |
| rs10954841 | 32389156 | T | G | 0.65 | 0.22 | 0.00027 | 381 |
| s.32389495 | 32389495 | C | T | 0.62 | 0.22 | 0.00018 | 382 |
| s.32389510 | 32389510 | G | A | 0.62 | 0.22 | 0.00018 | 383 |
| s.32390451 | 32390451 | T | C | 1 | 0.3 | 8.70E−11 | 384 |
| rs10087829 | 32393020 | A | C | 0.65 | 0.22 | 0.00027 | 385 |
| s.32393071 | 32393071 | G | A | 0.76 | 0.24 | 4.40E−05 | 386 |
| s.32393723 | 32393723 | T | C | 0.6 | 0.26 | 4.70E−05 | 387 |
| s.32394495 | 32394495 | T | A | 0.6 | 0.26 | 4.70E−05 | 388 |
| s.32394502 | 32394502 | G | T | 0.6 | 0.26 | 4.70E−05 | 389 |
| s.32394666 | 32394666 | A | C | 0.6 | 0.26 | 4.70E−05 | 390 |
| s.32394703 | 32394703 | T | C | 0.6 | 0.26 | 4.70E−05 | 391 |
| rs10112870 | 32394807 | C | T | 0.64 | 0.21 | 0.00041 | 392 |
| s.32394907 | 32394907 | T | C | 0.6 | 0.26 | 4.70E−05 | 393 |
| rs6993436 | 32395545 | A | G | 0.64 | 0.21 | 0.00041 | 394 |
| s.32398798 | 32398798 | T | A | 0.9 | 0.25 | 8.10E−06 | 395 |
| rs10503914 | 32400369 | C | T | 0.9 | 0.25 | 8.10E−06 | 396 |
| rs10503915 | 32404719 | T | C | 1 | 0.34 | 5.60E−13 | 397 |
| rs55899624 | 32407548 | G | A | 1 | 0.37 | 6.70E−14 | 398 |
| rs4733332 | 32407916 | G | T | 0.87 | 0.37 | 8.80E−08 | 399 |
| rs17642104 | 32408872 | T | C | 1 | 0.33 | 7.80E−12 | 400 |
| rs17642273 | 32411773 | C | A | 1 | 0.3 | 8.70E−11 | 401 |
| rs59861679 | 32414829 | A | G | 1 | 0.37 | 6.70E−14 | 402 |
| rs10808324 | 32415445 | C | T | 0.62 | 0.25 | 4.40E−05 | 403 |
| rs7009168 | 32416267 | C | T | 0.65 | 0.35 | 5.10E−07 | 404 |
| rs12678982 | 32416336 | G | A | 1 | 0.39 | 2.30E−14 | 405 |
| rs4129579 | 32417393 | A | G | 0.9 | 0.57 | 9.30E−13 | 406 |
| rs4129580 | 32417397 | A | C | 1 | 0.44 | 7.30E−16 | 407 |
| rs1579033 | 32417722 | C | G | 0.9 | 0.57 | 9.30E−13 | 408 |
| rs6981660 | 32418018 | C | T | 0.9 | 0.57 | 9.30E−13 | 409 |
| rs2347485 | 32419113 | C | G | 1 | 0.37 | 6.70E−14 | 410 |
| rs6468103 | 32420866 | T | C | 0.9 | 0.54 | 2.60E−12 | 411 |
| s.32421461 | 32421461 | G | A | 1 | 0.37 | 6.70E−14 | 412 |
| rs2347486 | 32422127 | C | T | 1 | 0.37 | 6.70E−14 | 413 |
| rs6468104 | 32423154 | T | G | 1 | 0.57 | 7.80E−20 | 414 |
| s.32423185 | 32423185 | T | C | 0.9 | 0.57 | 9.30E−13 | 415 |
| s.32424375 | 32424375 | C | T | 0.74 | 0.46 | 2.20E−09 | 416 |
| s.32424376 | 32424376 | C | T | 0.74 | 0.46 | 2.20E−09 | 417 |
| rs12707703 | 32424493 | C | T | 0.62 | 0.22 | 0.00033 | 418 |
| rs12707704 | 32424548 | G | A | 0.6 | 0.35 | 4.60E−07 | 419 |
| s.32424613 | 32424613 | T | G | 1 | 0.33 | 1.50E−12 | 420 |
| rs12707706 | 32424940 | G | T | 0.6 | 0.35 | 4.60E−07 | 421 |
| rs13439435 | 32424952 | T | A | 0.9 | 0.57 | 9.30E−13 | 422 |
| s.32424971 | 32424971 | T | G | 0.9 | 0.54 | 2.60E−12 | 423 |
| rs11993611 | 32425262 | C | T | 0.62 | 0.22 | 0.00033 | 424 |
| rs10103930 | 32425497 | A | G | 0.65 | 0.31 | 6.80E−06 | 425 |
| s.32426216 | 32426216 | G | A | 0.9 | 0.57 | 9.30E−13 | 426 |
| rs6996957 | 32426526 | C | T | 1 | 0.49 | 1.80E−17 | 427 |
| rs2347497 | 32428367 | A | C | 1 | 0.37 | 6.70E−14 | 428 |
| rs10503916 | 32428808 | A | T | 1 | 0.34 | 5.60E−13 | 429 |
| s.32428858 | 32428858 | A | G | 1 | 0.37 | 6.70E−14 | 430 |
| s.32428864 | 32428864 | T | C | 1 | 0.37 | 6.70E−14 | 431 |
| rs4733336 | 32428933 | C | G | 1 | 0.37 | 6.70E−14 | 432 |
| rs4733337 | 32429010 | T | A | 1 | 0.37 | 6.70E−14 | 433 |
| rs10113795 | 32429422 | T | A | 1 | 0.37 | 6.70E−14 | 434 |
| s.32429426 | 32429426 | C | G | 1 | 0.37 | 6.70E−14 | 435 |
| rs10098640 | 32429440 | A | G | 0.9 | 0.54 | 2.60E−12 | 436 |
| s.32431870 | 32431870 | G | C | 1 | 0.3 | 8.70E−11 | 437 |
| rs13439816 | 32432027 | A | G | 0.9 | 0.57 | 9.30E−13 | 438 |
| s.32432504 | 32432504 | C | T | 0.9 | 0.57 | 9.30E−13 | 439 |
| rs59299558 | 32432858 | T | C | 1 | 0.37 | 6.70E−14 | 440 |
| rs6981184 | 32433447 | A | G | 0.9 | 0.57 | 9.30E−13 | 441 |
| s.32434360 | 32434360 | A | G | 0.9 | 0.57 | 9.30E−13 | 442 |
| s.32435032 | 32435032 | G | A | 0.9 | 0.57 | 9.30E−13 | 443 |
| rs12216802 | 32460509 | A | G | 1 | 0.37 | 6.70E−14 | 505 |
| rs6468112 | 32461872 | C | T | 0.62 | 0.25 | 4.40E−05 | 506 |
| rs6468113 | 32462013 | T | C | 0.62 | 0.25 | 4.40E−05 | 507 |
| s.32462233 | 32462233 | T | C | 0.62 | 0.25 | 4.40E−05 | 508 |
| s.32463110 | 32463110 | T | C | 0.62 | 0.25 | 4.40E−05 | 509 |
| s.32463111 | 32463111 | G | A | 0.62 | 0.25 | 4.40E−05 | 510 |
| rs4621766 | 32463337 | T | A | 0.64 | 0.33 | 1.70E−06 | 511 |
| s.32463374 | 32463374 | A | C | 0.62 | 0.25 | 4.40E−05 | 512 |
| s.32463686 | 32463686 | A | T | 0.62 | 0.25 | 4.40E−05 | 513 |
| s.32463701 | 32463701 | T | G | 1 | 0.25 | 2.70E−09 | 514 |
| rs2881647 | 32464334 | C | T | 0.62 | 0.25 | 4.40E−05 | 515 |
| s.32464348 | 32464348 | G | A | 0.62 | 0.25 | 4.40E−05 | 516 |
| s.32464690 | 32464690 | T | C | 0.62 | 0.25 | 4.40E−05 | 517 |
| rs7844698 | 32465235 | C | T | 0.62 | 0.25 | 4.40E−05 | 518 |
| rs10097555 | 32465837 | A | G | 0.62 | 0.25 | 4.40E−05 | 519 |
| rs10087952 | 32465974 | C | T | 1 | 0.37 | 6.70E−14 | 520 |
| s.32466269 | 32466269 | A | G | 0.62 | 0.25 | 4.40E−05 | 521 |
| rs10099043 | 32466396 | G | C | 0.62 | 0.25 | 4.40E−05 | 522 |
| rs7002732 | 32466772 | G | C | 1 | 0.33 | 1.50E−12 | 523 |
| rs7001605 | 32466789 | C | G | 0.62 | 0.25 | 4.40E−05 | 524 |
| rs17645111 | 32466934 | T | C | 1 | 0.3 | 8.70E−11 | 525 |
| rs4370489 | 32467991 | G | A | 0.62 | 0.25 | 4.40E−05 | 526 |
| rs4278115 | 32468135 | T | C | 0.62 | 0.25 | 4.40E−05 | 527 |
| s.32468324 | 32468324 | A | G | 0.64 | 0.27 | 1.60E−05 | 528 |
| rs7821497 | 32468416 | G | A | 0.62 | 0.25 | 4.40E−05 | 529 |
| s.32469196 | 32469196 | C | A | 0.62 | 0.25 | 4.40E−05 | 530 |
| rs10503918 | 32469588 | G | A | 0.62 | 0.25 | 4.40E−05 | 531 |
| s.32470099 | 32470099 | C | G | 0.62 | 0.25 | 4.40E−05 | 532 |
| rs10093464 | 32470677 | A | G | 0.62 | 0.25 | 4.40E−05 | 533 |
| rs17645417 | 32470875 | C | T | 0.65 | 0.35 | 5.10E−07 | 534 |
| s.32471286 | 32471286 | C | T | 0.62 | 0.25 | 4.40E−05 | 535 |
| s.32471908 | 32471908 | G | C | 1 | 0.26 | 8.80E−10 | 536 |
| s.32471909 | 32471909 | C | T | 0.94 | 0.46 | 3.80E−10 | 537 |
| rs6468114 | 32473283 | G | T | 0.62 | 0.25 | 4.40E−05 | 538 |
| s.32473512 | 32473512 | T | G | 0.64 | 0.27 | 1.60E−05 | 539 |
| rs6468115 | 32473686 | G | T | 1 | 0.44 | 7.30E−16 | 540 |
| rs6468116 | 32473912 | G | T | 0.62 | 0.25 | 4.40E−05 | 541 |
| s.32474728 | 32474728 | C | T | 1 | 0.44 | 7.30E−16 | 542 |
| s.32474734 | 32474734 | T | C | 0.62 | 0.25 | 4.40E−05 | 543 |
| rs10755889 | 32474912 | G | A | 1 | 0.37 | 6.70E−14 | 544 |
| s.32475163 | 32475163 | A | G | 0.62 | 0.25 | 4.40E−05 | 545 |
| rs11506112 | 32475346 | C | G | 0.9 | 0.57 | 9.30E−13 | 546 |
| s.32475577 | 32475577 | T | C | 0.62 | 0.25 | 4.40E−05 | 547 |
| s.32477465 | 32477465 | T | C | 0.9 | 0.57 | 9.30E−13 | 548 |
| s.32478249 | 32478249 | G | A | 0.64 | 0.33 | 1.70E−06 | 549 |
| s.32478250 | 32478250 | T | A | 0.64 | 0.33 | 1.70E−06 | 550 |
| s.32478285 | 32478285 | C | T | 0.64 | 0.33 | 1.70E−06 | 551 |
| s.32478354 | 32478354 | C | T | 0.65 | 0.35 | 5.10E−07 | 552 |
| rs6996494 | 32479178 | C | T | 0.64 | 0.33 | 1.70E−06 | 553 |
| s.32479243 | 32479243 | T | C | 0.9 | 0.57 | 9.30E−13 | 554 |
| rs4733343 | 32479762 | G | T | 1 | 0.44 | 7.30E−16 | 555 |
| s.32479818 | 32479818 | T | C | 0.64 | 0.33 | 1.70E−06 | 556 |
| s.32480380 | 32480380 | C | T | 0.64 | 0.33 | 1.70E−06 | 557 |
| s.32481357 | 32481357 | A | C | 0.65 | 0.35 | 5.10E−07 | 558 |
| s.32481523 | 32481523 | T | C | 0.64 | 0.33 | 1.70E−06 | 559 |
| s.32482237 | 32482237 | G | A | 1 | 0.44 | 7.30E−16 | 560 |
| rs7013361 | 32482830 | C | A | 1 | 0.44 | 7.30E−16 | 561 |
| rs13259892 | 32485334 | T | A | 1 | 0.44 | 7.30E−16 | 562 |
| s.32485989 | 32485989 | T | C | 0.65 | 0.35 | 5.10E−07 | 563 |
| s.32486180 | 32486180 | T | C | 0.65 | 0.35 | 5.10E−07 | 564 |
| rs17645692 | 32489443 | A | C | 1 | 0.37 | 6.70E−14 | 565 |
| s.32494041 | 32494041 | A | C | 0.9 | 0.51 | 2.80E−11 | 566 |
| s.32494042 | 32494042 | A | T | 0.89 | 0.49 | 7.00E−11 | 567 |
| s.32494043 | 32494043 | A | T | 0.89 | 0.49 | 7.00E−11 | 568 |
| s.32494044 | 32494044 | G | T | 0.9 | 0.51 | 2.80E−11 | 569 |
| s.32494047 | 32494047 | G | T | 0.9 | 0.51 | 2.80E−11 | 570 |
| rs7844425 | 32495159 | G | T | 0.9 | 0.57 | 9.30E−13 | 571 |
| rs4733347 | 32495552 | G | A | 1 | 0.37 | 6.70E−14 | 572 |
| s.32499261 | 32499261 | T | C | 0.9 | 0.57 | 9.30E−13 | 573 |
| rs10088648 | 32500377 | T | A | 0.61 | 0.33 | 1.30E−06 | 574 |
| rs10092055 | 32500953 | G | A | 1 | 0.47 | 6.40E−17 | 575 |
| rs10954855 | 32501778 | T | A | 1 | 0.47 | 6.40E−17 | 576 |
| rs62500191 | 32501806 | C | A | 1 | 0.47 | 6.40E−17 | 577 |
| rs6651144 | 32502210 | T | C | 0.86 | 0.56 | 7.70E−12 | 578 |
| s.32502452 | 32502452 | G | T | 1 | 0.3 | 8.70E−11 | 579 |
| rs7000397 | 32503405 | G | A | 0.86 | 0.53 | 2.50E−11 | 580 |
| s.32503977 | 32503977 | C | T | 0.9 | 0.54 | 5.30E−12 | 581 |
| s.32504317 | 32504317 | C | T | 0.85 | 0.5 | 6.70E−11 | 582 |
| rs6651140 | 32504458 | A | G | 1 | 0.47 | 6.40E−17 | 583 |
| rs10108197 | 32505122 | G | A | 1 | 0.47 | 6.40E−17 | 584 |
| rs10111443 | 32505416 | C | T | 1 | 0.47 | 6.40E−17 | 585 |
| rs60550537 | 32509000 | T | A | 0.86 | 0.56 | 7.70E−12 | 586 |
| s.32509434 | 32509434 | G | A | 0.9 | 0.54 | 5.30E−12 | 587 |
| rs66963240 | 32511825 | T | C | 0.86 | 0.56 | 7.70E−12 | 588 |
| rs10099620 | 32512108 | A | G | 1 | 0.47 | 6.40E−17 | 589 |
| rs12334435 | 32513049 | C | T | 1 | 0.44 | 7.30E−16 | 590 |
| s.32513709 | 32513709 | T | C | 0.86 | 0.56 | 7.70E−12 | 591 |
| rs28594215 | 32515060 | A | G | 0.87 | 0.59 | 1.50E−12 | 592 |
| s.32515069 | 32515069 | C | A | 1 | 0.47 | 6.40E−17 | 593 |
| rs4733126 | 32515321 | A | C | 0.86 | 0.54 | 3.70E−11 | 594 |
| rs3934586 | 32516397 | G | A | 1 | 0.47 | 6.40E−17 | 595 |
| rs3934585 | 32516627 | G | A | 1 | 0.47 | 6.40E−17 | 596 |
| rs7819333 | 32517263 | C | G | 0.85 | 0.5 | 6.70E−11 | 597 |
| rs7838347 | 32517443 | G | A | 0.86 | 0.54 | 3.70E−11 | 598 |
| rs6468118 | 32518832 | G | C | 0.61 | 0.28 | 1.60E−05 | 599 |
| rs4489283 | 32519204 | C | T | 0.61 | 0.28 | 1.60E−05 | 600 |
| s.32519205 | 32519205 | A | G | 1 | 0.31 | 2.60E−11 | 601 |
| rs4422737 | 32519391 | A | G | 0.61 | 0.28 | 1.60E−05 | 602 |
| rs7826312 | 32519657 | C | T | 0.58 | 0.27 | 2.30E−05 | 603 |
| rs7000590 | 32520170 | C | T | 1 | 0.51 | 4.90E−18 | 604 |
| rs6996585 | 32520345 | G | A | 0.96 | 0.86 | 1.30E−20 | 605 |
| rs7005606 | 32521043 | G | T | 1 | 1 | — | 606 |
| rs6468119 | 32521103 | C | T | 1 | 0.73 | 7.20E−25 | 607 |
| s.32521783 | 32521783 | T | A | 1 | 0.46 | 7.10E−16 | 608 |
| rs7823498 | 32523115 | T | C | 1 | 0.25 | 4.80E−10 | 609 |
| s.32523368 | 32523368 | T | C | 1 | 0.47 | 6.40E−17 | 610 |
| s.32524171 | 32524171 | G | A | 1 | 1 | 5.70E−36 | 611 |
| s.32524438 | 32524438 | T | C | 1 | 1 | 5.70E−36 | 612 |
| s.32525059 | 32525059 | T | G | 1 | 1 | 5.70E−36 | 613 |
| s.32525690 | 32525690 | A | C | 1 | 1 | 5.70E−36 | 614 |
| s.32525924 | 32525924 | C | T | 1 | 1 | 5.70E−36 | 615 |
| s.32525989 | 32525989 | G | C | 1 | 0.48 | 1.60E−16 | 616 |
| s.32526144 | 32526144 | T | C | 1 | 1 | 5.70E−36 | 617 |
| s.32526310 | 32526310 | C | T | 1 | 1 | 5.70E−36 | 618 |
| rs4733130 | 32526536 | C | T | 1 | 1 | 5.70E−36 | 619 |
| s.32526995 | 32526995 | T | A | 1 | 1 | 5.70E−36 | 620 |
| s.32527279 | 32527279 | C | T | 1 | 1 | 5.70E−36 | 621 |
| s.32528362 | 32528362 | G | A | 1 | 1 | 5.70E−36 | 622 |
| rs4236709 | 32529652 | A | G | 1 | 0.37 | 6.70E−14 | 623 |
| rs4541858 | 32529851 | G | A | 1 | 1 | 5.70E−36 | 624 |
| rs12543882 | 32530235 | T | C | 1 | 1 | 5.70E−36 | 625 |
| rs2466104 | 32530254 | G | C | 1 | 0.23 | 2.80E−09 | 626 |
| rs7835688 | 32531041 | C | G | 1 | 1 | 5.70E−36 | 627 |
| s.32531198 | 32531198 | C | T | 1 | 1 | 5.70E−36 | 628 |
| s.32531622 | 32531622 | A | T | 1 | 0.48 | 1.60E−16 | 629 |
| rs2466103 | 32531846 | T | G | 1 | 0.26 | 1.90E−10 | 630 |
| rs2439312 | 32531901 | G | A | 1 | 0.33 | 1.50E−12 | 631 |
| s.32532554 | 32532554 | T | C | 1 | 0.48 | 1.60E−16 | 632 |
| s.32532563 | 32532563 | G | A | 1 | 1 | 5.70E−36 | 633 |
| s.32532822 | 32532822 | G | C | 1 | 1 | 5.70E−36 | 634 |
| rs4568578 | 32532829 | C | T | 1 | 0.51 | 4.90E−18 | 635 |
| rs11991474 | 32532852 | T | C | 1 | 1 | 5.70E−36 | 636 |
| rs9642727 | 32533574 | C | A | 1 | 0.97 | 1.00E−33 | 637 |
| rs17646936 | 32533616 | A | G | 1 | 0.47 | 6.40E−17 | 638 |
| rs17719687 | 32533708 | G | A | 1 | 0.25 | 2.70E−09 | 639 |
| s.32533874 | 32533874 | T | A | 1 | 1 | 5.70E−36 | 640 |
| s.32534156 | 32534156 | G | A | 1 | 0.25 | 2.70E−09 | 641 |
| s.32535043 | 32535043 | C | T | 1 | 0.48 | 1.60E−16 | 642 |
| rs6989777 | 32535224 | A | G | 1 | 0.48 | 1.60E−16 | 643 |
| rs7825175 | 32535816 | A | G | 1 | 0.25 | 2.70E−09 | 644 |
| s.32535941 | 32535941 | T | A | 1 | 0.48 | 1.60E−16 | 645 |
| s.32536084 | 32536084 | A | G | 1 | 0.48 | 1.60E−16 | 646 |
| rs11777396 | 32536776 | T | G | 1 | 0.48 | 1.60E−16 | 647 |
| s.32536914 | 32536914 | A | G | 1 | 0.48 | 1.60E−16 | 648 |
| rs10101464 | 32537004 | C | T | 1 | 0.39 | 2.30E−14 | 649 |
| rs13260545 | 32537142 | T | C | 1 | 0.39 | 2.30E−14 | 650 |
| s.32538611 | 32538611 | A | G | 1 | 0.48 | 1.60E−16 | 651 |
| rs11776203 | 32538661 | G | T | 1 | 0.48 | 1.60E−16 | 652 |
| s.32539338 | 32539338 | T | C | 1 | 0.46 | 7.10E−16 | 653 |
| rs4316112 | 32539889 | A | C | 1 | 0.48 | 1.60E−16 | 654 |
| s.32540276 | 32540276 | G | A | 1 | 0.48 | 1.60E−16 | 655 |
| s.32540531 | 32540531 | T | G | 1 | 0.48 | 1.60E−16 | 656 |
| rs12679578 | 32540667 | T | C | 1 | 0.48 | 1.60E−16 | 657 |
| s.32540813 | 32540813 | G | A | 1 | 0.48 | 1.60E−16 | 658 |
| s.32540929 | 32540929 | G | A | 1 | 0.48 | 1.60E−16 | 659 |
| rs12682268 | 32541497 | A | G | 1 | 0.49 | 1.80E−17 | 660 |
| s.32541620 | 32541620 | C | T | 1 | 0.46 | 7.10E−16 | 661 |
| s.32541642 | 32541642 | T | G | 1 | 0.48 | 1.60E−16 | 662 |
| s.32542073 | 32542073 | C | T | 1 | 0.48 | 1.60E−16 | 663 |
| s.32542399 | 32542399 | G | T | 1 | 0.48 | 1.60E−16 | 664 |
| s.32542400 | 32542400 | G | C | 1 | 0.48 | 1.60E−16 | 665 |
| s.32542428 | 32542428 | T | C | 1 | 0.48 | 1.60E−16 | 666 |
| rs13258892 | 32543079 | C | T | 1 | 0.45 | 2.20E−16 | 667 |
| s.32543080 | 32543080 | T | G | 1 | 0.48 | 1.60E−16 | 668 |
| s.32543180 | 32543180 | G | A | 1 | 0.46 | 7.10E−16 | 669 |
| s.32543188 | 32543188 | A | G | 1 | 0.48 | 1.60E−16 | 670 |
| s.32543446 | 32543446 | T | C | 1 | 0.48 | 1.60E−16 | 671 |
| rs11775204 | 32543629 | G | A | 1 | 0.48 | 1.60E−16 | 672 |
| s.32543699 | 32543699 | G | T | 1 | 0.48 | 1.60E−16 | 673 |
| s.32543963 | 32543963 | G | A | 1 | 0.48 | 1.60E−16 | 674 |
| s.32544371 | 32544371 | A | G | 1 | 0.26 | 8.80E−10 | 675 |
| rs35525180 | 32544681 | G | A | 1 | 0.47 | 6.40E−17 | 676 |
| rs4733132 | 32545285 | G | C | 1 | 0.48 | 1.60E−16 | 677 |
| rs11787271 | 32545488 | T | C | 1 | 0.48 | 1.60E−16 | 678 |
| s.32545704 | 32545704 | T | G | 1 | 0.48 | 1.60E−16 | 679 |
| rs13252144 | 32546324 | G | T | 1 | 0.51 | 4.90E−18 | 680 |
| rs13252431 | 32546426 | G | A | 1 | 0.39 | 2.30E−14 | 681 |
| s.32546942 | 32546942 | G | T | 1 | 0.48 | 1.60E−16 | 682 |
| s.32547121 | 32547121 | C | T | 1 | 0.48 | 1.60E−16 | 683 |
| rs4733360 | 32547745 | C | G | 1 | 0.48 | 1.60E−16 | 684 |
| rs10503920 | 32548231 | A | G | 1 | 0.47 | 6.40E−17 | 685 |
| rs2466100 | 32548891 | T | A | 1 | 1 | 5.70E−36 | 686 |
| rs2439305 | 32549006 | G | A | 1 | 1 | 5.70E−36 | 687 |
| rs35233333 | 32549276 | T | C | 1 | 0.51 | 4.90E−18 | 688 |
| s.32549381 | 32549381 | T | G | 1 | 0.25 | 2.70E−09 | 689 |
| rs2466098 | 32549458 | A | G | 1 | 1 | 5.70E−36 | 690 |
| rs2439304 | 32549913 | A | G | 1 | 0.78 | 1.30E−26 | 691 |
| rs2439303 | 32549917 | T | C | 1 | 1 | 5.70E−36 | 692 |
| s.32550116 | 32550116 | G | T | 1 | 0.48 | 1.60E−16 | 693 |
| rs2466097 | 32550203 | A | T | 1 | 0.25 | 2.70E−09 | 694 |
| s.32550232 | 32550232 | G | A | 1 | 0.25 | 2.70E−09 | 695 |
| rs2466096 | 32550274 | A | T | 1 | 0.25 | 2.70E−09 | 696 |
| rs2466095 | 32550391 | C | T | 1 | 1 | 5.70E−36 | 697 |
| rs2919373 | 32551401 | T | C | 1 | 0.25 | 2.70E−09 | 698 |
| rs2439302 | 32551911 | G | C | 1 | 1 | 5.70E−36 | 699 |
| rs2466077 | 32552295 | G | T | 0.92 | 0.79 | 7.40E−18 | 700 |
| rs2466076 | 32552338 | G | T | 0.93 | 0.86 | 6.60E−20 | 701 |
| rs2466075 | 32552491 | A | G | 0.93 | 0.86 | 6.60E−20 | 702 |
| s.32552499 | 32552499 | G | A | 0.91 | 0.31 | 7.00E−06 | 703 |
| rs2466074 | 32552680 | C | T | 0.96 | 0.83 | 2.60E−19 | 704 |
| rs17720837 | 32552708 | T | C | 0.93 | 0.4 | 2.70E−08 | 705 |
| rs2466073 | 32552854 | G | A | 0.9 | 0.51 | 5.20E−11 | 706 |
| rs2439299 | 32553227 | A | C | 0.95 | 0.57 | 4.00E−12 | 707 |
| s.32553256 | 32553256 | G | T | 0.93 | 0.4 | 2.70E−08 | 708 |
| rs2466072 | 32553435 | G | A | 0.84 | 0.66 | 4.40E−14 | 709 |
| rs2466071 | 32553664 | A | T | 0.96 | 0.83 | 2.60E−19 | 710 |
| s.32553918 | 32553918 | G | A | 1 | 0.25 | 2.70E−09 | 711 |
| rs2466070 | 32554159 | C | T | 0.96 | 0.83 | 2.60E−19 | 712 |
| s.32555334 | 32555334 | G | A | 0.93 | 0.4 | 2.70E−08 | 713 |
| rs11783278 | 32556075 | A | T | 0.93 | 0.4 | 2.70E−08 | 714 |
| s.32556327 | 32556327 | C | T | 0.93 | 0.4 | 2.70E−08 | 715 |
| rs17721043 | 32556417 | A | G | 0.93 | 0.4 | 2.70E−08 | 716 |
| s.32559506 | 32559506 | C | T | 0.76 | 0.3 | 9.10E−07 | 717 |
| s.32559771 | 32559771 | C | T | 0.76 | 0.3 | 9.10E−07 | 718 |
| s.32561481 | 32561481 | C | T | 0.93 | 0.42 | 1.40E−08 | 719 |
| rs2439292 | 32566424 | G | A | 0.76 | 0.3 | 9.10E−07 | 720 |
| rs2919381 | 32683466 | G | A | 0.59 | 0.24 | 2.70E−05 | 721 |

### Association Analysis

For single marker association to a disease, the Fisher exact test can be used to calculate two-sided p-values for each individual allele. Correcting for relatedness among patients can be done by extending a variance adjustment procedure previously described (Risch, N. & Teng, J. Genome Res., 8:1273-1288 (1998)) for sibships so that it can be applied to general familial relationships. The method of genomic controls (Devlin, B. & Roeder, K. Biometrics 55:997 (1999)) can also be used to adjust for the relatedness of the individuals and possible stratification.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(fⱼ/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, for example a cohort from a different population (e.g., different region of same country, or a different country) of the same or different ethnicity. The advantage of replication studies is that the number of tests performed in the replication study is usually quite small, and hence the less stringent the statistical measure that needs to be applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold (i.e., more significant) are a measure of a true genetic effect, and replication in additional cohorts is not necessary from a statistical point of view. Importantly, however, signals with P-values that are greater than this threshold may also be due to a true genetic effect. The sample size in the first study may not have been sufficiently large to provide an observed P-value that meets the conservative threshold for genome-wide significance, or the first study may not have reached genome-wide significance due to inherent fluctuations due to sampling. Since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations.

Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

### Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

### Determining Risk

In the present context, an individual who is at an increased susceptibility (i.e., increased risk) for Thyroid Cancer is an individual who is carrying at least one at-risk allele in marker rs7005606 or marker rs966423. Alternatively, an individual who is at an increased susceptibility for Thyroid Cancer is an individual who is carrying at least one at-risk allele in a correlated marker in linkage disequilibrium with rs7005606 or marker rs966423. The correlated marker may in certain embodiments be selected from the polymorphic marksers described herein. In certain embodiments, an at-risk allele of a marker correlated with rs966423 is selected from the group consisting of the risk alleles shown in Table 1 herein. In certain embodiments, an at-risk allele of a marker correlated with rs7005606 is selected from the group consisting of the risk alleles shown in Table 2 herein. In certain embodiments, risk alleles are selected from the risk alleles shown in Table 7 and Table 8 herein. For example, Table 8 shows risk alleles associated with risk of thyroid cancer for surrogate markers of rs966423, and Table 7 shows risk alleles for thyroid cancer for surrogate markers of rs7005606. In one embodiment, significance associated with a marker is measured by a relative risk (RR). In another embodiment, significance associated with a marker or haplotye is measured by an odds ratio (OR). In a further embodiment, the significance is measured by a percentage. In one embodiment, a significant increased risk is measured as a risk (relative risk and/or odds ratio) of at least 1.10, including but not limited to: at least 1.15, at least 1.20, at least 1.25, at least 1.30, at least 1.35, at least 1.40, at least 1.45, at least 1.50, at least 1.55, at least 1.60, and at least 1.65. In a particular embodiment, a risk (relative risk and/or odds ratio) of at least 1.25 is significant. In another particular embodiment, a risk of at least 1.30 is significant.

An at-risk polymorphic marker as described herein is one where at least one allele of at least one marker is more frequently present in an individual diagnosed with, or at risk for, Thyroid Cancer (affected), compared to the frequency of its presence in a comparison group (control), such that the presence of the marker allele is indicative of increased susceptibility to Thyroid Cancer. The control group may in one embodiment be a population sample, i.e. a random sample from the general population. In another embodiment, the control group is represented by a group of individuals who are disease-free, i.e. individuals who have not been diagnosed with Thyroid Cancer.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

### Database

Determining susceptibility can alternatively or additionally comprise comparing nucleic acid sequence data and/or genotype data to a database containing correlation data between polymorphic markers and susceptibility to Thyroid Cancer. The database can be part of a computer-readable medium described herein.

In a specific aspect of the invention, the database comprises at least one measure of susceptibility to the condition for the polymorphic markers. For example, the database may comprise risk values associated with particular genotypes at such markers. The database may also comprise risk values associated with particular genotype combinations for multiple such markers.

In another specific aspect of the invention, the database comprises a look-up table containing at least one measure of susceptibility to the condition for the polymorphic markers.

### Further Steps

The methods disclosed herein can comprise additional steps which may occur before, after, or simultaneously with one of the aforementioned steps of the method of the invention. In a specific embodiment of the invention, the method of determining a susceptibility to Thyroid Cancer further comprises reporting the susceptibility to at least one entity selected from the group consisting of the individual, a guardian of the individual, a genetic service provider, a physician, a medical organization, and a medical insurer. The reporting may be accomplished by any of several means. For example, the reporting can comprise sending a written report on physical media or electronically or providing an oral report to at least one entity of the group, which written or oral report comprises the susceptibility. Alternatively, the reporting can comprise providing the at least one entity of the group with a login and password, which provides access to a report comprising the susceptibility posted on a password-protected computer system.

### Study Population

In a general sense, the methods and kits described herein can be utilized from samples containing nucleic acid material (DNA or RNA) from any source and from any individual, or from genotype or sequence data derived from such samples. In preferred embodiments, the individual is a human individual. The individual can be an adult, child, or fetus. The nucleic acid source may be any sample comprising nucleic acid material, including biological samples, or a sample comprising nucleic acid material derived therefrom. The present invention also provides for assessing markers in individuals who are members of a target population. Such a target population is in one embodiment a population or group of individuals at risk of developing Thyroid Cancer, based on other genetic factors, biomarkers, biophysical parameters, history of Thyroid Cancer, family history of Thyroid Cancer or a related disease. In certain embodiments, a target population is a population with abnormal levels (high or low) of TSH, T4 or T3.

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Sulem, P., et al. Nat Genet May 17, 2009 (Epub ahead of print); Rafnar, T., et al. Nat Genet. 41:221-7 (2009); Gretarsdottir, S., et al. Ann Neurol 64:402-9 (2008); Stacey, S, N., et al. Nat Genet. 40:1313-18 (2008); Gudbjartsson, D. F., et al. Nat Genet. 40:886-91 (2008); Styrkarsdottir, U., et al. N Engl J Med 358:2355-65 (2008); Thorgeirsson, T., et al. Nature 452:638-42 (2008); Gudmundsson, J., et al. Nat. Genet. 40:281-3 (2008); Stacey, S, N., et al., Nat. Genet. 39:865-69 (2007); Helgadottir, A., et al., Science 316:1491-93 (2007); Steinthorsdottir, V., et al., Nat. Genet. 39:770-75 (2007); Gudmundsson, J., et al., Nat. Genet. 39:631-37 (2007); Frayling, T M, Nature Reviews Genet. 8:657-662 (2007); Amundadottir, L. T., et al., Nat. Genet. 38:652-58 (2006); Grant, S. F., et al., Nat. Genet. 38:320-23 (2006)). Thus, genetic findings in the Icelandic population have in general been replicated in other populations, including populations from Africa and Asia.

It is thus believed that the markers described herein to be associated with risk of Thyroid Cancer will show similar association in other human populations. Particular embodiments comprising individual human populations are thus also contemplated and within the scope of the invention. Such embodiments relate to human subjects that are from one or more human population including, but not limited to, Caucasian populations, European populations, American populations, Eurasian populations, and Asian populations.

The racial contribution in individual subjects may also be determined by genetic analysis. Genetic analysis of ancestry may be carried out using unlinked microsatellite markers such as those set out in Smith et al. (Am J Hum Genet. 74, 1001-13 (2004)).

In certain embodiments, the invention relates to markers identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as thought herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Screening Methods

The invention also provides a method of screening candidate markers for assessing susceptibility to Thyroid Cancer. The invention also provides a method of identification of a marker for use in assessing susceptibility to Thyroid Cancer. The method may comprise analyzing the frequency of at least one allele of a polymorphic marker in a population of human individuals diagnosed with Thyroid Cancer, wherein a significant difference in frequency of the at least one allele in the population of human individuals diagnosed with Thyroid Cancer as compared to the frequency of the at least one allele in a control population of human individuals is indicative of the allele as a marker of the Thyroid Cancer. In certain embodiments, the candidate marker is a marker in linkage disequilibrium with marker rs7005606 or marker rs966423.

In one embodiment, the method comprises (i) identifying at least one polymorphic marker in linkage disequilibrium, as determined by values of r² of greater than 0.5, with marker rs7005606 or marker rs966423; (ii) obtaining sequence information about the at least one polymorphic marker in a group of individuals diagnosed with Thyroid Cancer; and (iii) obtaining sequence information about the at least one polymorphic marker in a group of control individuals; wherein determination of a significant difference in frequency of at least one allele in the at least one polymorphism in individuals diagnosed with Thyroid Cancer as compared with the frequency of the at least one allele in the control group is indicative of the at least one polymorphism being useful for assessing susceptibility to Thyroid Cancer.

In one embodiment, an increase in frequency of the at least one allele in the at least one polymorphism in individuals diagnosed with Thyroid Cancer, as compared with the frequency of the at least one allele in the control group, is indicative of the at least one polymorphism being useful for assessing increased susceptibility to Thyroid Cancer. In another embodiment, a decrease in frequency of the at least one allele in the at least one polymorphism in individuals diagnosed with Thyroid Cancer, as compared with the frequency of the at least one allele in the control group, is indicative of the at least one polymorphism being useful for assessing decreased susceptibility to, or protection against, Thyroid Cancer.

### Thyroid Stimulating Hormone

Thyroid-stimulating hormone (also known as TSH or thyrotropin) is a peptide hormone synthesized and secreted by thyrotrope cells in the anterior pituitary gland which regulates the endocrine function of the thyroid gland. TSH stimulates the thyroid gland to secrete the hormones thyroxine (T₄) and triiodothyronine (T₃). TSH production is controlled by a Thyrotropin Releasing Hormone, (TRH), which is manufactured in the hypothalamus and transported to the anterior pituitary gland via the superior hypophyseal artery, where it increases TSH production and release. Somatostatin is also produced by the hypothalamus, and has an opposite effect on the pituitary production of TSH, decreasing or inhibiting its release.

The level of thyroid hormones (T₃ and T₄) in the blood have an effect on the pituitary release of TSH; when the levels of T₃ and T₄ are low, the production of TSH is increased, and conversely, when levels of T₃ and T₄ are high, then TSH production is decreased. This effect creates a regulatory negative feedback loop.

Thyroxine, or 3,5,3′,5′-tetraiodothyronine (often abbreviated as T₄), is the major hormone secreted by the follicular cells of the thyroid gland. T₄ is transported in blood, with 99.95% of the secreted T₄ being protein bound, principally to thyroxine-binding globulin (TBG), and, to a lesser extent, to transthyretin and serum albumin. T₄ is involved in controlling the rate of metabolic processes in the body and influencing physical development. Administration of thyroxine has been shown to significantly increase the concentration of nerve growth factor in the brains of adult mice.

In the hypothalamus, T₄ is converted to Triiodothyronine, also known as T₃. TSH is inhibited mainly by T₃. The thyroid gland releases greater amounts of T₄ than T₃, so plasma concentrations of T₄ are 40-fold higher than those of T₃. Most of the circulating T₃ is formed peripherally by deiodination of T₄ (85%), a process that involves the removal of iodine from carbon 5 on the outer ring of T₄. Thus, T₄ acts as prohormone for T₃.

### Utility of Genetic Testing

As discussed in the above, the primary known risk factor for thyroid cancer is radiation exposure. Thyroid cancer incidence within the US has been rising for several decades (Davies, L. and Welch, H. G., Jama, 295, 2164 (2006)), which may be attributable to increased detection of sub-clinical cancers, as opposed to an increase in the true occurrence of thyroid cancer (Davies, L. and Welch, H. G., Jama, 295, 2164 (2006)). The introduction of ultrasonography and fine-needle aspiration biopsy in the 1980s improved the detection of small nodules and made cytological assessment of a nodule more routine (Rojeski, M. T. and Gharib, H., N Engl J Med, 313, 428 (1985), Ross, D. S., J Clin Endocrinol Metab, 91, 4253 (2006)). This increased diagnostic scrutiny may allow early detection of potentially lethal thyroid cancers. However, several studies report thyroid cancers as a common autopsy finding (up to 35%) in persons without a diagnosis of thyroid cancer (Bondeson, L. and Ljungberg, O., Cancer, 47, 319 (1981), Harach, H. R., et al., Cancer, 56, 531 (1985), Solares, C. A., et al., Am J Otolaryngol, 26, 87 (2005) and Sobrinho-Simoes, M. A., Sambade, M. C., and Goncalves, V., Cancer, 43, 1702 (1979)). This suggests that many people live with sub-clinical forms of thyroid cancer which are of little or no threat to their health.

Physicians use several tests to confirm the suspicion of thyroid cancer, to identify the size and location of the lump and to determine whether the lump is non-cancerous (benign) or cancerous (malignant). Blood tests such as the thyroid stimulating hormone (TSH) test check thyroid function.

TSH levels are tested in the blood of patients suspected of suffering from excess (hyperthyroidism), or deficiency (hypothyroidism) of thyroid hormone. Generally, a normal range for TSH for adults is between 0.2 and 10 uIU/mL (equivalent to mIU/L). The optimal TSH level for patients on treatment ranges between 0.3 to 3.0 mIU/L. The interpretation of TSH measurements depends also on what the blood levels of thyroid hormones (T₃ and T₄) are. The National Health Service in the UK considers a “normal” range to be more like 0.1 to 5.0 uIU/mL.

TSH levels for children normally start out much higher. In 2002, the National Academy of Clinical Biochemistry (NACB) in the United States recommended age-related reference limits starting from about 1.3-19 uIU/mL for normal term infants at birth, dropping to 0.6-10 uIU/mL at 10 weeks old, 0.4-7.0 uIU/mL at 14 months and gradually dropping during childhood and puberty to adult levels, 0.4-4.0 uIU/mL. The NACB also stated that it expected the normal (95%) range for adults to be reduced to 0.4-2.5 uIU/mL, because research had shown that adults with an initially measured TSH level of over 2.0 uIU/mL had an increased odds ratio of developing hypothyroidism over the [following] 20 years, especially if thyroid antibodies were elevated.

In general, both TSH and T₃ and T₄ should be measured to ascertain where a specific thyroid dysfunction is caused by primary pituitary or by a primary thyroid disease. If both are up (or down) then the problem is probably in the pituitary. If the one component (TSH) is up, and the other (T₃ and T₄) is down, then the disease is probably in the thyroid itself. The same holds for a low TSH, high T3 and T4 finding.

The knowledge of underlying genetic risk factors for thyroid cancer can be utilized in the application of screening programs for thyroid cancer. Thus, carriers of at-risk variants for thyroid cancer may benefit from more frequent screening than do non-carriers. Homozygous carriers of at-risk variants are particularly at risk for developing thyroid cancer.

It may be beneficial to determine TSH, T3 and/or T4 levels in the context of a particular genetic profile, e.g. the presence of particular at-risk alleles for thyroid cancer as described herein (e.g., rs7005606 allele G and/or rs966423 allele C). Since TSH, T3 and T4 are measures of thyroid function, a diagnostic and preventive screening program will benefit from analysis that includes such clinical measurements. For example, an abnormal (increased or decreased) level of TSH together with determination of the presence of an at-risk genetic variant for thyroid cancer (e.g., rs7005606 and/or rs966423) is indicative that an individual is at risk of developing thyroid cancer. In one embodiment, determination of a decreased level of TSH in an individual in the context of the presence of rs7005606 allele G and/or rs966423 allele C is indicative of an increased risk of thyroid cancer for the individual.

Also, carriers may benefit from more extensive screening, including ultrasonography and/or fine needle biopsy. The goal of screening programs is to detect cancer at an early stage. Knowledge of genetic status of individuals with respect to known risk variants can aid in the selection of applicable screening programs. In certain embodiments, it may be useful to use the at-risk variants for thyroid cancer described herein together with one or more diagnostic tool selected from Radioactive Iodine (RAI) Scan, Ultrasound examination, CT scan (CAT scan), Magnetic Resonance Imaging (MRI), Positron Emission Tomography (PET) scan, Fine needle aspiration biopsy and surgical biopsy.

The invention provides in one diagnostic aspect a method for identifying a subject who is a candidate for further diagnostic evaluation for thyroid cancer, comprising the steps of (a) determining, in the genome of a human subject, the allelic identity of at least one polymorphic marker, wherein different alleles of the at least one marker are associated with different susceptibilities to thyroid cancer, and wherein the at least one marker is selected from the group consisting of rs966423 and rs7005606, and correlated markers in linkage disequilibrium therewith; and (b) identifying the subject as a subject who is a candidate for further diagnostic evaluation for thyroid cancer based on the allelic identity at the at least one polymorphic marker. Thus, the identification of individuals who are at increased risk of developing thyroid cancer may be used to select those individuals for follow-up clinical evaluation, as described in the above.

### Prognostic Methods

In addition to the utilities described above, the polymorphic markers of the invention are useful in determining prognosis of a human individual experiencing symptoms associated with, or an individual diagnosed with, thyroid cancer. Accordingly, the invention provides a method of predicting prognosis of an individual experiencing symptoms associated with, or an individual diagnosed with, thyroid cancer. The method comprises analyzing sequence data about a human individual for at least one polymorphic marker selected from the group consisting of rs7005606 and rs966423, and markers in linkage disequilibrium therewith, wherein different alleles of the at least one polymorphic marker are associated with different susceptibilities thyroid cancer in humans, and predicting prognosis of the individual from the sequence data.

The prognosis can be any type of prognosis relating to the progression of thyroid cancer, and/or relating to the chance of recovering from thyroid cancer. The prognosis can, for instance, relate to the severity of the cancer, when the cancer may take place (e.g., the likelihood of recurrence), or how the cancer will respond to therapeutic treatment.

With regard to the prognostic methods described herein, the sequence data obtained to establish a prognostic prediction is suitably nucleic acid sequence data. For example, in one embodiment, determination of the presence of an at-risk allele of thyroid cancer (e.g., rs7005606 allele G and/or rs966423 allele C) is useful for prognostic applications. Suitable methods of detecting particular at-risk alleles are known in the art, some of which are described herein.

### Therapeutic Agents

Treatment options for thyroid cancer include current standard treatment methods and those that are in clinical trials.

Current treatment options for thyroid cancer include:

Surgery—including lobectomy, where the lobe in which thyroid cancer is found is removed, thyroidectomy, where all but a very small part of the thyroid is removed, total thyroidectomoy, where the entire thyroid is removed, and lymphadenectomoy, where lymph nodes in the neck that contain cancerous growth are removed;
Radiation therapy—including externation radiation therapy and internal radiation therapy using a radioactive compound. Radiation therapy may be given after surgery to remove any surviving cancer cells. Also, follicular and papillary thyroid cancers are sometimes treated with radioactive iodine (RAI) therapy;
Chemotherapy—including the use of oral or intravenous administration of the chemotherapy compound;
Thyroid hormone therapy—this therapy includes administration of drugs preventing generation of thyroid-stimulating hormone (TSH) in the body.

A number of clinical trials for thyroid cancer therapy and treatment are currently ongoing, including but not limited to trials for ¹⁸F-fluorodeoxyglucose (FluGlucoScan); ¹¹¹In-Pentetreotide (NeuroendoMedix); Combretastatin and Paclitaxel/Carboplatin in the treatment of anaplastic thyroid cancer, ¹³¹I with or without thyroid-stimulating hormone for post-surgical treatment, XL184-301 (Exelixis), Vandetanib (Zactima; Astra Zeneca), CS-7017 (Sankyo), Decitabine (Dacogen; 5-aza-2′-deoxycytidine), Irinotecan (Pfizer, Yakult Honsha), Bortezomib (Velcade; Millenium Pharmaceuticals); 17-AAG (17-N-Allylamino-17-demethoxygeldanamycin), Sorafenib (Nexavar, Bayer), recombinant Thyrotropin, Lenalidomide (Revlimid, Celgene), Sunitinib (Sutent), Sorafenib (Nexavar, Bayer), Axitinib (AG-013736, Pfizer), Valproic Acid (2-propylpentanoic acid), Vandetanib (Zactima, Astra Zeneca), AZD6244 (Astra Zeneca), Bevacizumab (Avastin, Genetech/Roche), MK-0646 (Merck), Pazopanib (GlaxoSmithKline), Aflibercept (Sanofi-Aventis & Regeneron Pharmaceuticals), and FR901228 (Romedepsin).

### Methods for Predicting Response to Therapeutic Agents

As is known in the art, individuals can have differential responses to a particular therapy (e.g., a therapeutic agent or therapeutic method). Pharmacogenomics addresses the issue of how genetic variations (e.g., the variants (markers and/or haplotypes) of the invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the invention), or therapeutic failure of the drug. Therefore, the variants of the invention may determine the manner in which a therapeutic agent and/or method acts on the body, or the way in which the body metabolizes the therapeutic agent.

Accordingly, in one embodiment, the presence of a particular allele at a polymorphic site (e.g., rs7005606 allele G and/or rs966423 allele C) is indicative of a different response, e.g. a different response rate, to a particular treatment modality, for thyroid cancer. This means that a patient diagnosed with thyroid cancer and carrying such risk alleles would respond better to, or worse to, a specific therapeutic, drug and/or other therapy used to treat the cancer. Therefore, the presence or absence of the marker allele could aid in deciding what treatment should be used for the patient. If the patient is positive for the marker allele, then the physician recommends one particular therapy, while if the patient is negative for the at least one allele of a marker, then a different course of therapy may be recommended (which may include recommending that no immediate therapy, other than serial monitoring for progression of symptoms, be performed). Thus, the patient's carrier status could be used to help determine whether a particular treatment modality should be administered. In one embodiment, the presence of an at-risk allele for thyroid cancer, e.g. rs7005606 allele G and/or rs966423 allele C, is indicative of a positive response to a particular therapy for thyroid cancer. In certain embodiments, the therapy is selected from the group consisting of surgery, radiation therapy, chemotherapy and thyroid hormone therapy.

Another aspect of the invention relates to methods of selecting individuals suitable for a particular treatment modality, based on the their likelihood of developing particular complications or side effects of the particular treatment. It is well known that many therapeutic agents can lead to certain unwanted complications or side effects. Likewise, certain therapeutic procedures or operations may have complications associated with them. Complications or side effects of these particular treatments or associated with specific therapeutic agents can, just as diseases do, have a genetic component. It is therefore contemplated that selection of the appropriate treatment or therapeutic agent can in part be performed by determining the genotype of an individual, and using the genotype status (e.g., the presence or absence of rs7005606 allele G and/or rs966423 allele C) of the individual to decide on a suitable therapeutic procedure or on a suitable therapeutic agent to treat thyroid cancer. It is therefore contemplated that the polymorphic markers of the invention can be used in this manner. Indiscriminate use of such therapeutic agents or treatment modalities may lead to unnecessary and needless adverse complications.

In view of the foregoing, the invention provides a method of assessing an individual for probability of response to a therapeutic agent for preventing, treating, and/or ameliorating symptoms associated thyroid cancer. In one embodiment, the method comprises: analyzing nucleic acid sequence data from a human individual for at least one polymorphic marker selected from the group consisting of rs7005606 and rs966423, and markers in linkage disequilibrium therewith, wherein determination of the presence of the rs7005606 allele G and/or rs966423 allele C, or a marker allele in linkage disequilibrium therewith, indicative of a probability of a positive response to the therapeutic agent.

In a further aspect, the markers of the invention can be used to increase power and effectiveness of clinical trials. Thus, individuals who are carriers of particular at-risk variants for thyroid cancer (e.g., rs7005606 allele G and/or rs966423 allele C) may be more likely to respond to a particular treatment modality. For some treatments, the genetic risk may correlate with less responsiveness to therapy. This application can improve the safety of clinical trials, but can also enhance the chance that a clinical trial will demonstrate statistically significant efficacy, which may be limited to a certain sub-group of the population. Thus, one possible outcome of such a trial is that carriers of the at-risk markers of the invention are statistically significantly likely to show positive response to the therapeutic agent, i.e. experience alleviation of symptoms associated with thyroid cancer, when taking the therapeutic agent or drug as prescribed. Another possible outcome is that genetic carriers show less favorable response to the therapeutic agent, or show differential side-effects to the therapeutic agent compared to the non-carrier. An aspect of the invention is directed to screening for such pharmacogenetic correlations.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies, means for amplification of nucleic acids, means for analyzing the nucleic acid sequence of nucleic acids, means for analyzing the amino acid sequence of a polynucleotides, etc. The kits can for example include necessary buffers, nucleic acid primers for amplifying nucleic acids (e.g., a nucleic acid segment comprising one or more of the polymorphic markers as described herein), and reagents for allele-specific detection of the fragments amplified using such primers and necessary enzymes (e.g., dna polymerase). Additionally, kits can provide reagents for assays to be used in combination with the methods of the present invention, e.g., reagents for use with other diagnostic assays for thyroid cancer.

In one embodiment, the invention pertains to a kit for assaying a sample from a subject to detect a susceptibility to thyroid cancer in the subject, wherein the kit comprises reagents necessary for selectively detecting at least one at-risk variant for thyroid cancer in the individual, wherein the at least one at-risk variant is selected from the group consisting of rs7005606 and rs966423, and markers in linkage disequilibrium therewith. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with thyroid cancer risk. In one such embodiment, the polymorphism is selected from the group consisting of rs7005606 and rs966423, and polymorphic markers in linkage disequilibrium therewith. In yet another embodiment the fragment is at least 20 base pairs in size. Such oligonucleotides or nucleic acids (e.g., oligonucleotide primers) can be designed using portions of the nucleic acid sequence flanking the polymorphism. In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In one embodiment, the DNA template is amplified before detection by PCR. The DNA template may also be amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

In certain embodiments, determination of the presence of a particular marker allele (e.g. allele G of rs7005606 and/or allele C of rs966423) is indicative of an increased susceptibility of thyroid cancer. In another embodiment, determination of the presence of a particular marker allele is indicative of prognosis of thyroid cancer. In another embodiment, the presence of a marker allele is indicative of response to a therapeutic agent for thyroid cancer. In yet another embodiment, the presence of a marker allele is indicative of progress of treatment of thyroid cancer.

In certain embodiments, the kit comprises reagents for detecting no more than 100 alleles in the genome of the individual. In certain other embodiments, the kit comprises reagents for detecting no more than 20 alleles in the genome of the individual.

In a further aspect of the present invention, a pharmaceutical pack (kit) is provided, the pack comprising a therapeutic agent and a set of instructions for administration of the therapeutic agent to humans diagnostically tested for an at-risk variant for thyroid cancer. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention (e.g., an at-risk variant) is instructed to take a prescribed dose of the therapeutic agent. In another embodiment, an individual identified as a non-carrier of at least one variant of the present invention (e.g., an at-risk variant) is instructed to take a prescribed dose of the therapeutic agent.

In certain embodiments, the kit further comprises a set of instructions for using the reagents comprising the kit. In certain embodiments, the kit further comprises a collection of data comprising correlation data between the at least one at-risk variant and susceptibility to thyroid cancer.

### Antisense Agents

The nucleic acids and/or variants described herein, e.g. the rs7005606 and rs966423 variants or correlated variants in linkage disequilibrium therewith, or nucleic acids comprising their complementary sequence, may be used as antisense constructs to control gene expression in cells, tissues or organs. The methodology associated with antisense techniques is well known to the skilled artisan, and is for example described and reviewed in AntisenseDrug Technology: Principles, Strategies, and Applications, Crooke, ed., Marcel Dekker Inc., New York (2001). In general, antisense agents (antisense oligonucleotides) are comprised of single stranded oligonucleotides (RNA or DNA) that are capable of binding to a complimentary nucleotide segment. By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide binds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Layery et al., Curr. Opin. Drug Discov. Devel. 6:561-569 (2003), Stephens et al., Curr. Opin. Mol. Ther. 5:118-122 (2003), Kurreck, Eur. J. Biochem. 270:1628-44 (2003), Dias et al., Mol. Cancer. Ter. 1:347-55 (2002), Chen, Methods Mol. Med. 75:621-636 (2003), Wang et al., Curr. Cancer Drug Targets 1:177-96 (2001), and Bennett, Antisense Nucleic Acid Drug. Dev. 12:215-24 (2002).

In certain embodiments, the antisense agent is an oligonucleotide that is capable of binding to a particular nucleotide segment. In certain embodiments, the nucleotide segment comprises the a marker selected from the group consisting of rs7005606 and rs966423, and markers in linkage disequilibrium therewith. In certain embodiments, the nucleotide segment comprises a sequence as set forth in any of SEQ ID NO:1-771. Antisense nucleotides can be from 5-400 nucleotides in length, including 5-200 nucleotides, 5-100 nucleotides, 10-50 nucleotides, and 10-30 nucleotides. In certain preferred embodiments, the antisense nucleotides is from 14-50 nucleotides in length, including 14-40 nucleotides and 14-30 nucleotides.

The variants described herein can also be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention can be inhibited or blocked. In one embodiment, the antisense molecules are designed to specifically bind a particular allelic form of the target nucleic acid, thereby inhibiting translation of a product originating from this specific allele, but which do not bind other or alternate variants at the specific polymorphic sites of the target nucleic acid molecule. In one embodiment, the antisense molecule is designed to specifically bind to nucleic acids comprising the G allele of rs7005606 and/or the C allele of rs966423. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al., Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al., Nature Biotechnol. 23:222-226 (2005); Siolas et al., Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al., Nature Biotechnol. 23:559-565 (2006); Brummelkamp et al., Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Layery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440-442 (1999).

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S, and Altschul, S., Proc. Natl. Acad. Sci. USA, 90:5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988). In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence as set forth in any one of SEQ ID NO:1-771, or a nucleotide sequence comprising, or consisting of, the complement of the nucleotide sequence of any one of SEQ ID NO:1-771. The nucleic acid fragments of the invention are suitably at least about 15, at least about 18, 20, 23 or 25 nucleotides, and can be up to 30, 40, 50, 100, 200, 300 or 400 nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al., Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

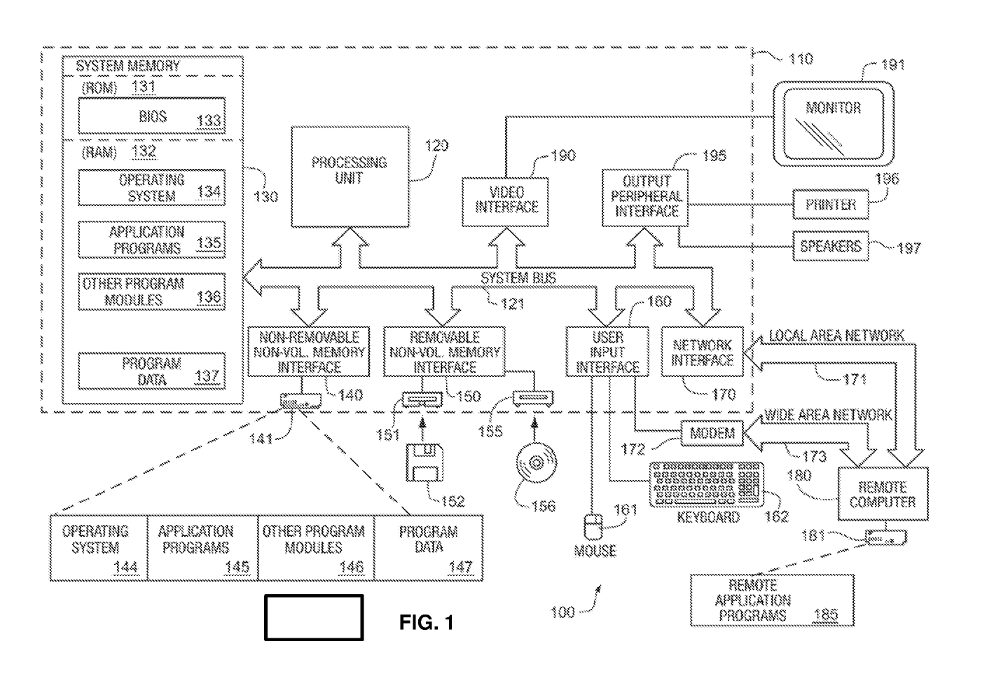

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, certain aspects of the invention relate to computer-implemented applications using the polymorphic markers and haplotypes described herein, and genotype and/or disease-association data derived therefrom. Such applications can be useful for storing, manipulating or otherwise analyzing genotype data that is useful in the methods of the invention. One example pertains to storing genotype and/or sequence data derived from an individual on readable media, so as to be able to provide the data to a third party (e.g., the individual, a guardian of the individual, a health care provider or genetic analysis service provider), or for deriving information from the data, e.g., by comparing the data to information about genetic risk factors contributing to increased susceptibility thyroid cancer, and reporting results based on such comparison.

In certain embodiments, computer-readable media suitably comprise capabilities of storing (i) identifier information for at least one polymorphic marker (e.g, marker names), as described herein; (ii) an indicator of the identity (e.g., presence or absence) of at least one allele of said at least one marker in individuals with thyroid cancer (e.g., rs7005606 and/or rs966423); and (iii) an indicator of the risk associated with a particular marker allele (e.g., the G allele of rs7005606 and/or the C allele of rs966423). The media may also suitably comprise capabilities of storing protein sequence data.

In one embodiment, the invention provides a computer-readable medium having computer executable instructions for determining susceptibility to thyroid cancer in a human individual, the computer readable medium comprising (i) sequence data identifying at least one allele of at least one polymorphic marker in the individual; and (ii) a routine stored on the computer readable medium and adapted to be executed by a processor to determine risk of developing thyroid cancer for the at least one polymorphic marker; wherein the at least one polymorphic marker is selected from the group consisting of rs7005606 and rs966523, and markers in linkage disequilibrium therewith. In one embodiment, the at least one polymorphic marker is rs7005606. In another embodiment, the at least one polymorphism is rs966423.

In certain embodiments, a report is prepared, which contains results of a determination of susceptibility of thyroid cancer. The report may suitably be written in any computer readable medium, printed on paper, or displayed on a visual display.

Another aspect of the invention is a system that is capable of carrying out a part or all of a method of the invention, or carrying out a variation of a method of the invention as described in herein in greater detail. Exemplary systems include, as one or more components, computing systems, environments, and/or configurations that may be suitable for use with the methods and include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like. In some variations, a system of the invention includes one or more machines used for analysis of biological material (e.g., genetic material), as described herein. In some variations, this analysis of the biological material involves a chemical analysis and/or a nucleic acid amplification.

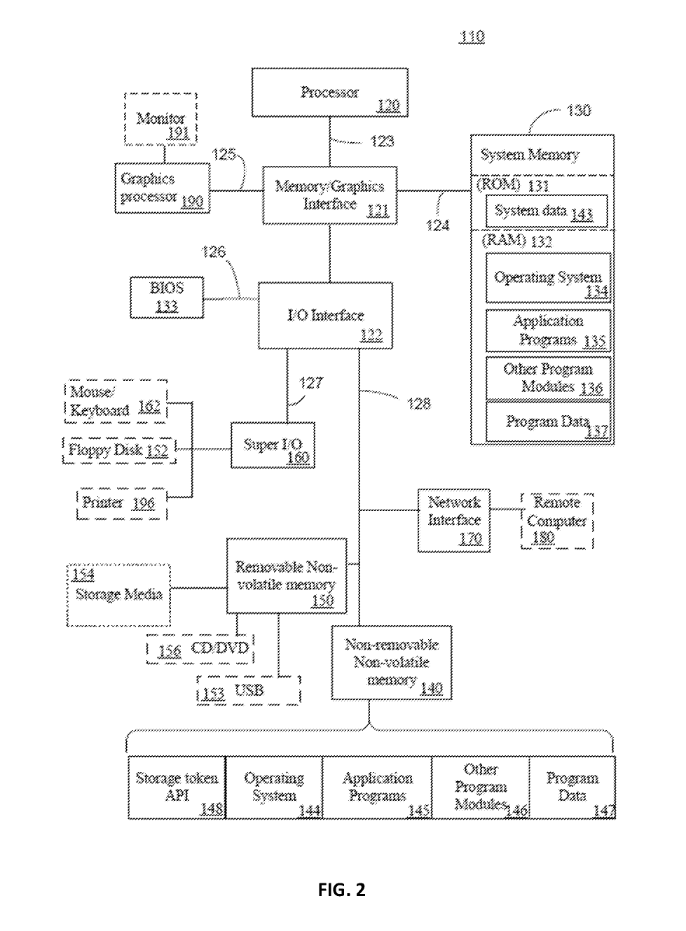

With reference to FIG. 2, an exemplary system of the invention, which may be used to implement one or more steps of methods of the invention, includes a computing device in the form of a computer 110. Components shown in dashed outline are not technically part of the computer 110, but are used to illustrate the exemplary embodiment of FIG. 2. Components of computer 110 may include, but are not limited to, a processor 120, a system memory 130, a memory/graphics interface 121, also known as a Northbridge chip, and an I/O interface 122, also known as a Southbridge chip. The system memory 130 and a graphics processor 190 may be coupled to the memory/graphics interface 121. A monitor 191 or other graphic output device may be coupled to the graphics processor 190.

A series of system busses may couple various system components including a high speed system bus 123 between the processor 120, the memory/graphics interface 121 and the I/O interface 122, a front-side bus 124 between the memory/graphics interface 121 and the system memory 130, and an advanced graphics processing (AGP) bus 125 between the memory/graphics interface 121 and the graphics processor 190. The system bus 123 may be any of several types of bus structures including, by way of example, and not limitation, such architectures include Industry Standard Architecture (USA) bus, Micro Channel Architecture (MCA) bus and Enhanced ISA (EISA) bus. As system architectures evolve, other bus architectures and chip sets may be used but often generally follow this pattern. For example, companies such as Intel and AMD support the Intel Hub Architecture (IHA) and the Hypertransport™ architecture, respectively.

The computer 110 typically includes a variety of computer-readable media. Computer-readable media can be any available media that can be accessed by computer 110 and includes both volatile and nonvolatile media, removable and non-removable media. By way of example, and not limitation, computer readable media may comprise computer storage media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other physical medium which can be used to store the desired information and which can accessed by computer 110.

The system memory 130 includes computer storage media in the form of volatile and/or nonvolatile memory such as read only memory (ROM) 131 and random access memory (RAM) 132. The system ROM 131 may contain permanent system data 143, such as identifying and manufacturing information. In some embodiments, a basic input/output system (BIOS) may also be stored in system ROM 131. RAM 132 typically contains data and/or program modules that are immediately accessible to and/or presently being operated on by processor 120. By way of example, and not limitation, FIG. 2 illustrates operating system 134, application programs 135, other program modules 136, and program data 137.

The I/O interface 122 may couple the system bus 123 with a number of other busses 126, 127 and 128 that couple a variety of internal and external devices to the computer 110. A serial peripheral interface (SPI) bus 126 may connect to a basic input/output system (BIOS) memory 133 containing the basic routines that help to transfer information between elements within computer 110, such as during start-up.

A super input/output chip 160 may be used to connect to a number of ‘legacy’ peripherals, such as floppy disk 152, keyboard/mouse 162, and printer 196, as examples. The super I/O chip 160 may be connected to the I/O interface 122 with a bus 127, such as a low pin count (LPC) bus, in some embodiments. Various embodiments of the super I/O chip 160 are widely available in the commercial marketplace.

In one embodiment, bus 128 may be a Peripheral Component Interconnect (PCI) bus, or a variation thereof, may be used to connect higher speed peripherals to the I/O interface 122. A PCI bus may also be known as a Mezzanine bus. Variations of the PCI bus include the Peripheral Component Interconnect-Express (PCI-E) and the Peripheral Component Interconnect—Extended (PCI-X) busses, the former having a serial interface and the latter being a backward compatible parallel interface. In other embodiments, bus 128 may be an advanced technology attachment (ATA) bus, in the form of a serial ATA bus (SATA) or parallel ATA (PATA).

The computer 110 may also include other removable/non-removable, volatile/nonvolatile computer storage media. By way of example only, FIG. 2 illustrates a hard disk drive 140 that reads from or writes to non-removable, nonvolatile magnetic media. The hard disk drive 140 may be a conventional hard disk drive.

Removable media, such as a universal serial bus (USB) memory 153, firewire (IEEE 1394), or CD/DVD drive 156 may be connected to the PCI bus 128 directly or through an interface 150. A storage media 154 may be coupled through interface 150. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

The drives and their associated computer storage media discussed above and illustrated in FIG. 2, provide storage of computer readable instructions, data structures, program modules and other data for the computer 110. In FIG. 2, for example, hard disk drive 140 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a mouse/keyboard 162 or other input device combination. Other input devices (not shown) may include a microphone, joystick, game pad, satellite dish, scanner, or the like. These and other input devices are often connected to the processor 120 through one of the I/O interface busses, such as the SPI 126, the LPC 127, or the PCI-128, but other busses may be used. In some embodiments, other devices may be coupled to parallel ports, infrared interfaces, game ports, and the like (not depicted), via the super I/O chip 160.

The computer 110 may operate in a networked environment using logical connections to one or more remote computers, such as a remote computer 180 via a network interface controller (NIC) 170. The remote computer 180 may be a personal computer, a server, a router, a network PC, a peer device or other common network node, and typically includes many or all of the elements described above relative to the computer 110. The logical connection between the NIC 170 and the remote computer 180 depicted in FIG. 2 may include a local area network (LAN), a wide area network (WAN), or both, but may also include other networks. Such networking environments are commonplace in offices, enterprise-wide computer networks, intranets, and the Internet. The remote computer 180 may also represent a web server supporting interactive sessions with the computer 110, or in the specific case of location-based applications may be a location server or an application server.

In some embodiments, the network interface may use a modem (not depicted) when a broadband connection is not available or is not used. It will be appreciated that the network connection shown is exemplary and other means of establishing a communications link between the computers may be used.

In some variations, the invention is a system for determining risk of thyroid cancer in a human subject. For example, in one variation, the system includes tools for performing at least one step, preferably two or more steps, and in some aspects all steps of a method of the invention, where the tools are operably linked to each other. Operable linkage describes a linkage through which components can function with each other to perform their purpose.

In some variations, the invention relates to a system for identifying susceptibility to thyroid cancer in a human subject, the system comprising (1) at least one processor; (2) at least one computer-readable medium; (3) a susceptibility database operatively coupled to a computer-readable medium of the system and containing population information correlating the presence or absence of at least one marker allele and susceptibility to thyroid cancer in a population of humans; (4) a measurement tool that receives an input about the human subject and generates information from the input about the presence or absence of the at least one allele in the human subject; and (5) an analysis tool that (a) is operatively coupled to the susceptibility database and the measurement tool; (b) is stored on a computer-readable medium of the system; (c) is adapted to be executed on a processor of the system, to compare the information about the human subject with the population information in the susceptibility database and generate a conclusion with respect to susceptibility to thyroid cancer for the human subject; wherein the at least one marker allele is an allele of a marker selected from the group consisting of rs7005606 and rs966423, and markers correlated therewith.

In certain embodiments, the at least one polymorphic marker correlated with rs7005606 is selected from the group consisting of the markers listed in table 2 herein. In certain embodiments, the at least one polymorphic marker correlated with rs966423 is selected from the group consisting of the markers listed in table 1 herein. In certain embodiments, the marker allele is a risk allele of the claimed marker as listed in table 1 or table 2. In certain embodiments, the marker allele is selected from the marker alleles set forth in table 7 and table 8 herein having a risk for thyroid cancer of greater than unity.

Exemplary processors (processing units) include all variety of microprocessors and other processing units used in computing devices. Exemplary computer-readable media are described above. When two or more components of the system involve a processor or a computer-readable medium, the system generally can be created where a single processor and/or computer readable medium is dedicated to a single component of the system; or where two or more functions share a single processor and/or share a single computer readable medium, such that the system contains as few as one processor and/or one computer readable medium. In some variations, it is advantageous to use multiple processors or media, for example, where it is convenient to have components of the system at different locations. For instance, some components of a system may be located at a testing laboratory dedicated to laboratory or data analysis, whereas other components, including components (optional) for supplying input information or obtaining an output communication, may be located at a medical treatment or counseling facility (e.g., doctor's office, health clinic, HMO, pharmacist, geneticist, hospital) and/or at the home or business of the human subject (patient) for whom the testing service is performed.

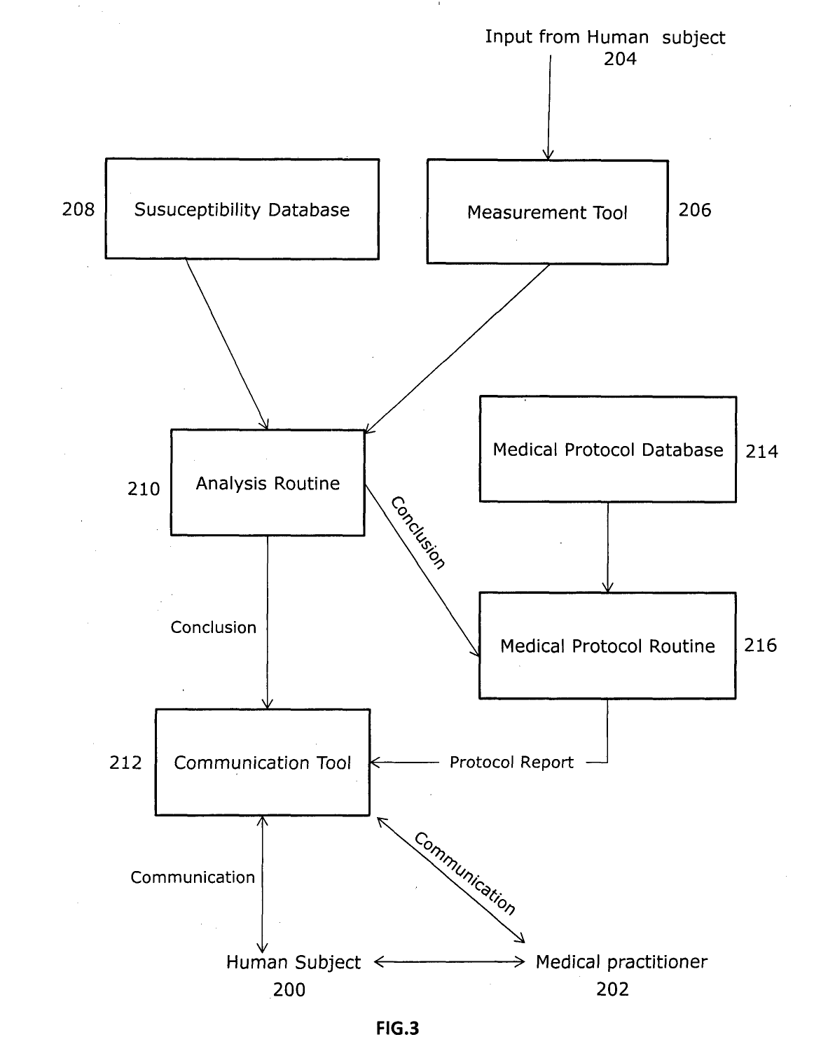

Referring to FIG. 3, an exemplary system includes a susceptibility database 208 that is operatively coupled to a computer-readable medium of the system and that contains population information correlating the presence or absence of one or more alleles of markers selected from the group consisting of rs966423 and rs7005606 and markers correlated therewith.

In a simple variation, the susceptibility database contains 208 data relating to the correlation between a particular marker allele and thyroid cancer in humans. The correlation may suitably be contained in a form of percentage or fractional increase for a particular marker allele. For SNPs, the alternate allele, by necessity, will then be correlated with decreased thyroid cancer by the same percentage or fraction. Such data provides an indication as to the genetic contribution of observed thyroid cancer for the subject having the allele in question. In another variation, the susceptibility database includes similar data with respect to two or more polymorphic markers, thus providing information about the contribution of two or more markers to thyroid cancer. In still another variation, the susceptibility database includes additional quantitative personal, medical, or genetic information about the individuals in the database diagnosed with thyroid cancer or those who are free of thyroid cancer. Such information includes, but is not limited to, information about parameters such as age, sex, ethnicity, race, medical history, weight, diabetes status, blood pressure, family history of thyroid cancer, smoking history, and alcohol use in humans and impact of the at least one parameter on susceptibility to thyroid cancer. The information also can include information about other genetic risk factors for thyroid cancer. These more robust susceptibility databases can be used by an analysis routine 210 to calculate risk of thyroid cancer, utilizing information about polymorphic markers as described herein and information about other genetic risk factors.

In addition to the susceptibility database 208, the system further includes a measurement tool 206 programmed to receive an input 204 from or about the human subject and generate an output that contains information about the presence or absence of the at least one allele of at least one polymorphic marker. (The input 204 is not part of the system per se but is illustrated in the schematic FIG. 3.) Thus, the input 204 will contain a specimen or contain data from which the presence or absence of the at least one allele can be directly read, or analytically determined. In a simple variation, the input contains annotated information about genotypes or allele counts for at least one polymorphic marker in the genome of the human subject, in which case no further processing by the measurement tool 206 is required, except possibly transformation of the relevant information about the presence/absence of the allele into a format compatible for use by the analysis routine 210 of the system.

In another variation, the input 204 from the human subject contains data that is unannotated or insufficiently annotated with respect to particular polymorphic markers, requiring analysis by the measurement tool 206. For example, the input can be genetic sequence of a chromosomal region or chromosome on which the particular polymorphic markers of interest reside, or whole genome sequence information, or unannotated information from a gene chip analysis of a variable loci in the human subject's genome. In such variations of the invention, the measurement tool 206 comprises a tool, preferably stored on a computer-readable medium of the system and adapted to be executed on a processor of the system, to receive a data input about a subject and determine information about the presence or absence of the at least one allele of at least one polymorphic marker in a human subject from the data. For example, the measurement tool 206 contains instructions, preferably executable on a processor of the system, for analyzing the unannotated input data and determining the presence or absence of at least one allele of interest in the human subject. Where the input data is genomic sequence information, and the measurement tool optionally comprises a sequence analysis tool stored on a computer readable medium of the system and executable by a processor of the system with instructions for determining the presence or absence of the at least one allele from the genomic sequence information.

In yet another variation, the input 204 from the human subject comprises a biological sample, such as a fluid (e.g., blood) or tissue sample, that contains genetic material that can be analyzed to determine the presence or absence of the allele of interest. In this variation, an exemplary measurement tool 206 includes laboratory equipment for processing and analyzing the sample to determine the presence or absence (or identity) of the allele(s) in the human subject. For instance, in one variation, the measurement tool includes: an oligonucleotide microarray (e.g., “gene chip”) containing a plurality of oligonucleotide probes attached to a solid support; a detector for measuring interaction between nucleic acid obtained from or amplified from the biological sample and one or more oligonucleotides on the oligonucleotide microarray to generate detection data; and an analysis tool stored on a computer-readable medium of the system and adapted to be executed on a processor of the system, to determine the presence or absence of the at least one allele of interest based on the detection data.

In another variation, the input 204_from the human subject comprises a biological sample that is suitable for determining risk of thyroid cancer, such as a fluid (e.g. blood) or tissue sample that can be analyzed to determine risk of thyroid cancer. In this variation the exemplary measurement tool 206 includes laboratory equipment and reagents for processing and analyzing the sample to determine risk of thyroid cancer in the human subject.

To provide another example, in some variations the measurement tool 206 includes: a nucleotide sequencer (e.g., an automated DNA sequencer) that is capable of determining nucleotide sequence information from nucleic acid obtained from or amplified from the biological sample; and an analysis tool stored on a computer-readable medium of the system and adapted to be executed on a processor of the system, to determine the presence or absence of the at least one allele associated with thyroid cancer, based on the nucleotide sequence information.

In some variations, the measurement tool 206 further includes additional equipment and/or chemical reagents for processing the biological sample to purify and/or amplify nucleic acid of the human subject for further analysis using a sequencer, gene chip, or other analytical equipment. In further variations, the measurement tool 206 further includes additional equipment and/or chemical reagents for processing the biological sample to purify protein of the human subject for determining thyroid cancer using appropriate analytical equipment.

The exemplary system further includes an analysis tool or routine 210 that: is operatively coupled to the susceptibility database 208 and operatively coupled to the measurement tool 206, is stored on a computer-readable medium of the system, is adapted to be executed on a processor of the system to compare the information about the human subject with the population information in the susceptibility database 208 and generate a conclusion with respect to corrected thyroid cancer for the human subject. In simple terms, the analysis tool 210 looks at the alleles identified by the measurement tool 206 for the human subject, and compares this information to the susceptibility database 208, to determine corrected thyroid cancer for the subject. The susceptibility can be based on the single parameter (the identity of one or more marker alleles), or can involve a calculation based on multiple genetic markers and/or other genetic and non-genetic data, as described above, that is collected and included as part of the input 204 from the human subject, and that also is stored in the susceptibility database 208 with respect to a population of other humans. Generally speaking, each parameter of interest is weighted to provide a conclusion with respect to susceptibility to thyroid cancer.

In some variations of the invention, the system as just described further includes a communication tool 212. For example, the communication tool is operatively connected to the analysis routine 210 and comprises a routine stored on a computer-readable medium of the system and adapted to be executed on a processor of the system, to: generate a communication containing the conclusion; and to transmit the communication to the human subject 200 or the medical practitioner 202, and/or enable the subject or medical practitioner to access the communication. (The subject and medical practitioner are depicted in the schematic FIG. 3, but are not part of the system per se, though they may be considered users of the system. The communication tool 212 provides an interface for communicating to the subject, or to a medical practitioner for the subject (e.g., doctor, nurse, genetic counselor), the conclusion generated by the analysis tool 210 with respect to thyroid cancer for the subject. Usually, if the communication is obtained by or delivered to the medical practitioner 202, the medical practitioner will share the communication with the human subject 200 and/or counsel the human subject about the medical significance of the communication. In some variations, the communication is provided in a tangible form, such as a printed report or report stored on a computer readable medium such as a flash drive or optical disk. In some variations, the communication is provided electronically with an output that is visible on a video display or audio output (e.g., speaker). In some variations, the communication is transmitted to the subject or the medical practitioner, e.g., electronically or through the mail. In some variations, the system is designed to permit the subject or medical practitioner to access the communication, e.g., by telephone or computer. For instance, the system may include software residing on a memory and executed by a processor of a computer used by the human subject or the medical practitioner, with which the subject or practitioner can access the communication, preferably securely, over the internet or other network connection. In some variations of the system, this computer will be located remotely from other components of the system, e.g., at a location of the human subject's or medical practitioner's choosing.

In some variations of the invention, the system as described (including embodiments with or without the communication tool) further includes components that add a treatment or prophylaxis utility to the system. For instance, value is added to a determination of susceptibility to thyroid cancer when a medical practitioner can prescribe or administer a standard of care that can reduce susceptibility to thyroid cancer; and/or delay onset of thyroid cancer; and/or increase the likelihood of detecting thyroid cancer at an early stage, to facilitate early treatment when the cancer has not spread and is most curable. Exemplary lifestyle change protocols include loss of weight, increase in exercise, cessation of unhealthy behaviors such as smoking, and change of diet. Exemplary medicinal and surgical intervention protocols include administration of pharmaceutical agents for prophylaxis; and surgery, including in extreme cases surgery to remove a tissue or organ before it has become cancerous. Exemplary diagnostic protocols include non-invasive and invasive imaging; monitoring metabolic biomarkers; and biopsy screening.

For example, in some variations, the system further includes a medical protocol database 214 operatively connected to a computer-readable medium of the system and containing information correlating the presence or absence of the at least one marker allele of interest and medical protocols for human subjects at risk for thyroid cancer. Such medical protocols include any variety of medicines, lifestyle changes, diagnostic tests, increased frequencies of diagnostic tests, and the like that are designed to achieve one of the aforementioned goals. The information correlating marker alleles with protocols could include, for example, information about thyroid cancer and the success with which thyroid cancer is avoided or delayed, or success with which thyroid cancer is detected early and treated, if a subject has particular corrected thyroid cancer and follows a protocol.

The system of this embodiment further includes a medical protocol tool or routine 216, operatively connected to the medical protocol database 214 and to the analysis tool or routine 210. The medical protocol tool or routine 216 preferably is stored on a computer-readable medium of the system, and adapted to be executed on a processor of the system, to: (i) compare (or correlate) the conclusion that is obtained from the analysis routine 210 (with respect to thyroid cancer risk for the subject) and the medical protocol database 214, and (ii) generate a protocol report with respect to the probability that one or more medical protocols in the medical protocol database will achieve one or more of the goals of reducing susceptibility to thyroid cancer; delaying onset of thyroid cancer; and increasing the likelihood of detecting thyroid cancer at an early stage to facilitate early treatment. The probability can be based on empirical evidence collected from a population of humans and expressed either in absolute terms (e.g., compared to making no intervention), or expressed in relative terms, to highlight the comparative or additive benefits of two or more protocols.

Some variations of the system just described include the communication tool 212. In some examples, the communication tool generates a communication that includes the protocol report in addition to, or instead of, the conclusion with respect to susceptibility.

Information about marker allele status not only can provide useful information about identifying thyroid cancer and/or determine susceptibility to thyroid cancer; it can also provide useful information about possible causative factors for a human subject identified with thyroid cancer, and useful information about therapies for thyroid cancer patient. In some variations, systems of the invention are useful for these purposes.

For instance, in some variations the invention is a system for assessing or selecting a treatment protocol for a subject diagnosed with thyroid cancer, comprising (1) at least one processor; (2) at least one computer-readable medium; (3) a medical treatment database operatively connected to a computer-readable medium of the system and containing information correlating the presence or absence of at least one allele of at least one marker selected from the group consisting of rs7005606 and rs966423, and markers correlated therewith, and efficacy of treatment regimens for thyroid cancer; (4) a measurement tool to receive an input about the human subject and generate information from the input about the presence or absence of the at least one marker allele in a human subject diagnosed with thyroid cancer; and (5) a medical protocol tool operatively coupled to the medical treatment database and the measurement tool, stored on a computer-readable medium of the system, and adapted to be executed on a processor of the system, to compare the information with respect to presence or absence of the at least one marker allele for the subject and the medical treatment database, and generate a conclusion with respect to at least one of (a) the probability that one or more medical treatments will be efficacious for treatment of thyroid cancer for the patient; and (b) which of two or more medical treatments for thyroid cancer will be more efficacious for the patient.

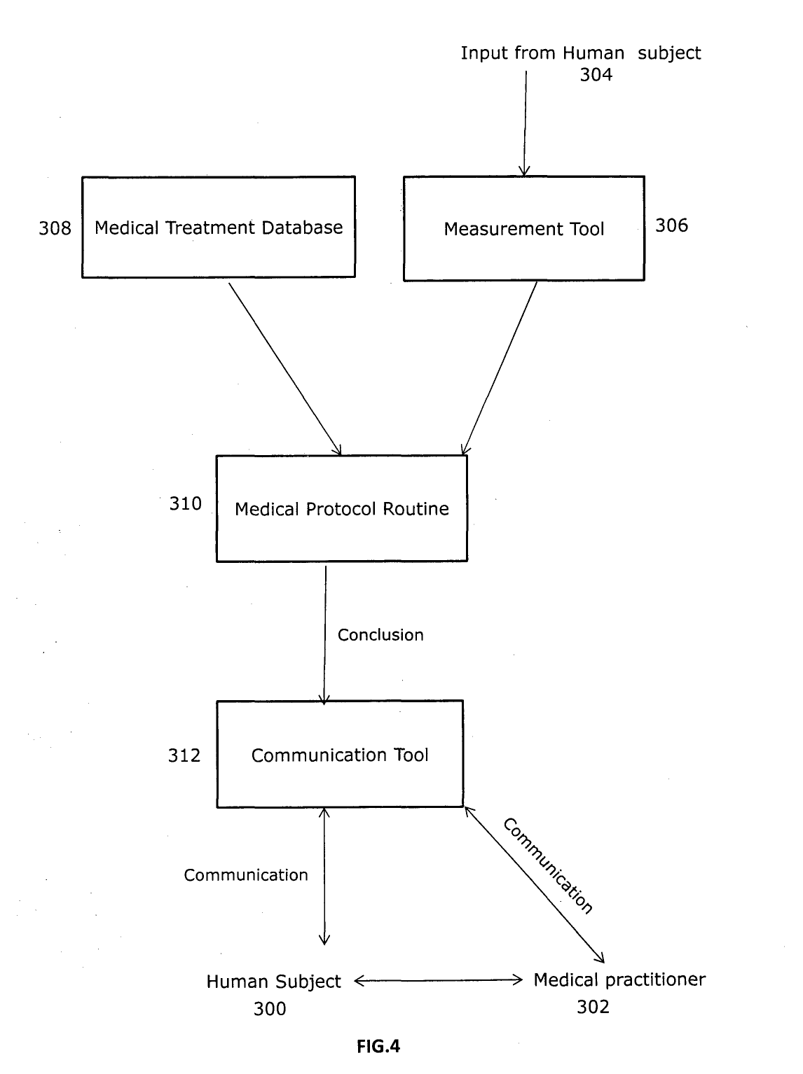

Preferably, such a system further includes a communication tool 312 operatively connected to the medical protocol tool or routine 310 for communicating the conclusion to the subject 300, or to a medical practitioner for the subject 302 (both depicted in the schematic of FIG. 4, but not part of the system per se). An exemplary communication tool comprises a routine stored on a computer-readable medium of the system and adapted to be executed on a processor of the system, to generate a communication containing the conclusion; and transmit the communication to the subject or the medical practitioner, or enable the subject or medical practitioner to access the communication.

In certain embodiments, the at least one polymorphic marker correlated with rs7005606 is selected from the group consisting of the markers listed in table 2 herein. In certain embodiments, the at least one polymorphic marker correlated with rs966423 is selected from the group consisting of the markers listed in table 1 herein. In certain embodiments, the marker allele is a risk allele of the claimed marker as listed in table 1 or table 2. In certain embodiments, the marker allele is selected from the marker alleles set forth in table 7 and table 8 herein having a risk for thyroid cancer of greater than unity.

The present invention will now be exemplified by the following non-limiting examples.

### Example 1

Association of markers on chromosome 2 (rs966423) and chromosome 8 (rs7005606) with thyroid cancer was investigated. Both markers were previously found to be associated with levels of thyroid stimulating hormone (TSH), leading to the speculation that they might also be associated with risk of thyroid cancer.

Data was generated based on genotyping using Centaurus assays, supplemented by results from imputation analysis (see below). A total of 544 samples from individuals with thyroid cancer were genotyped directly, and additional genotypes imputed for 110 cases (rs7005606) and 117 cases (rs966423), respectively. Genotypes for 37,668 (rs7005606) and 37,534 (rs966423) population controls were also determined.

Results of association analysis is shown below in Table 3. As can be seen, both markers were found to be significantly associated with thyroid cancer, with risk close to 1.3 for both markers.

TABLE 3
Association of markers on Chromosome 2 and 8 with Thyroid cancer.

| Marker | Chr | Pos (Build 36) | allele | freq cases | freq ctrls | OR | p-value |
|---|---|---|---|---|---|---|---|
| rs7005606 | 8 | 32,521,043 | G | 0.527 | 0.458 | 1.322 | 5.59 × 10⁻⁷ |
| rs966423 | 2 | 218,018,585 | C | 0.503 | 0.441 | 1.284 | 7.17 × 10⁻⁶ |

### Example 2

### Subjects

Approval for this study was granted by the National Bioethics Committee of Iceland and the Icelandic Data Protection Authority.

Our collection of samples used for the thyroid cancer study represents the overall distribution in Iceland quite well. Of the cases that we generated genotypes for either by directly genotyping or in-silico genotyping, about 80% are of papillary type, about 12% are of follicular type, about 2% are medullary thyroid cancer, and the remainder is of unknown or undetermined histological sub-phenotype.

The results presented above in Table 3 are for the combined results for all our cases since no statistically significant difference was observed between the different histological subgroups.

The Icelandic controls consist of up to 37,668 individuals from other ongoing genome-wide association studies at deCODE genetics. Individuals with a diagnosis of thyroid cancer were excluded. Both male and female genders were included.

### Genotyping

Markers in Table 3 were genotyped by Centaurus SNP genotyping (Kutyavin, et al., (2006), Nucleic Acids Res, 34, e128). Genotyping was carried out at the deCODE genetics facility.

### Imputation Analysis

We imputed genotypes for un-genotyped cases of genotyped individuals. For every un-genotyped case, it is possible to calculate the probability of the genotypes of its relatives given its four possible phased genotypes. In practice it may be preferable to include only the genotypes of the case's parents, children, siblings, half-siblings (and the half-sibling's parents), grand-parents, grand-children (and the grand-children's parents) and spouses. It will be assumed that the individuals in the small sub-pedigrees created around each case are not related through any path not included in the pedigree. It is also assumed that alleles that are not transmitted to the case have the same frequency—the population allele frequency. Let us consider a SNP marker with the alleles A and G. The probability of the genotypes of the case's relatives can then be computed by:

$$Pr ( genotypes of relatives ; θ ) = {\sum }_{h ∈ \{ AA , AG , GA , GG \}} Pr ( h ; θ ) Pr ( genotypes of relatives \vert h ) ,$$

where θ denotes the A allele's frequency in the cases. Assuming the genotypes of each set of relatives are independent, this allows us to write down a likelihood function for θ:

$$L ( θ ) = {\prod }_{i} Pr ( genotypesof relativesof case i ; θ ) . \qquad (* )$$

This assumption of independence is usually not correct. Accounting for the dependence between individuals is a difficult and potentially prohibitively expensive computational task. The likelihood function in (*) may be thought of as a pseudolikelihood approximation of the full likelihood function for θ which properly accounts for all dependencies. In general, the genotyped cases and controls in a case-control association study are not independent and applying the case-control method to related cases and controls is an analogous approximation. The method of genomic control (Devlin, B. et al., Nat Genet. 36, 1129-30; author reply 1131 (2004)) has proven to be successful at adjusting case-control test statistics for relatedness. We therefore apply the method of genomic control to account for the dependence between the terms in our pseudolikelihood and produce a valid test statistic.

Fisher's information can be used to estimate the effective sample size of the part of the pseudolikelihood due to un-genotyped cases. Breaking the total Fisher information, I, into the part due to genotyped cases, I_{g}, and the part due to ungenotyped cases, Iᵤ, I═I_{g}+Iᵤ, and denoting the number of genotyped cases with N, the effective sample size due to the un-genotyped cases is estimated by

$$\frac{{I}_{u}}{{I}_{g}} N .$$

### Example 3

We performed an association test using data from genotyping in combination with familial imputation for markers in linkage disequilibrium with the anchor markers rs7005606 on chromosome 8 and rs966423 on chromosome 2.

Results of this analysis are shown in Tables 4 and 5 below. As expected, a number of variants show significant association with thyroid cancer, and in general the significance of association correlates with the degree to which the surrogate is correlated with the anchor marker.

TABLE 4
Association analysis for surrogate markers of rs966423. Shown is marker name, its correlation with rs966423, p-value of the association
test, Odds Ratio, number of genotyped cases, frequency of the effect allele in those cases, number of cases available for imputation,
total number of imputed cases, frequency of effect allele in imputed cases, combined frequency of effect allele in cases, number of
controls, frequency of effect allele in controls, identity of effect allele, identity of alternate allele, and SEQ ID NO of the marker.

| | | | | | | | | | | | | | | Seq |
|---|---|---|---|---|---|---|---|---|---|---|---|---|---|---|
| | | | | Cases | | Cases for | Imputed | | Freq | No of | Freq. | Effect | Other | ID |
| SNP | r² | P-value | OR | w gt | Freq | imput | cases | Freq | total | controls | controls | allele | allele | NO: |
| rs2568158 | 0.48 | 6.07E−05 | 1.361 | 196 | 0.347 | 631 | 197.3 | 0.393 | 0.37 | 37882 | 0.301 | A | C | 198 |
| rs2618139 | 0.4 | 0.000763166 | 1.285 | 196 | 0.416 | 631 | 182.7 | 0.477 | 0.445 | 37899 | 0.384 | T | G | 186 |
| rs2618148 | 0.65 | 0.0127053 | 1.301 | 99 | 0.364 | 412 | 102.3 | 0.378 | 0.371 | 20753 | 0.312 | T | C | 130 |
| rs4372880 | 0.27 | 0.1748 | 1.153 | 196 | 0.135 | 631 | 208.9 | 0.158 | 0.147 | 37873 | 0.13 | T | C | 269 |
| rs4674167 | 0.5 | 0.521323 | 1.05 | 196 | 0.344 | 631 | 179 | 0.38 | 0.361 | 37859 | 0.35 | T | C | 46 |
| rs6754268 | 0.48 | 7.67E−05 | 1.356 | 196 | 0.344 | 631 | 197.5 | 0.389 | 0.367 | 37843 | 0.299 | G | A | 254 |
| rs750365 | 0.22 | 7.45E−05 | 1.343 | 192 | 0.414 | 629 | 191.1 | 0.451 | 0.432 | 37615 | 0.362 | A | C | 311 |
| rs768435 | 0.48 | 0.000125974 | 1.343 | 195 | 0.344 | 631 | 198.1 | 0.388 | 0.366 | 37870 | 0.3 | T | C | 221 |
| rs874840 | 0.48 | 7.58E−05 | 1.355 | 196 | 0.347 | 631 | 197.1 | 0.391 | 0.369 | 37865 | 0.301 | T | C | 248 |
| rs981938 | 0.5 | 0.402869 | 1.067 | 191 | 0.348 | 627 | 175.4 | 0.394 | 0.37 | 37301 | 0.355 | G | A | 47 |
| rs9989823 | 0.27 | 0.168099 | 1.155 | 196 | 0.135 | 630 | 215.4 | 0.164 | 0.15 | 37792 | 0.132 | T | C | 284 |
| rs1382435 | 0.46 | 6.05E−05 | 1.345 | 196 | 0.431 | 631 | 185.6 | 0.482 | 0.456 | 37897 | 0.384 | T | C | 114 |
| rs1478581 | 0.31 | 0.126681 | 1.152 | 196 | 0.199 | 630 | 192.5 | 0.198 | 0.198 | 37832 | 0.177 | A | G | 105 |
| rs1478583 | 0.25 | 0.118403 | 1.17 | 196 | 0.151 | 631 | 209.5 | 0.179 | 0.165 | 37866 | 0.144 | C | T | 192 |

TABLE 5
Association analysis for surrogate markers of rs7005606. Shown is marker name, its correlation with rs7005606, p-value of the association
test, Odds Ratio, number of genotyped cases, frequency of the effect allele in those cases, number of cases available for imputation, total
number of imputed cases, frequency of effect allele in imputed cases, combined frequency of effect allele in cases, number of controls,
frequency of effect allele in controls, identity of effect allele, identity of alternate allele, and SEQ ID NO of the marker. It should be noted
that for markers for which an OR value less than one is shown, the “alternate” allele is the at-risk allele (and the risk for the at-risk
allele equals 1/OR).

| | | | | | | | | | | | | | | Seq |
|---|---|---|---|---|---|---|---|---|---|---|---|---|---|---|
| | | | | Cases | | Cases for | Imputed | | Freq | No of | Freq. | Effect | Other | ID |
| SNP | r² | P-value | OR | w gt | Freq | imput | cases | Freq | total | controls | controls | allele | allele | NO: |
| rs1948098 | 0.31 | 0.202366 | 1.103 | 196 | 0.332 | 631 | 186.3 | 0.372 | 0.351 | 37867 | 0.329 | G | T | 330 |
| rs2439312 | 0.33 | 0.00141808 | 0.741 | 196 | 0.179 | 631 | 157.8 | 0.172 | 0.176 | 37873 | 0.223 | T | A | 631 |
| rs10503907 | 0.21 | 0.32835 | 1.077 | 196 | 0.355 | 631 | 184.5 | 0.371 | 0.363 | 37857 | 0.346 | G | A | 325 |
| rs10503914 | 0.25 | 0.0894367 | 1.175 | 196 | 0.191 | 630 | 185.6 | 0.171 | 0.181 | 37779 | 0.158 | C | T | 396 |
| rs10503915 | 0.34 | 0.106832 | 1.154 | 195 | 0.782 | 631 | 160.2 | 0.808 | 0.794 | 37888 | 0.77 | T | C | 397 |
| rs10503918 | 0.25 | 0.0391583 | 1.174 | 195 | 0.659 | 630 | 161.4 | 0.693 | 0.675 | 37763 | 0.638 | G | A | 531 |
| rs10503920 | 0.47 | 4.63E−05 | 0.718 | 195 | 0.272 | 631 | 163.6 | 0.26 | 0.266 | 37879 | 0.335 | G | G | 685 |
| rs4317533 | 0.26 | 0.680845 | 0.969 | 196 | 0.602 | 631 | 180.9 | 0.604 | 0.603 | 37888 | 0.611 | G | A | 324 |
| rs6651144 | 0.56 | 0.00473246 | 1.231 | 196 | 0.592 | 631 | 186.7 | 0.575 | 0.583 | 37881 | 0.532 | T | C | 578 |
| rs6985581 | 0.53 | 0.0298003 | 1.175 | 194 | 0.59 | 630 | 186.1 | 0.588 | 0.589 | 37617 | 0.549 | T | C | 450 |
| rs6992907 | 0.3 | 0.25831 | 1.088 | 194 | 0.41 | 628 | 177.8 | 0.422 | 0.416 | 37486 | 0.396 | C | T | 348 |
| rs6996957 | 0.49 | 0.00916761 | 0.812 | 196 | 0.296 | 631 | 163 | 0.271 | 0.284 | 37856 | 0.328 | T | T | 427 |
| rs7000590 | 0.51 | 0.00129214 | 0.753 | 196 | 0.214 | 630 | 155.2 | 0.197 | 0.206 | 37825 | 0.257 | T | T | 604 |
| rs7013361 | 0.44 | 0.0247055 | 1.22 | 195 | 0.779 | 630 | 154.3 | 0.812 | 0.794 | 37746 | 0.76 | C | A | 561 |
| rs7844597 | 0.33 | 0.00182806 | 1.261 | 195 | 0.569 | 631 | 167.9 | 0.612 | 0.589 | 37889 | 0.532 | T | C | 478 |
| rs7844698 | 0.25 | 0.0462856 | 1.167 | 196 | 0.658 | 630 | 162.8 | 0.69 | 0.673 | 37814 | 0.638 | C | T | 518 |
| rs9886497 | 0.22 | 0.621502 | 1.042 | 195 | 0.297 | 629 | 177 | 0.288 | 0.293 | 37809 | 0.284 | T | C | 371 |
| rs11776203 | 0.48 | 0.120453 | 0.881 | 196 | 0.699 | 631 | 179.7 | 0.716 | 0.707 | 37874 | 0.733 | T | T | 652 |
| rs10096770 | 0.54 | 4.81E−05 | 1.362 | 196 | 0.38 | 631 | 188.4 | 0.389 | 0.384 | 37870 | 0.314 | G | A | 461 |
| rs10103930 | 0.31 | 0.00390491 | 1.24 | 196 | 0.582 | 629 | 165.5 | 0.62 | 0.599 | 37710 | 0.547 | A | G | 425 |
| rs1545961 | 0.22 | 0.661625 | 0.968 | 196 | 0.406 | 631 | 169.3 | 0.371 | 0.389 | 37896 | 0.397 | T | T | 326 |

### Example 4

The association on chromosome 2q35 and 8p12 was investigated further. For this purpose, rs966423 on chromosome 2q35 and rs2439302, a perfect surrogate of rs7005606, on chromosome 8p12 in Caucasians (r²=1; see Table 2), were tested for association with thyroid cancer in Iceland and in three additional case-control groups of European descent, with populations from Ohio, United States, the Netherlands and Spain.

The association on both 2q35 and 8p12 replicated consistently in these cohorts, resulting in combined P-values of association of 1.3×10⁻⁹ for rs966423 (OR=1.34) and 2.0×10⁻⁹ for rs2439302 (OR=1.36) (Table 6).

### Methods

### Study Populations

### Icelandic Study Population.

All participants in this study are of European ancestry. Individuals diagnosed with thyroid cancer were identified based on a nationwide list from the Icelandic Cancer Registry (ICR) (http://www.krabbameinsskra.is) that contains all Icelandic thyroid cancer patients diagnosed from Jan. 1, 1955, to Dec. 31, 2009. Thereof, 1,018 were non-medullary thyroid cancers. Included in the present study are DNA samples from 572 non-medullary thyroid cancer patients, diagnosed from December 1974 to December 2009 and who were recruited from November 2000 until April 2010. The median time from diagnosis to blood sampling is 10 years (range 0 to 46 years). The mean age at diagnosis of recruited patients is 44 years (median 43 years) and the range was from 13 to 87 years, while the mean age at diagnosis is 56 years for all thyroid cancer patients in the ICR.

The thyroid cancer GWAS dataset used in the current study is comprised of results from 222 patients and 24,198 controls genotyped using Illumina Human Hap300-, HapCNV370-, Hap610-, 1M-, or Omni-1 Quad-bead chips (Illumina, San Diego, Calif., USA) as well as results from 627 patients and 71,613 controls with genotypes inferred using an imputation method making use of the Icelandic genealogy to propagate genotypic information into individuals for whom we have neither SNP chip nor sequence data, a process we refer to as “genealogy-based imputation”. We refer to the combined method of imputing sequence-derived data into Illumina chip-typed individuals and using genealogy-based imputation to infer the DNA sequence of ungenotyped individuals as two-way imputation.

For confirming thyroid cancer results, we used the Centaurs genotyping platform to attempt genotyping all 572 samples available from patients and a minimum of 1,500 controls. Thereof, 561 samples from patients and a minimum of 1,472 controls (−98%) were successfully genotyped in our study. Of the 561 patients genotyped using the Centaurus platform, 222 had previously been genotyped using the Illumina chips. The data overlap was used to confirm data consistency. The remaining 339 patients genotyped using the Cenataurus platform are a subset of the 627 patients contributing imputed genotypes to the initial thyroid cancer GWAS dataset.

The 40,013 controls (17,326 males (43.3%) and 22,687 females (56.7%)) consisted of individuals belonging to different genetic research projects at deCODE. The controls had a mean age of 61 years (standard deviation is 20.6 years). The controls were absent from the nationwide list of thyroid cancer patients according to the ICR. The DNA for both the Icelandic cases and controls was isolated from whole blood using standard methods.

The study was approved by the Data Protection Commission of Iceland and the National Bioethics Committee of Iceland. Written informed consent was obtained from all subjects. Personal identifiers associated with medical information and blood samples were encrypted with a third-party encryption system as previously described (Gulcher, J. R., et al. Eur J Hum Genet. 8:739-42 (2000)).

### The Netherlands.

The Dutch study population consists of 151 non-medullary thyroid cancer cases (75% are females) and 832 cancer-free individuals (54% females). The cases were recruited from the Department of Endocrinology, Radboud University Nijmegen Medical Centre (RUNMC), Nijmegen, The Netherlands from November 2009 to June 2010. All patients were of self-reported European descent. Demographic, clinical, tumor treatment and follow-up related characteristics were obtained from the patient's medical records. The average age at diagnosis for the patients was 39 years (SD 12.8). The DNA for both the Dutch cases and controls was isolated from whole blood using standard methods. The controls were recruited within a project entitled “Nijmegen Biomedical Study” (NBS). The details of this study were reported previously (Wetzels, J. F., et al. Kidney Int 72:632-7 (2007)). Control individuals from the NBS were invited to participate in a study on gene-environment interactions in multifactorial diseases such as cancer. They were all of self-reported European descent and fully informed about the goals and the procedures of the study. The study was approved by the Ethical Committee and the Institutional Review Board of the RUNMC, Nijmegen, The Netherlands and all study subjects gave written informed consent.

### Ohio, USA.

The study was approved by the Institutional Review Board of the Ohio State University. All subjects were of self-reported European descent and provided written informed consent. These patients (n=365; median age 40 years, range 13 to 80; 76% are females) were recruited from Ohio, US and were histologically confirmed papillary thyroid carcinoma (PTC) patients (including traditional PTC and follicular variant PTC). Controls (n=383; median age 49 years, range 18 to 87; 65% are females) were individuals without clinically diagnosed thyroid cancer from the central Ohio area. Genomic DNA was extracted from blood.

### Zaragoza, Spain.

The Spanish study population consisted of 90 non-medullary thyroid cancer cases. The cases were recruited from the Oncology Department of Zaragoza Hospital in Zaragoza, Spain, from October 2006 to June 2007. All patients were of self-reported European descent. Clinical information including age at onset, grade and stage was obtained from medical records. The average age at diagnosis for the patients was 48 years (median 49 years) and the range was from 22 to 79 years. The 1,399 Spanish control individuals 798 (57%) males and 601 (43%) females had a mean age of 51 (median age 50 and range 12-87 years) were approached at the University Hospital in Zaragoza, Spain, and were not known to have thyroid cancer. The DNA for both the Spanish cases and controls was isolated from whole blood using standard methods. Study protocols were approved by the Institutional Review Board of Zaragoza University Hospital. All subjects gave written informed consent.

TABLE 6
Association results for variants on 2q35 and 8p12 and thyroid
cancer in Iceland, the Netherlands, Spain and the United States.
Shown are the results for SNPs directly genotyped in cases and
controls (n), the allelic odds ratio (OR) with 95% confidence
interval (95% CI) and P values based on the multiplicative model,
allelic frequencies of risk variants in affected and control
individuals. All P values shown are two-sided.

| | | | | | |
|---|---|---|---|---|---|
| Study population | | | | Case | Controls |
| (n cases/n controls) | OR | 95% CI | P-value | (freq) | (freq) |

rs966423_C on 2q35^{a}

| | | | | | |
|---|---|---|---|---|---|
| Iceland | 1.26 | (1.11, 1.43) | 3.8 × 10⁻⁴ | 0.499 | 0.442 |
| (546/38,854)^{a} | | | | | |
| The Netherlands | 1.80 | (1.40, 2.31) | 4.2 × 10⁻⁶ | 0.554 | 0.408 |
| (149/814) | | | | | |
| Ohio, US | 1.36 | (1.11, 1.67) | 3.5 × 10⁻³ | 0.471 | 0.396 |
| (365/383) | | | | | |
| Spain | 1.20 | (0.89, 1.62) | 0.24 | 0.450 | 0.406 |
| (90/1,397) | | | | | |
| All combined | 1.34 | (1.22, 1.47) | 1.3 × 10⁻⁹ | — | — |
| (1,150/41,448)^{c} | | | | | |
| Pₕₑₜ | | 0.079 | | | |
| I² | | 0.55 | | | |

rs2439302_G on 8p12 ^{b}

| | | | | | |
|---|---|---|---|---|---|
| Iceland | 1.41 | (1.23, 1.62) | 1.3 × 10⁻⁶ | 0.535 | 0.449 |
| (532/3,094)^{a} | | | | | |
| The Netherlands | 1.24 | (0.97, 1.60) | 0.088 | 0.520 | 0.466 |
| (149/806) | | | | | |
| Ohio, US | 1.33 | (1.08, 1.63) | 6.1 × 10⁻³ | 0.547 | 0.475 |
| (365/383) | | | | | |
| Spain | 1.34 | (0.97, 1.85) | 0.073 | 0.420 | 0.351 |
| (88/1,342) | | | | | |
| All combined | 1.36 | (1.23, 1.50) | 2.0 × 10⁻⁹ | — | — |
| (1,134/5,625)^{c} | | | | | |
| Pₕₑₜ | | 0.85 | | | |
| I² | | 0.0 | | | |

^{a}For rs966423, a SNP that is present on the Illumina chips used to genotype the Icelandic GWAS population, results are included for chip-genotyped individuals. Other results for all study groups are based on single-track assay genotyping.
^{b} rs2439302 is a G/C-SNP and the coding of the alleles here is as on the plus (+) strand of the human reference sequence in Build 36
^{c}For the combined study populations, the OR and the P value were estimated using the Mantel-Haenszel model.

### Example 5

The following methods were used for obtaining the data shown in the above under Example 4.

### Genotyping Methods

### Illumina Genotyping.

The Icelandic chip-typed samples were assayed with the Illumina Human Hap300, Hap CNV370, Hap 610, 1M or Omni-1 Quad bead chips at deCODE genetics. Only the 317,503 SNPs from the Human Hap300 chip were used in the long range phasing and the subsequent SNP imputations. SNPs were excluded if they had (i) yield lower than 95%, (ii) minor allele frequency less than 1% in the population or (iii) significant deviation from Hardy-Weinberg equilibrium in the controls (P<0.001), (iv) if they produced an excessive inheritance error rate (over 0.001), (v) if there was substantial difference in allele frequency between chip types (from just a single chip if that resolved all differences, but from all chips otherwise). All samples with a call rate below 97% were excluded from the analysis. The final set of SNPs used for long range phasing and GWAS was composed of 297,835 autosomal SNPs.

### Single Track Assay SNP Genotyping.

Genotyping of the SNPs reported in Table 1 of the main text for the three case-control groups from Iceland, the Netherlands and Spain was carried out by deCODE Genetics in Reykjavik, Iceland, applying the Centaurus' (Nanogen) platform or the Illumin SNP-chips. Using the Centaurus single-track assay, we genotyped the Spanish cases and controls, the Dutch cases and controls and all the 561 Icelandic patients. Of the Icelandic patients, 222 had been previously chip genotyped for the SNPs on 1p31.3 and 2q35 which are present on the Illuimina SNP-chips used in our initial GWAS genotyping effort. These 222 patients were re-genotyped using Centaurus single-track assay for confirming data consistency of the two genotyping platforms. We used Centaurus single-track assay to genotype between 1,472 and 3,190 Icelandic controls for the 21 TSH-associated SNPs. For the four TSH-associated SNPs that are present on the Illumina chips we included genotype data from 40,013 Icelandic controls GWAS study population. The 3,190 single-track assay genotyped controls are among the 40,013 Illumin chip genotyped controls and the overlap of genotype results was used to check for data consistency. Furthermore, the quality of each Centaurus SNP assay was evaluated by genotyping it in the CEU and/or YRI HapMap samples and comparing the results with the HapMap publicly released data. Assays with >1.5% mismatch rate were not used and a linkage disequilibrium (LD) test was used for markers known to be in LD.

Genotyping of samples from the Ohio study populations was done using the SNaPshot (PE Applied Biosystems, Foster City, Calif.) genotyping platform at the Ohio State University, as previously described².

### Whole Genome Sequencing.

SNPs were imputed based on unpublished data from the Icelandic whole genomic sequencing project (457 Icelandic individuals) selected for various neoplasic, cardiovascular and psychiatric conditions. All of the individuals were sequenced to a depth of at least 10×. Sixteen million SNPs were imputed based on this set of individuals.

### Sample Preparation.

Paired-end libraries for sequencing were prepared according to the manufacturer's instructions (Illumina). In short, approximately 5 μg of genomic DNA, isolated from frozen blood samples, was fragmented to a mean target size of 300 bp using a Covaris E210 instrument. The resulting fragmented DNA was end repaired using T4 and Klenow polymerases and T4 polynucleotide kinase with 10 mM dNTP followed by addition of an ‘A’ base at the ends using Klenow exo fragment (3′ to 5′-exo minus) and dATP (1 mM). Sequencing adaptors containing ‘T’ overhangs were ligated to the DNA products followed by agarose (2%) gel electrophoresis. Fragments of about 400 bp were isolated from the gels (QIAGEN Gel Extraction Kit), and the adaptor-modified DNA fragments were PCR enriched for ten cycles using Phusion DNA polymerase (Finnzymes Oy) and PCR primers PE 1.0 and PE 2.0 (Illumina). Enriched libraries were further purified using agarose (2%) gel electrophoresis as described above. The quality and concentration of the libraries were assessed with the Agilent 2100 Bioanalyzer using the DNA 1000 LabChip (Agilent). Barcoded libraries were stored at −20° C. All steps in the workflow were monitored using an in-house laboratory information management system with barcode tracking of all samples and reagents.

### DNA Sequencing.

Template DNA fragments were hybridized to the surface of flow cells (Illumina PE flowcell, v4) and amplified to form clusters using the Illumina cBot. In brief, DNA (8-10 μM) was denatured, followed by hybridization to grafted adaptors on the flowcell. Isothermal bridge amplification using Phusion polymerase was then followed by linearization of the bridged DNA, denaturation, blocking of 3 ends and hybridization of the sequencing primer. Sequencing-by-synthesis was performed on Illumina GAIIx instruments equipped with paired-end modules. Paired-end libraries were sequenced using 2×101 cycles of incorporation and imaging with Illumina sequencing kits, ≧4. Each library or sample was initially run on a single lane for validation followed by further sequencing of lanes with targeted cluster densities of 250-300 k/mm². Imaging and analysis of the data was performed using the SCS 2.6 and RTA 1.6 software packages from Illumina, respectively. Real-time analysis involved conversion of image data to base-calling in real-time.

### Alignment.

For each lane in the DNA sequencing output, the resulting qseq files were converted into fastq files using an in-house script. All output from sequencing was converted, and the Illumina quality filtering flag was retained in the output. The fastq files were then aligned against Build 36 of the human reference sequence using bwa version 0.5.7 (ref. ³).

### BAM File Generation.

SAM file output from the alignment was converted into BAM format using SAMtools version 0.1.8 (ref. ⁴), and an in-house script was used to carry the Illumina quality filter flag over to the BAM file. The BAM files for each sample were then merged into a single BAM file using SAMtools. Finally, Picard version 1.17 (see http://picard.sourceforge.net/) was used to mark duplicates in the resulting sample BAM files.

### SNP Calling and Genotyping in Whole-Genome Sequencing.

A two-step approach was applied. The first step was to detect SNPs by identifying sequence positions where at least one individual could be determined to be different from the reference sequence with confidence (quality threshold of 20) based on the SNP calling feature of the pileup tool SAMtools⁴. SNPs that always differed heterozygous or homozygous from the reference were removed. The second step was to use the pileup tool to genotype the SNPs at the positions that were flagged as polymorphic. Because sequencing depth varies and hence the certainty of genotype calls also varies, genotype likelihoods rather than deterministic calls were calculated (see below). Of the 2.5 million SNPs reported in the HapMap2 CEU samples, 96.3% were observed in the Icelandic whole-genome sequencing data. Of the 6.9 million SNPs reported in the 1000 Genomes Project data, 89.4% were observed in the Icelandic whole-genome sequencing data.

### Statistical Analysis

### Long Range Phasing.

Long range phasing of all chip-genotyped individuals was performed with methods described previously⁵⁻⁹. In brief, phasing is achieved using an iterative algorithm which phases a single proband at a time given the available phasing information about everyone else that shares a long haplotype identically by state with the proband. Given the large fraction of the Icelandic population that has been chip-typed, accurate long range phasing is available genome-wide for all chip-typed Icelanders.

### Genotype Imputation.

We imputed the SNPs identified and genotyped through sequencing into all Icelanders who had been phased with long range phasing using the same model as used by IMPUTE¹⁹. The genotype data from sequencing can be ambiguous due to low sequencing coverage. In order to phase the sequencing genotypes, an iterative algorithm was applied for each SNP with alleles 0 and 1. We let H be the long range phased haplotypes of the sequenced individuals and applied the following algorithm:
1. For each haplotype h in H, use the Hidden Markov Model of IMPUTE to calculate for every other k in H, the likelihood, denoted γ_{h,k}, of h having the same ancestral source as k at the SNP. For every h in H, initialize the parameter θₕ, which specifies how likely the one allele of the SNP is to occur on the background of h from the genotype likelihoods obtained from sequencing. The genotype likelihood L_{g} is the probability of the observed sequencing data at the SNP for a given individual assuming g is the true genotype at the SNP. If L₀, L₁ and L₂ are the likelihoods of the genotypes 0, 1 and 2 in the individual that carries h, then set

$${θ}_{h} = \frac{{L}_{2} + \frac{1}{2} {L}_{1}}{{L}_{2} + {L}_{1} + {L}_{0}} .$$

2. For every pair of haplotypes h and k in H that are carried by the same individual, use the other haplotypes in H to predict the genotype of the SNP on the backgrounds of h and k: τₕ=Σ_{lεH\{h}}γ_{h,l}θₗ and τₖ=Σ_{lεH\{k}}γ_{k,l}θₗ. Combining these predictions with the genotype likelihoods from sequencing gives un-normalized updated phased genotype probabilities: P₀₀=(1−Σₕ)(1−τₖ)L₀, P₁₀=τₕ(1−τₖ)½L₁, P₀₁=(1−τₕ)τₖ½L₁ and P₁₁=τₕτₖL₂.
3. Now use these values to update θₕ and θₖ to

$${θ}_{h} = \frac{{P}_{10} + {P}_{11}}{{P}_{00} + {P}_{01} + {P}_{10} + {P}_{11}} and {θ}_{k} = \frac{{P}_{01} + {P}_{11}}{{P}_{00} + {P}_{01} + {P}_{10} + {P}_{11}} .$$

4. Repeat step 3 when the maximum difference between iterations is greater than a convergence threshold ε. We used ε=10⁻⁷.

Given the long range phased haplotypes and e, the allele of the SNP on a new haplotype h not in H, is imputed τ_{lεH}γ_{h,l}θₗ.

The above algorithm can easily be extended to handle simple family structures such as parent-offspring pairs and triads by letting the P distribution run over all founder haplotypes in the family structure. The algorithm also extends trivially to the X-chromosome. If source genotype data are only ambiguous in phase, such as chip genotype data, then the algorithm is still applied, but all but one of the Ls will be 0. In some instances, the reference set was intentionally enriched for carriers of the minor allele of a rare SNP in order to improve imputation accuracy. In this case, expected allele counts will be biased toward the minor allele of the SNP. Call the enrichment of the minor allele E and let θ′ be the expected minor allele count calculated from the naĩve imputation method, and let θ be the unbiased expected allele count, then

$${θ}^{′} = \frac{E θ}{1 - θ + E θ}$$

and hence

$$θ = \frac{{θ}^{′}}{E + ( 1 - E ) {θ}^{′}} ,$$

This adjustment was applied to all imputations based on enriched imputations sets. We note that if θ′ is 0 or 1, then θ will also be 0 or 1, respectively.

### In-Silico Genotyping.

In addition to imputing sequence variants from the whole genome sequencing effort into chip genotyped individuals, we also performed a second imputation step where genotypes were imputed into relatives of chip genotyped individuals, creating in-silico genotypes. The inputs into the second imputation step are the fully phased (in particular every allele has been assigned a parent of origin) imputed and chip type genotypes of the available chip typed individuals. The algorithm used to perform the second imputation step consists of:
1. For each ungenotyped individual (the proband), find all chip genotyped individuals within two meiosis of the individual. The six possible types of two meiosis relatives of the proband are (ignoring more complicated relationships due to pedigree loops): Parents, full and half siblings, grandparents, children and grandchildren. If all pedigree paths from the proband to a genotyped relative go through other genotyped relatives, then that relative is excluded. E.g. if a parent of the proband is genotyped, then the proband's grandparents through that parent are excluded. If the number of meiosis in the pedigree around the proband exceeds a threshold (we used 12), then relatives are removed from the pedigree until the number of meiosis falls below 12, in order to reduce computational complexity.
2. At every point in the genome, calculate the probability for each genotyped relative sharing with the proband based on the autosomal SNPs used for phasing. A multipoint algorithm based on the hidden Markov model Lander-Green multipoint linkage algorithm using fast Fourier transforms is used to calculate these sharing probabilities^{34,35}. First single point sharing probabilities are calculated by dividing the genome into 0.5 cM bins and using the haplotypes over these bins as alleles. Haplotypes that are the same, except at most at a single SNP, are treated as identical. When the haplotypes in the pedigree are incompatible over a bin, then a uniform probability distribution was used for that bin. The most common causes for such incompatibilities are recombinations in member belonging to the pedigree, phasing errors and genotyping errors. Note that since the input genotypes are fully phased, the single point information is substantially more informative than for unphased genotyped, in particular one haplotype of the parent of a genotyped child is always known. The single point distributions are then convolved using the multipoint algorithm to obtain multipoint sharing probabilities at the center of each bin. Genetic distances were obtained from the most recent version of the deCODE genetic map⁶.
3. Based on the sharing probabilities at the center of each bin, all the SNPs from the whole genome sequencing are imputed into the proband. To impute the genotype of the paternal allele of a SNP located at x, flanked by bins with centers at x_{left} and x_{right}, Starting with the left bin, going through all possible sharing patterns v, let Iᵥ be the set of haplotypes of genotyped individuals that share identically by descent within the pedigree with the proband's paternal haplotype given the sharing pattern v and P(v) be the probability of v at the left bin—this is the output from step 2 above—and let eᵢ be the expected allele count of the SNP for haplotype i. Then

$${e}_{v} = \frac{{\sum }_{i ∈ {l}_{v}} {e}_{i}}{{\sum }_{i ∈ {l}_{v}} 1}$$

is the expected allele count of the paternal haplotype of the proband given v and an overall estimate of the allele count given the sharing distribution at the left bin is obtained from e_{left}=ΣᵥP(v)eᵥ. If Iᵥ is empty then no relative shares with the proband's paternal haplotype given v and thus there is no information about the allele count. We therefore store the probability that some genotyped relative shared the proband's paternal haplotype, O_{left}=Σ_{v,I}_{ᵥ}_{=Ø}P(v) and an expected allele count, conditional on the proband's paternal haplotype being shared by at least one genotyped relative:

$${c}_{left} = \frac{{\sum }_{v , {l}_{v \neq Ø}} P ( v ) {e}_{v}}{{\sum }_{v , {l}_{v \neq Ø}} P ( v )} .$$

In the same way calculate O_{right} and c_{right}. Linear interpolation is then used to get an estimates at the SNP from the two flanking bins:

$$O = {O}_{left} + \frac{x - {x}_{left}}{{x}_{right} - {x}_{left}} ( {O}_{right} - {O}_{left} ) , \\ c = {c}_{left} + \frac{x - {x}_{left}}{{x}_{right} - {x}_{left}} ( {c}_{right} - {c}_{left} ) .$$

If θ is an estimate of the population frequency of the SNP then 0c+(1−0)θ is an estimate of the allele count for the proband's paternal haplotype. Similarly, an expected allele count can be obtained for the proband's maternal haplotype.

### Genotype Imputation Information.

The informativeness of genotype imputation was estimated by the ratio of the variance of imputed expected allele counts and the variance of the actual allele counts:

$$\frac{Var ( E ( θ \vert chip data ) )}{Var ( θ )} ,$$

where θε{0, 1} is the allele count. Var(E(θ|chip data)) was estimated by the observed variance of the imputed expected counts and var(θ) was estimated by p(1−p), where p is the allele frequency. For the present study, when imputed genotypes are used, the information value for all SNPs is between 0.92 and 0.99.

### Case Control Association Testing.

Logistic regression was used to test for association between SNPs and disease, treating disease status as the response and expected genotype counts from imputation or allele counts from direct genotyping as covariates. Testing was performed using the likelihood ratio statistic. When testing for association based on the in silico genotypes, controls were matched to cases based on the informativeness of the imputed genotypes, such that for each case C controls of matching informativeness where chosen. Failing to match cases and controls will lead to a highly inflated genomic control factor, and in some cases may lead to spurious false positive findings. The informativeness of each of the imputation of each one of an individual's haplotypes was estimated by taking the average of

$$a ( e , θ ) = \{ \begin{matrix}\frac{e - θ}{1 - θ} , & e \geq θ \\ \frac{θ - e}{θ} , & e < θ\end{matrix}$$

over all SNPs imputed for the individual, where e is the expected allele count for the haplotype at the SNP and θ is the population frequency of the SNP. Note that a(θ,θ)=0 and a(0,θ)=a(1,θ)=1. The mean informativeness values cluster into groups corresponding to the most common pedigree configurations used in the imputation, such as imputing from parent into child or from child into parent. Based on this clustering of imputation informativeness we divided the haplotypes of individuals into seven groups of varying informativeness, which created 27 groups of individuals of similar imputation informativeness; 7 groups of individuals with both haplotypes having similar informativeness, 21 groups of individuals with the two haplotypes having different informativeness, minus the one group of individuals with neither haplotype being imputed well. Within each group we calculate the ratio of the number of controls and the number of cases, and choose the largest integer C that was less than this ratio in all the groups. For example, if in one group there are 10.3 times as many controls as cases and if in all other groups this ratio was greater, then we would set C=10 and within each group randomly select ten times as many controls as there are cases. For thyroid cancer we used C=109 and for goiter we used C=186.

### Sibling Recurrence Risk Ratio:

The sibling recurrence risk ratio is defined as

$${λ}_{sibling} = \frac{P ( A \vert B )}{P ( A )} = \frac{P ( AB )}{P ( A ) P ( B )} ,$$

Where A is the event that a person gets a disease and B is the event that a particular sibling of the person gets the disease. Assuming a multiplicative model, the λ—sibling accounted for by a variant with frequency f and relative risk of r is equal to

$$\frac{{\frac{1}{4} [ {fr}^{2} + 1 - f + {( fr + 1 - f )}^{2} ]}^{2}}{{( fr + 1 - f )}^{4 }}$$

### Inflation Factor Adjustment.

In order to account for the relatedness and stratification within our case and control sample sets we applied the method of genomic control based on chip markers. For the thyroid cancer GWAS the correction factor based on the genomic control is 1.14.

### REFERENCES

1. Kutyavin, I. V. et al. A novel endonuclease IV post-PCR genotyping system. Nucleic Acids Research 34, e128 (2006).
2. He, H. et al. Allelic variation in gene expression in thyroid tissue. Thyroid 15, 660-7 (2005).
3. Li, H. & Durbin, R. Fast and accurate short read alignment with Burrows-Wheeler transform. Bioinformatics 25, 1754-60 (2009).
4. L¹, H. et al. The Sequence Alignment/Map format and SAMtools. Bioinformatics 25, 2078-9 (2009).
5. Kong, A. et al. Detection of sharing by descent, long-range phasing and haplotype imputation. Nat Genet. 40, 1068-75 (2008).
6. Kong, A. et al. Fine-scale recombination rate differences between sexes, populations and individuals. Nature 467, 1099-103 (2010).
7. Sulem, P. et al. Identification of low-frequency variants associated with gout and serum uric acid levels. Nat Genet. 43, 1127-30 (2011).
8. Rafnar, T. et al. Mutations in BRIP1 confer high risk of ovarian cancer. Nat Genet. 43, 1104-7 (2011).
9. Stacey, S, N. et al. A germline variant in the TP53 polyadenylation signal confers cancer susceptibility. Nat Genet. 43, 1098-103 (2011).
10. Marchini, J., Howie, B., Myers, S., McVean, G. & Donnelly, P. A new multipoint method for genome-wide association studies by imputation of genotypes. Nat Genet. 39, 906-13 (2007).

### Example 6

The association on chromosome 2q35 and 8p12 was tested in surrogates of the markers rs966423 and rs2439302 by analysis of genotype data obtained by imputation. Imputed genotypes were obtained in Icelandic case-control material of thyroid cancer using methods as described in the above.

Results are shown in Table 7 and Table 8 below. The data illustrates that markers with high correlation with the anchor markers (rs966423 and rs2439302) are associated with risk for thyroid cancer with OR values comparable to those of the anchor marker. Less correlated markers are also associated with thyroid cancer, albeit with decreased OR values as the correlation decreases.

TABLE 7
Association results for correlated markers of marker rs7005606 on chromosome 8 based on imputation in Icelandic samples.
Shown are: marker identity, P-value of association with thyroid cancer in Iceland, value of the correlation coefficient
r² with rs7005606 in Icelandic samples, OR of association with thyroid cancer, frequency (%) of the at-risk allele in
Icelandic samples and in Caucasian samples from the 1000 genomes project (http://www.1000genomes.org) respectively,
information content of the imputed genotype data, position of the surrogate marker in NCBI Build 36, identity of the at-risk
allele and the other allele of each SNP, and reference to the flanking sequence of the SNP.

| | | | | f | f (1000 | | Pos in NCBI | Risk | Other | seq ID |
|---|---|---|---|---|---|---|---|---|---|---|
| Marker | P-value | r² | OR | (Ice) | genomes) | Info | Build 36 | Allele | Allele | no: |
| rs6468096 | 0.533536 | 0.216352 | 1.038 | 38.411 | 39.9 | 0.98699 | 32285229 | G | A | 722 |
| rs7012187 | 0.22958 | 0.206052 | 1.074 | 41.141 | 40.68 | 0.98632 | 32285834 | C | G | 319 |
| rs7005124 | 0.240568 | 0.213611 | 1.072 | 40.996 | 40.68 | 0.98473 | 32287272 | G | T | 723 |
| rs35110336 | 0.249517 | 0.213569 | 1.071 | 40.869 | 40.68 | 0.98386 | 32289082 | C | T | 321 |
| rs13250104 | 0.250287 | 0.21141 | 1.071 | 40.896 | 40.68 | 0.98079 | 32289325 | A | G | 724 |
| rs12543829 | 0.154173 | 0.202318 | 1.089 | 40.387 | 40.68 | 0.97648 | 32289366 | T | C | 725 |
| rs12678982 | 0.0014848 | 0.245347 | 1.248 | 74.918 | 74.93 | 0.98808 | 32416336 | G | A | 405 |
| rs4129579 | 0.00115302 | 0.381988 | 1.222 | 31.763 | 34.91 | 0.99233 | 32417393 | A | G | 406 |
| rs4129580 | 7.04E−05 | 0.247102 | 1.311 | 72.481 | 72.18 | 0.98972 | 32417397 | A | C | 407 |
| rs1579033 | 0.000503948 | 0.381513 | 1.239 | 31.624 | 34.91 | 0.99175 | 32417722 | C | G | 408 |
| rs6981660 | 0.000622791 | 0.378007 | 1.235 | 31.528 | 34.91 | 0.99352 | 32418018 | C | T | 409 |
| rs2347485 | 0.00155522 | 0.245582 | 1.247 | 74.87 | 74.93 | 0.99054 | 32419113 | C | G | 410 |
| rs6468103 | 0.000574302 | 0.381085 | 1.237 | 31.6 | 34.78 | 0.99245 | 32420866 | T | C | 411 |
| rs7833615 | 0.00168622 | 0.204111 | 1.258 | 77.701 | 78.22 | 0.98889 | 32421461 | G | A | 412 |
| rs2347486 | 0.00184902 | 0.203142 | 1.258 | 77.855 | 78.22 | 0.98624 | 32422127 | C | T | 413 |
| rs6468104 | 0.000587978 | 0.300724 | 1.252 | 69.274 | 70.08 | 0.9911 | 32423154 | T | G | 414 |
| rs6994625 | 0.000547458 | 0.38048 | 1.238 | 31.604 | 34.91 | 0.99182 | 32423185 | T | C | 415 |
| rs10090022 | 0.015213 | 0.247998 | 1.157 | 36.062 | 40.29 | 0.99193 | 32424375 | C | T | 416 |
| rs10090023 | 0.0141388 | 0.249001 | 1.159 | 36.065 | 40.42 | 0.99217 | 32424376 | C | T | 417 |
| rs12707705 | 0.00193725 | 0.203422 | 1.256 | 77.836 | 78.22 | 0.98562 | 32424613 | T | G | 420 |
| rs13439435 | 0.000586859 | 0.379979 | 1.236 | 31.761 | 34.91 | 0.9897 | 32424952 | T | A | 422 |
| rs28707398 | 0.000636593 | 0.376471 | 1.235 | 31.625 | 34.91 | 0.9913 | 32424971 | T | G | 423 |
| rs6992352 | 0.000609623 | 0.378531 | 1.235 | 31.661 | 34.91 | 0.993 | 32426216 | G | A | 426 |
| rs6996957 | 0.000573521 | 0.378091 | 1.245 | 67.195 | 67.32 | 0.99196 | 32426526 | C | T | 427 |
| rs2347497 | 0.00192774 | 0.203377 | 1.256 | 77.806 | 78.22 | 0.98592 | 32428367 | A | C | 428 |
| rs10503916 | 0.00212202 | 0.203972 | 1.253 | 77.854 | 78.22 | 0.98804 | 32428808 | A | T | 429 |
| rs12676317 | 0.00213162 | 0.203965 | 1.253 | 77.857 | 78.22 | 0.98805 | 32428864 | T | C | 431 |
| rs4733336 | 0.00216206 | 0.203572 | 1.253 | 77.89 | 78.22 | 0.9884 | 32428933 | C | G | 432 |
| rs10113795 | 0.00185694 | 0.203735 | 1.258 | 77.836 | 78.22 | 0.9862 | 32429422 | T | A | 434 |
| rs10098630 | 0.00185754 | 0.203739 | 1.258 | 77.837 | 78.22 | 0.98622 | 32429426 | C | G | 435 |
| rs10098640 | 0.000527084 | 0.379354 | 1.238 | 31.607 | 34.91 | 0.99361 | 32429440 | A | G | 436 |
| rs13439816 | 0.00058378 | 0.379602 | 1.236 | 31.602 | 34.91 | 0.9941 | 32432027 | A | G | 438 |
| rs10100933 | 0.000481074 | 0.378809 | 1.24 | 31.581 | 34.91 | 0.99293 | 32432504 | C | T | 439 |
| rs6981184 | 0.000694772 | 0.374141 | 1.233 | 31.693 | 34.91 | 0.99372 | 32433447 | A | G | 441 |
| rs59332083 | 0.000685338 | 0.383535 | 1.233 | 31.67 | 34.91 | 0.99337 | 32434095 | G | C | 726 |
| rs16879430 | 0.000567443 | 0.380599 | 1.237 | 31.695 | 34.91 | 0.99372 | 32434360 | A | G | 442 |
| rs7012019 | 0.000926262 | 0.38171 | 1.227 | 31.757 | 34.91 | 0.99164 | 32435032 | G | A | 443 |
| rs77542547 | 0.0321237 | 0.271771 | 1.135 | 54.532 | 59.06 | 0.97943 | 32436662 | T | A | 445 |
| rs17716295 | 0.000885216 | 0.386895 | 1.228 | 31.64 | 34.91 | 0.99277 | 32437459 | A | C | 446 |
| rs12542743 | 0.0127355 | 0.332646 | 1.157 | 52.148 | 56.04 | 0.99259 | 32437897 | C | T | 447 |
| rs12056349 | 0.000727168 | 0.386006 | 1.232 | 31.677 | 34.91 | 0.99266 | 32439015 | A | G | 449 |
| rs6985581 | 0.0199184 | 0.269615 | 1.147 | 55.084 | 59.19 | 0.99254 | 32439080 | T | C | 450 |
| rs11997114 | 0.000922388 | 0.385269 | 1.227 | 31.653 | 34.91 | 0.99357 | 32439500 | C | T | 451 |
| rs10954846 | 0.0133671 | 0.31279 | 1.156 | 51.192 | 56.04 | 0.99074 | 32439762 | A | G | 727 |
| rs12056398 | 0.000634689 | 0.384686 | 1.234 | 31.674 | 34.91 | 0.99306 | 32440271 | C | G | 452 |
| rs10954847 | 0.00394401 | 0.559227 | 1.183 | 45.478 | 48.82 | 0.99475 | 32440388 | G | A | 453 |
| rs12056895 | 0.000872171 | 0.384353 | 1.228 | 31.669 | 34.91 | 0.9936 | 32440649 | G | A | 454 |
| rs12056727 | 0.000736481 | 0.382664 | 1.232 | 31.575 | 34.91 | 0.99217 | 32440712 | T | C | 455 |
| rs12542857 | 0.0219704 | 0.268229 | 1.144 | 55.097 | 59.19 | 0.99073 | 32440840 | A | G | 456 |
| rs57993062 | 0.00256413 | 0.557338 | 1.193 | 45.042 | 48.82 | 0.9926 | 32441288 | G | A | 457 |
| rs4733121 | 0.0260438 | 0.286376 | 1.140 | 55.964 | 59.71 | 0.99162 | 32441822 | T | A | 458 |
| rs6997612 | 0.0018961 | 0.202887 | 1.256 | 77.78 | 78.35 | 0.98919 | 32443771 | A | T | 459 |
| rs10096770 | 0.000707642 | 0.382899 | 1.232 | 31.484 | 34.91 | 0.99546 | 32444762 | G | A | 461 |
| rs10112682 | 0.00015523 | 0.226489 | 1.314 | 76.381 | 75.98 | 0.98695 | 32445189 | C | G | 464 |
| rs7842667 | 0.00191634 | 0.202673 | 1.256 | 77.837 | 78.35 | 0.98726 | 32446897 | C | T | 468 |
| rs7821785 | 0.00197439 | 0.345315 | 1.219 | 30.841 | 35.17 | 0.93701 | 32447299 | T | C | 728 |
| rs7821944 | 0.00566489 | 0.378504 | 1.188 | 31.488 | 34.51 | 0.98599 | 32447421 | A | C | 470 |
| rs6997199 | 0.000151061 | 0.226907 | 1.316 | 76.338 | 75.85 | 0.98665 | 32449542 | C | T | 476 |
| rs62500187 | 0.00192561 | 0.20328 | 1.256 | 77.847 | 78.22 | 0.98695 | 32452249 | G | A | 483 |
| rs4733341 | 0.00139673 | 0.204952 | 1.266 | 77.804 | 78.08 | 0.98647 | 32459990 | C | T | 504 |
| rs10087952 | 0.00121356 | 0.205252 | 1.269 | 77.799 | 78.08 | 0.98623 | 32465974 | C | T | 520 |
| rs6468115 | 8.89E−05 | 0.227141 | 1.328 | 76.226 | 75.72 | 0.98438 | 32473686 | G | T | 540 |
| rs10099542 | 9.44E−05 | 0.22436 | 1.326 | 76.366 | 75.59 | 0.9882 | 32474728 | C | T | 542 |
| rs10755889 | 0.00122091 | 0.204106 | 1.269 | 77.782 | 78.08 | 0.98508 | 32474912 | G | A | 544 |
| rs11506112 | 0.000466853 | 0.387072 | 1.241 | 31.62 | 34.78 | 0.99079 | 32475346 | C | G | 546 |
| rs10808327 | 0.000424779 | 0.387335 | 1.243 | 31.575 | 34.78 | 0.99384 | 32475560 | C | T | 729 |
| rs2347487 | 5.43E−05 | 0.205782 | 1.333 | 75.371 | 75.2 | 0.98702 | 32475577 | T | C | 547 |
| rs28406305 | 0.000480204 | 0.38729 | 1.24 | 31.518 | 34.78 | 0.99226 | 32477465 | T | C | 548 |
| rs28570331 | 0.000273363 | 0.388803 | 1.252 | 31.411 | 34.78 | 0.99247 | 32479243 | T | C | 554 |
| rs4733343 | 5.86E−05 | 0.231267 | 1.337 | 76.083 | 75.72 | 0.98683 | 32479762 | G | T | 555 |
| rs28572535 | 4.71E−05 | 0.222579 | 1.340 | 75.869 | 72.7 | 0.98823 | 32480114 | C | T | 730 |
| rs1878917 | 6.04E−05 | 0.23127 | 1.335 | 76.008 | 75.72 | 0.98692 | 32482237 | G | A | 560 |
| rs7013361 | 5.63E−05 | 0.232067 | 1.337 | 76.024 | 75.85 | 0.9871 | 32482830 | C | A | 561 |
| rs13259892 | 3.62E−05 | 0.257562 | 1.340 | 74.552 | 75.85 | 0.97454 | 32485334 | T | A | 562 |
| rs17645692 | 0.000516729 | 0.213445 | 1.287 | 77.045 | 77.82 | 0.9859 | 32489443 | A | C | 565 |
| rs7844425 | 0.00020345 | 0.416949 | 1.259 | 30.677 | 34.51 | 0.99008 | 32495159 | G | T | 571 |
| rs4733347 | 0.00028122 | 0.224011 | 1.299 | 76.373 | 77.43 | 0.98475 | 32495552 | G | A | 572 |
| rs17718751 | 0.000194004 | 0.416749 | 1.26 | 30.625 | 34.65 | 0.98895 | 32499261 | T | C | 573 |
| rs10092055 | 6.00E−05 | 0.240839 | 1.332 | 75.367 | 76.9 | 0.98597 | 32500953 | G | A | 575 |
| rs10954855 | 5.69E−05 | 0.240988 | 1.332 | 75.336 | 76.9 | 0.98605 | 32501778 | T | A | 576 |
| rs62500191 | 5.61E−05 | 0.241211 | 1.332 | 75.297 | 76.77 | 0.98759 | 32501806 | C | A | 577 |
| rs6651144 | 0.00295051 | 0.330181 | 1.190 | 53.255 | 56.82 | 0.98982 | 32502210 | T | C | 578 |
| rs73234122 | 0.0140794 | 0.216371 | 1.197 | 21.948 | NA | 0.88808 | 32502452 | G | T | 579 |
| rs7000397 | 0.000562169 | 0.384991 | 1.241 | 30.17 | 34.65 | 0.98622 | 32503405 | G | A | 580 |
| rs73234123 | 9.07E−05 | 0.432191 | 1.28 | 28.756 | 32.94 | 0.98592 | 32503977 | C | T | 581 |
| rs6651145 | 0.000228494 | 0.330178 | 1.255 | 31.624 | 35.43 | 0.98622 | 32504317 | C | T | 582 |
| rs6651140 | 5.08E−05 | 0.242129 | 1.335 | 75.332 | 75.85 | 0.98589 | 32504458 | A | G | 583 |
| rs10108197 | 5.63E−05 | 0.241599 | 1.333 | 75.433 | 75.85 | 0.98708 | 32505122 | G | A | 584 |
| rs10111443 | 5.02E−05 | 0.242249 | 1.335 | 75.35 | 75.85 | 0.98503 | 32505416 | C | T | 585 |
| rs60550537 | 2.08E−05 | 0.698057 | 1.284 | 40.879 | 44.36 | 0.98922 | 32509000 | T | A | 586 |
| rs55758802 | 6.72E−05 | 0.429588 | 1.287 | 28.617 | 32.94 | 0.98325 | 32509434 | G | A | 587 |
| rs66963240 | 1.93E−05 | 0.698652 | 1.285 | 40.931 | 44.36 | 0.99154 | 32511825 | T | C | 588 |
| rs10099620 | 5.59E−05 | 0.240195 | 1.332 | 75.221 | 75.85 | 0.98397 | 32512108 | A | G | 589 |
| rs12334435 | 5.18E−05 | 0.2412 | 1.335 | 75.343 | 75.85 | 0.98573 | 32513049 | C | T | 590 |
| rs10105247 | 0.000223674 | 0.536892 | 1.24 | 45.167 | 48.56 | 0.99098 | 32513709 | T | C | 591 |
| rs28594215 | 2.33E−05 | 0.689904 | 1.282 | 40.985 | 44.09 | 0.98917 | 32515060 | A | G | 592 |
| rs6997848 | 0.000106002 | 0.234237 | 1.318 | 75.33 | 75.85 | 0.9843 | 32515069 | C | A | 593 |
| rs4733126 | 0.00026629 | 0.541543 | 1.237 | 45.143 | 48.56 | 0.98925 | 32515321 | A | C | 594 |
| rs3934586 | 6.16E−05 | 0.239657 | 1.330 | 75.235 | 75.85 | 0.98634 | 32516397 | G | A | 595 |
| rs3934585 | 5.23E−05 | 0.241378 | 1.333 | 75.337 | 75.85 | 0.98609 | 32516627 | G | A | 596 |
| rs7819333 | 0.000324336 | 0.327469 | 1.248 | 31.69 | 35.43 | 0.98724 | 32517263 | C | G | 597 |
| rs7838347 | 0.00266838 | 0.328511 | 1.193 | 53.169 | 56.82 | 0.98886 | 32517443 | G | A | 598 |
| rs73234126 | 0.116603 | 0.201957 | 1.13 | 15.894 | 18.24 | 0.99263 | 32519205 | A | G | 601 |
| rs7000590 | 1.09E−05 | 0.275951 | 1.362 | 74.315 | 75.33 | 0.98608 | 32520170 | C | T | 604 |
| rs6996585 | 4.21E−07 | 0.762312 | 1.347 | 39.734 | 42.26 | 0.98883 | 32520345 | G | A | 605 |
| rs7005606 | 5.86E−08 | 1 | 1.372 | 45.76 | 47.38 | 0.9913 | 32521043 | G | T | 606 |
| rs6468119 | 5.42E−05 | 0.572779 | 1.274 | 58.551 | 60.63 | 0.99011 | 32521103 | C | T | 607 |
| rs6468120 | 9.57E−05 | 0.550788 | 1.263 | 57.99 | 60.63 | 0.98828 | 32521636 | C | T | 731 |
| rs73234132 | 0.0192585 | 0.401852 | 1.164 | 27.149 | 27.69 | 0.98398 | 32521783 | T | A | 608 |
| rs7823498 | 3.77E−06 | 0.234608 | 1.412 | 77.774 | 81.23 | 0.98378 | 32523115 | T | C | 609 |
| rs4433107 | 2.16E−05 | — | 1.366 | 76.76 | — | 0.97034 | 32523368 | T | C | 770 |
| rs3802160 | 4.88E−08 | 0.99858 | 1.375 | 45.723 | 47.38 | 0.99045 | 32524171 | G | A | 611 |
| rs3802158 | 4.42E−08 | 0.995773 | 1.376 | 45.664 | 47.38 | 0.98949 | 32524438 | T | C | 612 |
| rs36213229 | 9.48E−08 | 0.813028 | 1.385 | 45.096 | 47.64 | 0.9063 | 32525059 | T | G | 613 |
| rs7834206 | 7.34E−07 | 0.772006 | 1.362 | 45.343 | 47.38 | 0.86816 | 32525690 | A | C | 614 |
| rs73234136 | 5.22E−08 | 0.852782 | 1.388 | 46.06 | 46.33 | 0.93196 | 32525924 | C | T | 615 |
| rs36213544 | 0.0255165 | 0.346465 | 1.161 | 27.518 | 27.69 | 0.92012 | 32525989 | G | C | 616 |
| rs113350646 | 7.68E−05 | 0.229026 | 1.339 | 19.832 | 19.69 | 0.88765 | 32526091 | A | G | 732 |
| rs4733128 | 1.03E−07 | 0.912267 | 1.378 | 44.984 | 46.06 | 0.92866 | 32526144 | T | C | 617 |
| rs4733129 | 6.42E−08 | 0.967923 | 1.374 | 45.708 | 47.38 | 0.97489 | 32526310 | C | T | 618 |
| rs4733130 | 3.95E−08 | 0.99535 | 1.378 | 45.72 | 47.38 | 0.99182 | 32526536 | C | T | 619 |
| rs4733356 | 5.07E−08 | 0.998281 | 1.374 | 45.725 | 47.38 | 0.9908 | 32526995 | T | A | 620 |
| rs4368937 | 5.82E−08 | 0.998735 | 1.372 | 45.736 | 47.38 | 0.99021 | 32527279 | C | T | 621 |
| rs12548687 | 4.96E−08 | 0.997759 | 1.375 | 45.724 | 47.38 | 0.99096 | 32528362 | G | A | 622 |
| rs11781019 | 6.13E−08 | 0.989424 | 1.372 | 45.831 | 47.38 | 0.98917 | 32529060 | A | T | 733 |
| rs4236709 | 8.63E−06 | 0.228205 | 1.393 | 77.65 | 81.23 | 0.98358 | 32529652 | A | G | 623 |
| rs4541858 | 4.20E−08 | 0.995322 | 1.377 | 45.795 | 47.38 | 0.98912 | 32529851 | G | A | 624 |
| rs12543882 | 5.29E−08 | 0.996814 | 1.373 | 45.656 | 47.38 | 0.99143 | 32530235 | T | C | 625 |
| rs2466104 | 7.28E−06 | 0.230329 | 1.397 | 77.657 | 81.23 | 0.98316 | 32530254 | G | C | 626 |
| rs7835688 | 4.56E−08 | 0.989811 | 1.376 | 45.659 | 47.38 | 0.99036 | 32531041 | C | G | 627 |
| rs17646763 | 4.31E−08 | 0.995125 | 1.376 | 45.705 | 47.38 | 0.99207 | 32531198 | C | T | 628 |
| rs17646781 | 0.0241866 | 0.410279 | 1.157 | 26.641 | 27.82 | 0.99148 | 32531622 | A | T | 629 |
| rs2466103 | 0.0835698 | 0.328866 | 1.121 | 71.478 | 70.21 | 0.98286 | 32531846 | T | G | 630 |
| rs2439312 | 8.90E−06 | 0.230573 | 1.393 | 77.727 | 81.23 | 0.98456 | 32531901 | G | A | 631 |
| rs4733357 | 0.0169449 | 0.408052 | 1.167 | 26.716 | 27.82 | 0.98978 | 32532554 | T | C | 632 |
| rs4733131 | 5.10E−08 | 0.985644 | 1.375 | 45.773 | 47.38 | 0.98869 | 32532563 | G | A | 633 |
| rs112852637 | 7.47E−07 | 0.916613 | 1.338 | 46.486 | 47.24 | 0.97651 | 32532782 | C | T | 734 |
| rs11991469 | 3.52E−08 | 0.965564 | 1.381 | 45.502 | 47.24 | 0.98622 | 32532822 | G | C | 634 |
| rs4568578 | 8.00E−09 | 0.430839 | 1.451 | 65.581 | 68.37 | 0.97749 | 32532829 | C | T | 635 |
| rs11991474 | 5.10E−08 | 0.974623 | 1.375 | 45.753 | 47.51 | 0.98978 | 32532852 | T | C | 636 |
| rs9642727 | 7.21E−08 | 0.979004 | 1.369 | 45.818 | 47.51 | 0.99071 | 32533574 | C | A | 637 |
| rs17646936 | 2.54E−08 | 0.425971 | 1.433 | 66.014 | 69.16 | 0.98376 | 32533616 | A | G | 638 |
| rs17719687 | 0.0578914 | 0.210783 | 1.161 | 15.315 | 17.85 | 0.99194 | 32533708 | G | A | 639 |
| rs17719705 | 6.41E−08 | 0.975797 | 1.371 | 45.758 | 47.51 | 0.99018 | 32533874 | T | A | 640 |
| rs9642699 | 4.00E−05 | 0.2791 | 1.341 | 18.901 | 19.69 | 0.98098 | 32534156 | G | A | 641 |
| rs7014349 | 0.0164172 | 0.409043 | 1.168 | 26.752 | 27.82 | 0.99102 | 32535043 | C | T | 642 |
| rs6989777 | 0.0203735 | 0.40819 | 1.162 | 26.805 | 27.82 | 0.99064 | 32535224 | A | G | 643 |
| rs7825175 | 2.61E−05 | 0.285175 | 1.349 | 19.038 | 19.69 | 0.98328 | 32535816 | A | G | 644 |
| rs35004034 | 0.018512 | 0.409521 | 1.165 | 26.751 | 27.82 | 0.98979 | 32535941 | T | A | 645 |
| rs35919297 | 0.0177644 | 0.402058 | 1.166 | 26.588 | 27.82 | 0.98737 | 32536084 | A | G | 646 |
| rs11777396 | 0.0163401 | 0.408378 | 1.168 | 26.755 | 27.82 | 0.98979 | 32536776 | T | G | 647 |
| rs12543602 | 0.0276392 | 0.398895 | 1.154 | 26.835 | NA | 0.98643 | 32536914 | A | G | 648 |
| rs10101464 | 1.65E−08 | 0.365806 | 1.462 | 69.867 | 73.23 | 0.98151 | 32537004 | C | T | 649 |
| rs13260545 | 2.16E−08 | 0.361688 | 1.458 | 69.965 | 73.23 | 0.9798 | 32537142 | T | C | 650 |
| rs73234144 | 0.0193172 | 0.408845 | 1.164 | 26.704 | 27.82 | 0.99 | 32538611 | A | G | 651 |
| rs11776203 | 0.0169506 | 0.406819 | 1.167 | 26.704 | 27.82 | 0.9918 | 32538661 | G | T | 652 |
| rs55927812 | 0.0129228 | 0.406588 | 1.174 | 26.695 | 27.82 | 0.99204 | 32539338 | T | C | 653 |
| rs7833971 | 2.19E−05 | 0.284584 | 1.351 | 19.176 | 19.69 | 0.98431 | 32539596 | G | A | 735 |
| rs4316112 | 0.0198415 | 0.408095 | 1.163 | 26.642 | 27.82 | 0.99173 | 32539889 | A | C | 654 |
| rs12681692 | 0.0217709 | 0.408815 | 1.16 | 26.689 | 27.82 | 0.99107 | 32540276 | G | A | 655 |
| rs12675358 | 0.0191426 | 0.408451 | 1.164 | 26.752 | 27.82 | 0.98957 | 32540531 | T | G | 656 |
| rs12679578 | 0.0199319 | 0.409527 | 1.162 | 26.803 | 27.82 | 0.99186 | 32540667 | T | C | 657 |
| rs73234146 | 0.0206186 | 0.408918 | 1.162 | 26.692 | 27.82 | 0.99033 | 32540813 | G | A | 658 |
| rs73234147 | 0.0204308 | 0.409503 | 1.162 | 26.679 | 27.82 | 0.99068 | 32540929 | G | A | 659 |
| rs60738472 | 2.43E−05 | 0.285283 | 1.35 | 19.035 | 19.69 | 0.98312 | 32541014 | T | C | 736 |
| rs12682268 | 1.51E−09 | 0.466587 | 1.468 | 64.01 | 66.8 | 0.98427 | 32541497 | A | G | 660 |
| rs56332814 | 0.0161807 | 0.406123 | 1.168 | 26.766 | 27.82 | 0.98989 | 32541620 | C | T | 661 |
| rs35830140 | 0.018181 | 0.408474 | 1.165 | 26.728 | 27.82 | 0.98985 | 32541642 | T | G | 662 |
| rs73234149 | 0.0190734 | 0.408624 | 1.164 | 26.77 | 27.82 | 0.98939 | 32542073 | C | T | 663 |
| rs11774911 | 0.0313889 | #N/A | 1.15 | 26.785 | 27.56 | 0.98747 | 32542399 | G | T | 771 |
| rs11784378 | 0.0276541 | 0.403259 | 1.154 | 26.736 | 27.69 | 0.98769 | 32542400 | G | C | 665 |
| rs11784382 | 0.0330114 | 0.402902 | 1.148 | 26.78 | 27.82 | 0.9915 | 32542428 | T | C | 666 |
| rs13258892 | 1.14E−08 | 0.444224 | 1.441 | 65.061 | 67.59 | 0.98464 | 32543079 | C | T | 667 |
| rs73234151 | 0.0315577 | 0.404302 | 1.15 | 26.82 | 27.82 | 0.98606 | 32543080 | T | G | 668 |
| rs112811550 | 0.0840856 | 0.348568 | 1.117 | 28.285 | 29.4 | 0.98736 | 32543180 | G | A | 669 |
| rs79949912 | 0.0286764 | 0.400906 | 1.152 | 26.927 | 27.82 | 0.98667 | 32543188 | A | G | 670 |
| rs76126400 | 0.0310647 | 0.402534 | 1.15 | 26.918 | 27.82 | 0.98748 | 32543274 | A | G | 737 |
| rs35190404 | 0.0385181 | 0.392664 | 1.145 | 26.64 | 27.82 | 0.98012 | 32543348 | C | G | 738 |
| rs11785360 | 0.0249565 | 0.400315 | 1.157 | 26.792 | 27.82 | 0.98733 | 32543446 | T | C | 671 |
| rs11775204 | 0.0350564 | 0.40186 | 1.147 | 26.765 | 27.82 | 0.98777 | 32543629 | G | A | 672 |
| rs11775972 | 0.0355893 | 0.403459 | 1.146 | 26.774 | 27.82 | 0.98803 | 32543699 | G | T | 673 |
| rs4733358 | 0.0362652 | 0.399394 | 1.146 | 26.739 | 27.82 | 0.98748 | 32543963 | G | A | 674 |
| rs73234154 | 0.0803786 | 0.205389 | 1.148 | 15.283 | 17.85 | 0.99266 | 32544371 | A | G | 675 |
| rs35525180 | 1.40E−07 | 0.417022 | 1.406 | 66.534 | 69.16 | 0.98403 | 32544681 | G | A | 676 |
| rs4733132 | 0.0346291 | 0.402 | 1.147 | 26.773 | 27.82 | 0.98732 | 32545285 | G | C | 677 |
| rs11787271 | 0.0321934 | 0.401762 | 1.149 | 26.776 | 27.82 | 0.98709 | 32545488 | T | C | 678 |
| rs73234158 | 0.0334904 | 0.402806 | 1.148 | 26.782 | 27.82 | 0.98686 | 32545704 | T | G | 679 |
| rs13252144 | 1.27E−08 | 0.459332 | 1.437 | 64.435 | 66.93 | 0.98139 | 32546324 | G | T | 680 |
| rs13252431 | 1.18E−07 | 0.353117 | 1.429 | 70.415 | 73.23 | 0.98306 | 32546426 | G | A | 681 |
| rs73234160 | 0.0313765 | 0.40285 | 1.15 | 26.852 | 27.95 | 0.9852 | 32546942 | G | T | 682 |
| rs111487384 | 0.0850343 | 0.348486 | 1.117 | 28.237 | 29.53 | 0.98651 | 32547121 | C | T | 683 |
| rs4733360 | 0.0370996 | 0.4027 | 1.145 | 26.777 | 27.82 | 0.98608 | 32547745 | C | G | 684 |
| rs10503920 | 2.08E−07 | 0.416497 | 1.399 | 66.586 | 69.29 | 0.98552 | 32548231 | A | G | 685 |
| rs2466100 | 8.45E−08 | 0.919731 | 1.369 | 45.697 | 48.03 | 0.9846 | 32548891 | T | A | 686 |
| rs2439305 | 7.37E−08 | 0.92214 | 1.37 | 45.752 | 48.03 | 0.98489 | 32549006 | G | A | 687 |
| rs35233333 | 1.66E−07 | 0.414187 | 1.404 | 66.687 | 69.29 | 0.97994 | 32549276 | T | C | 688 |
| rs78953577 | 1.79E−05 | 0.254901 | 1.356 | 18.992 | 20.08 | 0.98155 | 32549381 | T | G | 689 |
| rs2466098 | 8.01E−08 | 0.919791 | 1.369 | 45.86 | 48.03 | 0.9857 | 32549458 | A | G | 690 |
| rs2439304 | 8.58E−07 | 0.861818 | 1.335 | 47.188 | 49.61 | 0.98536 | 32549913 | A | G | 691 |
| rs2439303 | 9.04E−08 | 0.917896 | 1.368 | 45.81 | 48.03 | 0.9844 | 32549917 | T | C | 692 |
| rs17720634 | 0.0307864 | 0.402396 | 1.151 | 26.802 | 27.82 | 0.98575 | 32550116 | G | T | 693 |
| rs9642728 | 1.68E−05 | 0.256537 | 1.357 | 18.957 | 19.82 | 0.98217 | 32550232 | G | A | 695 |
| rs2466096 | 1.48E−05 | 0.255962 | 1.36 | 18.967 | 20.21 | 0.98058 | 32550274 | A | T | 696 |
| rs2466095 | 8.53E−08 | 0.921544 | 1.369 | 45.784 | 48.03 | 0.98377 | 32550391 | C | T | 697 |
| rs2919373 | 1.59E−05 | 0.256209 | 1.358 | 19.013 | 20.21 | 0.98172 | 32551401 | T | C | 698 |
| rs2439302 | 1.25E−07 | 0.917873 | 1.363 | 45.891 | 48.03 | 0.98494 | 32551911 | G | C | 699 |
| rs2466077 | 7.94E−08 | 0.865821 | 1.37 | 46.76 | 48.95 | 0.98472 | 32552295 | G | T | 700 |
| rs2466076 | 6.86E−08 | 0.865622 | 1.372 | 46.727 | 48.95 | 0.98398 | 32552338 | G | T | 701 |
| rs2466075 | 0.000125799 | 0.528287 | 1.254 | 49.677 | 48.29 | 0.97766 | 32552491 | A | G | 702 |
| rs71512640 | 4.50E−06 | 0.206522 | 1.433 | 79.778 | 81.63 | 0.9783 | 32552499 | G | A | 703 |
| rs2466074 | 1.50E−05 | 0.611186 | 1.292 | 51.93 | 54.07 | 0.97562 | 32552680 | C | T | 704 |
| rs17720837 | 0.0972413 | 0.324221 | 1.114 | 27.032 | 27.03 | 0.98175 | 32552708 | T | C | 705 |
| rs2466073 | 1.09E−05 | 0.532593 | 1.300 | 54.658 | 57.09 | 0.97749 | 32552854 | G | A | 706 |
| rs2439299 | 3.53E−05 | 0.549531 | 1.277 | 52.174 | 54.99 | 0.97884 | 32553227 | A | C | 707 |
| rs73234169 | 0.154946 | 0.315452 | 1.097 | 27.582 | 27.17 | 0.97575 | 32553256 | G | T | 708 |
| rs2466072 | 2.77E−05 | 0.554285 | 1.282 | 52.087 | 54.99 | 0.97893 | 32553435 | G | A | 709 |
| rs2466071 | 7.86E−05 | 0.547465 | 1.264 | 52.575 | 54.99 | 0.97465 | 32553664 | A | T | 710 |
| rs2466070 | 2.58E−05 | 0.564816 | 1.282 | 52.138 | 55.12 | 0.98185 | 32554159 | C | T | 712 |
| rs10954856 | 0.114328 | 0.322717 | 1.108 | 27.69 | 27.17 | 0.97878 | 32555334 | G | A | 713 |
| rs11783278 | 0.136506 | 0.320106 | 1.101 | 27.801 | 27.17 | 0.97657 | 32556075 | A | T | 714 |
| rs11783353 | 0.127574 | 0.32238 | 1.104 | 27.597 | 27.17 | 0.97813 | 32556327 | C | T | 715 |
| rs17721043 | 0.129438 | 0.323573 | 1.103 | 27.654 | 27.17 | 0.97855 | 32556417 | A | G | 716 |
| rs2466066 | 7.63E−07 | 0.273793 | 1.408 | 72.537 | 79.13 | 0.98578 | 32557958 | G | A | 739 |
| rs2439296 | 0.000105803 | 0.443122 | 1.263 | 57.364 | 60.1 | 0.97826 | 32559506 | C | T | 717 |
| rs2439295 | 0.000120451 | 0.443195 | 1.259 | 57.508 | 60.1 | 0.98055 | 32559771 | C | T | 718 |
| rs17721216 | 0.105226 | 0.308384 | 1.111 | 27.111 | 27.17 | 0.98059 | 32561481 | C | T | 719 |
| rs2439292 | 0.000143907 | 0.444544 | 1.256 | 57.544 | 60.1 | 0.98145 | 32566424 | G | A | 720 |

TABLE 8
Association results for correlated markers of marker rs966423 on chromosome 2 based on imputation in Icelandic samples.
Shown are: marker identity, P-value of association with thyroid cancer in Iceland, value of the correlation coefficient
r² with rs966423 in Icelandic samples, OR of association with thyroid cancer, frequency of the at-risk allele in Icelandic
samples and in Caucasian samples from the 1000 genomes project (http://www.1000genomes.org) respectively, information
content of the imputed genotype data, position of the surrogate marker in NCBI Build 36, identity of the at-risk allele and the other
allele of each SNP, and reference to the flanking sequence of the SNP.

| | | | | f | f (1000 | | Pos in NCBI Build | Risk | Other | seq ID |
|---|---|---|---|---|---|---|---|---|---|---|
| Marker | P-value | r² | OR | (Ice) | genomes) | info | 36 | Allele | Allele | no: |
| rs12151423 | 0.0113297 | 0.381252 | 1.16 | 49.929 | 49.74 | 0.98069 | 217945526 | A | G | 1 |
| rs12151670 | 0.00422491 | 0.439671 | 1.183 | 52.331 | 50.92 | 0.98079 | 217945682 | G | A | 2 |
| rs12620884 | 0.00417837 | 0.436833 | 1.183 | 52.342 | 51.05 | 0.98294 | 217947126 | G | A | 4 |
| rs143993754 | 0.00303583 | 0.421527 | 1.190 | 51.153 | 52.62 | 0.9828 | 217951552 | G | A | 5 |
| rs10211167 | 0.00287466 | 0.42153 | 1.192 | 51.319 | 52.36 | 0.98284 | 217952361 | T | G | 740 |
| rs7575155 | 0.00278195 | 0.420415 | 1.192 | 51.417 | 52.49 | 0.98431 | 217952389 | G | A | 6 |
| rs2373058 | 0.0224676 | 0.246077 | 1.182 | 18.106 | 18.37 | 0.98368 | 217958794 | C | G | 7 |
| rs6706673 | 2.39E−07 | 0.516908 | 1.37 | 30.96 | 30.84 | 0.98693 | 217959947 | A | G | 8 |
| rs34587525 | 0.0446791 | 0.273609 | 1.155 | 19.508 | 20.73 | 0.98676 | 217961934 | A | G | 10 |
| rs13389185 | 0.000278786 | 0.814705 | 1.236 | 48.078 | 47.38 | 0.98992 | 217963774 | C | T | 12 |
| rs4674161 | 1.47E−05 | 0.90965 | 1.287 | 43.655 | 43.04 | 0.98816 | 217964254 | C | T | 13 |
| rs6723847 | 3.92E−07 | 0.518485 | 1.362 | 31.051 | 30.84 | 0.98894 | 217964734 | T | C | 14 |
| rs12232972 | 0.0213578 | 0.245631 | 1.184 | 18.13 | 17.59 | 0.98307 | 217965517 | T | C | 15 |
| rs10932715 | 0.025874 | 0.250216 | 1.178 | 18.06 | 18.37 | 0.98384 | 217968028 | C | T | 16 |
| rs58933889 | 0.0176663 | 0.232131 | 1.196 | 16.719 | 16.8 | 0.98559 | 217970178 | A | G | 17 |
| rs17191752 | 0.000441385 | 0.816686 | 1.227 | 48.134 | 47.38 | 0.99105 | 217970985 | G | A | 18 |
| rs7579927 | 0.000385777 | 0.81928 | 1.23 | 48.134 | 47.38 | 0.99295 | 217971087 | C | T | 19 |
| rs17804901 | 0.000395894 | 0.818696 | 1.229 | 48.168 | 47.38 | 0.99232 | 217971121 | C | G | 21 |
| rs62176727 | 0.0004799 | 0.806294 | 1.226 | 48.254 | 44.88 | 0.98834 | 217972044 | C | T | 22 |
| rs12989997 | 1.75E−05 | 0.82786 | 1.284 | 41.541 | 39.9 | 0.99289 | 217974601 | C | T | 25 |
| rs55806820 | 1.51E−05 | 0.827216 | 1.287 | 41.501 | 40.03 | 0.99382 | 217974990 | C | T | 26 |
| rs1351163 | 0.0221674 | 0.249896 | 1.182 | 18.045 | 18.37 | 0.99004 | 217976237 | G | A | 27 |
| rs9752576 | 0.0257983 | 0.292294 | 1.17 | 20.237 | 20.73 | 0.99 | 217977690 | A | G | 28 |
| rs6759952 | 0.000220995 | 0.71994 | 1.239 | 46.057 | 44.75 | 0.99543 | 217979964 | T | C | 29 |
| rs73079697 | 0.0195823 | 0.251304 | 1.188 | 17.708 | NA | 0.98807 | 217980999 | T | G | 32 |
| rs10195077 | 0.019742 | 0.253991 | 1.186 | 17.987 | 17.59 | 0.98802 | 217981194 | T | C | 22 |
| rs6720623 | 0.544293 | 0.428028 | 1.037 | 35.183 | 34.51 | 0.99521 | 217981325 | A | G | 34 |
| rs6720752 | 0.000196813 | 0.725996 | 1.241 | 45.823 | 44.88 | 0.99322 | 217981456 | A | G | 35 |
| rs6720977 | 0.545265 | 0.42865 | 1.037 | 35.175 | 34.51 | 0.995 | 217981620 | A | G | 36 |
| rs6721000 | 0.541303 | 0.427939 | 1.038 | 35.177 | 34.51 | 0.99518 | 217981698 | A | G | 37 |
| rs1382430 | 0.531936 | 0.427423 | 1.039 | 35.118 | 34.51 | 0.99431 | 217982533 | T | C | 38 |
| rs1382431 | 0.507447 | 0.428477 | 1.041 | 34.997 | 34.51 | 0.99306 | 217982668 | T | C | 39 |
| rs4674163 | 0.520928 | 0.427641 | 1.04 | 35.096 | 34.51 | 0.99393 | 217982725 | G | A | 40 |
| rs10932716 | 0.528148 | 0.426984 | 1.039 | 35.145 | 34.51 | 0.99541 | 217982906 | G | A | 41 |
| rs11674838 | 0.529112 | 0.428316 | 1.039 | 35.118 | 34.51 | 0.99363 | 217982945 | T | C | 42 |
| rs4674164 | 0.526272 | 0.42729 | 1.039 | 35.133 | 34.51 | 0.99492 | 217983142 | T | C | 43 |
| rs4674165 | 0.530565 | 0.427983 | 1.039 | 35.132 | 34.51 | 0.99409 | 217983188 | T | C | 44 |
| rs4674167 | 0.531758 | 0.427737 | 1.039 | 35.149 | 34.51 | 0.99496 | 217983615 | T | C | 46 |
| rs981938 | 0.515512 | 0.429026 | 1.04 | 34.975 | 34.51 | 0.9936 | 217984063 | G | A | 47 |
| rs4674168 | 0.000205287 | 0.725355 | 1.241 | 45.828 | 44.88 | 0.99353 | 217984406 | T | C | 48 |
| rs4674169 | 0.529244 | 0.427939 | 1.039 | 35.12 | 34.51 | 0.99454 | 217984485 | T | C | 49 |
| rs6707903 | 0.530785 | 0.427707 | 1.039 | 35.123 | 34.51 | 0.99454 | 217985081 | G | A | 50 |
| rs6736742 | 0.174193 | 0.526621 | 1.089 | 30.57 | 30.05 | 0.99351 | 217985394 | A | G | 52 |
| rs1478575 | 0.498367 | 0.42876 | 1.042 | 35.037 | 34.51 | 0.99379 | 217986800 | T | A | 53 |
| rs2113832 | 0.492997 | 0.430334 | 1.042 | 34.951 | 34.51 | 0.99355 | 217986937 | A | G | 54 |
| rs2162001 | 0.000138037 | 0.726011 | 1.248 | 45.894 | 44.88 | 0.99469 | 217987015 | T | C | 55 |
| rs1600210 | 0.483254 | 0.429172 | 1.043 | 35.155 | 34.51 | 0.99345 | 217987026 | C | A | 56 |
| rs1600211 | 0.509576 | 0.428897 | 1.041 | 35.082 | 34.51 | 0.99448 | 217987248 | A | G | 57 |
| rs1600212 | 0.515218 | 0.429244 | 1.04 | 35.096 | 34.51 | 0.99448 | 217987355 | T | C | 58 |
| rs10191791 | 0.503596 | 0.428369 | 1.041 | 35.069 | 34.51 | 0.99488 | 217987492 | A | G | 59 |
| rs34413965 | 0.497774 | 0.430972 | 1.042 | 34.952 | 34.51 | 0.99252 | 217987716 | C | T | 60 |
| rs34756249 | 0.490482 | 0.431647 | 1.043 | 34.924 | 34.51 | 0.99232 | 217987731 | T | C | 61 |
| rs7567847 | 0.488907 | 0.425897 | 1.043 | 35.15 | 34.51 | 0.99319 | 217987818 | C | A | 62 |
| rs7570554 | 0.491138 | 0.430401 | 1.043 | 34.934 | 34.51 | 0.99415 | 217987934 | C | T | 63 |
| rs7584902 | 0.167229 | 0.524808 | 1.09 | 30.739 | 30.05 | 0.98961 | 217988183 | T | G | 65 |
| rs1118149 | 0.173594 | 0.525466 | 1.089 | 30.695 | 30.05 | 0.99027 | 217988491 | A | G | 66 |
| rs1118150 | 0.175678 | 0.524447 | 1.088 | 30.699 | 30.05 | 0.99011 | 217988513 | C | A | 67 |
| rs1118151 | 0.258562 | 0.522254 | 1.074 | 29.236 | 29.53 | 0.99175 | 217988663 | T | G | 68 |
| rs13388148 | 0.0170234 | 0.252899 | 1.191 | 17.955 | 17.72 | 0.98798 | 217989745 | G | T | 69 |
| rs13406698 | 0.0181006 | 0.251629 | 1.189 | 17.981 | 17.72 | 0.98815 | 217991330 | G | A | 70 |
| rs13395110 | 0.518885 | 0.427262 | 1.04 | 35.099 | 34.51 | 0.99465 | 217991548 | G | T | 71 |
| rs13432615 | 0.505333 | 0.4291 | 1.041 | 34.994 | 34.51 | 0.99113 | 217991684 | T | C | 72 |
| rs994532 | 0.53416 | 0.428833 | 1.038 | 35.13 | 34.51 | 0.99435 | 217992455 | G | A | 73 |
| rs994533 | 0.51652 | 0.427065 | 1.04 | 35.095 | 34.51 | 0.99479 | 217992523 | C | G | 74 |
| rs10490762 | 0.514048 | 0.427291 | 1.04 | 35.078 | 34.51 | 0.9941 | 217992642 | A | T | 75 |
| rs1478576 | 0.50855 | 0.427679 | 1.041 | 35.043 | 34.51 | 0.99324 | 217992769 | C | T | 76 |
| rs1478577 | 0.000249562 | 0.819431 | 1.237 | 48.21 | 48.16 | 0.99416 | 217992813 | A | G | 77 |
| rs1382432 | 0.000238848 | 0.820523 | 1.238 | 48.127 | 48.16 | 0.99297 | 217993044 | A | G | 78 |
| rs13401747 | 0.50119 | 0.427962 | 1.042 | 35.006 | 34.51 | 0.99233 | 217993059 | C | T | 79 |
| rs1382434 | 5.54E−05 | 0.696909 | 1.266 | 45.739 | 44.88 | 0.98028 | 217993357 | G | C | 81 |
| rs11676600 | 0.168427 | 0.522869 | 1.09 | 30.674 | 30.05 | 0.99056 | 217993634 | A | C | 82 |
| rs11678088 | 0.169358 | 0.523093 | 1.09 | 30.679 | 30.05 | 0.99037 | 217994344 | C | T | 83 |
| rs7603771 | 0.503878 | 0.43026 | 1.041 | 35.108 | 34.51 | 0.99114 | 217995359 | T | A | 84 |
| rs7577615 | 0.51044 | 0.426905 | 1.041 | 35.031 | 34.51 | 0.99233 | 217995426 | T | C | 85 |
| rs11890853 | 0.171152 | 0.523384 | 1.089 | 30.701 | 30.05 | 0.98957 | 217996436 | T | C | 86 |
| rs74723351 | 0.0159692 | 0.252341 | 1.193 | 17.934 | 17.72 | 0.98872 | 217996462 | A | G | 83 |
| rs11890939 | 0.167822 | 0.522869 | 1.09 | 30.675 | 30.05 | 0.99054 | 217996470 | T | G | 88 |
| rs13425215 | 0.0161877 | 0.253429 | 1.192 | 17.817 | 17.72 | 0.99285 | 217996825 | G | A | 89 |
| rs13428040 | 0.000168846 | 0.823247 | 1.244 | 48.039 | 48.16 | 0.9949 | 217997076 | A | T | 90 |
| rs2373061 | 0.538434 | 0.42951 | 1.038 | 35.128 | 34.51 | 0.99387 | 217997492 | G | T | 91 |
| rs12694415 | 0.00021581 | 0.72497 | 1.24 | 45.94 | 44.88 | 0.99458 | 217997602 | G | A | 92 |
| rs12694416 | 0.000193586 | 0.726411 | 1.242 | 45.91 | 44.88 | 0.99459 | 217997742 | A | C | 93 |
| rs10804259 | 0.000201457 | 0.725018 | 1.241 | 45.915 | 44.88 | 0.993 | 217998287 | C | T | 94 |
| rs10804260 | 0.50119 | 0.429305 | 1.042 | 35.144 | 34.51 | 0.99193 | 217998293 | C | T | 95 |
| rs62175475 | 0.483029 | 0.428614 | 1.044 | 35.069 | 34.51 | 0.99224 | 217998603 | T | C | 96 |
| rs12624106 | 0.0163534 | 0.253585 | 1.192 | 17.822 | 17.72 | 0.99259 | 217998690 | G | A | 97 |
| rs2194737 | 0.516814 | 0.429302 | 1.04 | 35.041 | 34.51 | 0.99266 | 217998914 | C | T | 98 |
| rs2194736 | 0.503153 | 0.428369 | 1.041 | 35.072 | 34.51 | 0.99469 | 217999216 | T | C | 99 |
| rs3732009 | 0.0163409 | 0.254744 | 1.192 | 17.87 | 17.72 | 0.98914 | 217999638 | A | G | 100 |
| rs1478579 | 0.15886 | 0.525544 | 1.092 | 30.534 | 29.92 | 0.99335 | 217999769 | T | C | 101 |
| rs1478580 | 0.0159064 | 0.255544 | 1.193 | 17.819 | 17.72 | 0.99234 | 217999894 | T | C | 102 |
| rs3821098 | 8.30E−08 | 0.461152 | 1.391 | 28.608 | 28.08 | 0.99456 | 218000386 | T | C | 103 |
| rs11693806 | 8.23E−08 | 0.461114 | 1.391 | 28.606 | 28.08 | 0.99449 | 218000403 | C | G | 104 |
| rs1478581 | 0.018771 | 0.25666 | 1.188 | 17.885 | 17.72 | 0.99163 | 218000897 | A | G | 105 |
| rs6745321 | 0.000214726 | 0.822778 | 1.24 | 48.168 | 48.03 | 0.99435 | 218001479 | T | C | 107 |
| rs34140398 | 0.00320366 | 0.355098 | 1.216 | 23.549 | 22.57 | 0.99554 | 218001673 | C | T | 741 |
| rs7594625 | 0.539433 | 0.495399 | 1.037 | 37.379 | 37.66 | 0.99488 | 218001809 | G | T | 108 |
| rs13016875 | 0.00023279 | 0.501997 | 1.242 | 38.455 | 35.3 | 0.99627 | 218002336 | T | A | 109 |
| rs12990503 | 6.80E−08 | 0.462697 | 1.394 | 28.571 | 28.08 | 0.9942 | 218002462 | C | G | 110 |
| rs6734808 | 0.0165138 | 0.25564 | 1.192 | 17.832 | 17.72 | 0.99291 | 218002816 | T | C | 111 |
| rs13388294 | 4.57E−07 | 0.424962 | 1.362 | 29.756 | 29.4 | 0.98805 | 218003651 | A | G | 113 |
| rs1382435 | 0.000240834 | 0.501871 | 1.241 | 38.489 | 35.3 | 0.99554 | 218004248 | T | C | 114 |
| rs13004333 | 0.000274672 | 0.5018 | 1.238 | 38.505 | 35.3 | 0.99644 | 218004386 | C | G | 115 |
| rs57481445 | 9.36E−08 | 0.461135 | 1.389 | 28.613 | 27.82 | 0.99439 | 218004619 | G | A | 116 |
| rs16857609 | 9.05E−08 | 0.461152 | 1.389 | 28.614 | 27.82 | 0.99432 | 218004753 | T | C | 117 |
| rs16857611 | 9.95E−08 | 0.459717 | 1.388 | 28.646 | 27.82 | 0.99208 | 218004977 | T | C | 118 |
| rs11680689 | 2.00E−07 | 0.524846 | 1.371 | 30.937 | 31.36 | 0.99217 | 218005945 | C | G | 119 |
| rs1233081 | 1.95E−05 | 0.677548 | 1.288 | 36.192 | 34.25 | 0.9937 | 218008489 | T | C | 120 |
| rs12478966 | 2.09E−05 | 0.677324 | 1.287 | 36.217 | 34.25 | 0.99302 | 218008808 | A | G | 122 |
| rs12473807 | 2.17E−05 | 0.676779 | 1.286 | 36.253 | 34.25 | 0.99252 | 218008967 | A | T | 123 |
| rs4674176 | 8.67E−07 | 0.539717 | 1.348 | 31.194 | 31.5 | 0.99282 | 218009364 | G | C | 124 |
| rs13002451 | 8.22E−07 | 0.539515 | 1.349 | 31.204 | 31.5 | 0.99239 | 218009586 | G | A | 125 |
| rs2618146 | 3.59E−05 | 0.443839 | 1.279 | 35.699 | 36.09 | 0.9911 | 218010258 | G | A | 126 |
| rs2618147 | 1.06E−06 | 0.538592 | 1.345 | 31.278 | 31.63 | 0.99008 | 218010383 | A | C | 127 |
| rs12617808 | 0.000636102 | 0.839907 | 1.22 | 48.555 | 48.29 | 0.99319 | 218010462 | T | C | 128 |
| rs2568176 | 1.71E−06 | 0.556773 | 1.336 | 31.748 | 31.76 | 0.9928 | 218012203 | A | G | 129 |
| rs2618148 | 5.65E−06 | 0.549494 | 1.318 | 31.462 | 31.76 | 0.99087 | 218012351 | T | C | 130 |
| rs2568175 | 0.000169279 | 0.691817 | 1.25 | 37.235 | 34.12 | 0.99095 | 218012753 | A | T | 132 |
| rs6715218 | 6.22E−06 | 0.578155 | 1.316 | 31.327 | 31.23 | 0.99649 | 218013309 | C | T | 133 |
| rs6729012 | 6.17E−06 | 0.578738 | 1.316 | 31.325 | 31.23 | 0.99631 | 218013638 | C | A | 134 |
| rs13382307* | 0.0437984 | 0.253259 | 1.166 | 16.667 | 17.45 | 0.97684 | 218013951 | C | A | 766 |
| rs6760809** | 4.03E−06 | 0.557064 | 1.326 | 30.938 | NA | 0.98671 | 218013960 | T | C | 767 |
| rs73069129 | 7.10E−06 | 0.565596 | 1.316 | 31.063 | 28.08 | 0.98746 | 218014146 | C | A | 139 |
| rs12694417 | 6.00E−06 | 0.577954 | 1.316 | 31.329 | 31.23 | 0.99546 | 218014334 | T | C | 141 |
| rs12988242 | 6.25E−06 | 0.578789 | 1.316 | 31.329 | 31.23 | 0.99647 | 218014439 | A | G | 142 |
| rs10084346 | 0.0331583 | 0.271979 | 1.171 | 17.24 | 17.45 | 0.99689 | 218014981 | T | C | 144 |
| rs2045932 | 6.24E−06 | 0.578155 | 1.316 | 31.327 | 31.23 | 0.99652 | 218015468 | C | T | 145 |
| rs35855755 | 0.00188056 | 0.341745 | 1.237 | 21.542 | 18.11 | 0.99122 | 218015572 | G | T | 146 |
| rs2045933 | 6.25E−06 | 0.578581 | 1.316 | 31.327 | 31.23 | 0.99646 | 218015701 | A | T | 147 |
| rs1318847 | 6.28E−06 | 0.57833 | 1.315 | 31.328 | 31.23 | 0.99652 | 218015940 | T | C | 148 |
| rs974405 | 5.75E−06 | 0.578222 | 1.317 | 31.331 | 31.5 | 0.99618 | 218016155 | C | T | 150 |
| rs974406 | 6.28E−06 | 0.578155 | 1.315 | 31.327 | 31.23 | 0.99653 | 218016283 | C | G | 151 |
| rs6712801 | 8.73E−06 | 0.575908 | 1.31 | 31.214 | 31.23 | 0.99528 | 218016746 | A | G | 152 |
| rs4672831 | 6.30E−06 | 0.578155 | 1.315 | 31.328 | 31.23 | 0.99657 | 218017265 | A | G | 154 |
| rs4674177 | 6.32E−06 | 0.57833 | 1.315 | 31.328 | 31.23 | 0.99657 | 218017466 | G | C | 155 |
| rs4672832 | 6.32E−06 | 0.578595 | 1.315 | 31.328 | 31.23 | 0.99656 | 218017473 | A | C | 156 |
| rs4674178 | 4.12E−06 | 0.584192 | 1.322 | 31.501 | 31.23 | 0.99583 | 218017503 | C | T | 157 |
| rs10211305 | 6.28E−06 | 0.578642 | 1.316 | 31.314 | 31.23 | 0.99595 | 218017512 | A | T | 158 |
| rs4142171 | 6.39E−06 | 0.578789 | 1.315 | 31.329 | 31.23 | 0.99656 | 218017985 | G | T | 159 |
| rs1478595 | 6.40E−06 | 0.57845 | 1.315 | 31.328 | 31.23 | 0.99661 | 218018144 | G | T | 160 |
| rs1478596 | 6.39E−06 | 0.57833 | 1.315 | 31.328 | 31.23 | 0.99662 | 218018181 | C | G | 161 |
| rs966423 | 0.000140295 | 1 | 1.247 | 44.112 | 43.7 | 0.99685 | 218018585 | C | T | 162 |
| rs4674179 | 0.0348921 | 0.272098 | 1.17 | 17.224 | 17.32 | 0.99538 | 218018931 | A | C | 163 |
| rs34098645 | 0.000699586 | 0.205652 | 1.299 | 15.024 | 14.83 | 0.99158 | 218019645 | G | T | 742 |
| rs2618150 | 5.09E−06 | 0.585629 | 1.319 | 31.612 | 31.23 | 0.99428 | 218019691 | G | A | 164 |
| rs12992201 | 0.000651029 | 0.205636 | 1.301 | 14.99 | 14.83 | 0.98965 | 218020281 | G | A | 743 |
| rs17194199 | 0.000782855 | 0.204325 | 1.296 | 15.073 | 14.83 | 0.99078 | 218020621 | G | A | 744 |
| rs17806990 | 0.000753379 | 0.202629 | 1.297 | 15.036 | 14.83 | 0.99043 | 218020643 | C | T | 745 |
| rs71430278 | 0.000723509 | 0.202531 | 1.298 | 15.017 | 14.83 | 0.99024 | 218020693 | T | C | 746 |
| rs2618152 | 0.163876 | 0.527187 | 1.092 | 28.884 | 28.87 | 0.99224 | 218020843 | G | C | 165 |
| rs7562072 | 0.000561252 | 0.20522 | 1.306 | 14.917 | 14.83 | 0.99167 | 218020936 | T | G | 747 |
| rs7588296 | 0.000561125 | 0.20522 | 1.306 | 14.917 | 14.83 | 0.99167 | 218020943 | G | A | 748 |
| rs7561906 | 0.000460121 | 0.204264 | 1.311 | 14.869 | 14.83 | 0.98995 | 218020976 | T | C | 749 |
| rs7562091 | 0.000607194 | 0.205357 | 1.303 | 14.954 | 14.83 | 0.98976 | 218020993 | A | G | 750 |
| rs7588626 | 0.000535436 | 0.201966 | 1.307 | 15.061 | 14.83 | 0.98756 | 218021223 | G | A | 751 |
| rs7562410 | 0.000552468 | 0.20033 | 1.305 | 15.062 | 14.83 | 0.9915 | 218021293 | A | G | 752 |
| rs34943654 | 0.000604803 | 0.205289 | 1.304 | 14.942 | 14.83 | 0.99045 | 218021962 | C | T | 753 |
| rs35782231 | 0.00060494 | 0.205289 | 1.304 | 14.942 | 14.83 | 0.99046 | 218021994 | A | G | 754 |
| rs13418112 | 0.000326523 | 0.718093 | 1.238 | 36.411 | 33.99 | 0.9931 | 218022274 | A | G | 166 |
| rs35467789 | 0.000534176 | 0.202347 | 1.306 | 15.024 | 14.83 | 0.99093 | 218022292 | A | G | 167 |
| rs35309975 | 0.000581244 | 0.202854 | 1.304 | 15.05 | 14.83 | 0.98943 | 218022328 | G | A | 755 |
| rs13418037 | 0.187953 | 0.356771 | 1.096 | 21.358 | 19.16 | 0.99124 | 218022386 | T | C | 168 |
| rs76469716 | 0.000581244 | 0.205424 | 1.305 | 14.924 | 14.83 | 0.99091 | 218022446 | C | T | 756 |
| rs6730813 | 0.00068484 | 0.205091 | 1.299 | 15.04 | 14.83 | 0.99143 | 218022712 | C | T | 757 |
| rs2568173 | 0.0170201 | 0.483078 | 1.153 | 38.665 | 39.24 | 0.99244 | 218023698 | A | G | 170 |
| rs2373062 | 0.0165162 | 0.589469 | 1.154 | 47.993 | 12.2 | 0.95658 | 218030382 | G | C | 180 |
| rs2373063 | 0.135096 | 0.214045 | 1.092 | 52.201 | 56.96 | 0.98585 | 218030522 | T | C | 758 |
| rs2618139 | 0.0122257 | 0.485485 | 1.161 | 38.551 | 38.45 | 0.99616 | 218035046 | A | G | 186 |
| rs74485028 | 0.950848 | 0.203649 | 1.004 | 79.118 | N/A | 0.94877 | 218040448 | C | T | 194 |
| rs2568160 | 8.15E−05 | 0.444516 | 1.276 | 30.224 | 30.58 | 0.99475 | 218042708 | A | C | 196 |
| rs2568159 | 8.13E−05 | 0.444529 | 1.276 | 30.224 | 30.58 | 0.99477 | 218042748 | T | C | 197 |
| rs2568158 | 9.43E−05 | 0.443589 | 1.274 | 30.201 | 30.58 | 0.99293 | 218042779 | T | C | 198 |
| rs1478585 | 8.17E−05 | 0.444504 | 1.276 | 30.225 | 30.58 | 0.99479 | 218043029 | A | G | 200 |
| rs1478586 | 7.85E−05 | 0.443385 | 1.277 | 30.245 | 30.58 | 0.99462 | 218043137 | A | G | 201 |
| rs1478587 | 8.03E−05 | 0.444041 | 1.276 | 30.237 | 30.58 | 0.99442 | 218043227 | T | C | 202 |
| rs2568156 | 8.62E−05 | 0.442413 | 1.275 | 30.285 | 30.71 | 0.99513 | 218043685 | C | T | 203 |
| rs2568155 | 8.16E−05 | 0.444406 | 1.276 | 30.224 | 30.71 | 0.99486 | 218044150 | A | G | 205 |
| rs2568154 | 8.76E−05 | 0.443913 | 1.275 | 30.189 | 30.71 | 0.99549 | 218044299 | G | A | 206 |
| rs1382436 | 8.16E−05 | 0.444332 | 1.276 | 30.225 | 30.71 | 0.99473 | 218044568 | A | G | 207 |
| rs2618141 | 0.117175 | 0.211997 | 1.096 | 52.197 | 56.3 | 0.98696 | 218044863 | T | C | 759 |
| rs2618142 | 8.41E−05 | 0.434366 | 1.275 | 30.295 | 30.84 | 0.99361 | 218044915 | G | A | 208 |
| rs2618143 | 8.11E−05 | 0.444052 | 1.276 | 30.211 | 30.71 | 0.9941 | 218044931 | T | C | 209 |
| rs1382438 | 0.000108153 | 0.439328 | 1.271 | 30.262 | 30.71 | 0.99525 | 218047074 | C | A | 212 |
| rs1382440 | 9.92E−05 | 0.44248 | 1.273 | 30.183 | 30.71 | 0.99482 | 218047213 | A | G | 214 |
| rs2568153 | 8.29E−05 | 0.444325 | 1.276 | 30.234 | 30.58 | 0.99415 | 218047749 | A | C | 216 |
| rs1963252 | 0.0137776 | 0.483719 | 1.158 | 38.453 | NA | 0.99574 | 218050313 | A | G | 217 |
| rs768435 | 0.000150108 | 0.441109 | 1.265 | 30.142 | 30.58 | 0.9932 | 218052123 | T | C | 221 |
| rs1478592 | 8.27E−05 | 0.439627 | 1.276 | 30.183 | 30.84 | 0.99503 | 218054352 | C | T | 229 |
| rs2068972 | 9.53E−05 | 0.442383 | 1.273 | 30.173 | 30.71 | 0.99493 | 218054696 | A | G | 232 |
| rs35856653 | 0.000141838 | 0.441592 | 1.266 | 30.149 | 30.84 | 0.99448 | 218058451 | A | C | 238 |
| rs1072086 | 0.000105477 | 0.441642 | 1.271 | 30.163 | 30.97 | 0.99469 | 218060195 | T | A | 243 |
| rs2373066 | 0.000130963 | 0.43874 | 1.267 | 30.223 | 30.84 | 0.99632 | 218060619 | T | C | 244 |
| rs874839 | 0.000121236 | 0.440634 | 1.268 | 30.203 | 31.23 | 0.99668 | 218062332 | G | T | 247 |
| rs874840 | 0.000125805 | 0.440053 | 1.268 | 30.218 | 30.97 | 0.99641 | 218062375 | T | C | 248 |
| rs6754268 | 8.23E−05 | 0.434889 | 1.276 | 30.026 | 30.97 | 0.9917 | 218065042 | G | A | 254 |
| rs6754393 | 6.44E−05 | 0.434979 | 1.281 | 29.978 | 30.97 | 0.9928 | 218065181 | G | A | 255 |
| rs6754399 | 6.65E−05 | 0.435168 | 1.28 | 29.999 | 30.97 | 0.99214 | 218065197 | G | A | 256 |
| rs12475467 | 6.77E−05 | 0.438062 | 1.28 | 29.963 | 30.97 | 0.9943 | 218065627 | G | A | 257 |
| rs7597975 | 0.057912 | 0.309567 | 1.119 | 41.175 | 42.65 | 0.98683 | 218076464 | A | G | 760 |
| rs13392909 | 0.076875 | 0.343031 | 1.11 | 42.847 | 43.31 | 0.9857 | 218076978 | G | A | 761 |
| rs12328323 | 0.0907369 | 0.339588 | 1.105 | 42.585 | 43.31 | 0.98623 | 218077983 | G | A | 279 |
| rs2373076 | 0.0335881 | 0.27 | 1.133 | 43.758 | 45.93 | 0.98461 | 218089732 | G | A | 762 |
| rs13008340 | 0.0312871 | 0.225457 | 1.135 | 41.249 | 44.75 | 0.98665 | 218098259 | C | T | 308 |
| rs12694419 | 0.00277591 | 0.325221 | 1.195 | 37.482 | 42.26 | 0.98434 | 218099262 | C | G | 310 |
| rs750365 | 0.00168282 | 0.302489 | 1.207 | 36.105 | 41.47 | 0.98777 | 218099708 | A | C | 311 |
| rs6729351 | 0.0596301 | 0.223415 | 1.118 | 41.43 | 45.01 | 0.98006 | 218101545 | A | G | 313 |
| rs11889534 | 0.0394304 | 0.243779 | 1.129 | 42.386 | 45.41 | 0.98522 | 218102220 | C | T | 315 |
| rs7582879 | 0.0375831 | 0.236536 | 1.13 | 42.751 | 44.49 | 0.9818 | 218102574 | T | C | 763 |
| rs17202771 | 0.0487765 | 0.236497 | 1.123 | 42.557 | 43.44 | 0.97811 | 218102775 | C | T | 764 |
| rs62175530 | 0.0599191 | 0.20946 | 1.119 | 44.814 | N/A | 0.94634 | 218102822 | A | C | 765 |

rs13382307* also known as rs148235399
rs6760809** also known as rs67655058

1. A method of determining a susceptibility to Thyroid Cancer, the method comprising:
analyzing nucleic acid sequence data from a human individual for at least one polymorphic marker selected from the group consisting of rs966423 and rs7005606, and markers in linkage disequilibrium therewith;
wherein different alleles of the at least one polymorphic marker are associated with different susceptibilities to Thyroid Cancer in humans, and
determining a susceptibility to Thyroid Cancer from the nucleic acid sequence data.

2. The method of claim 1, wherein the nucleic acid sequence data is obtained from a biological sample containing nucleic acid from the human individual.

3. The method of claim 2, wherein the nucleic acid sequence data is obtained using a method that comprises at least one procedure selected from:
(i) amplification of nucleic acid from the biological sample;
(ii) hybridization assay using a nucleic acid probe and nucleic acid from the biological sample;
(iii) hybridization assay using a nucleic acid probe and nucleic acid obtained by amplification of the biological sample, and
(iv) high-throughput sequencing.

4. The method of claim 1, wherein the nucleic acid sequence data is obtained from a preexisting record.

5. The method of claim 4, wherein the preexisting record comprises a genotype dataset.

6. The method of any one of the preceding claims, wherein the analyzing comprises determining the presence or absence of at least one at-risk allele for Thyroid Cancer of the at least one polymorphic marker.

7. The method of any one of the preceding claims, wherein the determining comprises comparing the sequence data to a database containing correlation data between the at least one polymorphic marker and susceptibility to Thyroid Cancer.

8. The method of any one of the preceding claims, wherein markers in linkage disequilibrium with rs7005606 are selected from the group consisting of the markers listed in Table 1.

9. The method of any one of the claims 1 to 7, wherein markers in linkage disequilibrium with rs966423 are selected from the group consisting of the markers listed in Table 2.

10. The method of claim 6, wherein the at least one at-risk allele is selected from the group consisting of the G allele of rs7005606 and the C allele of rs966423.

11. The method of any one of the preceding claims, further comprising a step of preparing a report containing results from the determination, wherein said report is written in a computer readable medium, printed on paper, or displayed on a visual display.

12. The method of any one of the previous claims, further comprising reporting the susceptibility to at least one entity selected from the group consisting of the individual, a guardian of the individual, a genetic service provider, a physician, a medical organization, and a medical insurer.

13. A method of identification of a marker for use in assessing susceptibility to Thyroid Cancer in human individuals, the method comprising
a. identifying at least one polymorphic marker in linkage disequilibrium with rs7005606 or rs966423;
b. obtaining sequence information about the at least one polymorphic marker in a group of individuals diagnosed with Thyroid Cancer; and
c. obtaining sequence information about the at least one polymorphic marker in a group of control individuals;
wherein determination of a significant difference in frequency of at least one allele in the at least one polymorphism in individuals diagnosed with Thyroid Cancer as compared with the frequency of the at least one allele in the control group is indicative of the at least one polymorphism being useful for assessing susceptibility to Thyroid Cancer.

14. The method of claim 13, wherein an increase in frequency of the at least one allele in the at least one polymorphism in individuals diagnosed with Thyroid Cancer, as compared with the frequency of the at least one allele in the control group, is indicative of the at least one polymorphism being useful for assessing increased susceptibility to Thyroid Cancer; and wherein a decrease in frequency of the at least one allele in the at least one polymorphism in individuals diagnosed with Thyroid Cancer, as compared with the frequency of the at least one allele in the control group, is indicative of the at least one polymorphism being useful for assessing decreased susceptibility to, or protection against, Thyroid Cancer.

15. A method of predicting prognosis of an individual diagnosed with Thyroid Cancer, the method comprising
obtaining sequence data about a human individual about at least one polymorphic marker selected from the group consisting of rs7005606 and rs966423, and markers in linkage disequilibrium therewith, wherein different alleles of the at least one polymorphic marker are associated with different susceptibilities to Thyroid Cancer in humans, and
predicting prognosis of Thyroid Cancer from the sequence data.

16. A method of assessing probability of response of a human individual to a therapeutic agent for preventing, treating and/or ameliorating symptoms associated with Thyroid Cancer, comprising:
obtaining sequence data about a human individual identifying at least one allele of at least one polymorphic marker rs7005606 and rs966423, and markers in linkage disequilibrium therewith, wherein different alleles of the at least one polymorphic marker are associated with different probabilities of response to the therapeutic agent in humans, and
determining the probability of a positive response to the therapeutic agent from the sequence data.

17. A kit for assessing susceptibility to Thyroid Cancer in human individuals, the kit comprising:
reagents for selectively detecting at least one at-risk variant for Thyroid Cancer in the individual, wherein the at least one at-risk variant is selected from the group consisting of rs7005606 and rs966423, and markers in linkage disequilibrium therewith, and
a collection of data comprising correlation data between the at least one at-risk variant and susceptibility to Thyroid Cancer.

18. The kit of claim 17, wherein the collection of data is on a computer-readable medium.

19. The kit of claim 17 or claim 18, wherein the kit comprises reagents for detecting no more than 100 alleles in the genome of the individual.

20. The kit of claim 19, wherein the kit comprises reagents for detecting no more than 20 alleles in the genome of the individual.

21. Use of an oligonucleotide probe in the manufacture of a diagnostic reagent for diagnosing and/or assessing a susceptibility to Thyroid Cancer, wherein the probe is capable of hybridizing to a segment of any one sequence as set forth in SEQ ID NO:1-771, and wherein the segment is 15-400 nucleotides in length.

22. The use of claim 21, wherein the segment of the nucleic acid to which the probe is capable of hybridizing comprises a polymorphic site.

23. The use of claim 33, wherein the polymorphic site is selected from the group consisting of rs7005606 and rs966423, and markers in linkage disequilibrium therewith.

24. An assay for determining a susceptibility to thyroid cancer in a human subject, the assay comprising steps of:
(i) obtaining a nucleic acid sample from the human subject
(ii) assaying the nucleic acid sample to determine the presence or absence of at least one allele of at least one polymorphic marker conferring increased susceptibility to thyroid cancer in humans, and
(iii) determining a susceptibility to thyroid cancer for the human subject from the presence or absence of the at least one allele,
wherein the at least one polymorphic marker is selected from the group consisting of rs7005606 and rs966423, and markers correlated therewith,
wherein determination of the presence of the at least one allele is indicative of an increased susceptibility to thyroid cancer for the subject.

25. The assay of claim 24, wherein the at least one polymorphic marker correlated with rs7005606 is selected from the group consisting of the markers listed in table 2.

26. The assay of claim 24, wherein the at least one polymorphic marker correlated with rs966423 is selected from the group consisting of the markers listed in table 1.

27. The assay of claim 24, wherein the at least one allele is selected from the group consisting of the marker alleles listed in Table 7 and Table 8 having an odds ratio of greater than 1.

28. The assay of any one of the claims 24 to 27, wherein obtaining a nucleic acid sample comprises obtaining a biological sample comprising nucleic acid from the individual.

29. The assay of claim 28, further comprising isolating nucleic acid from the biological sample.

30. A system for identifying susceptibility to thyroid cancer in a human subject, the system comprising:
at least one processor;
at least one computer-readable medium;
a susceptibility database operatively coupled to a computer-readable medium of the system and containing population information correlating the presence or absence of at least one marker allele and susceptibility to thyroid cancer in a population of humans;
a measurement tool that receives an input about the human subject and generates information from the input about the presence or absence of the at least one allele in the human subject; and
an analysis tool that:
is operatively coupled to the susceptibility database and the measurement tool,
is stored on a computer-readable medium of the system,
is adapted to be executed on a processor of the system, to compare the information about the human subject with the population information in the susceptibility database and generate a conclusion with respect to susceptibility to thyroid cancer for the human subject;
wherein
the at least one marker allele is an allele of a marker selected from the group consisting of rs7005606 and rs966423, and markers correlated therewith.

31. The system according to claims 30, further including:
a communication tool operatively coupled to the analysis tool, stored on a computer-readable medium of the system and adapted to be executed on a processor of the system to communicate to the subject, or to a medical practitioner for the subject, the conclusion with respect to susceptibility to thyroid cancer for the subject.

32. The system of claim 30 or 31, wherein markers correlated with rs7005606 are selected from the group consisting of the markers listed in table 2.

33. The assay of claim 30 or claim 31, wherein markers correlated with rs7005606 are selected from the group consisting of the markers listed in table 1.

34. The assay of claim 30, wherein the at least one marker allele is selected from the group consisting of the marker alleles listed in Table 7 and Table 8 having an odds ratio of greater than 1.

35. The system according to any one of claims 30-34, wherein the measurement tool comprises a tool stored on a computer-readable medium of the system and adapted to be executed by a processor of the system to receive a data input about a subject and determine information about the presence or absence of the at least marker allele in a human subject from the data.

36. The system according to claim 35, wherein the data is genomic sequence information, and the measurement tool comprises a sequence analysis tool stored on a computer readable medium of the system and adapted to be executed by a processor of the system to determine the presence or absence of the at least one marker allele from the genomic sequence information.

37. The system according to claim 35 or claim 36, wherein the input about the human subject is a biological sample from the human subject, and wherein the measurement tool comprises a tool to identify the presence or absence of the at least one marker allele in the biological sample, thereby generating information about the presence or absence of the at least one marker allele in a human subject.

38. The system according to claim 37, wherein the measurement tool includes:
an oligonucleotide microarray containing a plurality of oligonucleotide probes attached to a solid support;
a detector for measuring interaction between nucleic acid obtained from or amplified from the biological sample and one or more oligonucleotides on the oligonucleotide microarray to generate detection data; and
an analysis tool stored on a computer-readable medium of the system and adapted to be executed on a processor of the system, to determine the presence or absence of the at least one marker allele based on the detection data.

39. The system according to claim 38, wherein the measurement tool includes:
a nucleotide sequencer capable of determining nucleotide sequence information from nucleic acid obtained from or amplified from the biological sample; and
an analysis tool stored on a computer-readable medium of the system and adapted to be executed on a processor of the system, to determine the presence or absence of the at least one marker allele based on the nucleotide sequence information.

40. The system according to any one of claims 30 to 39, further comprising:
a medical protocol database operatively connected to a computer-readable medium of the system and containing information correlating the presence or absence of the at least one marker allele and medical protocols for human subjects at risk for thyroid cancer; and
a medical protocol routine, operatively connected to the medical protocol database and the analysis routine, stored on a computer-readable medium of the system, and adapted to be executed on a processor of the system, to compare the conclusion from the analysis routine with respect to susceptibility to thyroid cancer for the subject and the medical protocol database, and generate a protocol report with respect to the probability that one or more medical protocols in the database will:
reduce susceptibility to thyroid cancer; or
delay onset of thyroid cancer; or
increase the likelihood of detecting thyroid cancer at an early stage to facilitate early treatment.

41. The system according to any one of claims 31-40, wherein the communication tool is operatively connected to the analysis routine and comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

42. The system according to claim 41, wherein the communication expresses the susceptibility to thyroid cancer in terms of odds ratio or relative risk or lifetime risk.

43. The system according to claim 41 or claim 42, wherein the communication further includes the protocol report.

44. The system according to any one of claims 30-43, wherein the susceptibility database further includes information about at least one parameter selected from the group consisting of age, sex, ethnicity, race, medical history, weight, diabetes status, blood pressure, family history of thyroid cancer, and smoking history in humans and impact of the at least one parameter on susceptibility to thyroid cancer.

45. A system for assessing or selecting a treatment protocol for a subject diagnosed with thyroid cancer, comprising:
at least one processor;
at least one computer-readable medium;
a medical treatment database operatively connected to a computer-readable medium of the system and containing information correlating the presence or absence of at least one allele of at least one marker selected from the group consisting of rs7005606 and rs966423, and markers correlated therewith, and efficacy of treatment regimens for thyroid cancer;
a measurement tool to receive an input about the human subject and generate information from the input about the presence or absence of the at least one marker allele in a human subject diagnosed with thyroid cancer; and
a medical protocol tool operatively coupled to the medical treatment database and the measurement tool, stored on a computer-readable medium of the system, and adapted to be executed on a processor of the system, to compare the information with respect to presence or absence of the at least one marker allele for the subject and the medical treatment database, and generate a conclusion with respect to at least one of:
the probability that one or more medical treatments will be efficacious for treatment of thyroid cancer for the patient; and
which of two or more medical treatments for thyroid cancer will be more efficacious for the patient.

46. The system according to claim 45, wherein the measurement tool comprises a tool stored on a computer-readable medium of the system and adapted to be executed by a processor of the system to receive a data input about a subject and determine information about the presence or absence of the at least one marker allele in a human subject from the data.

47. The system according to claim 46, wherein the data is genomic sequence information, and the measurement tool comprises a sequence analysis tool stored on a computer readable medium of the system and adapted to be executed by a processor of the system to determine the presence or absence of the at least one marker allele from the genomic sequence information.

48. The system according to claim 45, wherein the input about the human subject is a biological sample from the human subject, and wherein the measurement tool comprises a tool to identify the presence or absence of the at least one marker allele in the biological sample, thereby generating information about the presence or absence of the at least one marker allele in a human subject.

49. The system according to any one of claims 45-48, further comprising a communication tool operatively connected to the medical protocol routine for communicating the conclusion to the subject, or to a medical practitioner for the subject.

50. The system according to claim 49, wherein the communication tool comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

51. The system according to any of the claims 45 to 50, wherein markers correlated with rs7005606 are selected from the group consisting of the markers listed in table 2.

52. The assay according to any of the claims 45 to 50, wherein markers correlated with rs7005606 are selected from the group consisting of the markers listed in table 1.

53. The assay of according to any of the claims 45 to 50, wherein the at least one marker allele is selected from the group consisting of the marker alleles listed in Table 7 and Table 8 having an odds ratio of greater than 1.

54. The method, kit, use, assay, medium or apparatus according to any one of the preceding claims, wherein linkage disequilibrium between markers is characterized by particular numerical values of the linkage disequilibrium measures r² and/or |D′|.

55. The method, kit, use, assay, medium or apparatus according to any of the preceding claims, wherein linkage disequilibrium between markers is characterized by values of r² of at least 0.2.

56. The method, kit, use, assay, medium or apparatus according to any of the preceding claims, wherein linkage disequilibrium between markers is characterized by values of r² of at least 0.5.
